(19) United States
(12) Patent Application Publication
(10) Pub. No.: US 2006/0093617 A1
(43) Pub. Date: May 4, 2006

(54) Peptides for inducing a CTL and/or HTL response to hepatitis C virus

(75) Inventors: Marie-Ange Buyse, Merelbeke (BE); Geert Maertens, Brugge (BE); Erik Depla, Destelbergen (BE); Ignace Lasters, Antwerpen (BE); Johan Desmet, Kortrijk (BE); Denise Baker, Poway, CA (US); Robert W. Chesnut, Cardiff-by-the-Sea, CA (US); Mark Newman, Carlsbad, CA (US); Alessandro Sette, La Jolla, CA (US); John Sidney, San Diego, CA (US); Scott Southwood, Santee, CA (US)

Correspondence Address: NIXON & VANDERHYE, PC, 901 NORTH GLEBE ROAD, 11TH FLOOR, ARLINGTON, VA, 22203, US

(73) Assignees: Innogenetics, N.V., Ghent (BE); EPIMMUNE INC., San Diego, CA (US)

(21) Appl. No.: 11/140,487

(22) Filed: May 31, 2005

Related U.S. Application Data
(60) Provisional application No. 60/576,310, filed on Jun. 3, 2004.
(60) Provisional application No. 60/622,782, filed on Oct. 29, 2004.
(60) Provisional application No. 60/665,395, filed on Mar. 25, 2005.

(30) Foreign Application Priority Data
Jun. 1, 2004 (EP) 04012951.2
Oct. 28, 2004 (EP) 04447239.7
Mar. 25, 2005 (EP) 05102441.2

(51) Int. Cl. A61K 39/29; C07K 14/18

(52) U.S. Cl. 424/189.1; 530/350

(57) ABSTRACT

The present invention is directed to peptides, and nucleic acids encoding them, derived from the Hepatitis C Virus (HCV). The peptides are those which elicit a CTL and/or HTL response in a host. The invention is also directed to compositions and vaccines for prevention and treatment of HCV infection and diagnostic methods for detection of HCV exposure in patients.

### FIELD OF THE INVENTION

The present invention is directed to peptides or nucleic acids encoding them, derived from the Hepatitis C Virus (HCV). The peptides are those which elicit a cytotoxic and/or helper T lymphocyte response in a host. The invention is also directed to vaccines for prevention and treatment of HCV infection and diagnostic methods for detection of HCV exposure in patients.

### BACKGROUND OF THE INVENTION

The about 9.6 kb single-stranded RNA genome of the HCV virus comprises a 5′- and 3′-non-coding region (NCRs) and, in between these NCRs a single long open reading frame of about 9 kb encoding an HCV polyprotein of about 3000 amino acids.

HCV polypeptides are produced by translation from the open reading frame and cotranslational proteolytic processing. Structural proteins are derived from the amino-terminal one-fourth of the coding region and include the capsid or Core protein (about 21 kDa), the E1 envelope glycoprotein (about 35 kDa) and the E2 envelope glycoprotein (about 70 kDa, previously called NS1), and p7 (about 7 kDa). The E2 protein can occur with or without a C-terminal fusion of the p7 protein (Shimotohno et al. 1995). Recently, an alternative open reading frame in the Core-region was found which is encoding and expressing a protein of about 17 kDa called F (Frameshift) protein (Xu et al. 2001; Ou & Xu in US Patent Application Publication No. US2002/0076415). In the same region, ORFs for other 14-17 kDa ARFPs (Alternative Reading Frame Proteins), A1 to A4, were discovered and antibodies to at least A1, A2 and A3 were detected in sera of chronically infected patients (Walewski et al. 2001). From the remainder of the HCV coding region, the non-structural HCV proteins are derived which include NS2 (about 23 kDa), NS3 (about 70 kDa), NS4A (about 8 kDa), NS4B (about 27 kDa), NS5A (about 58 kDa) and NS5B (about 68 kDa) (Grakoui et al. 1993).

HCV is the major cause of non-A, non-B hepatitis worldwide. Acute infection with HCV (20% of all acute hepatitis infections) frequently leads to chronic hepatitis (70% of all chronic hepatitis cases) and end-stage cirrhosis. It is estimated that up to 20% of HCV chronic carriers may develop cirrhosis over a time period of about 20 years and that of those with cirrhosis between 1 to 4%/year is at risk to develop liver carcinoma (Lauer & Walker 2001, Shiffman 1999). An option to increase the life-span of HCV-caused end-stage liver disease is liver transplantation (30% of all liver transplantations world-wide are due to HCV-infection).

Virus-specific, human leukocyte antigen (HLA) class I-restricted cytotoxic T lymphocytes (CTL) are known to play a major role in the prevention and clearance of virus infections in vivo (Houssaint et al., 2001; Gruters et al., 2002; Tsai et al., 1997; Marray et al., 1992; Lukacher et al, 1984; Tigges et al., 1993).

MHC molecules are classified as either class I or class II. Class I MHC molecules are expressed on virtually all nucleated cells. Peptide fragments presented in the context of Class I MHC molecules are recognized by CD8+ T lymphocytes (cytotoxic T lymphocytes or CTLs). CD8+ T lymphocytes frequently mature into cytotoxic effectors which can lyse cells bearing the stimulating antigen. CTLs are particularly effective in eliminating tumor cells and in fighting viral infections.

Class II MHC molecules are expressed primarily on activated lymphocytes and antigen-presenting cells. CD4+ T lymphocytes (helper T lymphocytes or HTLs) are activated with recognition of a unique peptide fragment presented by a class II MHC molecule, usually found on an antigen presenting cell like a macrophage or dendritic cell. CD4+ T lymphocytes proliferate and secrete cytokines that either support an antibody-mediated response through the production of IL-4 and IL-10 or support a cell-mediated response through the production of IL-2 and IFN-gamma.

T lymphocytes recognize an antigen in the form of a peptide fragment bound to the MHC class I or class II molecule rather than the intact foreign antigen itself. An antigen presented by a MHC class I molecule is typically one that is endogenously synthesized by the cell (e.g., an intracellular pathogen). The resulting cytoplasmic antigens are degraded into small fragments in the cytoplasm, usually by the proteasome (Niedermann et al., 1995). Antigens presented by MHC class II molecules are usually soluble antigens that enter the antigen presenting cell via phagocytosis, pinocytosis, or receptor-mediated endocytosis. Once in the cell, the antigen is partially degraded by acid-dependent proteases in endosomes (Blum et al., 1997; Arndt et al., 1997).

Functional HLAs are characterized by a deep binding groove to which endogenous as well as foreign, potentially antigenic peptides bind. The groove is further characterized by a well-defined shape and physico-chemical properties. HLA class I binding sites are closed, in that the peptide termini are pinned down into the ends of the groove. They are also involved in a network of hydrogen bonds with conserved HLA residues (Madden et al., 1992). In view of these restraints, the length of bound peptides is limited to 8-10 residues. However, it has been demonstrated by Henderson et al (1992) that peptides of up to 12 amino acid residues are also capable of binding HLA class I. Superposition of the structures of different HLA complexes confirmed a general mode of binding wherein peptides adopt a relatively linear, extended conformation.

At the same time, a significant variability in the conformation of different peptides was observed also. This variability ranges from minor structural differences to notably different binding modes. Such variation is not unexpected in view of the fact that class I molecules can bind thousands of different peptides, varying in length (8-12 residues) and in amino acid sequence. The different class I allotypes bind peptides sharing one or two conserved amino acid residues at specific positions. These residues are referred to as anchor residues and are accommodated in complementary pockets (Falk, K. et al., 1991). Besides primary anchors, there are also secondary anchor residues occupied in more shallow pockets (Matsumura et al., 1992). In total, six allele-specific pockets termed A-F have been characterized (Saper et al., 1991; Latron et al., 1992). The constitution of these pockets varies in accordance with the polymorphism of class I molecules, giving rise to both a high degree of specificity (limited cross reactivity) while preserving a broad binding capacity.

In contrast to HLA class I binding sites, class II sites are open at both ends. This allows peptides to extend from the actual region of binding, thereby “hanging out” at both ends (Brown et al., 1993). Class II HLAs can therefore bind peptide ligands of variable length, ranging from 9 to more than 25 amino acid residues. Similar to HLA class I, the affinity of a class II ligand is determined by a “constant” and a “variable” component. The constant part again results from a network of hydrogen bonds formed between conserved residues in the HLA class II groove and the main-chain of a bound peptide. However, this hydrogen bond pattern is not confined to the N- and C-terminal residues of the peptide but distributed over the whole of the chain. The latter is important because it restricts the conformation of complexed peptides to a strictly linear mode of binding. This is common for all class II allotypes. The second component determining the binding affinity of a peptide is variable due to certain positions of polymorphism within class II binding sites. Different allotypes form different complementary pockets within the groove, thereby accounting for subtype-dependent selection of peptides, or specificity. Importantly, the constraints on the amino acid residues held within class II pockets are in general “softer” than for class I. There is much more cross reactivity of peptides among different HLA class II allotypes. Unlike for class I, it has been impossible to identify highly conserved residue patterns in peptide ligands (so-called motifs) that correlate with the class II allotypes.

Peptides that bind some MHC complexes have been identified by acid elusion methods (Buus et al., 1988), chromatography methods (Jardetzky, et al., 1991 and Falk et al., 1991), and by mass spectrometry methods (Hunt, et al., 1992). A review of naturally processed peptides that bind MHC class I molecules is set forth in Rotzschke and Falk, 1991.

Of the many thousand possible peptides that are encoded by a complex foreign pathogen, only a small fraction ends up in a peptide form capable of binding to MHC class I or class II antigens and can thus be recognized by T cells if containing a matching T-cell receptor. This phenomenon is known as immunodominance (Yewdell et al., 1997). More simply, immunodominance describes the phenomenon whereby immunization or exposure to a whole native antigen results in an immune response directed to one or a few “dominant” epitopes of the antigen rather than every epitope that the native antigen contains. Immunodominance is influenced by a variety of factors that include MHC-peptide affinity, antigen processing and T-cell receptor recognition.

In view of the heterogeneous immune response observed with HCV infection, induction of a multi-specific cellular immune response directed simultaneously against multiple HCV epitopes appears to be important for the development of an efficacious vaccine against HCV. There is a need, however, to establish vaccine embodiments that elicit immune responses that correspond to responses seen in patients that clear HCV infection.

The large degree of HLA polymorphism is an important factor to consider with the epitope-based approach to vaccine development. To address this factor, epitope selection can include identification of peptides capable of binding at high or intermediate affinity to multiple HLA molecules or selection of peptides binding the most prevalent HLA types. Another important factor to be considered in HCV vaccine development is the existence of different HCV genotypes and subtypes. Therefore, HCV genotype- or subtype-specific immunogenic epitopes need to be identified for all considered genotypes or subtypes. However, it is preferred to identify epitopes covering more than one HCV genotype or subtype.

The different characteristics of class I and class II MHC molecules are responsible for specific problems associated with the prediction of potential T-cell epitopes. As discussed before, class I molecules bind short peptides that exhibit well-defined residue type patterns. This has led to various prediction methods that are based on experimentally determined statistical preferences for particular residue types at specific positions in the peptide. Although these methods work relatively well, uncertainties associated with non-conserved positions limit their accuracy.

Methods for MHC/peptide binding prediction can grossly be subdivided into two categories: “statistical methods” that are driven by experimentally obtained affinity data and “structure-related methods” that are based on available 3D structural information of MHC molecules. Alternatively, a molecular dynamics simulation is sometimes performed to model a peptide within an MHC binding groove (Lim et al., 1996). Another approach is to combine loop modeling with simulated annealing (Rognan et al., 1999). Most research groups emphasize the importance of the scoring function used in the affinity prediction step. Several MHC binding HCV peptides have already been disclosed, e.g. in WO02/34770 (Imperial College Innovations Ltd), WO01/21189 and WO02/20035 (Epimmune), WO04/024182 (Intercell), WO95/25122 (The Scripps Research Institute), WO95/27733 (Government of the USA, Department of Health and Human Services), EP 0935662 (Chiron), WO02/26785 (Immusystems GmbH), WO95/12677 (Innogenetics N.V) and WO97/34621 (Cytel Corp).

There is a need to substantially improve both the structure prediction and the affinity assessment steps of methods which predict the affinity of a peptide for a major histocompatibility (MHC) class I or class II molecule. The main problem encountered in this field is the poor performance of prediction algorithms with respect to MHC alleles for which experimentally determined data (both binding and structural information) are scarce. This is e.g. the case for HLA-C.

Accordingly, while some MHC binding peptides have been identified, there is a need in the art to identify novel MHC binding peptides from HCV that can be utilized to generate an immune response against HCV from which they originate. Also, peptides predicted to bind (and binding) with reasonable affinity need a slow off rate in order to be immunogenic (Micheletti et al., 1999; Brooks et al., 1998; van der Burg et al., 1996).

### SUMMARY OF THE INVENTION

The present invention is directed to peptides or epitopes derived from the Core, E1, E2, P7, NS2, NS3, NS4 (NS4A and NS4B) and NS5 (NS5A and NS5B) protein of the Hepatitis C Virus (HCV). The peptides are those which elicit a HLA class I and/or class II restricted T lymphocyte response in an immunized host. More specific, the HLA class I restricted peptides of the present invention bind to at least one HLA molecule of the following HLA class I groups: HLA-A*01, HLA-A*02, HLA-A*03, HLA-A*11, HLA-A*24, HLA-B*07, HLA-B*08, HLA-B*35, HLA-B*40, HLA-B*44, HLA-Cw03, HLA-Cw04, HLA-Cw06 or HLA-Cw07. Preferred peptides are summarized in Table 13. The HLA class II restricted peptides of the present invention bind to at least one HLA molecule of the following HLA class II groups: HLA-DRB1, -DRB2, -DRB3, -DRB4, -DRB5, -DRB6, -DRB7, -DRB8 or -DRB9. Said HLA class II groups are sometimes summarized as HLA-DRB1-9. Preferred class II restricted peptides are given in Table 14.

The HLA class I and II binding peptides of the invention have been identified by the method as described in WO03/105058-Algonomics, by the method as described by Epimmune in WO01/21189 and/or by three public database prediction servers, respectively Syfpeithi, BIMAS and nHLAPred. Each of the peptides per se (as set out in the Tables) is part of the present invention. Furthermore, it is also an inventive aspect of this application that each peptide may be used in combination with the same peptide as multiple repeats, or with any other peptide(s) or epitope(s), with or without additional linkers. Accordingly, the present invention also relates to a composition and more specific to a polyepitopic peptide.

In a further embodiment, the present invention relates to a polyepitopic peptide comprising at least three peptides selected from the HLA-B and/or HLA-C binding peptides as disclosed in Table 13.

In a further embodiment, the present invention relates to a polyepitopic peptide comprising at least two peptides derived from a HCV protein and capable of inducing a HLA class I and/or class II restricted T lymphocyte response, wherein at least one peptide is a HLA-C binding peptide.

In a further embodiment, the present invention relates to a polyepitopic peptide comprising at least two HLA class II binding peptides selected from the peptides as disclosed in Table 14.

In a specific embodiment of the invention, the peptides are characterized in that they are present in the HCV consensus sequence of genotype 1a, 1b and/or 3a.

Furthermore, the present invention relates to nucleic acids encoding the peptides described herein. More particular, the present invention relates to a “minigene” or a polynucleotide that encodes a polyepitopic peptide as described herein.

The current invention also relates to a vector, plasmid, recombinant virus and host cell comprising the nucleic acid(s) or minigene(s) as described herein.

The peptides, corresponding nucleic acids and compositions of the present invention are useful for stimulating an immune response to HCV by stimulating the production of CTL and/or HTL responses. The peptide epitopes of the present invention, which are derived from native HCV amino acid sequences, have been selected so as to be able to bind to HLA molecules and induce or stimulate an immune response to HCV. In a specific embodiment, the present invention provides “nested epitopes”. The present invention also relates to a polyepitopic peptide comprising a nested epitope.

In a further embodiment, the present invention provides polyepitopic peptides, polynucleotides, compositions and combinations thereof that enable epitope-based vaccines from which the epitopes are capable of interacting directly or indirectly with HLA molecules encoded by various genetic alleles to provide broader population coverage than prior vaccines.

In a preferred embodiment, the invention relates to a composition comprising HCV-specific CTL epitopes, HCV-specific HTL epitopes or a combination thereof. Said composition can be in the form of a minigene comprising one or more CTL epitopes, one or more HTL epitopes, or a combination thereof.

In a further embodiment, the peptides of the invention, or nucleic acids encoding them, are used in diagnostic methods such as the determination of a treatment regimen, the determination of the outcome of an HCV infection, evaluation of an immune response or evaluation of the efficacy of a vaccine.

### FIGURE LEGENDS

FIG. 1: HCV 1b consensus sequence (SEQ ID NO 769), based on a selection of available HCV sequences with identification (in bold) of the parts used for the 9-mer peptide design by the method as described by Algonomics N.V.; said parts are Core, NS3 and NS5; the amino acid numbering of the 9-mers present in Tables 1-11 is based on the HCV sequence disclosed in FIG. 1.

| | | | part of | | | |
|---|---|---|---|---|---|---|
| | AA start | AA end | interest | AA start | AA end | #AA |
| C | 1 | 191 | C | 1 | 191 | 191 |
| E1 | 192 | 383 | | | | |
| E2 | 384 | 746 | | | | |
| P7 | 747 | 809 | | | | |
| NS2 | 810 | 1026 | | | | |
| NS3 | 1027 | 1657 | NS3 | 1160 | 1657 | 498 |
| NS4A | 1658 | 1711 | | | | |
| NS4B | 1712 | 1972 | | | | |
| NS5A | 1973 | 2420 | | | | |
| NS5B | 2421 | 3011 | NS5B | 2560 | 2850 | 291 |

FIG. 2: HCV 1b consensus sequence (SEQ ID NO 770) with identification (in bold) of the parts used for the 10-mer peptide design by the method as described by Algonomics N.V., and used for determination of HCV genotype cross-reactivity; said parts are Core, NS3, NS4 and NS5. The amino acid numbering is the same as for FIG. 1. The amino acid numbering of the 10-mers present in Tables 1-11 is based on the HCV sequence disclosed in FIG. 2.

FIG. 3: Binding of HLA-A02 reference peptide FLPSDC(F1)FPSV on HLA-A02 in a cell-based binding assay.

FIG. 4: Example of a typical HLA-A02 competition experiment in a cell-based binding assay.

FIG. 5: HCV 1a consensus sequence (SEQ ID NO 771) used for determination of HCV genotype cross-reactivity.

FIG. 6: HCV 3a consensus sequence (SEQ ID NO 772) used for determination of HCV genotype cross-reactivity.

FIG. 7: Binding versus immunogenicity in HLA-DRB1*0401 Tg mice.

### DETAILED DESCRIPTION OF THE INVENTION

The present invention is directed to peptides derived from the Core, E1, E2, P7, NS2, NS3, NS4 (NS4A and NS4B) or NS5 (NS5A and NS5B) protein of the Hepatitis C Virus (HCV). The peptides are those which elicit a HLA class I and/or class II restricted T lymphocyte response in an immunized host.

More specific, the HLA class I restricted peptides (CTL epitopes) of the present invention bind at least one HLA molecule of the following HLA class I groups: HLA-A*01, HLA-A*02, HLA-A*03, HLA-A*11, HLA-A*24, HLA-B*07, HLA-B*08, HLA-B*35, HLA-B*40, HLA-B*44, HLA-Cw03, HLA-Cw04, HLA-Cw06 or HLA-Cw07. Preferred peptides are summarized in Table 13. The HLA class II restricted peptides (HTL epitopes) of the present invention bind at least one HLA molecule of the following HLA class II groups: HLA-DRB1, -DRB2, -DRB3, -DRB4, -DRB5, -DRB6, -DRB7, -DRB8 or -DRB9. Said HLA class II groups are sometimes summarized as HLA-DRB1-9. Preferred HTL epitopes are given in Table 14.

Each of the HLA class I and class II peptides per se (as set out in the Tables) is part of the present invention. Furthermore, it is an aspect of the invention that each epitope may be used in combination with any other epitope.

Identification of the Peptides

Based on the hundreds of known HCV genotypes and subtypes (at least 3000 amino acids per sequence), thousands of theoretical CTL and/or HTL epitopes are predicted according to the methods as described herein. Starting from said long list, a first selection of epitopes has been made based on the predicted binding affinity.

The HLA class I and II binding peptides of the invention have been identified by the method as described in WO03/105058-Algonomics, by the method as described by Epimmune in WO01/21189 and/or by three public epitope prediction servers respectively Syfpeithi, BIMAS and nHLAPred.

A first set of CTL peptides is derived by the method as described in WO03/105058 by Algonomics N.V., Zwijnaarde, Belgium, which is incorporated herein by reference. Said method is directed to a structure-based prediction of the affinity of potentially antigenic peptides for major histocompatibility (MHC) receptors.

Initially, a HCV consensus sequence is designed. To do this, a selection of HCV sequences from HCV type 1b present in the “Los Alamos” database are clustered and aligned. The HCV Sequence Database from the Los Alamos National laboratory can be found on: http://hcv.lanl.gov/content/hcv-db/HelpDocs/cluster-help.html.

The generated multiple sequence alignments have been used to identify interesting (i.e. conserved) regions in the HCV proteins for CTL epitope prediction.

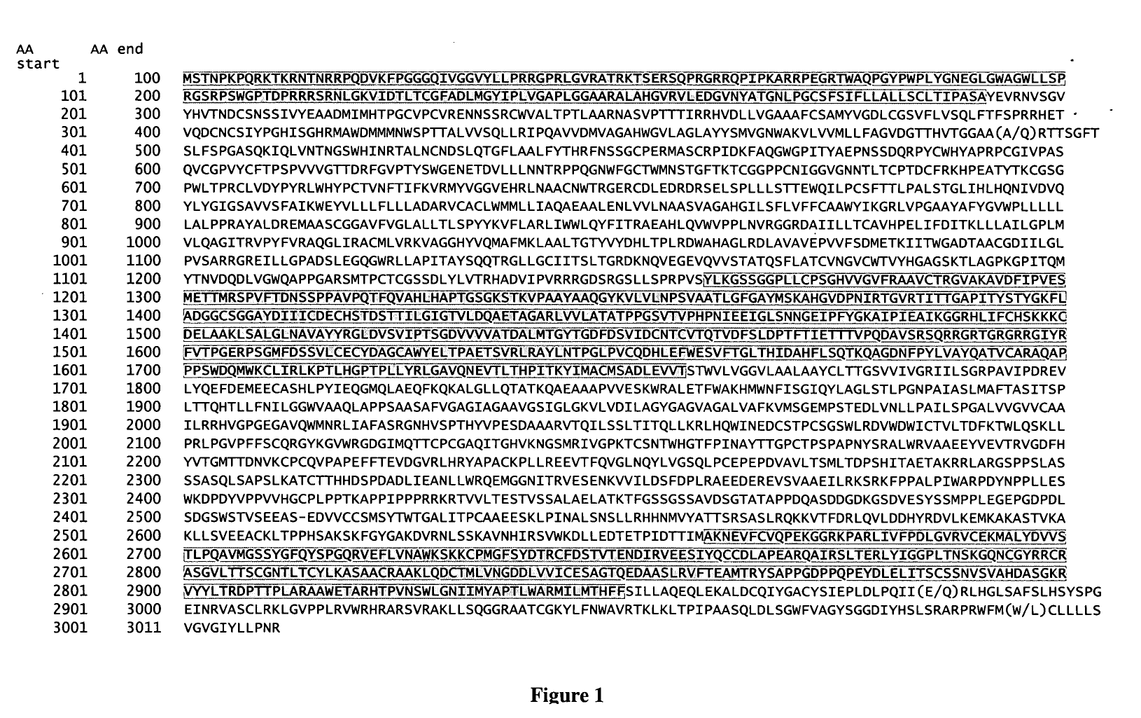

FIG. 1 discloses the HCV consensus sequence used for the 9-mer CTL epitope prediction in the present invention. Amino acid numbering for the 9-mers present in Tables 1-11 is based on said sequence.

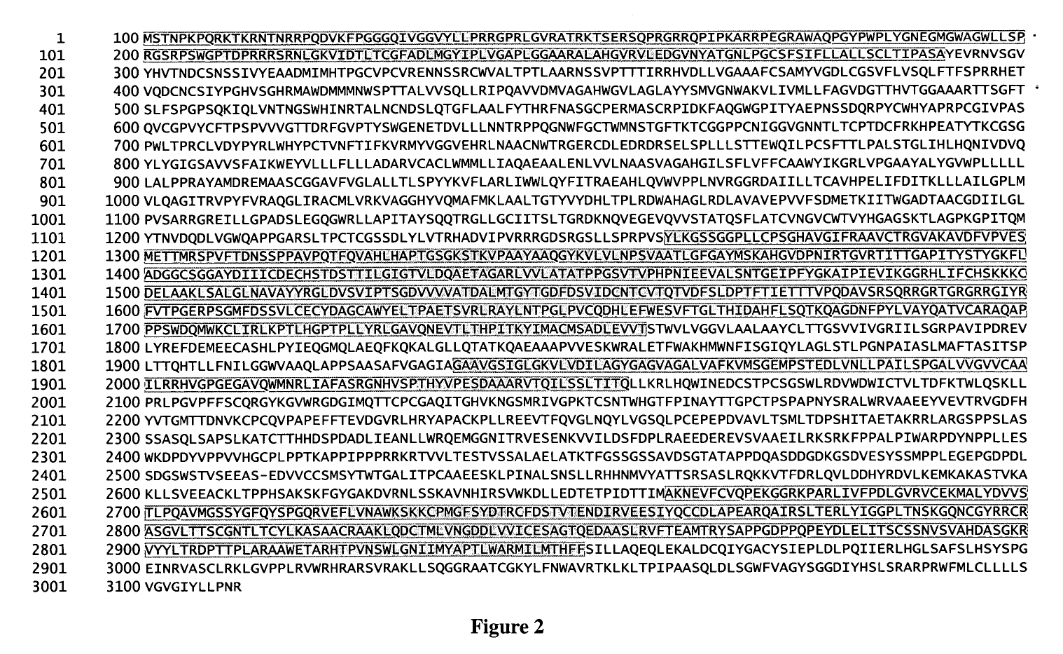

FIG. 2 discloses the HCV consensus sequence used for the 10-mer CTL epitope prediction in the present invention. Amino acid numbering for the 10-mers present in Tables 1-11 is based on said sequence.

Predictions were made for HLA-A0101, HLA-A0201, HLA-A0301, HLA-A2402, HLA-B0702, HLA-B0801, HLA-B3501, HLA-B4403, HLA-Cw0401, HLA-Cw0602 and HLA-Cw0702.

Tables 1-11 disclose the HLA-A, HLA-B and HLA-C binding peptides of the current invention derived by the above-described algorithm. Division is made between Strong binders (S) with Kdpred <0.1 μM, Medium binders (M) with Kdpred 0.1-1 μM and Weak binders (W) with Kdpred 1-10 μM. Kdpred is the affinity (dissociation constant) as predicted by the algorithm.

A further selection is made based upon the presence of the epitopes in the most prevalent genotypes. Accordingly, those peptides that are present in
at least genotype 3a, or
at least genotype 1b, or
at least genotype 1a and 1b, or
at least genotypes 1a, 1b and 3a,
   are retained for further testing. These peptides are summarized in Table 13.

Furthermore, other HCV genotypes (e.g. genotype 4a) can be retained in view of prevalence and/or importance.

A second set of peptides is identified by the method as described in WO01/21189 by Epimmune Inc., California, USA, which is incorporated herein by reference. Proprietary computer algorithms are used to rapidly identify potential epitopes from genomic or proteomic sequence data of viruses, bacteria, parasites or tumor-associated antigens. The program can also be used to modify epitopes (analogs) in order to enhance or suppress an immune response.

The algorithm is based on the conversion of coefficient-based scores into KD (IC50) predictions (PIC Score) thereby facilitating combined searches involving different peptide sizes or alleles. The combined use of scaling factors and exponential power corrections resulted in best goodness of fit between calculated and actual IC50 values. Because the algorithm predicts epitope binding with any given affinity, a more stringent candidate selection procedure of selecting only top-scoring epitopes, regardless of HLA-type, can be utilized.

Protein sequence data from 57 HCV isolates were evaluated for the presence of the designated supermotif or motif. The 57 strains include COLONEL-ACC-AF290978, H77-ACC-NC, HEC278830-ACC-AJ278830, LTD 1-2-XF222-ACC-AF511948, LTD6-2-XF224-ACC-AF511950, JP.HC-J1-ACC-D10749, US.HCV-H-ACC-M67463, US.HCV-PT-ACC-M62321, D89815-ACC-D89815, HC-J4-ACC-AF054250, HCR6-ACC-AY045702, HCV-CG1B-ACC-AF333324, HCV-JS-ACC-D85516, HCV-K1-R1-ACC-D50480, HCV-S 1-ACC-AF356827, HCVT050-ACC-AB049087, HPCHCPO-ACC-D45172, M1LE-ACC-AB080299, MD11-ACC-AF207752, Source-ACC-AF313916, TMORF-ACC-D89872, AU.HCV-A-ACC-AJ000009, CN.HC-C2-ACC-D10934, CN.HEBEI-ACC-L02836, DE.HCV-AD78-ACC-AJ132996, DE.HD-1-ACC-U45476, DE.NC1-ACC-AJ238800, JP.HCV-BK-ACC-M58335, JP.HCV-J-ACC-D90208, JP.HCV-N-ACC-AF139594, JP.J33-ACC-D14484, JP.JK1-full-ACC-X61596, JP.JT-ACC-D11355, JP.MD1-1-ACC-AF165045, KR.HCU16362-ACC-U16362, KR.HCV-L2-ACC-U01214, RU.274933RU-ACC-AF176573, TR.HCV-TR1-ACC-AF483269, TW.HCU89019-ACC-U89019, TW.HPCGENANTI-ACC-M84754, G2AK1-ACC-AF 169003, HC-J6CH-ACC-AF 177036, MD2A-1-ACC-AF238481, NDM228-ACC-AF169002, JP.JCH-1-ACC-AB047640, JP.JFH-1-ACC-AB047639, JP.Td-6-ACC-D00944, JPUT971017-ACC-AB030907, MD2B-1-ACC-AF238486, JP.HC-J8-ACC-D10988, BEBE1-ACC-D50409, CB-ACC-AF046866, K3A-ACC-D28917, NZL1-ACC-D17763, DE.HCVCENS 1-ACC-X76918, JP.HCV-Tr-ACC-D49374 and EG.ED43-ACC-Y11604.

Predictions were made for HLA-A0101, HLA-A0201, HLA-A1101, HLA-A2402, HLA-B0702, HLA-B-0801 and HLA-B4002. For B0801, no PIC algorithm is available but motif-positive sequences were selected.

Tables 1, 2, 3, 4, 5, 6 and 8 disclose the HLA-A and HLA-B peptides of the current invention yielding PIC Scores <100 derived by the above-described algorithm.

A further selection is made based upon the presence of the epitopes in the most prevalent genotypes. Accordingly, those peptides that are present in at least genotype 3a, or
at least genotype 1b, or
at least genotype 1a, 1b, or
at least genotypes 1a, 1b and 3a,
   are retained for further testing. These peptides are summarized in table 13. Furthermore, other HCV genotypes (e.g. genotype 4a) can be retained in view of prevalence and/or importance.

A third set of peptides is identified by three publicly available algorithms.

Initially, a HCV 1b consensus sequence is designed. HCV sequences from 80 HCV type 1b sequences were retrieved from the HCV sequence database http://hcv.lanl.gov/content/hcv-db/index of the Division of Microbiology and Infectious Diseases of the National Institute of Allergies and Infectious Diseases (NIAID).

The generated multiple sequence alignments are used to identify interesting regions in the HCV proteins for CTL epitope prediction. FIG. 2 discloses the HCV consensus sequence used for the CTL epitope prediction. Amino acid numbering throughout the specification is based on said sequence.

Based on said consensus sequence, three different prediction algorithms were used for CTL epitope prediction:

A) Syfpeithi:

Hans-Georg Rammensee, Jutta Bachmann, Niels Nikolaus Emmerich, Oskar Alexander Bachor, Stefan Stevanovic: SYFPEITHI: database for MHC ligands and peptide motifs. Immunogenetics (1999) 50: 213-219; www.syfpeithi.de)

The prediction is based on published motifs (pool sequencing, natural ligands) and takes into consideration the amino acids in the anchor and auxiliary anchor positions, as well as other frequent amino acids. The scoring system evaluates every amino acid within a given peptide. Individual amino acids may be given the arbitrary value 1 for amino acids that are only slightly preferred in the respective position, optimal anchor residues are given the value 15; any value between these two is possible. Negative values are also possible for amino acids which are disadvantageous for the peptide's binding capacity at a certain sequence position. The allocation of values is based on the frequency of the respective amino acid in natural ligands, T-cell epitopes, or binding peptides. The maximal scores vary between different MHC alleles. Only those MHC class I alleles for which a large amount of data is available are included in the “epitope prediction” section of SYFPEITHI. SYFPEITHI does not make predictions for HLA-C alleles.

Predictions were made for HLA-A01, A0201, A03, A2402, B0702, B08 and B44. For each class, both 9- and 10-mers were predicted, except for B08, where 8- and 9-mers were predicted, but no 10-mers.

B) BIMAS:

This algorithm allows users to locate and rank 8-mer, 9-mer, or 10-mer peptides that contain peptide-binding motifs for HLA class I molecules. Said rankings employ amino acid/position coefficient tables deduced from the literature by Dr. Kenneth Parker of the National Institute of Allergy and Infectious Diseases (NIAID) at the National Institutes of Health (NIH) in Bethesda, Md. The Web site (http://bimas.dcrt.nih.gov/molbio/hla_bind/) was created by Ronald Taylor of the Bioinformatics and Molecular Analysis Section (BIMAS), Computational Bioscience and Engineering Laboratory (CBEL), Division of Computer Research & Technology (CIT), National Institutes of Health, in collaboration with Dr. Parker. The initial (running) score is set to 1.0. For each residue position, the program examines which amino acid is appearing at that position. The running score is then multiplied by the coefficient for that amino acid type, at that position, for the chosen HLA molecule. These coefficients have been pre-calculated and are stored for use by the scoring algorithm in a separate directory as a collection of HLA coefficient files. The idea behind these tables is the assumption that, to the first approximation, each amino acid in the peptide contributes independently to binding to the class I molecule. Dominant anchor residues, which are critical for binding, have coefficients in the tables that are significantly different from 1. Highly favorable amino acids have coefficients substantially greater than 1, and unfavorable amino acids have positive coefficients that are less than one. Auxiliary anchor residues have coefficients that are different from 1 but smaller in magnitude than dominant anchor residues. Using 9-mers, nine multiplications are performed. Using 10-mers, nine multiplications are again performed, because the residue lying at the fifth position in the sequence is skipped. The resulting running score is multiplied by a final constant to yield an estimate of the half time of disassociation. The final multiplication yields the score reported in an output table. Predictions were made for HLA-A01, A0201, A03, A24, B07, B08, B3501, B4403, Cw0301, Cw0401, Cw0602 and Cw0702. For each class, both 9- and 10-mers were predicted, except for B08, where 8-, 9- and 10-mers were predicted.

C) nHLAPred

nHLAPred is a highly accurate MHC binders' prediction method for the large number of class I MHC alleles. (Dr. GPS Raghava, Coordinator, Bioinformatics Centre, Institute of Microbial Technology, Sector 39A, Chandigarh, India; http://imtech.rs.in/raghava). The algorithm is partitioned in two parts ComPred and ANNpred. In the ComPred part the prediction is based on the hybrid approach of Quantitative matrices and artificial neural network. In ANNPred the prediction is solely based on artificial neural network.

ComPred: This part of the algorithm can predict the MHC binding peptides for 67 MHC alleles. The method is systematically developed as follows:

Firstly, a quantitative matrix (QM) based method has been developed for 47 MHC class I alleles having minimum 15 binders available in the MHCBN database.

Quantitative matrices provide a linear model with easy to implement capabilities. Another advantage of using the matrix approach is that it covers a wider range of peptides with binding potential and it gives a quantitative score to each peptide.

Further, an artificial neural network (ANN) based method has been developed for 30 out of these 47 MHC alleles having 40 or more binders. The ANNs are self-training systems that are able to extract and retain the patterns present in submitted data and subsequently recognize them in previously unseen input. The ANNs are able to classify the data of MHC binders and non-binders accurately as compared to other. The ANNs are able to generalize the data very well. The major constraint of neural based prediction is that it requires large data for training. In addition, the method allows prediction of binders for 20 more MHC alleles using the quantitative matrices reported in the literature.

Predictions were made for HLA-A01, A0201, A0301, A24, B0702, B08, B3501, B4403, Cw0301, Cw0401, Cw0602 and Cw0702. nHLAPred can only predict 9-mers.

For each combination of prediction algorithm, protein and HLA allele, a list of the top ranking peptides (=predicted to have the highest affinity) is retrieved.

A list was created (not shown) with all peptides for all HLA alleles in descending order of affinity. In this list, the peptides were marked according to occurrence in different HCV genotypes (1b, 1a and/or 3 a consensus sequences) and to cross-reaction between HLA alleles. For each HLA class, all peptides predicted by the different prediction servers are combined in 1 table (not shown) with the ranknumbers for each of the predictionservers per column. For each peptide the number of predictionservers that assigned a ranknumber up to 60 or 100 are counted.

Those peptides that are predicted by 2 to 4 algorithms and that are within the 60 or 100 best are finally selected. If upon binding analysis (see below) only few high affinity binding peptides are identified, additional selections can be made (e.g. from peptides predicted by the Epimmune algorithm and yielding PIC scores <1000). All these peptides are given in Table 13.

As an example, the selection of the B07 peptides has been disclosed in Example 2. A comparable procedure was followed for the other HLA-binding peptides predicted by the Epimmune algorithm and the three public algorithms.

Table 13 discloses the selection of the HLA-A, HLA-B and HLA-C peptides of the current invention that are predicted to bind to a given HLA and that are derived by the above-described procedures. The peptide and corresponding nucleic acid compositions of the present invention are useful for inducing or stimulating an immune response to HCV by stimulating the production of CTL responses.

The HLA class II binding peptides of the present invention have been identified by the method as described in WO 01/21189A1 by Epimmune Inc., California, USA, which is incorporated herein by reference. Protein sequence data from 57 HCV isolates (as for the CTL prediction) were evaluated for the presence of the designated supermotif or motif. Predictions were made using the HLA DR-1-4-7 supermotif for peptides that bind to HLA-DRB1*0401, DRB1*0101 and DRB1*0701, and using HLA DR3 motifs for peptides that bind to DRB1*0301.

The predicted HTL peptides are given in Table 12.

A further selection is made based upon the presence of the core of the class II epitopes in the most prevalent genotypes. The “core” is defined as the central 9 (uneven amount of total amino acids) or 10 (even amount of total amino acids) amino acids of the total epitope sequence. As an example, the core (9aa) of the following epitope (15α-uneven) is indicated in bold/underlined: ADLMGYIPLVGAPLG.

Accordingly, those peptides that have a core present in
at least genotype 3a, or
at least genotype 1b, or
at least genotype 1a, 1b, or
at least genotypes 1a, 1b and 3a,
   are retained for further testing. These peptides are summarized in table 14.

Furthermore, other HCV genotypes (e.g. genotype 4a) can be retained in view of prevalence and/or importance.

The relationship between binding affinity for HLA class I and II molecules and immunogenicity of discrete peptides or epitopes on bound antigens (HLA molecules) can be analyzed in two different experimental approaches (see, e.g., Sette et al, 1994). E.g. as for HLA-A0201, in the first approach, the immunogenicity of potential epitopes ranging in HLA binding affinity over a 10.000-fold range can be analyzed in HLA-A0201 transgenic mice. In the second approach, the antigenicity of approximately 100 different hepatitis B virus (HBV)-derived potential epitopes, all carrying A0201 binding motifs, was assessed by using PBL from acute hepatitis patients. Pursuant to these approaches, it was determined that an affinity threshold value of approximately 500 nM (preferably 50 nM or less) determines the capacity of a peptide epitope to elicit a CTL response. Said values are not yet available for other HLA Class I alleles.

These data are true for class I binding affinity measurements for naturally processed peptides and for synthesized T cell epitopes.

An affinity threshold associated with immunogenicity in the context of HLA class II DR molecules has also been delineated (see, e.g., Southwood et al., 1998). In order to define a biologically significant threshold of DR binding affinity, a database of the binding affinities of 32 DR-restricted epitopes for their restricting element (i.e., the HLA molecule that binds the motif) was compiled. In this case, 1000 nM can be defined as an affinity threshold associated with immunogenicity in the context of DR molecules.

The predicted binding affinity (Score) of the peptides of the current invention are indicated in Tables 1-11. The experimentally determined binding affinity or inhibition constant (Ki) of peptides for HLA molecules can be determined as described in Example 3. The inhibition constant (Ki) is the affinity of the peptide as determined in a competition experiment with labeled reference peptide. The Ki is calculated from the experimentally determined IC50 value according to the formula:

$${K}_{i} = \frac{IC50}{1 + [ F1 \text{-} pep ] / Kd}$$

The binding affinities (K1 or IC50) of the peptides of the present invention to the respective HLA class I and II alleles are indicated in Tables 13 and 14.

“IC50” is the concentration of peptide in a binding assay at which 50% inhibition of binding of a reference peptide is observed. Throughout the specification, “binding data” results are often expressed in terms of IC50. Given the conditions in which the assays are run (i.e. limiting HLA proteins and labeled peptide concentrations), these values approximate Ki values. It should also be noted that the calculated Ki values are indicative values and are no absolute values as such, as these values depend on the quality/purity of the peptide/MHC preparations used and the type of non-linear regression used to analyze the binding data.

Binding may be determined using assay systems including those using: live cells (e.g., Ceppellini et al., 1989; Christnick et al., 1991; Busch et al., 1990; Hill et al., 1991; del Guercio et al., 1995), cell free systems using detergent lysates (e.g., Cerundolo et al., 1991), immobilized purified MHC (e.g., Hill et al., 1994; Marshall et al., 1994), ELISA systems (e.g., Reay et al., 1992), surface plasmon resonance (e.g. Khilko et al., 1993); high flux soluble phase assays (Hammer et al., 1994), and measurement of class I MHC stabilization or assembly (e.g., Ljunggren et al., 1990; Schumacher et al., 1990; Townsend et al., 1990; Parker et al., 1992). The binding assays used in the present invention are demonstrated in Examples 3 and 4. The results as shown in Table 13 and 14 are either results of individual experiments or are the mean of a number of experiments.

As used herein, “high affinity” or “strong binder” with respect to HLA class I and II molecules is defined as binding with a K1 or IC50 value of 100 nM or less; “intermediate affinity” or “mediate binder” is binding with a K1 or IC50 value of between about 100 and about 1000 nM.

As used herein, “threshold affinity” is the minimal affinity a peptide needs to display for a given HLA type that assures immunogenicity with high certainty in humans and/or animals. The threshold affinity can—but must not—be different for different HLA types.

Based on the data derived from the binding experiments, a further selection of candidate epitopes is made. Higher HLA binding affinity is typically correlated with higher immunogenicity. Immunogenicity can be manifested in several different ways. Immunogenicity corresponds to whether an immune response is elicited at all, and to the vigor of any particular response, as well as to the extent of a population in which a response is elicited. For example, a peptide might elicit an immune response in a diverse array of the population, yet in no instance produce a vigorous response. In accordance with these principles, close to 90% of high affinity binding peptides have been found to be immunogenic, as contrasted with about 50% of the peptides that bind with intermediate affinity (Sette et al., 1994; Alexander et al., 2003). Moreover, higher binding affinity peptides lead to more vigorous immunogenic responses. As a result, less peptide is required to elicit a similar biological effect if a high affinity binding peptide is used. Thus, in preferred embodiments of the invention, high affinity binding peptides (strong binders) and medium affinity peptides (medium binders) are particularly useful.

Various strategies can be utilized to evaluate immunogenicity, including:

1) Evaluation of primary T cell cultures from normal individuals (see, e.g., Wentworth et al., 1995; Celis et al., 1994; Tsai et al., 1997; Kawashima et al., 1998). This procedure involves the stimulation of peripheral blood lymphocytes (PBL) from normal subjects with a test peptide in the presence of antigen presenting cells in vitro over a period of several weeks. T cells specific for the peptide become activated during this time and are detected using, e.g., a ⁵¹Cr-release assay involving peptide sensitized target cells.

2) Immunization of HLA transgenic mice (see, e.g., Wentworth et al., 1996; Wentworth et al., 1996; Alexander et al., 1997) or surrogate mice. In this method, peptides (e.g. formulated in incomplete Freund's adjuvant) are administered subcutaneously to HLA transgenic mice or surrogate mice. Several weeks following immunization, splenocytes are removed and cultured in vitro in the presence of test peptide for approximately one week. Peptide-specific T cells are detected using, e.g., a ⁵¹Cr-release assay involving peptide sensitized target cells and target cells expressing endogenously generated antigen.

3) Demonstration of recall T cell responses from immune individuals who have effectively been vaccinated, recovered from infection, and/or from chronically infected patients (see, e.g., Rehermann et al., 1995; Doolan et al., 1997; Bertoni et al., 1997; Threlkeld et al., 1997; Diepolder et al., 1997). In applying this strategy, recall responses are detected by culturing PBL from subjects that have been naturally exposed to the antigen, for instance through infection, and thus have generated an immune response “naturally”, or from patients who were vaccinated with a vaccine comprising the peptide of interest. PBL from subjects are cultured in vitro for 1-2 weeks in the presence of test peptide plus antigen presenting cells (APC) to allow activation of “memory” T cells, as compared to “naive” T cells. At the end of the culture period, T cell activity is detected using assays for T cell activity including ⁵¹Cr release involving peptide-sensitized targets, T cell proliferation, or lymphokine release.

A given epitope is stated to be immunogenic if T cell reactivity can be shown to targets sensitized with that peptide. Immunogenicity for a given epitope can further be described by the number of individuals in a group of HLA matched infected or vaccinated subjects (e.g. human, transgenic mice, surrogate mice) that show T cell reactivity to that particular epitope, or e.g. by the number of spots detected in an ELISPOT assay, as described in examples 5-8. Based on the data derived from one of these experiments, a further selection of candidate epitopes is made according to their immunogenicity. Immunogenicity for the peptides of the invention is indicated in Tables 13 and 14. A “+” indicates T cell reactivity in at least one subject.

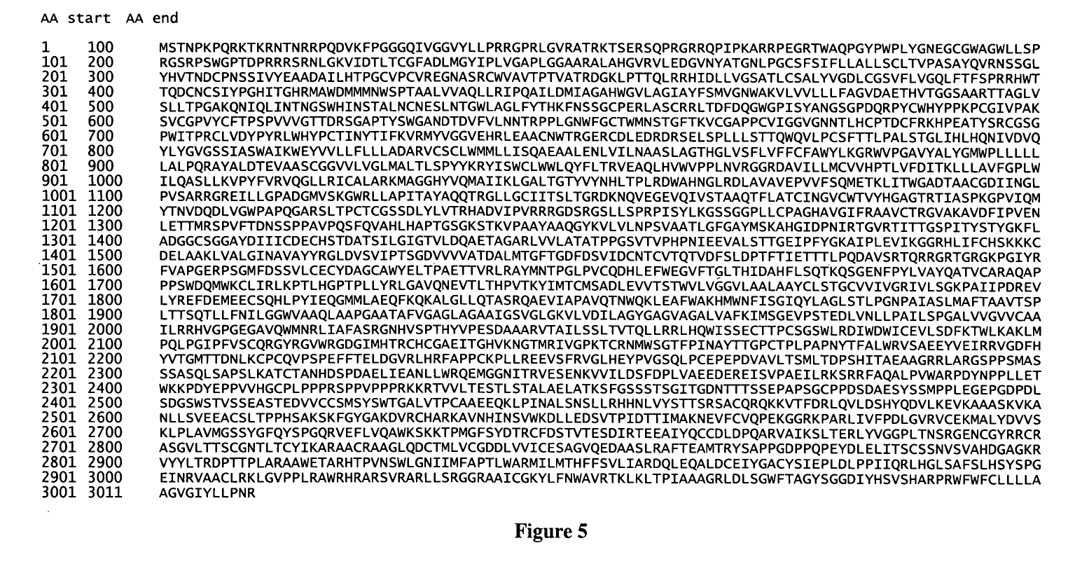

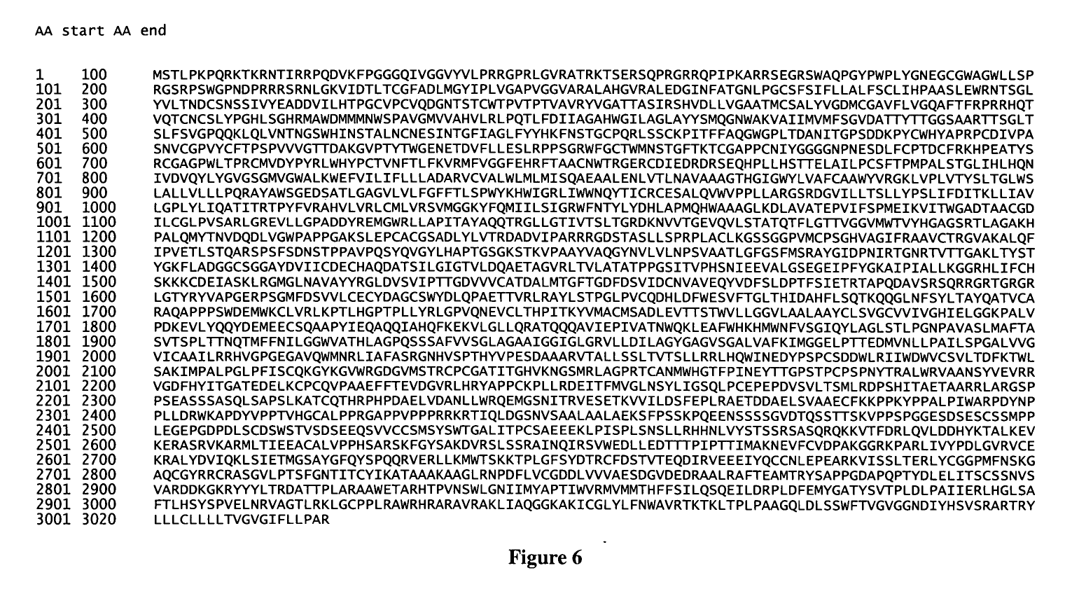

Vaccines having a broad coverage of the existing HCV genotypes or subtypes are preferred. Genotypes 1a, 1b and 3a are the most prevalent HCV genotypes (among HCV infected individuals) and thus important to be taken into consideration. Other genotypes (e.g. genotype 4a) can be retained in view of their prevalence and/or importance. The present invention contains all selected CTL and HTL epitopes for which immunogenicity has been shown and that are present in the consensus sequence of genotype 1b, 1a and/or genotype 3a. Said consensus sequences are shown in FIGS. 2, 5 and 6. Accordingly, the peptides of the present invention are present in the consensus sequence of:
at least genotype 1a,
at least genotype 1b,
at least genotype 3a,
at least genotype 1a, 1b,
at least genotype 1a and 3a,
at least genotype 1b and 3a, or
at least genotype 1a, 1b and 3a.

The epitopes obtained by the methods as described herein can additionally be evaluated on the basis of their conservancy among and/or within different HCV strains or genotypes.

In a further step of the invention, an array of epitopes is selected for inclusion in a polyepitopic composition for use in a vaccine, or for selecting discrete epitopes to be included in a vaccine and/or to be encoded by nucleic acids such as a minigene. It is preferred that each of the following principles are balanced in order to make the selection:
1) Selection of either HCV native or analoged epitopes.
2) Selection of native HCV epitopes that are present in the most prevalent and/or important HCV genotypes or subtypes.
3) Epitopes are selected that have the requisite binding affinity established to be correlated with immunogenicity: for HLA class I an IC50 or Ki of 1000 nM or less, or for HLA class II an IC50 or Ki of 1000 nM or less.
4) Epitopes are selected which, upon administration, induce a T cell response (CTL and/or HTL).
5) Sufficient supermotif bearing-peptides and/or a sufficient array of allele-specific peptides are selected to give broad population coverage. It is a serious hurdle to find, for a given pathogen with a specific sequence, enough immunogenic epitopes so as to cover a complete HLA-locus and consequently a complete population. As such, considering immunogenic peptides for two or three HLA class I loci, i.e. HLA-A, -B and/or -C, significantly increases population coverage for a given pathogen.
6) Of relevance are epitopes referred to as “nested epitopes”. Nested epitopes occur where at least two epitopes overlap partly or completely in a given peptide sequence. A nested peptide sequence can comprise both HLA class I and HLA class II epitopes, 2 or more HLA class I epitopes or 2 or more HLA class II epitopes.
7) It is important to screen the epitope sequence (e.g. comparing with mammal genome sequence) in order to ensure that it does not have pathological or other deleterious biological properties in the treated subject e.g. by inducing auto-antibodies.
8) When used in a polyepitopic composition, spacer amino acid residues can be introduced to avoid junctional epitopes (an epitope recognized by the immune system, not present in the target antigen, and only created by the man-made juxtaposition of epitopes), or to facilitate cleavage between epitopes and thereby enhance epitope presentation. Junctional epitopes are generally to be avoided because the recipient may generate an immune response to that non-native epitope. Of particular concern is a junctional epitope that is a “dominant epitope.” A dominant epitope may lead to such a strong response that immune responses to other epitopes are diminished or suppressed.

The term “peptide” is used interchangeably with “oligopeptide” and “polypeptide” and designates a series of amino acids, connected one to the other, typically by peptide bonds between the amino and carboxyl groups of adjacent amino acids. The preferred CTL-inducing peptides of the invention are 13 residues or less in length and usually consist of 8, 9, 10, 11 or 12 residues, preferably 9 or 10 residues. The preferred HLA class II binding peptides are less than 50 residues in length and usually consist of between 6 and 30 residues, more usually between 12 and 25, and often between 15 and 20 residues. More preferred, an HLA class II binding peptide consists of 12, 13, 14, 15, 16, 17, 18, 19, 20, 21, 22, 23, 24, 25 or more amino acid residues.

The peptides of the invention can be prepared by classical chemical synthesis. “Synthetic peptide” refers to a peptide that is man-made using such methods as chemical synthesis or recombinant DNA technology. The synthesis can be carried out in homogeneous solution or in solid phase. For instance, the synthesis technique in homogeneous solution which can be used is the one described by Houbenweyl in the book entitled “Methode der organischen chemie” (Method of organic chemistry) edited by E. Wunsh, vol. 15-I et II. THIEME, Stuttgart 1974. The polypeptides of the invention can also be prepared in solid phase according to the methods described by Atherton and Shepard in their book entitled “Solid phase peptide synthesis” (IRL Press, Oxford, 1989). The polypeptides according to this invention can also be prepared by means of recombinant DNA techniques as documented below.

Conservative substitutions may be introduced in these HCV polypeptides according to the present invention. The term “conservative substitution” as used herein denotes that one amino acid residue has been replaced by another, biologically similar residue. Peptides having conservative substitutions bind the HLA molecule with a similar affinity as the original peptide and CTL's and/or HTL's generated to or recognizing the original peptide are activated in the presence of cells presenting the altered peptide (and/or vice versa). Examples of conservative substitutions include the substitution of one hydrophobic residue such as isoleucine, valine, leucine or methionine for another, or the substitution of one polar residue for another such as between arginine and lysine, between glutamic and aspartic acids or between glutamine and asparagine and the like. Other substitutions can be introduced as long as the peptide containing said one or more amino acid substitutions is still immunogenic. This can be analysed in ELISPOT assays as described in examples 5 and 6. Accordingly, the current invention also relates to a peptide consisting of an amino acid sequence which is at least 70, 75, 80, 85 or 90% identical to the amino acid sequence of the peptide as disclosed in Tables 13 and 14, and wherein said peptide is still capable of inducing a HLA class I and/or class II restricted T lymphocyte response to cells presenting the original peptides.

A strategy to improve the cross-reactivity of peptides between different HLA types or within a given supermotif or allele is to delete one or more of the deleterious residues present within a peptide and substitute a small “neutral” residue such as Ala, that may not influence T cell recognition of the peptide. Such an improved peptide is sometimes referred to as an analoged peptide.

The peptides can be in their natural (uncharged) forms or in forms which are salts, and either free of modifications such as glycosylation, side chain oxidation, or phosphorylation or containing these modifications. Also included in the definition are peptides modified by additional substituents attached to the amino acids side chains, such as glycosyl units, lipids, or inorganic ions such as phosphates, as well as modifications relating to chemical conversions of the chains, such as oxidation of sulfhydryl groups. Thus, “polypeptide” or its equivalent terms is intended to include the appropriate amino acid sequence referenced, and may be subject to those of the foregoing modifications as long as its functionality is not destroyed.

With regard to a particular amino acid sequence, an “epitope” is a set of amino acid residues which is involved in recognition by a particular immunoglobulin, or in the context of T cells, those residues necessary for recognition by T cell receptor proteins and/or Major Histocompatibility Complex (MHC) molecules. In an immune system setting, in vivo or in vitro, an epitope is the collective features of a molecule, such as primary, secondary and tertiary peptide structure, and charge, that together form a site recognized by an immunoglobulin, T cell receptor or HLA molecule. Throughout this specification “epitope” and “peptide” are used interchangeably.

The phrases “isolated” or “biologically pure” refer to material which is substantially or essentially free from components which normally accompany the material as it is found in its native state. Thus, isolated peptides in accordance with the invention preferably do not contain materials normally associated with the peptides in their in situ environment. An “isolated” epitope refers to an epitope that does not include the whole sequence of the antigen or polypeptide from which the epitope was derived.

It is to be understood that protein or peptide molecules that comprise an epitope of the invention as well as additional amino acid(s) are still within the bounds of the invention.

An “immunogenic peptide” is a peptide that comprises a sequence as disclosed in Tables 13 and/or 14, or a peptide comprising an allele-specific motif or supermotif, such that the peptide will bind an HLA molecule and induce a CTL and/or HTL response. Immunogenic peptides of the invention comprise a peptide capable of binding to an appropriate HLA molecule and the immunogenic peptide can induce an HLA-restricted cytotoxic and/or helper T cell response to the antigen from which the immunogenic peptide is derived. A CTL response is a set of different biological responses of T cells activated by cells presenting the immunogenic peptide in the MHC-I context and includes but is not limited to cellular cytotoxicity, IFN-gamma production and proliferation. An HTL response is a set of different biological responses of T cells activated by APC presenting the immunogenic peptide in the MHC-II context and includes but is not limited to cytokine production (such as IFN-gamma or IL-4) and proliferation. In a preferred embodiment of the invention, the immunogenic peptide consists of less than 50 amino acid residues. Even more particularly, the immunogenic peptide consists of less than 45, 40, 35, 30, 29, 28, 27, 26, 25, 24, 23, 22, 21, 20, 19, 18, 17, 16, 15, 14, 13, 12, 11, 10 or 9 amino acid residues.

Sette and Sidney (1999) (incorporated herein by reference) describe the epitope approach to vaccine development and identified several HLA supermotifs, each of which corresponds to the ability of peptide ligands to bind several different HLA alleles. The HLA allelic variants that bind peptides possessing a particular HLA supermotif are collectively referred to as an HLA supertype.

A “supermotif “is a peptide binding specificity shared by HLA molecules encoded by two or more HLA alleles. Preferably, a supermotif-bearing peptide is recognized with high or intermediate affinity (as defined herein) by two or more HLA antigens. The term “motif” refers to the pattern of residues in a peptide of defined length, usually a peptide of 8, 9, 10, 11, 12 or 13 amino acids for a class I HLA motif and from about 6 to about 50 amino acids, or more specific a peptide of 13, 14, 15, 16, 17, 18, 19, 20, 21, 22, 22, 24, 25, 30, 35, 40 or 50 amino acids for a class II HLA motif, which is recognized by a particular HLA molecule. The family of HLA molecules that bind to the A1 supermotif (i.e. the HLA-A1 supertype) includes at least A0101, A2601, A2602, A2501 and A3201. The family of HLA molecules that bind to the A2 supermotif (i.e. the HLA-A2 supertype) is comprised of at least: A0201 A0202, A0203, A0204, A0205, A0206, A0207, A0209, A0214, A6802 and A6901. Members of the family of HLA molecules that bind the A3 supermotif (the HLA-A3 supertype) include at least A0301, A1101, A3101, A3301 and A6801. The family of HLA molecules that bind to the A24 supermotif (i.e. the A24 supertype) includes at least A2402, A3001 and A2301. The family of HLA molecules that bind the B7 supermotif (i.e., the HLA-B7 supertype) is comprised of at least twenty six HLA-B proteins including: B0702, B0703, B0704, B0705, B1508, B3501, B3502, B3503, B3504, B3505, B3506, B3507, B3508, B5101, B5102, B5103, B5104, B5105, B5301, B5401, B5501, B5502, B5601, B5602, B6701 and B7801. Members of the family of HLA molecules that bind to the B44 supermotif (i.e., the B44 supertype) include at least: B1801, B1802, B3701, B4001, B4002, B4006, B4402, B4403 and B4006 (WO01/21189).

According to a preferred embodiment, the immunogenic peptide of the present invention is less than 50, less than 25, less than 20 or less than 15 amino acids. Peptide motifs are typically different for each protein encoded by each human HLA allele and differ in the pattern of the primary and secondary anchor residues.

“Cross-reactive binding” indicates that a peptide is bound by more than one HLA molecule derived from more than one HLA allele group or locus; a synonym is degenerate binding. “Human Leukocyte Antigen” or “HLA” is a human class I or class II Major Histocompatibility Complex (see, e.g., Stites, et al, IMMUNOLOGY, 8 ED, Lange Publishing, Los Altos, Calif. (1994)). “Major Histocompatibility Complex” or “MHC” is a cluster of genes that plays a role in control of the cellular interactions responsible for physiologic immune responses. In humans, the MHC complex is also known as the HLA complex. For a detailed description of the MHC and HLA complexes, see, Paul, FUNDAMENTAL IMMUNOLOGY, PDED, Raven Press, New York, 1993. The HLA nomenclature used herein is generally known in the art and e.g. as described in “The HLA Factsbook, ed. Marsh et al., Academic Press, 2000”.

Also, information on HLA sequences and the currently used nomenclature can be found on http://www.anthonynolan.org.uk/HIG/.

Polyepitopic Peptides

The present invention also relates to the use of the peptides as described herein for the preparation of an HCV immunogenic composition and more specific to a composition comprising at least one of the peptides as provided in Tables 13-14, possibly in combination with one or more of the same or other peptides or epitopes. The peptides of the invention can be combined via linkage to form polymers (multimers), or can be formulated in a composition without linkage, as an admixture. In a specific embodiment, the peptides of the invention can be linked as a polyepitopic peptide. The linkage of the different peptides in the polyepitopic peptide is such that the overall amino acid sequence differs from a naturally occurring sequence. Hence, the polyepitopic peptide sequence of the present invention is a non-naturally occurring sequence. Accordingly, the present invention relates to a composition or polyepitopic peptide comprising at least one peptide selected from the peptides disclosed in Tables 13 and 14. Of particular interest are the peptides with K1 or IC50 <1000 nM. More preferably, the peptides of interest are these peptides having a positive immunogenicity after evaluation by the herein described strategies. Particularly preferred are the HLA class I binding peptides identified by:
for HLA-A: SEQ ID NO 557, 1241, 1456, 1478, 1833, 1887, 67, 922, 66, 361, 1070, 1072, 1151, 71, 1233, 1269, 75, 73, 1396, 5, 87, 91, 238, 265, 1661, 1753, 76, 81, 92, 1933, 1934, 69, 2043, 2047, 74, 63, 2053, 83, 56, 155, 156, 1205, 1206, 167, 1350, 47, 146, 1609, 144, 3, 39, 158, 16, 122, 1034, 1095, 1096, 1150, 246, 1406, 23, 1483, 1512, 87, 93, 1625, 1626, 59, 1710, 250, 81, 1885, 1916, 1938, 2048, 271, 2083, 1, 877, 17, 7, 1086, 1087, 1468, 1700 and 1894;
for HLA-B: SEQ ID NO 402, 836, 381, 371, 853, 370, 387, 307, 1237, 1289, 1343, 1418, 1419, 375, 1430, 380, 450, 1582, 390, 1677, 1687, 121, 386, 372, 95, 443, 396, 455, 1441, 436, 1719, 92, 394, 1969, 287, 1237, 1289, 375, 1430, 1444, 582, 1117 and 59;
for HLA-C: SEQ ID NO 1048, 1095, 1730, 349, 475, 111, 2066, 1511, 1454, 1100 and 907.

Preferred HLA class II binding peptides are the peptides with IC50 <500 nM identified by SEQ ID NO 2142, 2213, 2157, 2245, 2162, 2164, 2235, 2113, 2182, 2111, 2180, 2236, 2112, 2132, 2192, 2107, 2137, 2125, 2229, 2166, 2136, 2177, 2153, 2110, 2156, 2241, 2228, 2219, 2187, 2249, 2194, 2207 and 2237.

Particularly preferred HLA class II peptides are identified by SEQ ID NO 2235, 2164, 2162, 2113, 2182, 2180, 2236, 2149, 2112, 2201, 2249, 2158, 2108, 2107, 2229, 2194, 2156, 2228, 2207 and 2232.

More preferably, the composition or polyepitopic peptide comprises at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60 or more peptides. Preferably, the peptides are selected from Tables 13 and 14. Any combination of peptides is possible, e.g., the composition can comprise at least one HLA-A binding peptide and at least one HLA-B or HLA-C binding peptide. Furthermore, the composition can also comprise at least one HLA-B binding peptide and at least one HLA-C binding peptide. More specific, the composition comprises at least one HLA-A, at least one HLA-B and at least one HLA-C binding peptide. In a preferred embodiment, the polyepitopic peptide or composition comprises at least two peptides derived from a HCV protein and capable of inducing a HLA class I and/or class II restricted T lymphocyte response, wherein at least one peptide is a HLA-C binding peptide. In a further embodiment, the composition comprises at least two HLA-DRB binding peptides, preferably selected from Table 14.

A “HLA-A binding peptide” is defined as a peptide capable of binding at least one molecule of the HLA-A locus. Said definition can be extrapolated to the other loci, i.e. HLA-B, HLA-C, HLA-DRB1-9, etc.

In a particular, the epitopes of the invention can be combined in an HLA-group restricted polyepitope. The term “HLA-group restricted polyepitope” refers to a polyepitopic peptide comprising at least two epitopes binding to an allele or molecule of the same HLA group. The HLA nomenclature used herein is generally known in the art and e.g. as described in “The HLA Factsbook, ed. Marsh et al., Academic Press, 2000”. In a preferred embodiment, the HLA-group restricted polyepitope is a HLA-A01 restricted polyepitope, a HLA-A02 restricted polyepitope, a HLA-A03 restricted polyepitope, a HLA-A11 restricted polyepitope, a HLA-A24 restricted polyepitope, a HLA-B07 restricted polyepitope, a HLA-B08 restricted polyepitope, a HLA-B35 restricted polyepitope, a HLA-B40 restricted polyepitope, a HLA-B44 restricted polyepitope, a HLA-Cw03 restricted polyepitope, a HLA-Cw04 restricted polyepitope, a HLA-Cw06 restricted polyepitope, a HLA-Cw07 restricted polyepitope, a HLA-DRB1*01 restricted polyepitope, HLA-DRB1*03 restricted polyepitope or HLA-DRB1*04 restricted polyepitope.

The number of epitopes in a HLA-group restricted polyepitope is not limited and can be 2, 3, 4, 5, 6, 7, 8, 9, 10, 11, 12, 13, 14, 15, 20, 25 or more. An HLA-group restricted polyepitope can be used in a first phase of establishing the immunogenicity of a subset of epitopes in a construct. The advantage of using such an HLA-group restricted polyepitope is that a considerable number of HLA restricted epitopes can be evaluated in one and the same construct. Furthermore, a specific selection of more than one HLA-group restricted polyepitope can be administered in order to customize treatment. More specific, the selection can comprise more than one HLA-group restricted polyepitope within a given HLA-locus or covering 2, 3 or more HLA-loci.

More particular, the composition as described herein comprises linked peptides that are either contiguous or are separated by a linker or a spacer amino acid or spacer peptide. This is referred to as a polyepitopic or multi-epitopic peptide.

“Link” or join” refers to any method known in the art for functionally connecting peptides (direct of via a linker), including, without limitation, recombinant fusion, covalent bonding, non-covalent bonding, disulfide bonding, ionic bonding, hydrogen bonding, polymerization, cyclization, electrostatic bonding and connecting through a central linker or carrier. Polymerization can be accomplished for example by reaction between glutaraldehyde and the —NH2 groups of the lysine residues using routine methodology. The peptides may also be linked as a branched structure through synthesis of the desired peptide directly onto a central carrier, e.g. a poly-lysyl core resin.

This larger, preferably poly- or multi-epitopic, peptide can be generated synthetically, recombinantly, or via cleavage from the native source.

The polyepitopic peptide can exist as a homopolymer comprising multiple copies of the same peptide, or as a heteropolymer of various peptides. Polymers have the advantage of increased immunological reaction and, where different peptide epitopes are used to make up the polymer, the additional ability to induce antibodies, HTL's and/or CTLs that react with different antigenic determinants of the pathogenic organism targeted for an immune response. Multi-epitope constructs can for example be prepared according to the methods set forth in Ishioka et al., 1999; Velders et al., 2001; or as described in WO04/031210—Epimmune. The polyepitopic peptide can be expressed as one protein. In order to carry out the expression of the polyepitopic peptide in bacteria, in eukaryotic cells (including yeast) or in cultured vertebrate hosts such as Chinese Hamster Ovary (CHO), Vero cells, RK13, COS1, BHK, and MDCK cells, or invertebrate hosts such as insect cells, the following steps are carried out:
transformation of an appropriate cellular host with a recombinant vector, or by means of adenoviruses, influenza viruses, BCG, and any other live carrier systems, in which a nucleotide sequence coding for one of the polypeptides of the invention has been inserted under the control of the appropriate regulatory elements, particularly a promoter recognized by the polymerases of the cellular host or of the live carrier system and in the case of a prokaryotic host, an appropriate ribosome binding site (RBS), enabling the expression in said cellular host of said nucleotide sequence,
culture of said transformed cellular host under conditions enabling the expression of said insert.

The polyepitopic peptide can be purified by methods well known to the person skilled in the art.

Vaccines that have broad population coverage are preferred because they are more commercially viable and generally applicable to most people. Broad population coverage can be obtained through selecting peptides that bind to HLA alleles which, when considered in total, are present in most of the individuals of the population. The A2-, A3-, and B7 supertypes are each present on the average of over 40% in each of the five major ethnic groups, i.e. Caucasian, North American Black, Japanese, Chinese and Hispanic. Coverage in excess of 80% is achieved with a combination of these supermotifs. The B44-, A1-, and A24-supertypes are present, on average, in a range from 25% to 40% in these major ethnic populations. The HLA groups Cw04, Cw03, Cw06 and Cw07 are each present, on average, in a range from 13% to 54% in these major ethnic populations. Thus, by including epitopes from most frequent HLA-A, -B and/or -C alleles, an average population coverage of 90-99% is obtained for five major ethnic groups. Especially in the field of HLA-C, experimentally determined data (both binding and immunogenic) for HCV epitopes are scarce. Accordingly, the present invention relates to a composition or polyepitopic peptide comprising at least two peptides derived from a HCV protein and capable of inducing a HLA class I and/or class II restricted T lymphocyte response, wherein at least one peptide is a HLA-C binding peptide. More preferred, said composition or polyepitopic peptide comprises at least 2, 3, 4, 5 or more HLA-C binding peptide(s). More particularly, the one or more HLA-C binding peptides are derived from at least one of the following HCV regions: Core, E1, E2/NS1, NS2, NS3, NS4A, NS4B, NS5A and NSSB. Even more preferred is that the HLA-C binding peptides are furthermore characterized in that they are present in the HCV consensus sequence of genotype 1a, 1b and/or 3a. Optionally, the composition or polyepitopic peptide can furthermore comprise at least 1, 2, 3, 4 or more HLA-B binding peptide(s) and/or at least 1, 2, 3, 4 or more HLA-A binding peptide(s) and/or at least 1, 2, 3, 4 or more HLA-DRB1-9 binding peptide(s). More preferred, the composition or the polyepitopic peptide of the present invention comprises at least 1, 2, 3, 4 or more HLA-A binding peptide(s), at least 1, 2, 3, 4 or more HLA-B binding peptide(s) and at least 1, 2, 3, 4 or more HLA-C binding peptide(s), optionally in combination with a HLA class II binding peptide. In a specific embodiment, the peptides are selected from Table 13 or 14.

Furthermore, the present invention relates to a composition comprising at least one peptide selected from Tables 13 and 14 and at least one other HLA class I binding peptide, a HLA class II binding peptide or a HCV derived peptide. Said “other” HLA class I binding peptide and said HLA class II binding peptide to be used in combination with the peptides of the present invention can be derived from HCV or from a foreign antigen or organism (non-HCV). There is no limitation on the length of said other peptides, these can have a length of e.g. 5, 6, 7, 8, 9, 10, 11, 12, 13, 14, 15, 16, 17, 18, 19, 20, 21, 22, 23, 24, 25, 30 or more amino acids. The “at least one” can include, e.g., at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95 or 100 or more peptides. Preferably, said HLA class I binding peptide is a peptide capable of binding one or more HLA class I alleles. More specific, said peptide is selected from the group consisting of peptides binding a molecule of the following HLA groups: HLA-A1, HLA-A2, HLA-A3, HLA-A11, HLA-A24, HLA-B7, HLA-B8, HLA-B27, HLA-B35, HLA-B40, HLA-B44, HLA-B58, HLA-B62, HLA-Cw03, HLA-Cw04, HLA-Cw06 and/or HLA-Cw07.

For HLA class II, the peptides, also called HTL epitopes, are preferably selected from the group consisting of peptides binding a molecule of the HLA-loci HLA-DR, HLA-DQ and/or HLA-DP, or as described in e.g. WO95/27733, WO02/26785, WO01/21189, WO02/23770, WO03/084988, WO04/024182, Hoffmann et al., 1995, Diepolder et al., 1997, Werheimer et al, 2003 and Lamonaca et al, 1999 (incorporated herein by reference). The preferred HLA class II binding peptides are less than about 50 residues in length and usually consist of between about 6 and about 30 residues, more usually between about 12 and 25, and often between about 15 and 20 residues. For example, a HLA class II binding peptide consists of 12, 13, 14, 15, 16, 17, 18, 19, 20, 21, 22, 23, 24, 25 or more amino acid residues. Further and preferred examples of candidate HTL epitopes to include in a polyepitopic construct for use in a vaccine, or for selecting discrete epitopes to be included in a vaccine and/or to be encoded by nucleic acids such as a minigene are enclosed in Table 14.

A “CTL inducing peptide” is a HLA Class I binding peptide that is capable of inducing a CTL response. A “HTL inducing peptide” is a HLA Class II binding peptide that is capable of inducing a HTL response.

In a specific embodiment, the present invention relates to a composition or polyepitopic peptide comprising at least two HLA class I binding peptides selected from Table 13 or at least two HLA class II binding peptides selected from Table 14. Any combination is possible. More preferred, the at least two peptides are selected to bind HLA molecules derived from the same or a different HLA locus, i.e. HLA-A, -B, -C or DRB1. Alternatively, the at least two peptides are selected to bind HLA molecules derived from the same or a different HLA-group. Preferred HLA-groups are: HLA-A01, A02, A03, A11, A24, B07, B08, B35, B40, B44, Cw03, Cw04, Cw06, Cw07, DRB1*01, DRB1*03 and DRB1*04.

In a more preferred embodiment, the present invention relates to a composition or polyepitopic peptide comprising at least three HLA class I binding peptides selected from Table 13. Any combination is possible, for example:
at least 3 HLA-A binding peptides,
at least 3 HLA-B binding peptides,
at least 3 HLA-C binding peptides,
at least 2 HLA-A binding peptides and at least 1 HLA-B or HLA-C binding peptide,
at least 2 HLA-B binding peptides and at least 1 HLA-A or HLA-C binding peptide,
at least 2 HLA-C binding peptides and at least 1 HLA-A or HLA-B binding peptide, or
at least one HLA-A, at least one HLA B and at least one HLA-C binding peptide.

More preferred and for each combination, the at least three peptides are selected to bind HLA molecules derived from the same or a different HLA-group. Preferred HLA-groups are: HLA-A01, A02, A03, A11, A24, B07, B08, B35, B40, B44, Cw03, Cw04, Cw06 and Cw07. More specifically, the composition or polyepitopic peptide comprises at least three peptides selected from Table 13, said at least three peptides being:
at least one HLA-A binding peptide selected from a HLA-A01, A02, A3, A11 or A24 binding peptide,
at least one HLA-B binding peptide selected from a HLA-B07, B08, B35, B40 or B44 binding peptide, and/or
at least one HLA-C binding peptide selected from a HLA-Cw03, Cw04, Cw06 or Cw07 binding peptide.

An HLA-A01 binding peptide is defined as a peptide capable of binding at least one molecule of the HLA-01 group. Said definition can be extrapolated to the other allele groups, i.e. A02, A03, A11, A24, B07, B08, B35, B40, B44, Cw03, Cw04, Cw06, Cw07 etc.

HLA class I binding peptides of the invention can be admixed with, or linked to, HLA class II binding peptides, to facilitate activation of both cytotoxic T lymphocytes and helper T lymphocytes. Accordingly, the composition or polyepitopic peptide of the present invention further comprises at least one HLA class II binding peptide. Alternatively, the composition or polyepitopic peptide of the present invention comprises at least one HLA class II binding peptide. More specific, said HLA class II binding peptide is selected from Table 14. The amount of HTL epitopes is not limiting, i.e., 1, 2, 3, 4, 5, 6, 7, 8, 9, 10, 11, 12, 13, 14, 15, 16, 17, 18, 19, 20, 21, 22, 23, 24, 25 or more HTL epitopes can be comprised in the composition or polyepitopic peptide of the present invention. In a specific embodiment, the composition or polyepitopic peptide comprises at least three CTL peptides selected from Table 13 and at least one HTL peptide selected from Table 14.

In a further embodiment, the composition or polyepitopic peptide can also comprise the universal T cell epitope called PADRE® (Epimmune, San Diego; described, for example in U.S. Pat. No. 5,736,142 or International Application WO95/07707, which are enclosed herein by reference). A ‘PanDR binding peptide or PADRE® peptide” is a member of a family of molecules that binds more that one HLA class II DR molecule. The pattern that defines the PADRE® family of molecules can be thought of as an HLA Class II supermotif. PADRE® binds to most HLA-DR molecules and stimulates in vitro and in vivo human helper T lymphocyte (HTL) responses. Alternatively T-help epitopes can be used from universally used vaccines such as tetanos toxoid.

In a further embodiment, the peptides in the composition or polyepitopic peptide are characterized in that they are derived from a HCV protein, and more specific from at least one of the following HCV regions selected from the group consisting of Core, E1, E2/NS1, NS2, NS3, NS4A, NS4B, NS5A and NS5B. Even more preferred is that peptides are characterized in that they are present in the HCV consensus sequence of genotype 1a, 1b and/or 3a.

In a further embodiment the two or more epitopes in the polyepitopic peptide consist of discrete HCV amino acid sequences (discrete epitopes) or nested HCV amino acid sequences (nested epitopes). Particularly preferred are “nested epitopes”. Nested epitopes occur where at least two individual or discrete epitopes overlap partly or completely in a given peptide sequence. A nested epitope can comprise both HLA class I and HLA class II epitopes, 2 or more HLA class I epitopes (whereby the epitopes bind two or more alleles of class I loci, supertypes or groups), or 2 or more HLA class II epitopes (whereby the epitopes bind two or more alleles of class II loci, supertypes or groups). A nested epitope can comprise 2, 3, 4, 5, 6, 7, 8, 9, 10, 11, 12, 13, 14, 15, 16, 17, 18, 19, 20, 21, 22, 23, 24, 25 or more individual epitopes. Nested epitopes enable epitope-based vaccines with broad population coverage as they provide a high number of epitopes by a limited number of amino acids. This is particular advantageous since the number of epitopes of a vaccine is limited by constraints originating from manufacturing, formulation and product stability. The length of the nested epitope varies according to the amount of individual epitopes included. Usually, a nested epitope consists of 9 to 35 amino acids. Preferably, the nested epitope consists of 35 amino acids or less, i.e 34, 33, 32, 31, 30, 29, 28, 27, 26, 25, 24, 23, 22, 20, 19, 18, 17, 16, 15, 14, 13, 12, 11, 10 or 9 amino acids. More preferred, the nested epitope consists of 9 to 30 amino acids, 9 to 25 amino acids, 10 to 30 amino acids or 10 to 25 amino acids.

Examples of nested epitopes based on 3 or more individual epitopes identified in the present invention and whereby the individual epitopes have a binding affinity of less than 11000 nM for a given HLA are shown in Table A. Said individual epitopes have an overlap of at least 3 amino acids.

TABLE A

The nested epitopes are indicated in bold. The individual
epitopes are indicated in normal font.

| | | | |
|---|---|---|---|
| SEQ | | | HLA |
| ID | | HLA class I | Class II |
| NO | Sequence | coverage | coverage |

| | | | |
|---|---|---|---|
| 2277 | GQIVGGVYLLPRRGPRLGVRATRKSER | | |
| 2254 | QIVGGVYLLPRRGPRLGVRATRKSER | | |
| 127 | GQIVGGVYL | Cw03 | |
| 616 | QIVGGVYLL | A02, Cw03 | |
| 149 | YLLPRRGPR | A03 | |
| 2047 | YLLPRRGPRL | A02; B08 | |
| 132 | LLPRRGPRL | A24; B08 | |
| 1442 | LPRRGPRL | B07; B08 | |
| 380 | LPRRGPRLG | B07 | |
| 450 | LPRRGPRLGV | B07 | |
| 2149 | GPRLGVRATRKSER | | DRB1 |
| 387 | GPRLGVRAT | B07 | |
| 144 | RLGVRATRK | A03 | |
| 2255 | KTSERSQPRGRRQPIPKARR | | |
| 167 | KTSERSQPR | A03 | |
| 390 | QPRGRRQPI | B07; B08 | |
| 159 | RQPIPKARR | A03 | |
| 2256 | LYGNEGLGWAGWLL | | |
| 1487 | LYGNEGLGW | A24 | |
| 1150 | GLGWAGWLL | A24; A02 | |
| 2257 | VIDTLTCGFADLMGYIPLVGAPLGGAARAL | | |
| 1914 | VIDTLTCGFA | A01 | |
| 2 | LTCGFADLM | A01 | |
| 1465 | LTCGFADLMGY | A01 | |
| 236 | GFADLMGYI | A24; Cw04 | |
| 1048 | FADLMGYIPL | Cw04 | |
| 66 | DLMGYIPLV | A02 | |
| 2038 | YIPLVGAPL | A02; A24 | |
| 1289 | IPLVGAPL | B07; B08 | |
| 384 | APLGGAARA | B07 | |
| 836 | APLGGAARAL | B07 | |
| 2258 | NLPGCSFSIFLLALLSCLT | | |
| 93 | NLPGCSFSI | A24; A02 | |
| 1425 | LPGCSFSI | B07 | |
| 375 | LPGCSFSIF | B07; B35 | |
| 1426 | LPGCSFSIFL | B07 | |
| 250 | SFSIFLLAL | A24; Cw04, −07 | |
| 361 | FLLALLSCL | A02 | |
| 1070 | FLLALLSCLT | A02 | |
| 2259 | AAYAAQGYKVLVLNPSVAATLGFGAYMSKAHGV | | |
| 56 | AAYAAQGYK | A03 | |
| 277 | AYAAQGYKV | A24 | |
| 95 | YAAQGYKVL | B07 | |
| 2107 | AQGYKVLVLNPSVAA | | DRB1, −4 |
| 2157 | GYKVLVLNPSVAATL | | DRB1, −4 |
| 2235 | VLVLNPSVAATLGFG | | DRB1 |
| 73 | KVLVLNPSV | A02 | |
| 1887 | VAATLGFGAY | A01 | |
| 557 | AATLGFGAY | A01 | |
| 1831 | TLGFGAYMSK | A03 | |
| 244 | AYMSKAHGV | A24 | |
| 2260 | GEIPFYGKAIPI | | |
| 1117 | GEIPFYGKAI | B44 | |
| 1283 | IPFYGKAI | B07 | |
| 1553 | PFYGKAIPI | A24 | |
| 2261 | HLIFCHSKKKCDEL | | |
| 148 | HLIFCHSKK | A03 | |
| 1228 | HLIFCHSKKK | A03 | |
| 151 | LIFCHSKKK | A03 | |
| 455 | HSKKKCDEL | B08 | |
| 2262 | GLNAVAYYRGLDVSVI | | |
| 145 | GLNAVAYYR | A03 | |
| 394 | VAYYRGLDV | B08; Cw06 | |
| 907 | AYYRGLDVSV | Cw07 | |
| 271 | YYRGLDVSV | Cw07; A24 | |
| 2083 | YYRGLDVSVI | A24; Cw07, −06 | |
| 2263 | TPGERPSGMFDSSVLCECY | | |
| 372 | TPGERPSGM | B07 | |
| 1687 | RPSGMFDSSV | B07 | |
| 71 | GMFDSSVLC | A02 | |
| 17 | DSSVLCECY | A01 | |
| 2264 | LRAYLNTPGLPVCQDHLEF | | |
| 1454 | LRAYLNTPGL | Cw07 | |
| 434 | RAYLNTPGL | Cw03 | |
| 2048 | YLNTPGLPV | A02; A24 | |
| 1444 | LPVCQDHLEF | B35 | |
| 2265 | EFWESVFTGLTHIDAHFL | | |
| 1010 | EFWESVFTGL | Cw04 | |
| 234 | FWESVFTGL | Cw04; A24 | |
| 76 | SVFTGLTHI | A02 | |
| 258 | GLTHIDAHF | A24 | |
| 5 | LTHIDAHFL | A02 | |
| 2266 | FPYLVAYQATVCARA | | |
| 443 | FPYLVAYQA | B08; B35 | |
| 2052 | YLVAYQATV | A02 | |
| 83 | YQATVCARA | A02 | |
| 2267 | APPPSWDQMWKCLIRLKPTLHGPTPLLYRLGAV | | |
| 381 | APPPSWDQM | B35; B07 | |
| 279 | SWDQMWKCL | Cw04; A24 | |
| 1804 | SWDQMWKCLI | Cw04 | |
| 238 | QMWKCLIRL | A02; A24 | |
| 122 | CLIRLKPTL | A24 | |
| 205 | LIRLKPTLH | B08 | |
| 2164 | KPTLHGPTPLLYRLG | | DRB1 |
| 1343 | KPTLHGPTPL | B07; B35 | |
| 1587 | PTLHGPTPLLY | A01 | |
| 81 | TLHGPTPLL | A02; A24 | |
| 1833 | TLHGPTPLLY | A01 | |
| 219 | LHGPTPLLY | Cw07 | |
| 307 | GPTPLLYRL | B35; B07 | |
| 389 | TPLLYRLGA | B07 | |
| 1851 | TPLLYRLGAV | B07 | |
| 2268 | VTLTHPITKYIMA | | |
| 21 | VTLTHPITK | A03 | |
| 23 | LTHPITKYI | A24 | |
| 396 | HPITKYIMA | B08; B35 | |
| 2269 | FWAKHMWNFISGIQYLAGLSTLPGNPAIASLMAF | | |
| 2278 | FWAKHMWNFISGIQYLAGLSTLPGNPA | | |
| 1095 | FWAKHMWNF | A24; Cw04 | |
| 1096 | FWAKHMWNFI | A24 | |
| 1993 | WAKHMWNFI | B08 | |
| 1233 | HMWNFISGI | A02 | |
| 1521 | NFISGIQYL | A24; Cw04 | DRB1, −4, −5 |
| 2162 | IQYLAGLSTLPGNPA | | |
| 1625 | QYLAGLSTL | A24 | |
| 1428 | LPGNPAIASL | B07 | |
| 1527 | NPAIASLMA | B07 | |
| 1528 | NPAIASLMAF | B07; B35 | |
| 2270 | KVLVDILAGYGAGVAGALVAFK | | |
| 1350 | KVLVDILAGY | A03 | |
| 1478 | LVDILAGYGA | A01 | |
| 1269 | ILAGYGAGV | A02 | |
| 2166 | LAGYGAGVAGALVAF | | DRB1 |
| 1193 | GVAGALVAFK | A03 | |
| 1890 | VAGALVAFK | A03 | |
| 2271 | VNLLPAILSPGALVVGV | | |
| 2236 | VNLLPAILSPGALVVG | | DRB1, −4 |
| 1418 | LPAILSPGAL | B07; B35 | |
| 1275 | ILSPGALVV | A02 | |
| 1759 | SPGALVVGV | B07 | |
| 2272 | GRKPARLIVFPDLGVRVCEKMALYDVVSTL | | |
| 1182 | GRKPARLIVF | Cw07 | |
| 1336 | KPARLIVF | B07 | |
| 643 | ARLIVFPDL | Cw07 | |
| 1661 | RLIVFPDLGV | A02 | |
| 349 | VFPDLGVRV | Cw04 | |
| 632 | VRVCEKMAL | Cw07 | |
| 3 | RVCEKMALY | A03 | |
| 67 | ALYDVVSTL | A02; A24 | |
| 2273 | VMGSSYGFQYSPGQRVEFLVNAWKSKKCPMGFSY | | |
| 1938 | VMGSSYGF | A24 | |
| 2153 | GSSYGFQYSPGQRVE | | DRB1, −3, −5 |
| 111 | FQYSPGQRV | Cw06 | |
| 1626 | QYSPGQRVEF | A24 | |
| 373 | SPGQRVEFL | B07; B08 | |
| 1710 | RVEFLVNAW | A24 | |
| 146 | LVNAWKSKK | A03 | |
| 1739 | SKKCPMGFSY | Cw07 | |
| 2274 | EARQAIRSLTERLYIGGPLT | | |
| 388 | EARQAIRSL | B08; B07 | |
| 624 | IRSLTERLY | Cw07; Cw06 | |
| 79 | RLYIGGPLT | A02 | |
| 2275 | YRRCRASGVL | | |
| 475 | YRRCRASGV | B08; Cw06 | |
| 2066 | YRRCRASGVL | Cw07; Cw06 | |
| 2276 | PVNSWLGNIIMYAPTLWARMILMTHFFS | | |
| 256 | PVNSWLGNI | A24 | |
| 62 | WLGNIIMYA | A02 | |
| 87 | NIIMYAPTL | A02; A24; Cw03 | |
| 246 | IIMYAPTLW | A24 | |
| 84 | IMYAPTLWA | A02 | |
| 1511 | MYAPTLWARM | Cw07 | |
| 852 | APTLWARM | B07 | |
| 371 | APTLWARMI | B07 | |
| 853 | APTLWARMIL | B07 | |
| 854 | APTLWARMILM | B07 | |
| 2194 | PTLWARMILMTHFFS | | DRB1, −4 |
| 92 | TLWARMILM | A02; B08 | |
| 1483 | LWARMILMTHF | A24 | |
| 287 | WARMILMTH | B08 | |
| 1997 | WARMILMTHF | Cw07 | |
| 641 | ARMILMTHF | Cw07 | |
| 864 | ARMILMTHFF | Cw07 | |
| 59 | RMILMTHFF | A24; B44 | |

Accordingly, the present invention encompasses a nested epitope consisting of 9 to 35 amino acids and comprising at least 2 epitopes selected from Tables 13 and 14. More specific, the nested epitope comprises 2 or more individual epitopes as given in Table A. More preferred, the nested epitope comprises 3, 4, 5, 6, 7, 8, 9, 10, 11, 12, 13, 14, 15, 16, 17, 18, 19, 20, 21, 22, 23, 24, 25 or more epitopes selected from Tables 13 and 14. Examples of such nested epitopes are presented in Table A. The present invention thus relates to a nested epitope consisting of 9 to 35 amino acids and selected from the group consisting of SEQ ID NO 2254 to 2278, or a part thereof, characterized in that the nested epitope or the part thereof comprises at least 2 individual CTL and/or HTL epitopes. More preferred, said nested epitope or part thereof comprises at least 3, 4, 5, 6, 7, 8, 9, 10, 11, 12, 13, 14, 15, 16, 17, 18, 19, 20, 21, 22, 23, 24, 25 or more individual CTL and/or HTL epitopes as presented in Table A.

The applications of the nested epitopes in the present invention, i.e. possible combinations, modifications, compositions, kits, therapeutic and diagnostic use, are the same as described for the (polyepitopic) peptides of the present invention.

In a preferred embodiment, the present invention relates to a polyepitopic peptide comprising at least one nested epitope or a fragment thereof as described herein.

The peptides or polypeptides or polyepitopic peptides can optionally be modified, such as by lipidation (e.g. a peptide joined to a lipid), addition of targeting or other sequences. In the HCV peptides as described herein, one cysteine residue, or 2 or more cysteine residues comprised in said peptides may be “reversibly or irreversibly blocked”.

An “reversibly blocked cysteine” is a cysteine of which the cysteine thiol-group is irreversibly protected by chemical means. In particular, “irreversible protection” or “irreversible blocking” by chemical means refers to alkylation, preferably alkylation of a cysteine in a protein by means of alkylating agents, such as, for example, active halogens, ethylenimine or N-(iodoethyl)trifluoro-acetamide. In this respect, it is to be understood that alkylation of cysteine thiol-groups refers to the replacement of the thiol-hydrogen by (CH₂)ₙR, in which n is 0, 1, 2, 3 or 4 and R═H, COOH, NH₂, CONH₂, phenyl, or any derivative thereof. Alkylation can be performed by any method known in the art, such as, for example, active halogens X(CH₂)ₙR in which X is a halogen such as I, Br, Cl or F. Examples of active halogens are methyliodide, iodoacetic acid, iodoacetamide, and 2-bromoethylamine.

A “reversibly blocked cysteine” is a cysteine of which the cysteine thiol-groups is reversibly protected. In particular, the term “reversible protection” or “reversible blocking” as used herein contemplates covalently binding of modification agents to the cysteine thiol-groups, as well as manipulating the environment of the protein such, that the redox state of the cysteine thiol-groups remains (shielding). Reversible protection of the cysteine thiol-groups can be carried out chemically or enzymatically. The term “reversible protection by enzymatical means” as used herein contemplates reversible protection mediated by enzymes, such as for example acyl-transferases, e.g. acyl-transferases that are involved in catalysing thio-esterification, such as palmitoyl acyltransferase. The term “reversible protection by chemical means” as used herein contemplates reversible protection:
1. by modification agents that reversibly modify cysteinyls such as for example by sulphonation and thio-esterification;
2. by modification agents that reversibly modify the cysteinyls of the present invention such as, for example, by heavy metals, in particular Zn²⁺, Cd²⁺, mono-, dithio- and disulfide-compounds (e.g. aryl- and alkylmethanethiosulfonate, dithiopyridine, dithiomorpholine, dihydrolipoamide, Ellmann reagent, aldrothiol™ (Aldrich) (Rein et al. 1996), dithiocarbamates), or thiolation agents (e.g. gluthathion, N-Acetyl cysteine, cysteineamine). Dithiocarbamate comprise a broad class of molecules possessing an R₁R₂NC(S)SR₃ functional group, which gives them the ability to react with sulphydryl groups. Thiol containing compounds are preferentially used in a concentration of 0,1-50 mM, more preferentially in a concentration of 1-50 mM, and even more preferentially in a concentration of 10-50 mM;
3. by the presence of modification agents that preserve the thiol status (stabilise), in particular antioxidantia, such as for example DTT, dihydroascorbate, vitamins and derivates, mannitol, amino acids, peptides and derivates (e.g. histidine, ergothioneine, camosine, methionine), gallates, hydroxyanisole, hydoxytoluene, hydroquinon, hydroxymethylphenol and their derivates in concentration range of 10 μM-10 mM, more preferentially in a concentration of 1-10 mM;
4. by thiol stabilising conditions such as, for example, (i) cofactors as metal ions (Zn²⁺, Mg²⁺), ATP, (ii) pH control (e.g. for proteins in most cases pH ˜5 or pH is preferentially thiol pKₐ −2; e.g. for peptides purified by Reversed Phase Chromatography at pH ˜2).

Combinations of reversible protection as described in (1), (2), (3) and (4) may be applied.

The reversible protection and thiol stabilizing compounds may be presented under a monomeric, polymeric or liposomic form.

The removal of the reversibly protection state of the cysteine residues can chemically or enzymatically accomplished by e.g.:
a reductant, in particular DTT, DTE, 2-mercaptoethanol, dithionite, SnCl₂, sodium borohydride, hydroxylamine, TCEP, in particular in a concentration of 1-200 mM, more preferentially in a concentration of 50-200 mM;
removal of the thiol stabilising conditions or agents by e.g. pH increase;
enzymes, in particular thioesterases, glutaredoxine, thioredoxine, in particular in a concentration of 0,01-5 μM, even more particular in a concentration range of 0,1-5 μM.;
combinations of the above described chemical and/or enzymatical conditions.

The removal of the reversibly protection state of the cysteine residues can be carried out in vitro or in vivo, e.g. in a cell or in an individual.

Alternatively, one cysteine residue, or 2 or more cysteine residues comprised in the HCV peptides as described herein may be mutated to a natural amino acid, preferentially to methionine, glutamic acid, glutamine or lysine.

The peptides of the invention can be combined via linkage or via a spacer amino acid to form polymers (multimers: homopolymers or heteropolymers), or can be formulated in a composition without linkage, as an admixture. The “spacer amino acid” or “spacer peptide” is typically comprised of one or more relatively small, neutral molecules, such as amino acids or amino acid mimetics, which are substantially uncharged under physiological conditions. The spacers are typically selected from, e.g., Ala, Gly, Leu, Ile, or other neutral spacers of nonpolar amino acids or neutral polar amino acids. It will be understood that the optionally present spacer need not be comprised of the same residues and thus may be a hetero- or homo-oligomer. When present, the spacer will be at least 1 residue, more usually 2, 3, 4, 5 or 6 residues, or even up to 7, 8, 9, 10, 15, 20, 30, or 50 residues. Spacer amino acid residues can be introduced to avoid junctional epitopes (an epitope recognized by the immune system, not present in the target antigen, and only created by the man-made juxtaposition of epitopes), or to facilitate cleavage between epitopes and thereby enhance epitope presentation. Generally, the spacer sequence will include nonpolar amino acids, though polar residues such as Glu, Gln, Ser, His, and Asn could also be present, particularly for spacer sequences longer than three residues. The only outer limit on the total length and nature of each spacer sequence derives from considerations of ease of synthesis, proteolytic processing, and manipulation of the polypeptide.

Moreover, the present invention also contemplates a polypeptide comprising or consisting of multiple repeats of any of the peptides as defined above or combinations of any of the peptides as defined above.

Minigene

A further embodiment of the present invention relates to a nucleic acid encoding a peptide selected from Tables 13 and 14. Said nucleic acids are “isolated” or “synthetic”. The term “isolated” refers to material that is substantially free from components that normally accompany it as found in its naturally occurring environment. However, it should be clear that the isolated nucleic acid of the present invention might comprise heterologous cell components or a label and the like. The terms “nucleic acid” or “polynucleic acid” are used interchangeable throughout the present application and refer to a deoxyribonucleotide or ribonucleotide polymer in either single- or double stranded form, which may encompass known analogues of natural nucleotides.

More particular, the present invention relates to a “minigene” or a polynucleotide that encodes a polyepitopic peptide as described herein. The term “multi-epitope construct” when referring to nucleic acids can be used interchangeably with the terms “polynucleotides”, “minigene” and “multi-epitope nucleic acid vaccine,” and other equivalent phrases, and comprises multiple epitope nucleic acids that encode peptide epitopes of any length that can bind to a molecule functioning in the immune system, preferably a HLA class I and a T-cell receptor or a HLA class II and a T-cell receptor. The epitope nucleic acids in a multi-epitope construct can encode HLA class I epitopes, HLA class II epitopes, a combination of HLA class I and class II epitopes or a nested epitope. HLA class I-encoding epitope nucleic acids are referred to as CTL epitope nucleic acids, and HLA class II-encoding epitope nucleic acids are referred to as HTL epitope nucleic acids. Some multi-epitope constructs can have a subset of the multi-epitope nucleic acids encoding HLA class I epitopes and another subset of the multi-epitope nucleic acids encoding HLA class II epitopes. A multi-epitope construct may have one or more spacer nucleic acids. A spacer nucleic acid may flank each epitope nucleic acid in a construct. The spacer nucleic acid may encode one or more amino acids (spacer amino acids). Alternatively, minigenes can be constructed using the technology as described by Qi-Liang Cai et al., 2004.

Accordingly, the present invention relates to a polynucleotide or minigene encoding a polyepitopic peptide comprising at least one peptide selected from Tables 13 and 14 or comprising at least one nested epitope selected from Table A.

Furthermore, the invention also encompasses a polynucleotide or minigene encoding a polyepitopic peptide comprising at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60 or more peptides. Preferably, the peptides are selected from Tables 13 and 14. Any combination of peptides is possible as described for the polyepitopic peptide. Hence, the polynucleotide or minigene can also encode one or more nested epitopes, or fragments thereof, for example as given in Table A.

More particular, the nucleic acids of the invention can be incorporated in an HLA-group restricted construct. Said “HLA-group restricted construct” comprises at least two nucleic acid epitopes encoding peptides binding to an allele or molecule of the same HLA group. The number of epitopes in a HLA-group restricted construct is not limited and can be 2, 3, 4, 5, 6, 7, 8, 9, 10, 11, 12, 13, 14, 15, 20, 25 or more. The same combinations are possible as described for the HLA-group restricted polyepitopic peptide.

In a preferred embodiment, the polyepitopic peptide encoded by the polynucleotide further comprises at least one HLA-class I binding peptide, a HLA class II binding peptide or a HCV derived peptide. Said HLA Class I binding peptide and said HLA Class II binding peptide can be derived from a foreign antigen or organism (non-HCV). There is no limitation on the length of said peptide, this can have a length of e.g. 5, 6, 7, 8, 9, 10, 11, 12, 13, 14, 15, 16, 17, 18, 19, 20, 21, 22, 23, 24, 25, 30 or more amino acids.

In a further embodiment, the polynucleotide or minigene as described herein can further comprise one or more spacer nucleic acids, i.e. 1, 2, 3, 4, 5, 6, 7, 8, 9, 10 or more. In a particular embodiment, the minigene further comprises one or more regulatory sequences and/or one or more signal sequences and/or one or more promotor sequences.

Polynucleotides or nucleic acids that are not commercially available can be chemically synthesized according to the solid phase phosphoramidite triester method first described by Beaucage & Caruthers, 1981, using an automated synthesizer, as described in Van Devanter et. al., 1984. Purification of polynucleotides is by either native acrylamide gel electrophoresis or by anion-exchange HPLC as described in Pearson & Reanier, 1983. Other purification methods are reversed phase separation and hydroxyapatite and are well known to the skilled person. Chemically synthesized and purified polynucleotides can be assembled into longer polynucleotides by PCR-based methods (Stemmer et al., 1995; Kriegler et al., 1991). The epitopes of the multi-epitope constructs are typically subcloned into an expression vector that contains a promoter to direct transcription, as well as other regulatory sequences such as enhancers and polyadenylation sites. Additional elements of the vector are e.g. signal or target sequences, translational initiation and termination sequences, 5′ and 3′ untranslated regions and introns, required for expression of the multi-epitope construct in host cells.

For therapeutic or prophylactic immunization purposes, the (polyepitopic) peptides of the invention can be expressed by plasmid vectors as well as viral or bacterial vectors as already described herein. The term “vector” may comprise a plasmid, a cosmid, a prokaryotic organism, a phage, or an eukaryotic organism such as a virus, an animal or human cell or a yeast cell. The expression vector typically contains a transcription unit or expression cassette that contains all the additional elements required for the expression of the multi-epitope construct in host cells. A typical expression cassette thus contains a promoter operably linked to the multi-epitope construct and signals required for efficient polyadenylation of the transcript. Additional elements of the cassette may include enhancers and introns with functional splice donor and acceptor sites.

Suitable promoters are well known in the art and described, e.g., in Sambrook et al., Molecular cloning, A Laboratory Manual (2^{nd} ed. 1989) and in Ausubel et al, Current Protocols in Molecular Biology (1994). Eukaryotic expression systems for mammalian cells are well known in the art and are commercially available. Such promoter elements include, for example, cytomegalovirus (CMV), Rous sarcoma virus long terminal repeats (RSV LTR) and Simian Virus 40 (SV40). See, e.g., U.S. Pat. Nos. 5,580,859 and 5,589,466 for other suitable promoter sequences.

In addition to a promoter sequence, the expression cassette can also contain a transcription termination region downstream of the structural gene to provide for efficient termination. The termination region may be obtained from the same gene as the promoter sequence or may be obtained from different genes.

Medical Use

In a further embodiment, the present invention also relates to the (polyepitopic) peptide, nested epitope, nucleic acid, minigene or composition of the present invention for use as a medicament. Preferably, said medicament is a vaccine. In a specific embodiment the invention also relates to a vector, a plasmid, a recombinant virus or host cell comprising the nucleic acid or minigene as described herein for use a medicament. More specifically, the present invention relates to the use of at least one of the peptides selected from Tables 13 and 14 or the nucleic acid sequence encoding said peptide for the manufacture of a medicament for preventing or treating a HCV infection. In a specific embodiment the invention also relates to a vector, a plasmid, a recombinant virus or host cell comprising the nucleic acid or minigene as described herein for the manufacture of a medicament for preventing or treating a HCV infection.

Vaccines and Vaccine Compositions

The invention furthermore relates to compositions comprising any of the HCV (polyepitopic) peptides as described herein or the corresponding nucleic acids. In a specific embodiment, the composition furthermore comprises at least one of a pharmaceutically acceptable carrier, adjuvant or vehicle. The terms “composition”, “immunogenic composition” and “pharmaceutical composition” are used interchangeable with “vaccine composition” or “vaccine”. There are numerous embodiments of vaccines in accordance with the invention, such as by a cocktail of one or more peptides, one or more epitopes of the invention comprised in a polyepitopic peptide, and/or nucleic acids that encode such peptides or polypeptides, e.g., a minigene that encodes a polyepitopic peptide. Vaccines can also comprise peptide-pulsed antigen presenting cells, e.g., the epitope can be bound to an HLA molecule on dendritic cells. More particularly, said immunogenic composition is a vaccine composition. Even more particularly, said vaccine composition is a prophylactic vaccine composition. Alternatively, said vaccine composition may also be a therapeutic vaccine composition. The prophylactic vaccine composition refers to a vaccine composition aimed for preventing HCV infection and to be administered to healthy persons who are not yet infected with HCV. The therapeutic vaccine composition refers to a vaccine composition aimed for treatment of HCV infection and to be administered to patients being infected with HCV.

A vaccine or vaccine composition is an immunogenic composition capable of eliciting an immune response sufficiently broad and vigorous to provoke at least one or both of:
a stabilizing effect on the multiplication of a pathogen already present in a host and against which the vaccine composition is targeted. A vaccine composition may also induce an immune response in a host already infected with the pathogen against which the immune response leading to stabilization, regression or resolving of the disease; and
an increase of the rate at which a pathogen newly introduced in a host, after immunization with a vaccine composition targeted against said pathogen, is resolved from said host.

A vaccine composition may also provoke an immune response broad and strong enough to exert a negative effect on the survival of a pathogen already present in a host or broad and strong enough to prevent an immunized host from developing disease symptoms caused by a newly introduced pathogen. In particular the vaccine composition of the invention is a HCV vaccine composition. In particular, the vaccine or vaccine composition comprises an effective amount of the peptides or nucleic acids of the present invention. In a specific embodiment, said vaccine composition comprises a vector, a plasmid, a recombinant virus or host cell comprising the nucleic acid or minigene of the present invention. Said vaccine composition may additionally comprise one or more further active substances and/or at least one of a pharmaceutically acceptable carrier, adjuvant or vehicle.

An “effective amount” of a peptide or nucleic acid in a vaccine or vaccine composition is referred to as an amount required and sufficient to elicit an immune response. It will be clear to the skilled artisan that the immune response sufficiently broad and vigorous to provoke the effects envisaged by the vaccine composition may require successive (in time) immunizations with the vaccine composition as part of a vaccination scheme or vaccination schedule. The “effective amount” may vary depending on the health and physical condition of the individual to be treated, the age of the individual to be treated (e.g. dosing for infants may be lower than for adults) the taxonomic group of the individual to be treated (e.g. human, non-human primate, primate, etc.), the capacity of the individual's immune system to mount an effective immune response, the degree of protection desired, the formulation of the vaccine, the treating doctor's assessment, the strain of the infecting pathogen and other relevant factors. It is expected that the effective amount of the vaccine composition will fall in a relatively broad range that can be determined through routine trials, i.e. 0,01-50 mg/dose; more preferably between 0,1-5 mg/dose. Usually, the amount will vary from 0,01 to 1000 μg/dose, more particularly from 0,1 to 100 μg/dose. Dosage treatment may be a single dose schedule or a multiple dose schedule. The vaccine may be administered in conjunction with other immunoregulatory agents. The dosages, routes of administration, and dose schedules are adjusted in accordance with methodologies known in the art.

A composition or vaccine composition may comprise more than one peptide or nucleic acid, i.e., a plurality thereof, e.g. 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34 or more, e.g., up to 35, 36, 37, 38, 39, 40, 41, 42, 43, 44, 45, 46, 47, 48, 49, 50 or more distinct peptides or nucleic acids.

Carriers, Adjuvants and Vehicles—Delivery

Once appropriately immunogenic peptides, or the nucleic acids encoding them, have been defined, they can be sorted and delivered by various means, herein referred to as “compositions”, “vaccine compositions” or “pharmaceutical compositions”. The peptides of the present invention and pharmaceutical and vaccine compositions of the invention are usefull for administration to mammals, particularly humans, to treat and/or prevent HCV infection. Vaccine compositions containing the peptides of the invention, or the DNA encoding them, are administered to a patient infected with HCV or to an individual susceptible to, or otherwise at risk for, HCV infection to elicit an immune response against HCV antigens and thus enhance the patient's own immune response capabilities.

Various art-recognized delivery systems may be used to deliver peptides, polyepitopic polypeptides, or polynucleotides encoding peptides or polyepitope polypeptides, into appropriate cells. The peptides and nucleic acids encoding them can be delivered in a pharmaceutically acceptable carrier or as colloidal suspensions, or as powders, with or without diluents. They can be “naked” or associated with delivery vehicles and delivered using delivery systems known in the art.

A “pharmaceutically acceptable carrier” or “pharmaceutically acceptable adjuvant” is any suitable excipient, diluent, carrier and/or adjuvant which, by themselves, do not induce the production of antibodies harmful to the individual receiving the composition nor do they elicit protection. Preferably, a pharmaceutically acceptable carrier or adjuvant enhances the immune response elicited by an antigen. Suitable carriers or adjuvantia typically comprise one or more of the compounds included in the following non-exhaustive list: large slowly metabolized macromolecules such as proteins, polysaccharides, polylactic acids, polyglycolic acids, polymeric amino acids, amino acid copolymers and inactive virus particles; aluminium hydroxide, aluminium phosphate (see International Patent Application Publication No. WO93/24148), alum (KA1(SO₄)₂.12H₂O), or one of these in combination with 3-0-deacylated monophosphoryl lipid A (see International Patent Application Publication No. WO93/19780); N-acetyl-muramyl-L-threonyl-D-isoglutamine (see U.S. Pat. No. 4,606,918), N-acetyl-normuramyl-L-alanyl-D-isoglutamine, N-acetylmuramyl-L-alanyl-D-isoglutamyl-L-alanine2-(1′,2′-dipalmitoyl-sn-glycero-3-hydroxyphosphoryloxy) ethylamine; RIBI (ImmunoChem Research Inc., Hamilton, Mont., USA) which contains monophosphoryl lipid A (i.e., a detoxified endotoxin), trehalose-6,6-dimycolate, and cell wall skeleton (MPL+TDM+CWS) in a 2% squalene/Tween 80 emulsion. Any of the three components MPL, TDM or CWS may also be used alone or combined 2 by 2; adjuvants such as Stimulon (Cambridge Bioscience, Worcester, Mass., USA), SAF-1 (Syntex); adjuvants such as combinations between QS21 and 3-de-O-acetylated monophosphoryl lipid A (see International Application No. WO94/00153) which may be further supplemented with an oil-in-water emulsion (see, e.g., International Application Nos. WO95/17210, WO97/01640 and WO9856414) in which the oil-in-water emulsion comprises a metabolisable oil and a saponin, or a metabolisable oil, a saponin, and a sterol, or which may be further supplemented with a cytokine (see International Application No. WO98/57659); adjuvants such as MF-59 (Chiron), or poly[di(carboxylatophenoxy) phosphazene] based adjuvants (Virus Research Institute); blockcopolymer based adjuvants such as Optivax (Vaxcel, Cytrx) or inulin-based adjuvants, such as Algammulin and Gammalnulin (Anutech); Complete or Incomplete Freund's Adjuvant (CFA or IFA, respectively) or Gerbu preparations (Gerbu Biotechnik); a saponin such as QuilA, a purified saponin such as QS21, QS7 or QS17, β-escin or digitonin; immunostimulatory oligonucleotides comprising unmethylated CpG dinucleotides such as [purine-purine-CG-pyrimidine-pyrimidine] oligonucleotides. These immunostimulatory oligonucleotides include CpG class A, B, and C molecules (Coley Pharmaceuticals), ISS (Dynavax), Immunomers (Hybridon). Immunostimulatory oligonucleotides may also be combined with cationic peptides as described, e.g., by Riedl et al. (2002); Immune Stimulating Complexes comprising saponins, for example Quil A (ISCOMS); excipients and diluents, which are inherently non-toxic and non-therapeutic, such as water, saline, glycerol, ethanol, wetting or emulsifying agents, pH buffering substances, preservatives, and the like; a biodegradable and/or biocompatible oil such as squalane, squalene, eicosane, tetratetracontane, glycerol, peanut oil, vegetable oil, in a concentration of, e.g., 1 to 10% or 2,5 to 5%; vitamins such as vitamin C (ascorbic acid or its salts or esters), vitamin E (tocopherol), or vitamin A; carotenoids, or natural or synthetic flavanoids; trace elements, such as selenium; any Toll-like receptor ligand as reviewed in Barton and Medzhitov (2002).

Any of the afore-mentioned adjuvants comprising 3-de-O-acetylated monophosphoryl lipid A, said 3-de-O-acetylated monophosphoryl lipid A may be forming a small particle (see International Application No. WO94/21292).

In any of the aforementioned adjuvants MPL or 3-de-O-acetylated monophosphoryl lipid A can be replaced by a synthetic analogue referred to as RC-529 or by any other amino-alkyl glucosaminide 4-phosphate (Johnson et al. 1999, Persing et al. 2002). Alternatively it can be replaced by other lipid A analogues such as OM-197 (Byl et al. 2003).

A “pharmaceutically acceptable vehicle” includes vehicles such as water, saline, physiological salt solutions, glycerol, ethanol, etc. Auxiliary substances such as wetting or emulsifying agents, pH buffering substances, preservatives may be included in such vehicles. Delivery systems known in the art are e.g. lipopeptides, peptide compositions encapsulated in poly-DL-lactide-co-glycolide (“PLG”), microspheres, peptide compositions contained in immune stimulating complexes (ISCOMS), multiple antigen peptide systems (MAPs), viral delivery vectors, particles of viral or synthetic origin, adjuvants, liposomes, lipids, microparticles or microcapsules, gold particles, nanoparticles, polymers, condensing agents, polysaccharides, polyamino acids, dendrimers, saponins, QS21, adsorption enhancing materials, fatty acids or, naked or particle absorbed cDNA.

Typically, a vaccine or vaccine composition is prepared as an injectable, either as a liquid solution or suspension. Injection may be subcutaneous, intramuscular, intravenous, intraperitoneal, intrathecal, intradermal, intraepidermal, or by “gene gun”. Other types of administration comprise electroporation, implantation, suppositories, oral ingestion, enteric application, inhalation, aerosolization or nasal spray or drops. Solid forms, suitable for dissolving in, or suspension in, liquid vehicles prior to injection may also be prepared. The preparation may also be emulsified or encapsulated in liposomes for enhancing adjuvant effect.

A liquid formulation may include oils, polymers, vitamins, carbohydrates, amino acids, salts, buffers, albumin, surfactants, or bulking agents. Preferably carbohydrates include sugar or sugar alcohols such as mono-, di-, or polysaccharides, or water-soluble glucans. The saccharides or glucans can include fructose, dextrose, lactose, glucose, mannose, sorbose, xylose, maltose, sucrose, dextran, pullulan, dextrin, alpha and beta cyclodextrin, soluble starch, hydroxethyl starch and carboxymethylcellulose, or mixtures thereof. Sucrose is most preferred. “Sugar alcohol” is defined as a C4 to C8 hydrocarbon having an —OH group and includes galactitol, inositol, mannitol, xylitol, sorbitol, glycerol, and arabitol. Mannitol is most preferred. These sugars or sugar alcohols mentioned above may be used individually or in combination. There is no fixed limit to the amount used as long as the sugar or sugar alcohol is soluble in the aqueous preparation. Preferably, the sugar or sugar alcohol concentration is between 1,0% (w/v) and 7,0% (w/v), more preferable between 2,0 and 6,0% (w/v). Preferably amino acids include levorotary (L) forms of carnitine, arginine, and betaine; however, other amino acids may be added. Preferred polymers include polyvinylpyrrolidone (PVP) with an average molecular weight between 2,000 and 3,000, or polyethylene glycol (PEG) with an average molecular weight between 3,000 and 5,000. It is also preferred to use a buffer in the composition to minimize pH changes in the solution before lyophilization or after reconstitution. Any physiological buffer may be used, but citrate, phosphate, succinate, and glutamate buffers or mixtures thereof are preferred. Most preferred is a citrate buffer. Preferably, the concentration is from 0,01 to 0,3 molar. Surfactants that can be added to the formulation are shown in EP patent applications No. EP 0 270 799 and EP 0 268 110.

Additionally, polypeptides can be chemically modified by covalent conjugation to a polymer to increase their circulating half-life, for example. Preferred polymers, and methods to attach them to peptides, are shown in U.S. Pat. Nos. 4,766,106; 4,179,337; 4,495,285; and 4,609,546. Preferred polymers are polyoxyethylated polyols and polyethylene glycol (PEG). PEG is soluble in water at room temperature and has the general formula: R(O—CH₂—CH₂)ₙO—R where R can be hydrogen, or a protective group such as an alkyl or alkanol group. Preferably, the protective group has between 1 and 8 carbons, more preferably it is methyl. The symbol n is a positive integer, preferably between 1 and 1.000, more preferably between 2 and 500. The PEG has a preferred average molecular weight between 1000 and 40.000, more preferably between 2000 and 20.000, most preferably between 3.000 and 12.000. Preferably, PEG has at least one hydroxy group, more preferably it is a terminal hydroxy group. It is this hydroxy group which is preferably activated. However, it will be understood that the type and amount of the reactive groups may be varied to achieve a covalently conjugated PEG/polypeptide of the present invention.

Water soluble polyoxyethylated polyols are also useful in the present invention. They include polyoxyethylated sorbitol, polyoxyethylated glucose, polyoxyethylated glycerol (POG), etc. POG is preferred. One reason is because the glycerol backbone of polyoxyethylated glycerol is the same backbone occurring naturally in, for example, animals and humans in mono-, di-, triglycerides. Therefore, this branching would not necessarily be seen as a foreign agent in the body. The POG has a preferred molecular weight in the same range as PEG. The structure for POG is shown in Knauf et al., 1988, and a discussion of POG/IL-2 conjugates is found in U.S. Pat. No. 4,766,106.

Another drug delivery system for increasing circulatory half-life is the liposome. The peptides and nucleic acids of the invention may also be administered via liposomes, which serve to target a particular tissue, such as lymphoid tissue, or to target selectively infected cells, as well as to increase the half-life of the peptide and nucleic acids composition. Liposomes include emulsions, foams, micelles, insoluble monolayers, liquid crystals, phospholipid dispersions, lamellar layers and the like. In these preparations, the peptide or nucleic acids to be delivered is incorporated as part of a liposome or embedded, alone or in conjunction with a molecule which binds to a receptor prevalent among lymphoid cells, such as monoclonal antibodies which bind to the CD45 antigen, or with other therapeutic or immunogenic compositions. Thus, liposomes either filled or decorated with a desired peptide or nucleic acids of the invention can be directed to the site of lymphoid cells, where the liposomes then deliver the peptide and nucleic acids compositions. Liposomes for use in accordance with the invention are formed from standard vesicle-forming lipids, which generally include neutral and negatively charged phospholipids and a sterol, such as cholesterol. The selection of lipids is generally guided by consideration of, e.g., liposome size, acid lability and stability of the liposomes in the blood stream. A variety of methods are available for preparing liposomes, as described in, e.g., Szoka et al, 1980, and U.S. Pat. Nos. 4,235,871, 4,501,728, 4,837,028, and 5,019,369.

For targeting cells of the immune system, a ligand to be incorporated into the liposome can include, e.g., antibodies or fragments thereof specific for cell surface determinants of the desired immune system cells. A liposome suspension containing a peptide may be administered intravenously, locally, topically, etc. in a dose which varies according to, inter alia, the manner of administration, the peptide being delivered, and the stage of the disease being treated. For example, liposomes carrying either immunogenic polypeptides or nucleic acids encoding immunogenic epitopes are known to elicit CTL responses in vivo (Reddy et al., 1992; Collins et al., 1992; Fries et al., 1992; Nabel et al., 1992).

After the liquid pharmaceutical composition is prepared, it is preferably lyophilized to prevent degradation and to preserve sterility. Methods for lyophilizing liquid compositions are known to those of ordinary skill in the art. Just prior to use, the composition may be reconstituted with a sterile diluent (Ringer's solution, distilled water, or sterile saline, for example) which may include additional ingredients. Upon reconstitution, the composition is preferably administered to subjects using those methods that are known to those skilled in the art.

The approach known as “naked DNA” is currently being used for intramuscular (IM) administration in clinical trials. To maximize the immunotherapeutic effects of minigene DNA vaccines, an alternative method for formulating purified plasmid DNA may be desirable. A variety of methods have been described, and new techniques may become available. Cationic lipids can also be used in the formulation (see, e.g., as described by WO 93/24640; Mannino & Gould-Fogerite 1988; U.S. Pat. No. 5,279,833; WO 91/06309; and Felgner et al., 1987. In addition, glycolipids, fusogenic liposomes, peptides and compounds referred to collectively as protective, interactive, non-condensing compounds could also be complexed to purified plasmid DNA to influence variables such as stability, intramuscular dispersion, or trafficking to specific organs or cell types.

Further examples of DNA-based delivery technologies include facilitated (bupivicaine, polymers, peptide-mediated) delivery, cationic lipid complexes, particle-mediated (“gene gun”) or pressure-mediated delivery (see, e.g., U.S. Pat. No. 5,922,687), DNA formulated with charged or uncharged lipids, DNA formulated in liposomes, emulsified DNA, DNA included in a viral vector, DNA formulated with a transfection-facilitating protein or polypeptide, DNA formulated with a targeting protein or polypeptide, DNA formulated with calcium precipitating agents, DNA coupled to an inert carrier molecule, and DNA formulated with an adjuvant. In this context it is noted that practically all considerations pertaining to the use of adjuvants in traditional vaccine formulation apply to the formulation of DNA vaccines.

Recombinant virus or live carrier vectors may also be directly used as live vaccines in humans. Accordingly the present invention also relates to a recombinant virus, an expression vector or a plasmid, and a host cell comprising the nucleic acid encoding at least one of the peptides as disclosed in Tables 13 and 14.

In a preferred embodiment of the invention, the nucleic acid or minigene is introduced in the form of a vector wherein expression is under control of a viral promoter. Therefore, further embodiments of the present invention are an expression vector which comprises a polynucleotide encoding at least one of the herein described peptides and which is capable of expressing the respective peptides, a host cell comprising the expression vector and a method of producing and purifying herein described peptides, pharmaceutical compositions comprising the herein described peptides and a pharmaceutically acceptable carrier and/or adjuvants. The “peptides as described herein” refer to the peptides disclosed in Tables 13 and 14.

Detailed disclosures relating to the formulation and use of nucleic acid vaccines are available, e.g. by Donnelly J. J. et al, 1997 and 1997a. Examples of expression vectors include attenuated viral hosts, such as vaccinia or fowlpox. As an example of this approach, vaccinia virus is used as a vector to express nucleotide sequences that encode the peptides of the invention. Upon introduction into a host, the recombinant vaccinia virus expresses the immunogenic peptide, and thereby elicits a host CTL and/or HTL response. Vaccinia vectors, for example Modified Vaccinia Ankara (MVA), and methods useful in immunization protocols are described in, e.g., U.S. Pat. No. 4,722,848. Another vector is BCG (Bacille Calmette Guerin). BCG vectors are described in Stover et al., 1991. Further examples are: Alphaviruses (Semliki Forest Virus, Sindbis Vrius, Venezuelan Equine Encephalitis Virus (VEE)), Transgene Herpes simplex Virus (HSV), replication-deficient strains of Adenovirus (human or simian), SV40 vectors, CMV vectors, papilloma virus vectors, and vectors derived from Epstein Barr virus. A wide variety of other vectors useful for therapeutic administration or immunization of the peptides of the invention, e.g. retroviral vectors, Salmonella typhi vectors, detoxified anthrax toxin vectors, and the like, will be apparent to those skilled in the art from the description herein.

Additional vector modifications may be desired to optimize minigene expression and immunogenicity. In some cases, introns are required for efficient gene expression, and one or more synthetic or naturally-occurring introns could be incorporated into the transcribed region of the minigene. The inclusion of mRNA stabilization sequences and sequences for replication in mammalian cells may also be considered for increasing minigene expression. In addition, immunostimulatory sequences (ISSs or CpGs) appear to play a role in the immunogenicity of nucleic acid vaccines. These sequences may be included in the vector, outside the minigene coding sequence, if desired to enhance immunogenicity. In some embodiments, a bi-cistronic expression vector which allows production of both the minigene-encoded epitopes and a second protein (included to enhance or decrease immunogenicity) can be used. Examples of proteins or polypeptides that could beneficially enhance the immune response if co-expressed include cytokines (e.g., IL-2, IL-12, GM-CSF), cytokine-inducing molecules (e.g., LeIF), costimulatory molecules, or for HTL responses, pan-DR binding proteins (PADRE®, Epimmune, San Diego, Calif.). Helper (HTL) epitopes can be joined to intracellular targeting signals and expressed separately from expressed CTL epitopes; this allows direction of the HTL epitopes to a cell compartment different than that of the CTL epitopes. If required, this could facilitate more efficient entry of HTL epitopes into the HLA class II pathway, thereby improving HTL induction. In contrast to HTL or CTL induction, specifically decreasing the immune response by co-expression of immunosuppressive molecules (e.g. TGF-P) may be beneficial in certain diseases.

The use of multi-epitope minigenes is described in, e.g., U.S. Pat. No. 6,534,482; An and Whitton, 1997; Thomson et al., 1996; Whitton et al., 1993; Hanke et al., 1998. For example, a multi-epitope DNA plasmid encoding supermotif- and/or motif-bearing HCV epitopes derived from multiple regions of the HCV polyprotein sequence, the PADRE® universal helper T cell epitope (or multiple HTL epitopes from HCV), and an endoplasmic reticulum-translocating signal sequence can be engineered.

The nucleic acids or minigenes encoding the peptides or polyepitopic polypeptides, or the peptides or polyepitopic peptides themselves, can be administered alone or in combination with other therapies known in the art. In addition, the polypeptides and nucleic acids of the invention can be administered in combination with other treatments designed to enhance immune responses, e.g., by co-administration with adjuvants or cytokines (or nucleic acids encoding cytokines), as is well known in the art. Accordingly, the peptides or nucleic acids or vaccine compositions of the invention can also be used in combination with antiviral drugs such as interferon, or other treatments for viral infection.

All disclosures herein which relate to use of adjuvants in the context of protein or (poly)peptide based pharmaceutical compositions apply mutatis mutandis to their use in nucleic acid vaccination technology. The same holds true for other considerations relating to formulation and mode and route of administration and, hence, also these considerations discussed herein in connection with a traditional pharmaceutical composition apply mutatis mutandis to their use in nucleic acid vaccination technology.

In a further embodiment, the present invention relates to the use of the peptide and/or nucleic acid as described herein for inducing immunity against HCV, characterized in that said peptide and/or nucleic acid is used as part of a series of time and compounds. In this regard, it is to be understood that the term “a series of time and compounds” refers to administering with time intervals to an individual the compounds used for eliciting an immune response. The latter compounds may comprise any of the following components: a peptide or polyepitopic peptide, a nucleic acid or minigene or a vector. In this respect, a series comprises administering, either:
(i) a peptide or polyepitopic peptide, or
(ii) a nucleic acid, minigene or vector, wherein said nucleic acid, minigene or vector can be administered simultaneously, or at different time intervals, including at alternating time intervals, or
(iii) a peptide or polyepitopic peptide in combination with a nucleic acid, minigene or vector, wherein said peptide or polyepitopic peptide and said nucleic acid, minigene or vector can be administered simultaneously, or at different time intervals, including at alternating time intervals, or
(iv) either (i) or (ii), possibly in combination with other peptides or nucleic acids or vectors, with time intervals.

The peptide and nucleic acid compositions of this invention can be provided in kit form together with instructions for vaccine administration. Typically the kit would include desired peptide compositions in a container, preferably in unit dosage form and instructions for administration. An alternative kit would include a minigene construct with desired nucleic acids of the invention in a container, preferably in unit dosage form together with instructions for administration. Lymphokines such as IL-2 or IL-12 may also be included in the kit. Other kit components that may also be desirable include, for example, a sterile syringe, booster dosages, and other desired excipients.

Use of the Peptides for Evaluating Immune Responses.

The peptides may also find use as diagnostic reagents. For example, a peptide of the invention may be used to determine the susceptibility of a particular individual to a treatment regimen which employs the peptide, related peptides or any other HCV vaccine, and thus may be helpful in modifying an existing treatment protocol or in determining a prognosis for an affected individual. In addition, the peptides may also be used to predict which individuals will be at substantial risk for developing chronic HCV infection.

Accordingly, the present invention relates to a method of determining the outcome for a subject exposed to HCV, comprising the steps of determining whether the subject has an immune response to one or more peptides selected from Tables 13 and 14.

In a preferred embodiment of the invention, the peptides as described herein can be used as reagents to evaluate an immune response. The immune response to be evaluated can be induced by the natural infection or by using as an immunogen any agent that may result in the production of antigen-specific CTLs or HTLs that recognize and bind to the peptide(s) to be employed as the reagent. The peptide reagent need not be used as the immunogen. Assay systems that can be used for such an analysis include relatively recent technical developments such as tetramers, staining for intracellular lymphokines and interferon release assays, or ELISPOT assays.

For example, a peptide of the invention may be used in a tetramer staining assay to assess peripheral blood mononuclear cells for the presence of antigen-specific CTLs following exposure to an antigen or an immunogen. The HLA-tetrameric complex is used to directly visualize antigen-specific CTLS (see, e.g., Ogg et al., 1998; and Altman et al., 1996) and determine the frequency of the antigen-specific CTL population in a sample of peripheral blood mononuclear cells. A tetramer reagent using a peptide of the invention may be generated as follows: a peptide that binds to an HLA molecule is refolded in the presence of the corresponding HLA heavy chain and beta2-microglobulin to generate a trimolecular complex. The complex is biotinylated at the carboxyl terminal end of the heavy chain at a site that was previously engineered into the protein. Tetramer formation is then induced by the addition of streptavidin. By means of fluorescently labeled streptavidin, the tetramer can be used to stain antigen-specific cells. The cells may then be identified, for example, by flow cytometry. Such an analysis may be used for diagnostic or prognostic purposes. Cells identified by the procedure can also be used for therapeutic purposes. As an alternative to tetramers also pentamers or dimers can be used (Current Protocols in Immunology (2000) unit 17.2 supplement 35)

Peptides of the invention may also be used as reagents to evaluate immune recall responses. (see, e.g., Bertoni et al., 1997 and Perma et al., 1991.). For example, patient PBMC samples from individuals with HCV infection may be analyzed for the presence of antigen-specific CTLs or HTLs using specific peptides. A blood sample containing mononuclear cells may be evaluated by cultivating the PBMCs and stimulating the cells with a peptide of the invention. After an appropriate cultivation period, the expanded cell population may be analyzed, for example, for cytotoxic activity (CTL) or for HTL activity.

The peptides may also be used as reagents to evaluate the efficacy of a vaccine. PBMCs obtained from a patient vaccinated with an immunogen may be analyzed using, for example, either of the methods described above. The patient is HLA typed, and peptide epitope reagents that recognize the allele-specific molecules present in that patient are selected for the analysis. The immunogenicity of the vaccine is indicated by the presence of epitope-specific CTLs and/or HTLs in the PBMC sample.

The peptides of the invention may also be used to make antibodies, using techniques well known in the art (see, e.g. CURRENT PROTOCOLS IN IMMUNOLOGY, Wiley/Greene, NY; and Antibodies A Laboratory Manual, Harlow and Lane, Cold Spring Harbor Laboratory Press, 1989). Such antibodies include those that recognize a peptide in the context of an HLA molecule, i.e., antibodies that bind to a peptide-MHC complex.

Tables

The peptides of current invention are set out in Tables 1-14. As used herein, “CS_fr” and “CS_to” means Consensus Sequence “from” and “to” residue numbers of the HCV consensus sequence as disclosed in FIG. 1 or 2.

S: Strong, Kdpred <0, 1 μM; M: Medium, Kdpred 0,1-1 μM; W: Weak, Kdpred 1-10 μM

TABLE 1

Predicted HLA-A*0101 binding peptides

| | | | | | |
|---|---|---|---|---|---|
| | | | | | SEQ ID |
| Protein | CS_fr | CS_to | pep_seq | Score | NO |

Algonomics
9-mer

| | | | | | | |
|---|---|---|---|---|---|---|
| 1 | NS3 | 1436 | 1444 | ATDALMTGY | S | 1 |
| 2 | C | 126 | 134 | LTCGFADLM | S | 2 |
| 3 | NS5B | 2588 | 2596 | RVCEKMALY | S | 3 |
| 4 | C | 130 | 138 | FADLMGYIP | M | 4 |
| 5 | NS3 | 1565 | 1573 | LTHIDAHFL | M | 5 |
| 6 | NS3 | 1285 | 1293 | ITTGAPITY | M | 6 |
| 7 | NS3 | 1210 | 1218 | FTDNSSPPA | M | 7 |
| 8 | NS3 | 1581 | 1589 | DNFPYLVAY | M | 8 |
| 9 | NS5B | 2759 | 2767 | FTEAMTRYS | M | 9 |
| 10 | NS5B | 2795 | 2803 | DASGKRVYY | M | 10 |
| 11 | NS3 | 1288 | 1296 | GAPITYSTY | M | 11 |
| 12 | NS3 | 1241 | 1249 | PAAYAAQGY | M | 12 |
| 13 | NS3 | 1520 | 1528 | CYDAGCAWY | M | 13 |
| 14 | NS5B | 2835 | 2843 | YAPTLWARM | M | 14 |
| 15 | NS3 | 1197 | 1205 | PVESMETTM | M | 15 |
| 16 | NS5B | 2605 | 2613 | AVMGSSYGF | M | 16 |
| 17 | NS3 | 1513 | 1521 | DSSVLCECY | M | 17 |
| 18 | NS3 | 1410 | 1418 | LGLNAVAYY | M | 18 |
| 19 | NS5B | 2770 | 2778 | PGDPPQPEY | M | 19 |
| 20 | NS3 | 1370 | 1378 | NGEIPFYGK | M | 20 |
| 21 | NS3 | 1635 | 1643 | VILTHPITK | M | 21 |
| 22 | NS5B | 2607 | 2615 | MGSSYGFQY | M | 22 |
| 23 | NS3 | 1637 | 1645 | LTHPITKYI | M | 23 |
| 24 | NS3 | 1579 | 1587 | AGDNFPYLV | M | 24 |
| 25 | NS3 | 1236 | 1244 | KSTKVPAAY | M | 25 |
| 26 | NS3 | 1291 | 1299 | ITYSTYGKF | M | 26 |
| 27 | NS3 | 1532 | 1540 | PAETSVRLR | M | 27 |
| 28 | C | 122 | 130 | VIDTLTCGF | M | 28 |
| 29 | NS3 | 1420 | 1428 | GLDVSVIPT | M | 29 |
| 30 | NS3 | 1466 | 1474 | LDPTFTIET | M | 30 |
| 31 | C | 158 | 166 | LEDGVNYAT | W | 31 |
| 32 | NS3 | 1260 | 1268 | ATLGFGAYM | W | 32 |
| 33 | NS3 | 1602 | 1610 | PSWDQMWKC | W | 33 |
| 34 | NS5B | 2837 | 2845 | PTLWARMIL | W | 34 |
| 35 | NS3 | 1468 | 1476 | PTFTIETTT | W | 35 |
| 36 | NS5B | 2758 | 2766 | VFTEAMTRY | W | 36 |
| 37 | NS5B | 2603 | 2611 | PQAVMGSSY | W | 37 |
| 38 | NS5B | 2792 | 2800 | VAHDASGKR | W | 38 |
| 39 | NS5B | 2757 | 2765 | RVFTEAMTR | W | 39 |
| 40 | NS5B | 2710 | 2718 | GNTLTCYLK | W | 40 |
| 41 | NS5B | 2563 | 2571 | EVFCVQPEK | W | 41 |
| 42 | C | 172 | 180 | CSFSIFLLA | W | 42 |
| 43 | NS5B | 2615 | 2623 | YSPGQRVEF | W | 43 |
| 44 | NS3 | 1434 | 1442 | VVATDALMT | W | 44 |
| 45 | C | 156 | 164 | RVLEDGVNY | W | 45 |
| 46 | NS3 | 1534 | 1542 | ETSVRLRAY | W | 46 |
| 47 | NS3 | 1391 | 1399 | LIFCHSKKK | W | 47 |
| 48 | NS5B | 2662 | 2670 | CCDLAPEAR | W | 48 |
| 49 | NS5B | 2826 | 2834 | NSWLGNIIM | W | 49 |
| 50 | NS3 | 1262 | 1270 | LGFGAYMSK | W | 50 |
| 51 | NS3 | 1409 | 1417 | ALGLNAVAY | W | 51 |
| 52 | NS3 | 1199 | 1207 | ESMETTMRS | W | 52 |
| 53 | NS3 | 1437 | 1445 | TDALMTGYT | W | 53 |
| 54 | NS3 | 1195 | 1203 | FIPVESMET | W | 54 |
| 55 | C | 109 | 117 | PTDPRRRSR | W | 55 |
| 56 | NS3 | 1242 | 1250 | AAYAAQGYK | W | 56 |
| 57 | NS3 | 1203 | 1211 | TTMRSPVFT | W | 57 |
| 58 | NS3 | 1569 | 1577 | DAHFLSQTK | W | 58 |
| 59 | NS5B | 2842 | 2850 | RMILMTHFF | W | 59 |
| 60 | NS3 | 1335 | 1343 | QAETAGARL | W | 60 |
| 61 | NS3 | 1649 | 1657 | MSADLEVVT | W | 61 |

| | | | | | |
|---|---|---|---|---|---|
| | | | | Score | SEQ ID |
| Protein | CS_fr | CS_to | pep_seq | PIC | NO |

Epimmune

| | | | |
|---|---|---|---|
| | MSATLCSALY | 22 | 1507 |
| E1 | VQDCNCSIY | 16 | 1961 |
| E1 | VQECNCSIY | 24 | 1962 |
| E1 | TQDCNCSIY | 12 | 1864 |
| | DMRPYCWHY | 68 | 970 |
| | ASSVCGPVY | 56 | 874 |
| | TTDRSGAPTY | 88 | 1872 |
| | CTWMNSTGY | 21 | 947 |
| | CGAPPCNIY | 74 | 914 |
| E2 | LTPRCLVDY | 65 | 1474 |
| E2 | LTPRCLIDY | 32 | 1473 |
| E2 | FTIFKVRMY | 34 | 1089 |
| E2 | YTIFKIRMY | 26 | 2070 |
| E2 | FTIFKIRMY | 33 | 1088 |
| | GLSPAITKY | 15 | 1156 |
| | VLALPQQAY | 56 | 1926 |
| | LIAVLGPLY | 31 | 1384 |
| | LLALLGPAY | 30 | 1389 |
| | ISGVLWTVY | 42 | 1304 |
| NS3 | CTCGSSDLY | 18 | 940 |
| NS3 | CTCGAVDLY | 17 | 938 |
| NS3 | CTCGSADLY | 21 | 939 |
| NS3 | LLSPRPISY | 15 | 1408 |
| NS3 | KSTKVPAAY | 71 | 25 |
| NS3 | PAAYAAQGY | 29 | 12 |
| NS3 | PAAYVAQGY | 42 | 1544 |
| NS3 | ITIGAPITY | 13 | 6 |
| NS3 | ITTGSPITY | 15 | 1309 |
| NS3 | STTGEIPFY | 50 | 1791 |
| NS3 | GSEGEIPFY | 42 | 1185 |
| NS3 | GMGLNAVAY | 47 | 1159 |
| NS3 | ATDALMTGY | 32 | 1 |
| NS3 | DSSVLCECY | 31 | 17 |
| NS3 | DSVVLCECY | 83 | 987 |
| | ETTVRLRAY | 46 | 1035 |
| NS5A | CTPSPAPNY | 78 | 943 |
| NS5A | EVDGVRLHRY | 17 | 1036 |
| NS5A | ELDGVRLHRY | 23 | 1017 |
| | PLSNSLLRY | 21 | 1560 |
| NS5B | HSAKSKFGY | 24 | 1241 |
| NS5B | HSARSKFGY | 25 | 1242 |
| NS5B | MGSSYGFQY | 91 | 22 |
| NS5B | MGSAYGFQY | 98 | 1495 |
| NS5B | KKDPMGFSY | 77 | 1328 |
| NS5B | TSCGNTLTCY | 41 | 1867 |
| NS5B | TSFGNTITCY | 45 | 1868 |
| NS5B | DASGKRVYY | 55 | 10 |
| NS5B | GLSAFSLHSY | 47 | 1153 |
| NS5B | GLDAFSLHTY | 28 | 1148 |
| NS5B | GLSAFTLHSY | 43 | 1155 |
| NS5B | LSAFSLHSY | 9 | 1456 |
| NS5B | LDAFSLHTY | 32 | 1367 |
| NS5B | LSAFTLHSY | 9 | 1457 |
| NS5B | GRAAICGKY | 95 | 1179 |
| NS5B | LLSVGVGIY | 47 | 1411 |

| | | | | | |
|---|---|---|---|---|---|
| | | | | | SEQ ID |
| Protein | CS_fr | CS_to | pep_seq | Score | NO |

Algonomics
10-mer

| | | | | | |
|---|---|---|---|---|---|
| Ns5b | 2759 | 2768 | FTEAMTRYSA | S | 1087 |
| Ns5b | 2826 | 2835 | NSWLGNIIMY | M | 1534 |
| Ns4b | 1848 | 1857 | LVDILAGYGA | M | 1478 |
| Ns3 | 1436 | 1445 | ATDALMTGYT | M | 877 |
| Ns3 | 1617 | 1626 | TLHGPTPLLY | M | 1833 |
| Ns3 | 1435 | 1444 | VATDALMTGY | M | 1894 |
| Ns3 | 1210 | 1219 | FTDNSSPPAV | M | 1086 |
| Ns5b | 2757 | 2766 | RVFTEAMTRY | M | 1712 |
| Ns3 | 1635 | 1644 | VTLTHPITKY | M | 1976 |
| Ns3 | 1258 | 1267 | VAATLGFGAY | M | 1887 |
| Ns3 | 1409 | 1418 | ALGLNAVAYY | M | 822 |
| Ns3 | 1637 | 1646 | LTHPITKYIM | M | 1469 |
| Ns3 | 1240 | 1249 | VPAAYAAQGY | M | 1943 |
| Ns3 | 1519 | 1528 | ECYDAGCAWY | M | 1002 |
| Core | 122 | 131 | VIDTLTCGFA | M | 1914 |
| Ns3 | 1578 | 1587 | QAGDNFPYLV | W | 1596 |
| Ns5b | 2794 | 2803 | HDASGKRVYY | W | 1216 |
| Ns3 | 1408 | 1417 | SALGLNAVAY | W | 1717 |
| Ns5b | 2606 | 2615 | VMGSSYGFQY | W | 1939 |
| Ns3 | 1554 | 1563 | HLEFWESVFT | W | 1225 |
| Ns3 | 1367 | 1376 | LSNTGEIPFY | W | 1459 |
| Core | 127 | 136 | TCGFADLMGY | W | 1815 |
| Core | 130 | 139 | FADLMGYIPL | W | 1048 |
| Ns3 | 1433 | 1442 | VVVATDALMT | W | 1987 |
| Ns3 | 1465 | 1474 | SLDPTFTIET | W | 1741 |
| Ns5b | 2832 | 2841 | IIMYAPTLWA | W | 1268 |
| Ns3 | 1369 | 1378 | NTGEIPFYGK | W | 1535 |
| Ns5b | 2620 | 2629 | RVEFLVNAWK | W | 1711 |
| Ns5b | 2602 | 2611 | LPQAVMGSSY | W | 1437 |
| Core | 157 | 166 | VLEDGVNYAT | W | 1927 |
| Ns3 | 1197 | 1206 | PVESMETTMR | W | 1588 |
| Ns3 | 1634 | 1643 | EVTLTHPITK | W | 1041 |
| Ns5b | 2835 | 2844 | YAPTLWARMI | W | 2028 |
| Ns3 | 1567 | 1576 | HIDAHFLSQT | W | 1222 |
| Ns3 | 1490 | 1499 | RIGRGRRGIY | W | 1704 |
| Ns3 | 1530 | 1539 | LTPAETSVRL | W | 1471 |
| Ns5b | 2589 | 2598 | VCEKMALYDV | W | 1897 |
| Ns3 | 1568 | 1577 | IDAHFLSQTK | W | 1256 |
| Ns3 | 1522 | 1531 | DAGCAWYELT | W | 953 |
| Ns3 | 1580 | 1589 | GDNFPYLVAY | W | 1112 |
| Ns3 | 1192 | 1201 | AVDFIPVESM | W | 882 |
| Ns5b | 2707 | 2716 | TSCGNTLTCY | W | 1867 |
| Ns3 | 1284 | 1293 | TITTGAPITY | W | 1829 |
| Ns4b | 1944 | 1953 | VTQILSSLTI | W | 1977 |
| Ns5b | 2796 | 2805 | ASGKRVYYLT | W | 870 |
| Ns5b | 2713 | 2722 | LTCYLKASAA | W | 1466 |
| Ns3 | 1172 | 1181 | PSGHAVGIFR | W | 1584 |
| Core | 182 | 191 | LSCLTIPASA | W | 1458 |
| Ns5b | 2833 | 2842 | IMYAPTLWAR | W | 1279 |
| Ns3 | 1260 | 1269 | ATLGFGAYMS | W | 878 |
| Ns5b | 2754 | 2763 | ASLRVFTEAM | W | 871 |

TABLE 2

Predicted HLA-A*0201 binding peptides

| | | | | | |
|---|---|---|---|---|---|
| | | | | | SEQ ID |
| Protein | CS_fr | CS_to | pep_seq | Score | NO |

Algonomics
9-mer

| | | | | | | |
|---|---|---|---|---|---|---|
| 1 | NS5B | 2828 | 2836 | WLGNIIMYA | S | 62 |
| 2 | NS3 | 1585 | 1593 | YLVAYQATV | S | 63 |
| 3 | NS3 | 1565 | 1573 | LTHIDAHFL | S | 64 |
| 4 | C | 77 | 85 | AQPGYPWPL | S | 65 |
| 5 | C | 132 | 140 | DLMGYIPLV | S | 66 |
| 6 | NS5B | 2594 | 2602 | ALYDVVSTL | S | 67 |
| 7 | NS5B | 2598 | 2606 | VVSTLPQAV | S | 68 |
| 8 | C | 136 | 144 | YIPLVGAPL | S | 69 |
| 9 | C | 181 | 189 | LLSCLTIPA | S | 70 |
| 10 | NS3 | 1510 | 1518 | GMFDSSVLC | M | 71 |
| 11 | C | 150 | 158 | ALAHGVRVL | M | 72 |
| 12 | NS3 | 1250 | 1258 | KVLVLNPSV | M | 73 |
| 13 | NS3 | 1542 | 1550 | YLNTPGLPV | M | 74 |
| 14 | NS5B | 2727 | 2735 | KLQDCTMLV | M | 75 |
| 15 | NS3 | 1560 | 1568 | SVFTGLTHI | M | 76 |
| 16 | NS3 | 1434 | 1442 | VVATDALMT | M | 77 |
| 17 | C | 90 | 98 | GLGWAGWLL | M | 78 |
| 18 | NS5B | 2679 | 2687 | RLYIGGPLT | M | 79 |
| 19 | NS3 | 1195 | 1203 | FIPVESMET | M | 80 |
| 20 | NS3 | 1617 | 1625 | TLHGPTPLL | M | 81 |
| 21 | NS3 | 1252 | 1260 | LVLNPSVAA | M | 82 |
| 22 | NS3 | 1589 | 1597 | YQATVCARA | M | 83 |
| 23 | NS5B | 2833 | 2841 | IMYAPTLWA | M | 84 |
| 24 | NS5B | 2593 | 2601 | MALYDVVST | M | 85 |
| 25 | NS3 | 1342 | 1350 | RLVVLATAT | M | 86 |
| 26 | NS5B | 2831 | 2839 | NIIMYAPTL | M | 87 |
| 27 | NS5B | 2748 | 2756 | GTQEDAASL | M | 88 |
| 28 | NS3 | 1325 | 1333 | TILGIGTVL | M | 89 |
| 29 | NS3 | 1645 | 1653 | IMACMSADL | M | 90 |
| 30 | C | 29 | 37 | QIVGGVYLL | M | 91 |
| 31 | NS5B | 2838 | 2846 | TLWARMILM | M | 92 |
| 32 | C | 168 | 176 | NLPGCSFSI | M | 93 |
| 33 | NS5B | 2733 | 2741 | MLVNGDDLV | W | 94 |
| 34 | NS3 | 1244 | 1252 | YAAQGYKVL | W | 95 |
| 35 | NS3 | 1188 | 1196 | GVAKAVDFI | W | 96 |
| 36 | NS5B | 2842 | 2850 | RMILMTHFF | W | 97 |
| 37 | NS3 | 1331 | 1339 | TVLDQAETA | W | 98 |
| 38 | NS3 | 1637 | 1645 | LTHPITKYI | W | 99 |
| 39 | NS3 | 1253 | 1261 | VLNPSVAAT | W | 100 |
| 40 | NS3 | 1210 | 1218 | FTDNSSPPA | W | 101 |
| 41 | NS3 | 1345 | 1353 | VLATATPPG | W | 102 |
| 42 | NS3 | 1251 | 1259 | VLVLNPSVA | W | 103 |
| 43 | NS3 | 1169 | 1177 | LLCPSGHVV | W | 104 |
| 44 | NS3 | 1420 | 1428 | GLDVSVIPT | W | 105 |
| 45 | NS3 | 1464 | 1472 | FSLDPTFTI | W | 106 |
| 46 | NS3 | 1260 | 1268 | ATLGFGAYM | W | 107 |
| 47 | NS5B | 2835 | 2843 | YAPTLWARM | W | 108 |
| 48 | NS3 | 1284 | 1292 | TITTGAPIT | W | 109 |
| 49 | NS3 | 1203 | 1211 | TTMRSPVFT | W | 110 |
| 50 | NS5B | 2613 | 2621 | FQYSPGQRV | W | 111 |
| 51 | NS3 | 1224 | 1232 | QVAHLHAPT | W | 112 |
| 52 | NS3 | 1218 | 1226 | AVPQTFQVA | W | 113 |
| 53 | NS3 | 1283 | 1291 | RTITTGAPI | W | 114 |
| 54 | NS3 | 1245 | 1253 | AAQGYKVLV | W | 115 |
| 55 | NS3 | 1586 | 1594 | LVAYQATVC | W | 116 |
| 56 | NS3 | 1178 | 1186 | GVFRAAVCT | W | 117 |
| 57 | C | 133 | 141 | LMGYIPLVG | W | 118 |
| 58 | NS3 | 1630 | 1638 | AVQNEVTLT | W | 119 |
| 59 | NS3 | 1497 | 1505 | GIYRFVTPG | W | 120 |
| 60 | NS5B | 2720 | 2728 | SAACRAAKL | W | 121 |
| 61 | NS3 | 1610 | 1618 | CLIRLKPTL | W | 122 |
| 62 | NS3 | 1572 | 1580 | FLSQTKQAG | W | 123 |
| 63 | NS3 | 1450 | 1458 | SVIDCNTCV | W | 124 |
| 64 | NS3 | 1349 | 1357 | ATPPGSVTV | W | 125 |
| 65 | NS5B | 2815 | 2823 | AAWETARHT | W | 126 |
| 66 | C | 28 | 36 | GQIVGGVYL | W | 127 |
| 67 | C | 157 | 165 | VLEDGVNYA | W | 128 |
| 68 | NS3 | 1555 | 1563 | LEFWESVFT | W | 129 |
| 69 | NS3 | 1246 | 1254 | AQGYKVLVL | W | 130 |
| 70 | NS5B | 2734 | 2742 | LVNGDDLVV | W | 131 |
| 71 | C | 36 | 44 | LLPRRGPRL | W | 132 |
| 72 | NS5B | 2600 | 2608 | STLPQAVMG | W | 133 |
| 73 | NS3 | 1425 | 1433 | VIPTSGDVV | W | 134 |
| 74 | NS3 | 1509 | 1517 | SGMFDSSVL | W | 135 |
| 75 | NS3 | 1648 | 1656 | CMSADLEVV | W | 136 |
| 76 | NS3 | 1376b | 1384b | YGKAIPIEV | W | 137 |
| 77 | NS3 | 1649 | 1657 | MSADLEVVT | W | 138 |
| 78 | NS5B | 2830 | 2838 | GNIIMYAPT | W | 139 |
| 79 | NS3 | 1328 | 1336 | GIGTVLDQA | W | 140 |
| 80 | NS3 | 1175 | 1183 | HVVGVFRAA | W | 141 |
| 81 | NS3 | 1406 | 1414 | KLSALGLNA | W | 142 |
| 82 | NS3 | 1379b | 1387b | AIPIEVIKG | W | 143 |

| | | | | | |
|---|---|---|---|---|---|
| | | | | Score | SEQ ID |
| Protein | CS_fr | CS_to | pep_seq | PIC | NO |

Epimmune

| | | | |
|---|---|---|---|
| C | AQPGYPWPL | 82 | 65 |
| C | GLGWAGWLL | 71 | 78 |
| C | DLMGYIPLV | 21 | 66 |
| C | DLMGYIPVV | 56 | 966 |
| C | NLPGCSFSI | 56 | 93 |
| C | FLLALLSCL | 24 | 361 |
| C | FLLALFSCL | 21 | 1068 |
| C | FLLALLSCI | 24 | 1069 |
| C | FLLALLSCLT | 87 | 1070 |
| | LLALLSCLTV | 63 | 1390 |
| | HLPGCVPCV | 75 | 1231 |
| E1 | MMMNWSPTA | 55 | 1498 |
| E1 | MMMNWSPTT | 91 | 1500 |
| E1 | MMMNWSPTAA | 99 | 1499 |
| E1 | MMMNWSPTTA | 90 | 1501 |
| | VMFGLAYFSM | 78 | 1937 |
| | SMQGAWAKV | 76 | 1752 |
| | LQTGFLASL | 34 | 1451 |
| E2 | CMVDYPYRL | 40 | 924 |
| E2 | CLVDYPYRL | 41 | 922 |
| E2 | CLIDYPYRL | 37 | 920 |
| E2 | CLVHYPYRL | 81 | 923 |
| E2 | RLWHYPCTI | 25 | 1667 |
| E2 | RLWHYPCTV | 14 | 1669 |
| E2 | RLWHYPCTL | 25 | 1668 |
| E2 | TLFKVRMYV | 89 | 1830 |
| E2 | ALSTGLIHL | 74 | 825 |
| E2 | ALSTGLLHL | 64 | 826 |
| E2 | YLYGVGSAV | 23 | 2056 |
| E2 | YLYGVGSAVV | 43 | 2057 |
| E2 | YVVLLFLLL | 42 | 2075 |
| E2 | YVVLLFLLLA | 77 | 2076 |
| E2 | VILLFLLLA | 60 | 1919 |
| E2 | VVLLFLLLA | 77 | 1983 |
| E2 | LLFLLLADA | 61 | 1395 |
| E2 | FLLLADARI | 36 | 1071 |
| E2 | FLLLADARV | 20 | 1072 |
| | LLLADARVCV | 98 | 1399 |
| | MLLISQAEA | 90 | 1497 |
| P7 | GVWPLLLLL | 61 | 1207 |
| | ALQVWVPPL | 72 | 824 |
| | LQVWVPPLL | 45 | 1453 |
| | KLLLAVLGPL | 83 | 1332 |
| | LLLAVLGPL | 50 | 1401 |
| | LLIAVLGPL | 57 | 1398 |
| | LLLAIFGPL | 44 | 1400 |
| | ALLGPAYLL | 46 | 823 |
| | AVLGPLYLI | 53 | 892 |
| | SLLRIPYFV | 18 | 1744 |
| | YIYNHLTPL | 51 | 2040 |
| | YIYDHLTPM | 37 | 2039 |
| NS2 | YVYNHLTPL | 66 | 2079 |
| NS2 | YVYDHLTPL | 26 | 2077 |
| | LLAPITAYA | 36 | 1391 |
| NS3 | GLLGCIITSL | 88 | 1151 |
| NS3 | LLGCIITSL | 57 | 1397 |
| NS3 | FLGTTVGGV | 62 | 1067 |
| NS3 | FLGTSISGV | 66 | 1066 |
| NS3 | FLATCINGV | 25 | 1065 |
| NS3 | CINGVCWTV | 84 | 919 |
| NS3 | SISGVLWTV | 61 | 1737 |
| NS3 | GVMWTVYHGA | 100 | 1198 |
| NS3 | VMWTVYHGA | 39 | 1942 |
| NS3 | VLWTVYHGA | 41 | 1935 |
| NS3 | YLVTRHADV | 79 | 2053 |
| NS3 | YLVTRNADV | 38 | 2055 |
| NS3 | ATLGFGAYM | 92 | 32 |
| NS3 | YLNTPGLPV | 45 | 74 |
| NS3 | YLSTPGLPV | 49 | 2049 |
| NS3 | YLVAYQATV | 3 | 63 |
| NS3 | YLTAYQATV | 5 | 2050 |
| NS3 | YQATVCARA | 49 | 83 |
| NS3 | QMWKCLIRL | 71 | 238 |
| NS3 | VMWKCLIRL | 36 | 1940 |
| NS3 | VMWKCLTRL | 61 | 1941 |
| NS3 | RLGAVQNEV | 82 | 265 |
| NS4A | VLVGGVLAA | 74 | 1933 |
| NS4A | VLAGGVLAA | 90 | 1922 |
| NS4A | VLVGGVLAAL | 89 | 1934 |
| NS4A | VLAGGVLAAV | 47 | 1923 |
| NS4A | LAGGVLAAV | 99 | 1360 |
| NS4A | ALAAYCLSV | 7 | 820 |
| NS4A | ALAAYCLTT | 26 | 821 |
| NS4B | HMWNFISGI | 53 | 1233 |
| NS4B | HMWNFVSGI | 49 | 1234 |
| NS4B | FISGIQYLA | 20 | 1060 |
| NS4B | FVSGIQYLA | 25 | 1093 |
| NS4B | SLMAFTASV | 6 | 1746 |
| NS4B | SMMAFSAAL | 19 | 1751 |
| NS4B | LLFNILGGWV | 51 | 1396 |
| NS4B | ILLNIMGGWL | 87 | 1272 |
| NS4B | FVVSGLAGA | 77 | 1094 |
| NS4B | ILAGYGAGV | 28 | 1269 |
| NS4B | VLAGYGAGV | 30 | 1925 |
| NS4B | VLAGYGAGI | 54 | 1924 |
| NS4B | WMNRLIAFA | 97 | 2015 |
| NS5A | NMWHGTFPI | 21 | 1525 |
| NS5A | NTWQGTFPI | 99 | 1537 |
| NS5A | NTWHGTFPI | 87 | 1536 |
| | FMGGDVTRI | 46 | 1075 |
| NS5B | RLIVFPDLGV | 83 | 1661 |
| NS5B | ALYDVIQKL | 56 | 829 |
| NS5B | ALYDITQKL | 63 | 828 |
| NS5B | ALYDVVSTL | 29 | 67 |
| NS5B | FLVCGDDLV | 43 | 1073 |
| NS5B | FLVCGDDLVV | 65 | 1074 |
| NS5B | IQYAPTIWV | 39 | 1299 |
| NS5B | IMYAPTLWA | 34 | 84 |
| NS5B | TLWARMILM | 40 | 92 |
| NS5B | ILMTHFFSI | 7 | 1273 |
| NS5B | VLMTHFFSI | 8 | 1928 |
| NS5B | VLMTHFFSIL | 90 | 1929 |
| NS5B | ILMTHFFSIL | 82 | 1274 |
| NS5B | EMYGATYSV | 30 | 1019 |
| NS5B | EMYGAVYSV | 24 | 1020 |
| NS5B | RLHGLSAFT | 74 | 1660 |
| NS5B | RLHGLEAFSL | 89 | 1658 |
| NS5B | RLHGLDAFSL | 73 | 1657 |
| NS5B | GLDAFSLHT | 67 | 1147 |
| NS5B | GLYLFNWAV | 33 | 1157 |
| NS5B | RLLDLSSWFT | 53 | 1663 |
| NS5B | RLLLLGLLLL | 40 | 1664 |
| NS5B | HLLLCLLLL | 42 | 1230 |
| NS5B | LLLLGLLLL | 38 | 1404 |
| NS5B | LLLCLLLLT | 27 | 1402 |
| NS5B | LLLCLLLLTV | 16 | 1403 |
| NS5B | LLCLLLLTV | 43 | 1393 |
| NS5B | LLLLTVGVGI | 70 | 1405 |
| NS5B | LTVGVGIFL | 89 | 1477 |

TABLE 3

Predicted HLA-A*0301 and HLA-A*1101 binding
peptides

| | | | | | |
|---|---|---|---|---|---|
| | | | | | SEQ ID |
| Protein | CS_fr | CS_to | pep_seq | Score | NO |

Algonomics
9-mer

| | | | | | | |
|---|---|---|---|---|---|---|
| 1 | C | 43 | 51 | RLGVRATRK | S | 144 |
| 2 | NS3 | 1411 | 1419 | GLNAVAYYR | S | 145 |
| 3 | NS5B | 2624 | 2632 | LVNAWKSKK | S | 146 |
| 4 | NS3 | 1242 | 1250 | AAYAAQGYK | S | 147 |
| 5 | NS3 | 1390 | 1398 | HLIFCHSKK | S | 148 |
| 6 | C | 35 | 43 | YLLPRRGPR | S | 149 |
| 7 | NS5B | 2623 | 2631 | FLVNAWKSK | S | 150 |
| 8 | NS3 | 1391 | 1399 | LIFCHSKKK | S | 151 |
| 9 | NS3 | 1635 | 1643 | VTLTHPITK | M | 152 |
| 10 | NS3 | 1409 | 1417 | ALGLNAVAY | M | 153 |
| 11 | NS5B | 2584 | 2592 | DLGVRVCEK | M | 154 |
| 12 | NS5B | 2719 | 2727 | ASAACRAAK | M | 155 |
| 13 | NS3 | 1183 | 1191 | AVCTRGVAK | M | 156 |
| 14 | NS5B | 2567 | 2575 | VQPEKGGRK | M | 157 |
| 15 | C | 2 | 10 | STNPKPQRK | M | 158 |
| 16 | C | 62 | 70 | RQPIPKARR | M | 159 |
| 17 | NS5B | 2757 | 2765 | RVFTEAMTR | M | 160 |
| 18 | NS5B | 2716 | 2724 | YLKASAACR | M | 161 |
| 19 | NS5B | 2710 | 2718 | GNTLTCYLK | M | 162 |
| 20 | C | 96 | 104 | WLLSPRGSR | M | 163 |
| 21 | C | 10 | 18 | KTKRNTNRR | M | 164 |
| 22 | NS5B | 2594 | 2602 | ALYDVVSTL | M | 165 |
| 23 | NS3 | 1262 | 1270 | LGFGAYMSK | M | 166 |
| 24 | C | 51 | 59 | KTSERSQPR | M | 167 |
| 25 | NS5B | 2580 | 2588 | IVFPDLGVR | M | 168 |
| 26 | C | 156 | 164 | RVLEDGVNY | M | 169 |
| 27 | NS5B | 2833 | 2841 | IMYAPTLWA | W | 170 |
| 28 | NS3 | 1288 | 1296 | GAPITYSTY | W | 171 |
| 29 | NS5B | 2798 | 2806 | GKRVYYLTR | W | 172 |
| 30 | NS3 | 1389 | 1397 | RHLIFCHSK | W | 173 |
| 31 | NS3 | 1492 | 1500 | GRGRRGIYR | W | 174 |
| 32 | NS5B | 2679 | 2687 | RLYIGGPLT | W | 175 |
| 33 | NS5B | 2634 | 2642 | PMGFSYDTR | W | 176 |
| 34 | C | 59 | 67 | RGRRQPIPK | W | 177 |
| 35 | NS5B | 2621 | 2629 | VEFLVNAWK | W | 178 |
| 36 | NS3 | 1510 | 1518 | GMFDSSVLC | W | 179 |
| 37 | NS3 | 1605 | 1613 | DQMWKCLIR | W | 180 |
| 38 | NS3 | 1378b | 1386b | KAIPIEVIK | W | 181 |
| 39 | NS3 | 1542 | 1550 | YLNTPGLPV | W | 182 |
| 40 | NS5B | 2588 | 2596 | RVCEKMALY | W | 183 |
| 41 | C | 93 | 101 | WAGWLLSPR | W | 184 |
| 42 | NS3 | 1585 | 1593 | YLVAYQATV | W | 185 |
| 43 | C | 90 | 98 | GLGWAGWLL | W | 186 |
| 44 | NS3 | 1607 | 1615 | MWKCLIRLK | W | 187 |
| 45 | NS5B | 2791 | 2799 | SVAHDASGK | W | 188 |
| 46 | C | 47 | 55 | RATRKTSER | W | 189 |
| 47 | NS5B | 2828 | 2836 | WLGNIIMYA | W | 190 |
| 48 | NS3 | 1378 | 1386 | KAIPIEAIK | W | 191 |
| 49 | NS3 | 1619 | 1627 | HGPTPLLYR | W | 192 |
| 50 | NS5B | 2563 | 2571 | EVFCVQPEK | W | 193 |
| 51 | C | 1 | 9 | MSTNPKPQR | W | 194 |
| 52 | NS3 | 1228 | 1236 | LHAPTGSGK | W | 195 |
| 53 | NS3 | 1482 | 1490 | VSRSQRRGR | W | 196 |
| 54 | C | 31 | 39 | VGGVYLLPR | W | 197 |
| 55 | NS3 | 1178 | 1186 | GVFRAAVCT | W | 198 |
| 56 | C | 15 | 23 | TNRRPQDVK | W | 199 |
| 57 | NS3 | 1624 | 1632 | LLYRLGAVQ | W | 200 |
| 58 | NS3 | 1636 | 1644 | TLTHPITKY | W | 201 |
| 59 | NS3 | 1420 | 1428 | GLDVSVIPT | W | 202 |
| 60 | C | 105 | 113 | PSWGPTDPR | W | 203 |
| 61 | NS3 | 1176 | 1184 | VVGVFRAAV | W | 204 |
| 62 | NS3 | 1611 | 1619 | LIRLKPTLH | W | 205 |
| 63 | C | 45 | 53 | GVRATRKTS | W | 206 |
| 64 | C | 141 | 149 | GAPLGGAAR | W | 207 |
| 65 | C | 132 | 140 | DLMGYIPLV | W | 208 |
| 66 | NS3 | 1221 | 1229 | QTFQVAHLH | W | 209 |
| 67 | NS3 | 1436 | 1444 | ATDALMTGY | W | 210 |
| 68 | NS3 | 1577 | 1585 | KQAGDNFPY | W | 211 |
| 69 | NS3 | 1581 | 1589 | DNFPYLVAY | W | 212 |
| 70 | C | 36 | 44 | LLPRRGPRL | W | 213 |
| 71 | NS3 | 1231 | 1239 | PTGSGKSTK | W | 214 |
| 72 | NS3 | 1291 | 1299 | ITYSTYGKF | W | 215 |
| 73 | C | 78 | 86 | QPGYPWPLY | W | 216 |
| 74 | NS5B | 2762 | 2770 | AMTRYSAPP | W | 217 |
| 75 | NS3 | 1328 | 1336 | GIGTVLDQA | W | 218 |
| 76 | NS3 | 1618 | 1626 | LHGPTPLLY | W | 219 |
| 77 | NS3 | 1530 | 1538 | LTPAETSVR | W | 220 |
| 78 | NS3 | 1485 | 1493 | SQRRGRTGR | W | 221 |
| 79 | NS3 | 1236 | 1244 | KSTKVPAAY | W | 222 |
| 80 | C | 30 | 38 | IVGGVYLLP | W | 223 |
| 81 | NS3 | 1490 | 1498 | RTGRGRRGI | W | 224 |
| 82 | NS3 | 1406 | 1414 | KLSALGLNA | W | 225 |
| 83 | NS5B | 2613 | 2621 | FQYSPGQRV | W | 226 |
| 84 | C | 74 | 82 | RTWAQPGYP | W | 227 |
| 85 | NS5B | 2692 | 2700 | QNCGYRRCR | W | 228 |
| | NS3 | 1513 | 1521 | DSSVLCECY | N | 229 |

| | | | | | |
|---|---|---|---|---|---|
| | | | | Score | SEQ ID |
| Protein | CS_fr | CS_to | pep_seq | PIC | NO |

Epimmune

| | | | |
|---|---|---|---|
| C | STNPKPQRK | 5.6 | 158 |
| C | STIPKPQRK | 17 | 1789 |
| C | KTSERSQPR | 70 | 167 |
| C | AQPGYPWPLY | 365 | 861 |
| | RVLEDGINY | 51 | 1713 |
| | GQAFTFRPR | 383 | 1177 |
| E1 | QLFTFSPRR | 109 | 1609 |
| E1 | TTQDCNCSIY | 67 | 1877 |
| E1 | ALVVSQLLR | 50 | 827 |
| E1 | GVLAGLAYY | 26 | 1197 |
| E1 | GILAGLAYY | 94 | 1142 |
| | FSMQGAWAK | 3.7 | 1084 |
| | QTGFLASLFY | 59 | 1620 |
| | GFIAGLFYY | 57 | 1134 |
| | FIAGLFYYHK | 11 | 1058 |
| | FLASLFYTHK | 50 | 1064 |
| | TLLCPTDCFR | 168 | 1834 |
| | LLCPTDCFRK | 125 | 1394 |
| E2 | CTVNFTIFK | 2.4 | 945 |
| E2 | CTVNFTLFK | 2.0 | 946 |
| E2 | CTVNFSIFK | 4.2 | 944 |
| | GQAEAALEK | 13 | 1176 |
| P7 | VFFCAAWYIK | 6.5 | 1908 |
| P7 | FFCAAWYIK | 30 | 1056 |
| P7 | GFFTLSPWYK | 44 | 1133 |
| P7 | FFTLSPWYK | 31 | 1057 |
| P7 | ILTLSPHYK | 32 | 1277 |
| | SLLRIPYFVR | 112 | 1745 |
| | LTRVPYFVR | 43 | 1476 |
| | LLRIPYFVR | 301 | 1407 |
| | FVRAHALLR | 71 | 1091 |
| | KLGALTGTY | 444 | 1329 |
| NS2 | YVYDHLTPLR | 26 | 2078 |
| NS2 | YVYNHLTPLR | 34 | 2080 |
| | VIFSPMEIK | 5.7 | 1915 |
| | RLLAPITAY | 466 | 1662 |
| | KLLAPITAY | 331 | 1330 |
| | ITAYAQQTR | 58 | 1307 |
| | TVYHGAGNK | 6.7 | 1882 |
| | AVDLYLVTR | 20 | 884 |
| NS3 | GIFRAAVCTR | 27 | 1141 |
| NS3 | GIFRAAVCSR | 47 | 1140 |
| NS3 | AVCTRGVAK | 5.4 | 156 |
| NS3 | AVCSRGVAK | 2.8 | 881 |
| NS3 | TLGFGAYMSK | 18 | 1831 |
| NS3 | TLGFGTYMSK | 22 | 1832 |
| NS3 | PITYSTYGK | 77 | 1556 |
| NS3 | KLTYSTYGK | 15 | 1334 |
| NS3 | SITYSTYGK | 2.1 | 1738 |
| NS3 | AITYSTYGK | 2.0 | 818 |
| NS3 | TTGEIPFYGK | 13 | 1873 |
| NS3 | HLIFCHSRK | 95 | 1229 |
| NS3 | LIFCHSKKK | 45 | 47 |
| NS3 | LIFCHSRKK | 80 | 1385 |
| NS3 | SLGLNAVAYY | 327 | 1742 |
| NS3 | GLNAVAYYR | 4.8 | 145 |
| NS3 | GINAVAYYR | 2.0 | 1143 |
| NS3 | GVNAVAYYR | 0.57 | 1199 |
| NS3 | ATDALMTGY | 48 | 1 |
| NS3 | KQSGENFPY | 244 | 1347 |
| NS3 | DVMWKCLTR | 62 | 991 |
| NS3 | DQMWKCLTR | 504 | 983 |
| NS3 | LQGPTPLLYR | 881 | 1448 |
| NS3 | VTLTHPITK | 13 | 21 |
| NS3 | VVLTHPITK | 9.9 | 1984 |
| NS4B | MQLAEQFKQK | 268 | 1506 |
| NS4B | QLAEQFKQK | 34 | 1608 |
| NS4B | RIAEMLKSK | 69 | 1654 |
| NS4B | AVGSIGLGK | 0.9 | 888 |
| NS4B | AVGSVGLGK | 1.2 | 889 |
| NS4B | GVAGALVAFK | 3.0 | 1192 |
| NS4B | GISGALVAFK | 16 | 1145 |
| NS4B | GVSGALVAFK | 5.2 | 1204 |
| NS4B | ISGALVAFK | 29 | 1302 |
| NS4B | VSGALVAFK | 29 | 1971 |
| NS4B | AFKIMSGEK | 326 | 801 |
| NS4B | GVVCAAILR | 19 | 1205 |
| NS4B | GVICAAILR | 20 | 1194 |
| NS4B | GVVCAAILRR | 17 | 1206 |
| NS4B | GVICAAILRR | 20 | 1195 |
| NS4B | VVCAAILRR | 6.5 | 1978 |
| NS4B | VICAAILRR | 23 | 1913 |
| | SLTITSLLR | 110 | 1747 |
| | SLTVTQLLR | 98 | 1748 |
| | SLTVTSLLR | 103 | 1749 |
| | GLPFISCQK | 68 | 1152 |
| | GIPFISCQK | 28 | 1144 |
| | GSMRITGPK | 2.4 | 1188 |
| | QIHRFAPTPK | 34 | 1605 |
| | ITAEAAARR | 70 | 1305 |
| NS5A | ASQLSAPSLK | 25 | 873 |
| NS5A | SQLSAPSLK | 15 | 1781 |
| NS5A | SQLSAPSLR | 151 | 1782 |
| NS5A | NLFMGGDVTR | 273 1524 | |
| NS5A | RQEMGGNITR | 587 1692 | |
| NS5A | RQEMGSNITR | 553 1693 | |
| NS5A | VSVPAEILRK | 23 | 1974 |
| NS5A | SVPAEILRK | 1.5 | 1800 |
| NS5A | SIPSEYLLPK | 18 | 1736 |
| NS5A | SSALAELATK | 28 | 1784 |
| NS5A | STALAELAAK | 13 | 1786 |
| NS5B | SLLRHHNMVY | 215 | 1743 |
| NS5B | TTSRSASQR | 26 | 1879 |
| NS5B | TTSRSASLR | 61 | 1878 |
| NS5B | RQKKVTFDR | 955 | 1694 |
| NS5B | RLQVLDDHYK | 48 | 1665 |
| NS5B | LQVLDDHYK | 139 | 1452 |
| NS5B | VQPEKGGRK | 595 | 157 |
| NS5B | RVCEKMALY | 75 | 3 |
| NS5B | RVFTEAMTR | 13 | 39 |
| NS5B | RVFTEAMTRY | 20 | 1712 |
| NS5B | SVAHDASGK | 5.7 | 188 |
| NS5B | SVAHDASGKR | 54 | 1797 |
| NS5B | SVALDPRGRR | 61 | 1798 |
| NS5B | IQYAPTIWVR | 702 | 1300 |
| NS5B | LLAQEQLEK | 151 | 1392 |
| NS5B | AVRASLISR | 26 | 894 |
| NS5B | SVRAKLLSR | 36 | 1801 |
| NS5B | GLYLFNWAVR | 78 | 1158 |
| NS5B | YLFNWAVRTK | 53 | 2044 |
| NS5B | YLFNWAVKTK | 54 | 2042 |
| NS5B | LFNWAVRTK | 124 | 1380 |
| NS5B | LFNWAVKTK | 69 | 1379 |

TABLE 4

Predicted HLA-A*2402 binding peptides

| | | | | | |
|---|---|---|---|---|---|
| | | | | | SEQ ID |
| Protein | CS_fr | CS_to | pep_seq | Score | NO |

Algonomics
9-mer

| | | | | | | |
|---|---|---|---|---|---|---|
| 1 | NS5B | 2842 | 2850 | RMILMTHFF | S | 230 |
| 2 | NS5B | 2838 | 2846 | TLWARMILM | S | 231 |
| 3 | NS3 | 1610 | 1618 | CLIRLKPTL | S | 232 |
| 4 | NS3 | 1617 | 1625 | TLHGPTPLL | S | 233 |
| 5 | NS3 | 1557 | 1565 | FWESVFTGL | S | 234 |
| 6 | C | 75 | 83 | TWAQPGYPW | S | 235 |
| 7 | C | 129 | 137 | GFADLMGYI | S | 236 |
| 8 | NS5B | 2831 | 2839 | NIIMYAPTL | S | 237 |
| 9 | NS3 | 1606 | 1614 | QMWKCLIRL | S | 238 |
| 10 | N53 | 1643 | 1651 | KYIMACMSA | S | 239 |
| 11 | NS3 | 1246 | 1254 | AQGYKVLVL | S | 240 |
| 12 | NS3 | 1292 | 1300 | TYSTYGKFL | S | 241 |
| 13 | N53 | 1270 | 1278 | KAHGVDPNI | S | 242 |
| 14 | C | 85 | 93 | LYGNEGLGW | S | 243 |
| 15 | NS3 | 1266 | 1274 | AYMSKAHGV | S | 244 |
| 16 | C | 90 | 98 | GLGWAGWLL | M | 245 |
| 17 | NS5B | 2832 | 2840 | IIMYAPTLW | M | 246 |
| 18 | C | 28 | 36 | GQIVGGVYL | M | 247 |
| 19 | NS5B | 2828 | 2836 | WLGNIIMYA | M | 248 |
| 20 | NS3 | 1338 | 1346 | TAGARLVVL | M | 249 |
| 21 | C | 173 | 181 | SFSIFLLAL | M | 250 |
| 22 | NS3 | 1464 | 1472 | FSLDPTFTI | M | 251 |
| 23 | NS3 | 1585 | 1593 | YLVAYQATV | M | 252 |
| 24 | NS3 | 1384b | 1392b | VIKGGRHLI | M | 253 |
| 25 | NS3 | 1623 | 1631 | PLLYRLGAV | M | 254 |
| 26 | NS3 | 1325 | 1333 | TILGIGTVL | M | 255 |
| 27 | NS5B | 2824 | 2832 | PVNSWLGNI | M | 256 |
| 28 | NS3 | 1202 | 1210 | ETTMRSPVF | M | 257 |
| 29 | NS3 | 1564 | 1572 | GLTHIDAHF | M | 258 |
| 30 | NS5B | 2605 | 2613 | AVMGSSYGF | M | 259 |
| 31 | NS3 | 1162 | 1170 | KGSSGGPLL | M | 260 |
| 32 | NS5B | 2727 | 2735 | KLQDCTMLV | M | 261 |
| 33 | NS3 | 1244 | 1252 | YAAQGYKVL | M | 262 |
| 34 | NS3 | 1637 | 1645 | LTHPITKYI | M | 263 |
| 35 | NS3 | 1374 | 1382 | PFYGKAIPI | M | 264 |
| 36 | NS3 | 1627 | 1635 | RLGAVQNEV | M | 265 |
| 37 | NS3 | 1384 | 1392 | AIKGGRHLI | M | 266 |
| 38 | NS5B | 2594 | 2602 | ALYDVVSTL | M | 267 |
| 39 | C | 149 | 157 | RALAHGVRV | M | 268 |
| 40 | C | 136 | 144 | YIPLVGAPL | M | 269 |
| 41 | C | 36 | 44 | LLPRRGPRL | M | 270 |
| 42 | NS3 | 1417 | 1425 | YYRGLDVSV | M | 271 |
| 43 | NS3 | 1402 | 1410 | ELAAKLSAL | M | 272 |
| 44 | NS3 | 1376b | 1384b | YGKAIPIEV | M | 273 |
| 45 | NS5B | 2607 | 2615 | MGSSYGFQY | M | 274 |
| 46 | NS3 | 1169 | 1177 | LLCPSGHVV | M | 275 |
| 47 | NS5B | 2627 | 2635 | AWKSKKCPM | M | 276 |
| 48 | NS3 | 1243 | 1251 | AYAAQGYKV | M | 277 |
| 49 | NS5B | 2620 | 2628 | RVEFLVNAW | M | 278 |
| 50 | NS3 | 1603 | 1611 | SWDQMWKCL | M | 279 |
| 51 | C | 168 | 176 | NLPGCSFSI | M | 280 |
| 52 | NS5B | 2636 | 2644 | GFSYDTRCF | M | 281 |
| 53 | NS3 | 1217 | 1225 | PAVPQTFQV | M | 282 |
| 54 | C | 118 | 126 | NLGKVIDTL | M | 283 |
| 55 | C | 23 | 31 | KFPGGGQIV | M | 284 |
| 56 | NS3 | 1542 | 1550 | YLNTPGLPV | M | 285 |
| 57 | NS3 | 1604 | 1612 | WDQMWKCLI | W | 286 |
| 58 | NS5B | 2840 | 2848 | WARMILMTH | W | 287 |
| 59 | C | 77 | 85 | AQPGYPWPL | W | 288 |
| 60 | C | 29 | 37 | QIVGGVYLL | W | 289 |
| 61 | NS3 | 1293 | 1301 | YSTYGKFLA | W | 290 |
| 62 | NS3 | 1510 | 1518 | GMFDSSVLC | W | 291 |
| 63 | NS5B | 2834 | 2842 | MYAPTLWAR | W | 292 |
| 64 | C | 172 | ‘180 | CSFSIFLLA | W | 293 |
| 65 | C | 171 | 179 | GCSFSIFLL | W | 294 |
| 66 | NS3 | 1188 | 1196 | GVAKAVDFI | W | 295 |
| 67 | NS5B | 2613 | 2621 | FQYSPGQRV | W | 296 |
| 68 | C | 150 | 158 | ALAHGVRVL | W | 297 |
| 69 | NS5B | 2821 | 2829 | RHTPVNSWL | W | 298 |
| 70 | NS5B | 2837 | 2845 | PTLWARMIL | W | 299 |
| 71 | NS3 | 1493 | 1501 | RGRRGIYRF | W | 300 |
| 72 | NS5B | 2629 | 2637 | KSKKCPMGF | W | 301 |
| 73 | C | 179 | 187 | LALLSCLTI | W | 302 |
| 74 | NS3 | 1354 | 1362 | SVTVPHPNI | W | 303 |
| 75 | NS5B | 2705 | 2713 | LTTSCGNTL | W | 304 |
| 76 | NS3 | 1641 | 1649 | ITKYIMACM | W | 305 |
| 77 | NS3 | 1375 | 1383 | FYGKAIPIE | W | 306 |
| 78 | NS3 | 1620 | 1628 | GPTPLLYRL | W | 307 |
| 79 | NS3 | 1440 | 1448 | LMTGYTGDF | W | 308 |
| 80 | NS5B | 2588 | 2596 | RVCEKMALY | W | 309 |
| 81 | C | 132 | 140 | DLMGYIPLV | W | 310 |
| 82 | NS3 | 1385 | 1393 | IKGGRHLIF | W | 311 |
| 83 | NS3 | 1220 | 1228 | PQTFQVAHL | W | 312 |
| 84 | NS5B | 2802 | 2810 | YYLTRDPTT | W | 313 |
| 85 | NS5B | 2839 | 2847 | LWARMILMT | W | 314 |
| 86 | NS3 | 1250 | 1258 | KVLVLNPSV | W | 315 |
| 87 | NS3 | 1283 | 1291 | RTITTGAPI | W | 316 |
| 88 | NS3 | 1187 | 1195 | RGVAKAVDF | W | 317 |
| 89 | C | 115 | 123 | RSRNLGKVI | W | 318 |
| 90 | NS5B | 2679 | 2687 | RLYIGGPLT | W | 319 |
| 91 | NS5B | 2715 | 2723 | CYLKASAAC | W | 320 |
| 92 | NS3 | 1527 | 1535 | WYELTPAET | W | 321 |
| 93 | NS3 | 1565 | 1573 | LTHIDAHFL | W | 322 |
| 94 | NS5B | 2617 | 2625 | PGQRVEFLV | W | 323 |
| 95 | NS3 | 1406 | 1414 | KLSALGLNA | W | 324 |
| 96 | NS3 | 1566 | 1574 | THIDAHFLS | W | 325 |
| 97 | NS5B | 2615 | 2623 | YSPGQRVEF | W | 326 |
| 98 | NS3 | 1579 | 1587 | AGDNFPYLV | W | 327 |
| 99 | NS3 | 1645 | 1653 | IMACMSADL | W | 328 |
| 100 | NS3 | 1549 | 1557 | PVCQDHLEF | W | 329 |
| 101 | NS3 | 1245 | 1253 | AAQGYKVLV | W | 330 |
| 102 | NS3 | 1365 | 1373 | IGLSNNGEI | W | 331 |
| 103 | NS5B | 2674 | 2682 | RSLTERLYI | W | 332 |
| 104 | NS3 | 1648 | 1656 | CMSADLEVV | W | 333 |
| 105 | NS5B | 2796 | 2804 | ASGKRVYYL | W | 334 |
| 106 | NS3 | 1376 | 1384 | YGKAIPIEA | W | 335 |
| 107 | NS5B | 2782 | 2790 | LITSCSSNV | W | 336 |
| 108 | NS3 | 1260 | 1268 | ATLGFGAYM | W | 337 |
| 109 | NS5B | 2665 | 2673 | LAPEARQAI | W | 338 |
| 110 | C | 161 | 169 | GVNYATGNL | W | 339 |
| 111 | C | 156 | 164 | RVLEDGVNY | W | 340 |
| 112 | NS3 | 1444 | 1452 | YTGDFDSVI | W | 341 |
| 113 | NS3 | 1640 | 1648 | PITKYIMAC | W | 342 |
| 114 | NS3 | 1433 | 1441 | VVVATDALM | W | 343 |
| 115 | NS3 | 1596 | 1604 | RAQAPPPSW | W | 344 |
| 116 | C | 170 | 178 | PGCSFSIFL | W | 345 |
| 117 | NS3 | 1560 | 1568 | SVFTGLTHI | W | 346 |
| 118 | NS3 | 1629 | 1637 | GAVQNEVTL | W | 347 |
| 119 | NS3 | 1577 | 1585 | KQAGDNFPY | W | 348 |
| 120 | NS5B | 2581 | 2589 | VFPDLGVRV | W | 349 |
| 121 | NS3 | 1462 | 1470 | VDFSLDPTF | W | 350 |
| 122 | NS3 | 1547 | 1555 | GLPVCQDHL | W | 351 |
| 123 | NS5B | 2735 | 2743 | VNGDDLVVI | W | 352 |
| 124 | NS3 | 1443 | 1451 | GYTGDFDSV | W | 353 |
| 125 | NS3 | 1172 | 1180 | PSGHVVGVF | W | 354 |
| 126 | NS5B | 2598 | 2606 | VVSTLPQAV | W | 355 |
| 127 | NS3 | 1291 | 1299 | ITYSTYGKF | W | 356 |
| 128 | C | 143 | 151 | PLGGAARAL | W | 357 |
| 129 | NS3 | 1584 | 1592 | PYLVAYQAT | W | 358 |
| 130 | NS3 | 1638 | 1646 | THPITKYIM | W | 359 |
| 131 | NS3 | 1264 | 1272 | FGAYMSKAH | W | 360 |
| 132 | C | 177 | 185 | FLLALLSCL | N | 361 |
| 133 | C | 174 | 182 | FSIFLLALL | N | 362 |
| 134 | C | 125 | 133 | TLTCGFADL | N | 363 |
| 135 | C | 133 | 141 | LMGYIPLVG | N | 364 |
| 136 | C | 83 | 91 | WPLYGNEGL | N | 365 |
| 137 | C | 135 | 143 | GYIPLVGAP | N | 366 |
| 138 | C | 89 | 97 | EGLGWAGWL | N | 367 |
| 139 | C | 181 | 189 | LLSCLTIPA | N | 368 |
| 140 | C | 80 | 88 | GYPWPLYGN | N | 369 |
| 141 | C | 111 | 119 | DPRRRSRNL | N | 370 |

| | | | | | |
|---|---|---|---|---|---|
| | | | | Score | SEQ ID |
| Protein | CS_fr | CS_to | pep_seq | PIC | NO |

Epimmune

| | | | |
|---|---|---|---|
| C | GFADLMGYI | 67 | 236 |
| | SFSIFLLALF | 69 | 1729 |
| E1 | CWVALTPTL | 11 | 950 |
| | AYFSMQGAW | 37 | 902 |
| | AWAKVVVIL | 8.8 | 896 |
| E2 | HYAPRPCGI | 9.9 | 1246 |
| E2 | HYPPRPCGI | 11 | 1251 |
| E2 | HYPPKPCGI | 13 | 1250 |
| E2 | HYPYRLWHY | 3.7 | 1252 |
| E2 | LWHYPCTVNF | 77 | 1484 |
| E2 | HYPCTVNFTI | 66 | 1247 |
| E2 | HYPCTVNYTI | 65 | 1249 |
| E2 | HYPCTVNFTL | 64 | 1248 |
| | NYTIFKIRM | 64 | 1543 |
| | EWAILPCSY | 76 | 1043 |
| | KWEYVVLLF | 6.5 | 1354 |
| | KWEWVVLLF | 6.5 | 1353 |
| | EYVVLLFLL | 23 | 1047 |
| | EWVVLLFLL | 70 | 1045 |
| | EWVILLFLL | 31 | 1044 |
| P7 | SFLVFFCAAW | 67 | 1728 |
| P7 | WYLVAFCAAW | 58 | 2023 |
| P7 | VFFCAAWYI | 6.9 | 1907 |
| | HWIGRLIWW | 13 | 1245 |
| | LYPSLIFDI | 21 | 1489 |
| | FYPGVVFDI | 2.7 | 1101 |
| | VFDITKWLL | 55 | 1906 |
| | PYFVRAHVL | 21 | 1592 |
| | PYFVRAHALL | 49 | 1591 |
| | YFVRAHALL | 1.4 | 2032 |
| | TYIYNHLTPL | 98 | 1884 |
| | PMEIKVITW | 73 | 1561 |
| | GYTSKGWKL | 52 | 1214 |
| | AYMSKAHGI | 76 | 904 |
| NS3 | TYSTYGKFL | 97 | 241 |
| NS3 | YYRGLDVSI | 80 | 2082 |
| NS3 | TFTIETTTL | 66 | 1823 |
| NS3 | FWESVFTGL | 52 | 234 |
| NS3 | FWEAVFTGL | 56 | 1099 |
| NS3 | SWDVMWKCLI | 59 | 1805 |
| NS3 | IMACMSADL | 94 | 90 |
| NS3 | VMACMSADL | 60 | 1936 |
| | PYIEQAQAI | 58 | 1593 |
| | NWQKLEAFW | 20 | 1542 |
| NS4B | TFWAKHMWNF | 50 | 1824 |
| NS4B | AFWAKHMWNF | 87 | 803 |
| NS4B | QFWAKHMWNF | 93 | 1604 |
| NS4B | VFWAKHMWNF | 28 | 1909 |
| NS4B | FWAKHMWNF | 0.23 | 1095 |
| NS4B | FWANDMWNF | 0.19 | 1097 |
| NS4B | FWARHMWNF | 0.16 | 1098 |
| NS4B | NFISGIQYL | 91 | 1521 |
| | SMMAFSAAL | 96 | 1751 |
| NS5A | SWLRDVWDW | 26 | 1807 |
| NS5A | RYAPPCKPL | 32 | 1715 |
| NS5A | RYAPPCKPLL | 20 | 1716 |
| NS5A | KFPPALPIW | 5.1 | 1322 |
| NS5A | KYPPALPIW | 0.75 | 1355 |
| | DYNPPLLETW | 77 | 996 |
| NS5B | SYTWTGALI | 20 | 1810 |
| NS5B | SYSWTGALI | 42 | 1809 |
| NS5B | HYRDVLKEM | 18 | 1253 |
| NS5B | LYDVIQKLSI | 68 | 1486 |
| NS5B | RMILMTHFF | 6.6 | 59 |
| NS5B | RMVLMTHFF | 13 | 1671 |
| NS5B | LMTHFFSILL | 80 | 1415 |

TABLE 5

Predicted HLA-B*0702 binding peptides

| | | | | | |
|---|---|---|---|---|---|
| | | | | | SEQ ID |
| Protein | CS_fr | CS_to | pep_seq | Score | NO |

Algonomics
9-mer

| | | | | | | |
|---|---|---|---|---|---|---|
| 1 | NS5B | 2836 | 2844 | APTLWARMI | S | 371 |
| 2 | NS3 | 1503 | 1511 | TPGERPSGM | S | 372 |
| 3 | NS5B | 2616 | 2624 | SPGQRVEFL | S | 373 |
| 4 | C | 111 | 119 | DPRRRSRNL | S | 374 |
| 5 | C | 169 | 177 | LPGCSFSIF | S | 375 |
| 6 | NS3 | 1383b | 1391b | EVIKGGRHL | M | 376 |
| 7 | NS3 | 1383 | 1391 | EAIKGGRHL | M | 377 |
| 8 | C | 83 | 91 | WPLYGNEGL | M | 378 |
| 9 | C | 150 | 158 | ALAHGVRVL | M | 379 |
| 10 | C | 37 | 45 | LPRRGPRLG | M | 380 |
| 11 | NS3 | 1599 | 1607 | APPPSWDQM | M | 381 |
| 12 | NS3 | 1620 | 1628 | GPTPLLYRL | M | 382 |
| 13 | NS3 | 1531 | 1539 | TPAETSVRL | M | 383 |
| 14 | C | 142 | 150 | APLGGAARA | M | 384 |
| 15 | NS3 | 1260 | 1268 | ATLGFGAYM | M | 385 |
| 16 | C | 99 | 107 | SPRGSRPSW | M | 386 |
| 17 | C | 41 | 49 | GPRLGVRAT | M | 387 |
| 18 | NS5B | 2668 | 2676 | EARQAIRSL | M | 388 |
| 19 | NS3 | 1622 | 1630 | TPLLYRLGA | M | 389 |
| 20 | C | 57 | 65 | QPRGRRQPI | M | 390 |
| 21 | NS3 | 1244 | 1252 | YAAQGYKVL | M | 391 |
| 22 | NS3 | 1357 | 1365 | VPHPNIEEI | M | 392 |
| 23 | NS5B | 2605 | 2613 | AVMGSSYGF | M | 393 |
| 24 | NS3 | 1415 | 1423 | VAYYRGLDV | M | 394 |
| 25 | NS3 | 1359 | 1367 | HPNIEEIGL | M | 395 |
| 26 | NS3 | 1639 | 1647 | HPITKYIMA | M | 396 |
| 27 | NS3 | 1230 | 1238 | APTGSGKST | M | 397 |
| 28 | NS3 | 1560 | 1568 | SVFTGLTHI | M | 398 |
| 29 | NS3 | 1171 | 1179 | CPSGHVVGV | M | 399 |
| 30 | NS3 | 1413 | 1421 | NAVAYYRGL | M | 400 |
| 31 | NS5B | 2720 | 2728 | SAACRAAKL | M | 401 |
| 32 | NS3 | 1404 | 1412 | AAKLSALGL | M | 402 |
| 33 | C | 147 | 155 | AARALAHGV | M | 403 |
| 34 | C | 4 | 12 | NPKPQRKTK | W | 404 |
| 35 | NS5B | 2666 | 2674 | APEARQAIR | W | 405 |
| 36 | C | 115 | 123 | RSRNLGKVI | W | 406 |
| 37 | C | 24 | 32 | FPGGGQIVG | W | 407 |
| 38 | NS3 | 1289 | 1297 | APITYSTYG | W | 408 |
| 39 | C | 65 | 73 | IPKARRPEG | W | 409 |
| 40 | NS3 | 1219 | 1227 | VPQTFQVAH | W | 410 |
| 41 | NS5B | 2826 | 2834 | NSWLGNIIM | W | 411 |
| 42 | C | 149 | 157 | RALAHGVRV | W | 412 |
| 43 | NS3 | 1490 | 1498 | RTGRGRRGI | W | 413 |
| 44 | NS3 | 1641 | 1649 | ITKYIMACM | W | 414 |
| 45 | NS3 | 1426 | 1434 | IPTSGDVVV | W | 415 |
| 46 | NS3 | 1616 | 1624 | PTLHGPTPL | W | 416 |
| 47 | NS3 | 1373 | 1381 | IPFYGKAIP | W | 417 |
| 48 | NS5B | 2835 | 2843 | YAPTLWARM | W | 418 |
| 49 | NS5B | 2796 | 2804 | ASGKRVYYL | W | 419 |
| 50 | NS3 | 1338 | 1346 | TAGARLVVL | W | 420 |
| 51 | NS5B | 2633 | 2641 | CPMGFSYDT | W | 421 |
| 52 | NS3 | 1384 | 1392 | AIKGGRHLI | W | 422 |
| 53 | NS3 | 1255 | 1263 | NPSVAATLG | W | 423 |
| 54 | NS3 | 1325 | 1333 | TILGIGTVL | W | 424 |
| 55 | NS3 | 1380 | 1388 | IPIEAIKGG | W | 425 |
| 56 | NS3 | 1637 | 1645 | LTHPITKYI | W | 426 |
| 57 | NS3 | 1252 | 1260 | LVLNPSVAA | W | 427 |
| 58 | NS5B | 2678 | 2686 | ERLYIGGPL | W | 428 |
| 59 | NS3 | 1188 | 1196 | GVAKAVDFI | W | 429 |
| 60 | NS5B | 2568 | 2576 | QPEKGGRKP | W | 430 |
| 61 | C | 104 | 112 | RPSWGPTDP | W | 431 |
| 62 | C | 161 | 169 | GVNYATGNL | W | 432 |
| 63 | NS3 | 1402 | 1410 | ELAAKLSAL | W | 433 |
| 64 | NS3 | 1540 | 1548 | RAYLNTPGL | W | 434 |
| 65 | NS5B | 2572 | 2580 | GGRKPARLI | W | 435 |
| 66 | NS3 | 1277 | 1285 | NIRTGVRTI | W | 436 |
| 67 | NS3 | 1433 | 1441 | VVVATDALM | W | 437 |
| 68 | NS3 | 1246 | 1254 | AQGYKVLVL | W | 438 |
| 69 | NS3 | 1337 | 1345 | ETAGARLVV | W | 439 |
| 70 | NS5B | 2725 | 2733 | AAKLQDCTM | W | 440 |
| 71 | NS3 | 1162 | 1170 | KGSSGGPLL | W | 441 |
| 72 | C | 137 | 145 | IPLVGAPLG | W | 442 |
| 73 | NS3 | 1583 | 1591 | FPYLVAYQA | N | 443 |
| 74 | C | 93 | 101 | WAGWLLSPR | N | 444 |
| 75 | C | 78 | 86 | QPGYPWPLY | N | 445 |
| 76 | C | 179 | 187 | LALLSCLTI | N | 446 |
| 77 | C | 154 | 162 | GVRVLEDGV | N | 447 |
| 78 | C | 77 | 85 | AQPGYPWPL | N | 448 |
| 79 | C | 38 | 46 | PRRGPRLGV | N | 449 |
| 80 | C | 37 | 46 | LPRRGPRLGV | S | 450 |

| | | | | | |
|---|---|---|---|---|---|
| | | | | Score | SEQ ID |
| Protein | CS_fr | CS_to | pep_seq | PIC | NO |

Epimmune

| | | | |
|---|---|---|---|
| C | LPRRGPRLGV | 4.9 | 450 |
| C | QPRGRRQPI | 3.0 | 390 |
| C | QPRRRRQPI | 3.9 | 1617 |
| C | QPGYPWPLY | 39918 | 216 |
| C | SPRGSRPSW | 1.6 | 386 |
| C | SPRGSRPNW | 11 | 1772 |
| C | SPRGSRPTW | 2.8 | 1774 |
| C | DPRRRSRNL | 12 | 370 |
| C | APLGGAARAL | 3.5 | 836 |
| C | APLGGVARAL | 5.5 | 837 |
| C | APVGGVARAL | 8.1 | 855 |
| C | LPGCSFSIF | 101 | 375 |
| C | LPGCSFSIFL | 32 | 1426 |
| E1 | YPGHVSGHRM | 264 | 2063 |
| E2 | APRPCGIVPA | 38 | 847 |
| E2 | RPCGIVPAL | 1.9 | 1675 |
| E2 | VPARSVCGPV | 48 | 1945 |
| E2 | VPASSVCGPV | 54 | 1947 |
| E2 | GPWLTPRCL | 64 | 1173 |
| E2 | GPWLTPRCM | 105 | 1175 |
| E2 | TPRCLVDYPY | 238 | 1857 |
| E2 | TPRCMVDYPY | 445 | 1858 |
| E2 | YPCTVNFTI | 71 | 2059 |
| E2 | YPCTVNFSI | 42 | 2058 |
| E2 | YPCTVNFTL | 17 | 2061 |
| E2 | YPCTVNFTIF | 307 | 2060 |
| | LPCSFSDLPA | 100 | 1423 |
| | LPALSTGLL | 41 | 1420 |
| P7 | VPGAAYALY | 27777 | 1949 |
| P7 | WPLLLLLLAL | 7.5 | 2018 |
| | VPYFVRAHAL | 9.1 | 1959 |
| | TPYFVRAHVL | 32 | 1863 |
| | SPMEKKVIV | 31 | 1769 |
| | GPKGPVTQM | 86 | 1166 |
| | CPSGHVVGI | 62 | 933 |
| | CPRGHAVGI | 3.6 | 929 |
| | CPAGHAVGIF | 56 | 925 |
| | CPRGHAVGIF | 6.9 | 930 |
| NS3 | VPAAYAAQGY | 2734 | 1943 |
| NS3 | NPSVAATLGF | 1610 | 1532 |
| NS3 | IPFYGKAIPI | 29 | 1284 |
| NS3 | IPFYGKAIPL | 7.9 | 1285 |
| NS3 | TPGERPSGM | 834 | 372 |
| NS3 | TPGERPSGMF | 699 | 1845 |
| NS3 | RPSGMFDSV | 9.4 | 1688 |
| NS3 | RPSGMFDSSV | 37 | 1687 |
| NS3 | RPSGMFDSVV | 58 | 1689 |
| NS3 | LPVCQDHLEF | 5715 | 1444 |
| NS3 | APPPSWDQM | 933 | 381 |
| NS3 | KPTLHGPTPL | 7.9 | 1343 |
| NS3 | KPTLVGPTPL | 34 | 1345 |
| NS3 | KPTLQGPTPL | 47 | 1344 |
| NS3 | HPITKYIMA | 39 | 396 |
| NS3 | HPVTKYIMA | 48 | 1238 |
| NS4B | LPYIEQGMQL | 43 | 1447 |
| NS4B | APYIEQAQAI | 44 | 857 |
| NS4B | NPAIASLMAF | 7.2 | 1528 |
| NS4B | NPAVASMMAF | 14 | 1530 |
| NS4B | NPAVASLMAF | 11 | 1529 |
| NS4B | SPLTTNQTM | 80 | 1766 |
| NS4B | APPSAASAFV | 76 | 845 |
| NS4B | LPAILSPGAL | 4.4 | 1418 |
| NS4B | GPGEGAVQWM | 976 | 1163 |
| | KPAPNFKTAI | 26 | 1335 |
| NS5A | EPDVAVLTSM | 597 | 1023 |
| NS5A | LPKSRFPPA | 50 | 1432 |
| NS5A | LPKSRFPPAL | 14 | 1433 |
| NS5A | LPIWARPDY | 4290 | 1431 |
| NS5A | RPDYNPPLL | 73 | 1677 |
| NS5A | VPPVVHGCPL | 26 | 1953 |
| | PPRKKRTVV | 31 | 1577 |
| NS5B | LPINALSNSL | 46 | 1430 |
| NS5B | TPPHSAKSKF | 699 | 1856 |
| NS5B | PPHSAKSKF | 9170 | 1568 |
| NS5B | PPHSARSKF | 4229 | 1569 |
| NS5B | SPGQRVEFL | 21 | 373 |
| NS5B | SPAQRVEFL | 7.6 | 1757 |
| NS5B | LPTSFGNTI | 61 | 1443 |
| NS5B | PPGDPPQPEY | 633519 | 1566 |
| NS5B | APTLWARMI | 14 | 371 |
| NS5B | APTIWVRMV | 19 | 850 |
| NS5B | APTLWARMIL | 1.2 | 853 |
| NS5B | APTIWVRMVL | 1.9 | 851 |
| NS5B | RPRLLLLGL | 0.17 | 1682 |
| NS5B | RPRLLLLGLL | 072 | 1683 |

TABLE 6

Predicted HLA-B*0801 binding peptides

| | | | | | |
|---|---|---|---|---|---|
| | | | | | SEQ ID |
| Protein | CS_fr | CS_to | pep_seq | Score | NO |

Algonomics
9-mer

| | | | | | | |
|---|---|---|---|---|---|---|
| 1 | C | 111 | 119 | DPRRRSRNL | S | 451 |
| 2 | C | 57 | 65 | QPRGRRQPI | S | 452 |
| 3 | C | 65 | 73 | IPKARRPEG | S | 453 |
| 4 | NS3 | 1639 | 1647 | HPITKYIMA | S | 454 |
| 5 | NS3 | 1395 | 1403 | HSKKKCDEL | S | 455 |
| 6 | NS3 | 1486 | 1494 | QRRGRTGRG | M | 456 |
| 7 | C | 4 | 12 | NPKPQRKTK | M | 457 |
| 8 | NS5B | 2798 | 2806 | GKRVYYLTR | M | 458 |
| 9 | NS3 | 1536 | 1544 | SVRLRAYLN | M | 459 |
| 10 | C | 132 | 140 | DLMGYIPLV | M | 460 |
| 11 | NS3 | 1413 | 1421 | NAVAYYRGL | M | 461 |
| 12 | C | 72 | 80 | EGRTWAQPG | M | 462 |
| 13 | NS3 | 1641 | 1649 | ITKYIMACM | M | 463 |
| 14 | NS3 | 1494 | 1502 | GRRGIYRFV | M | 464 |
| 15 | NS5B | 2838 | 2846 | TLWARMILM | M | 465 |
| 16 | NS5B | 2640 | 2648 | DTRCFDSTV | M | 466 |
| 17 | NS3 | 1606 | 1614 | QMWKCLIRL | M | 467 |
| 18 | NS3 | 1611 | 1619 | LIRLKPTLH | M | 468 |
| 19 | NS3 | 1415 | 1423 | VAYYRGLDV | M | 469 |
| 20 | NS3 | 1637 | 1645 | LTHPITKYI | M | 470 |
| 21 | NS5B | 2840 | 2848 | WARMILMTH | M | 471 |
| 22 | NS3 | 1583 | 1591 | FPYLVAYQA | M | 472 |
| 23 | NS5B | 2672 | 2680 | AIRSLTERL | M | 473 |
| 24 | NS5B | 2668 | 2676 | EARQAIRSL | M | 474 |
| 25 | NS5B | 2696 | 2704 | YRRCRASGV | M | 475 |
| 26 | C | 8 | 16 | QRKTKRNTN | W | 476 |
| 27 | NS3 | 1393 | 1401 | FCHSKKKCD | W | 477 |
| 28 | NS5B | 2627 | 2635 | AWKSKKCPM | W | 478 |
| 29 | C | 63 | 71 | QPIPKARRP | W | 479 |
| 30 | NS3 | 1553 | 1561 | DHLEFWESV | W | 480 |
| 31 | NS5B | 2797 | 2805 | SGKRVYYLT | W | 481 |
| 32 | NS3 | 1376 | 1384 | YGKAIPIEA | W | 482 |
| 33 | NS3 | 1234 | 1242 | SGKSTKVPA | W | 483 |
| 34 | NS3 | 1622 | 1630 | TPLLYRLGA | W | 484 |
| 35 | NS5B | 2831 | 2839 | NIIMYAPTL | W | 485 |
| 36 | NS3 | 1376b | 1384b | YGKAIPIEV | W | 486 |
| 37 | C | 33 | 41 | GVYLLPRRG | W | 487 |
| 38 | NS5B | 2761 | 2769 | EAMTRYSAP | W | 488 |
| 39 | C | 89 | 97 | EGLGWAGWL | W | 489 |
| 40 | NS3 | 1491 | 1499 | TGRGRRGIY | W | 490 |
| 41 | NS3 | 1384b | 1392b | VIKGGRHLI | W | 491 |
| 42 | NS3 | 1387 | 1395 | GGRHLIFCH | W | 492 |
| 43 | NS3 | 1569 | 1577 | DAHFLSQTK | W | 493 |
| 44 | NS5B | 2714 | 2722 | TCYLKASAA | W | 494 |
| 45 | NS5B | 2755 | 2763 | SLRVFTEAM | W | 495 |
| 46 | NS3 | 1480 | 1488 | DAVSRSQRR | W | 496 |
| 47 | NS3 | 1605 | 1613 | DQMWKCLIR | W | 497 |
| 48 | NS5B | 2586 | 2594 | GVRVCEKMA | W | 498 |
| 49 | NS3 | 1237 | 1245 | STKVPAAYA | W | 499 |
| 50 | NS5B | 2812 | 2820 | LARAAWETA | W | 500 |
| 51 | C | 36 | 44 | LLPRRGPRL | W | 501 |
| 52 | NS3 | 1294 | 1302 | STYGKFLAD | W | 502 |
| 53 | NS5B | 2572 | 2580 | GGRKPARLI | W | 503 |
| 54 | NS3 | 1384 | 1392 | AIKGGRHLI | W | 504 |
| 55 | C | 60 | 68 | GRRQPIPKA | W | 505 |
| 56 | NS3 | 1610 | 1618 | CLIRLKPTL | W | 506 |
| 57 | NS5B | 2573 | 2581 | GRKPARLIV | W | 507 |
| 58 | NS5B | 2833 | 2841 | IMYAPTLWA | W | 508 |
| 59 | C | 115 | 123 | RSRNLGKVI | W | 509 |
| 60 | NS3 | 1372 | 1380 | EIPFYGKAI | W | 510 |
| 61 | NS3 | 1277 | 1285 | NIRTGVRTI | W | 511 |
| 62 | C | 35 | 43 | YLLPRRGPR | W | 512 |
| 63 | C | 147 | 155 | AARALAHGV | W | 513 |
| 64 | C | 37 | 45 | LPRRGPRLG | W | 514 |

Epimmune

| | | |
|---|---|---|
| C | TNRRPQDVKF | 1838 |
| C | NRRPQDVKF | 685 |
| C | DVKFPGGGQI | 990 |
| C | YLLPRRGPRL | 2047 |
| C | LLPRRGPRL | 132 |
| C | QPRGRRQPI | 390 |
| C | TDPRRRSRNL | 1817 |
| C | DPRRRSRNL | 370 |
| C | RSRNLGKVI | 318 |
| C | RNLGKVIDTL | 1673 |
| E2 | WTRGERCDL | 2022 |
| E2 | DLEDRDRSEL | 964 |
| E2 | RDRSELSPL | 1636 |
| E2 | RDRSELSPLL | 1637 |
| E2 | LADARVCACL | 1358 |
| NS2 | NVRGGRDAI | 1538 |
| NS2 | NVRGGRDAII | 1539 |
| NS2 | VRGGRDAII | 1965 |
| NS2 | VRGGRDAIIL | 1966 |
| NS2 | GGRDAIILL | 1138 |
| NS2 | PVSARRGREI | 1589 |
| NS2 | VSARRGREI | 1969 |
| NS2 | VSARRGREIL | 1970 |
| NS2 | SARRGREIL | 1718 |
| NS2 | SARRGREILL | 1719 |
| NS2 | ARRGREILL | 865 |
| NS3 | QTRGLLGCI | 1621 |
| NS3 | QTRGLLGCII | 1622 |
| NS3 | YLVTRHADVI | 2054 |
| NS3 | RRRGDSRGSL | 1697 |
| NS3 | RGDSRGSLL | 1648 |
| NS3 | YLKGSSGGPL | 2046 |
| NS3 | RGVAKAVDF | 317 |
| NS3 | RGVAKAVDFI | 1653 |
| NS3 | ETTMRSPVF | 257 |
| NS3 | AQGYKVLVL | 130 |
| NS3 | AYMSKAHGI | 904 |
| NS3 | NIRTGVRTI | 436 |
| NS3 | TAGARLVVL | 249 |
| NS3 | PFYGKAIPI | 264 |
| NS3 | PFYGKAIPL | 1554 |
| NS3 | IKGGRHLIF | 311 |
| NS3 | CHSKKKCDEL | 918 |
| NS3 | HSKKKCDEL | 455 |
| NS3 | YYRGLDVSVI | 2083 |
| NS3 | TPGERPSGMF | 1845 |
| NS3 | DQMWKCLIRL | 982 |
| NS3 | KCLIRLKPTL | 1319 |
| NS4B | AEQFKQKAL | 794 |
| NS4B | QFKQKALGL | 1602 |
| NS4B | QFKQKALGLL | 1603 |
| NS4B | WAKHMWNFI | 1993 |
| NS4B | IGLGKVLVDI | 1267 |
| NS4B | LGKVLVDIL | 1382 |
| NS4B | QWMNRLIAF | 1623 |
| NS5A | KGVWRGDGI | 1326 |
| NS5A | LARGSPPSL | 1363 |
| NS5A | LWRQEMGGNI | 1485 |
| NS5A | ESENKWIL | 1030 |
| NS5A | ENKWILDSF | 1021 |
| NS5A | WARPDYNPPL | 1998 |
| NS5B | RQKKVTFDRL | 1695 |
| NS5B | QKKVTFDRL | 1607 |
| NS5B | VTFDRLQVL | 1975 |
| NS5B | TIMAKNEVF | 1827 |
| NS5B | EKGGRKPARL | 1015 |
| NS5B | KGGRKPARL | 1323 |
| NS5B | KGGRKPARLI | 1324 |
| NS5B | GGRKPARLI | 435 |
| NS5B | GRKPARLIVF | 1182 |
| NS5B | RKPARLIVF | 646 |
| NS5B | PARLIVFPDL | 1545 |
| NS5B | GVRVCEKMAL | 1203 |
| NS5B | SPGQRVEFL | 373 |
| NS5B | YRRCRASGVL | 2066 |
| NS5B | LTRDPTTPL | 1475 |
| NS5B | WARMILMTHF | 1997 |
| NS5B | IERLHGLSAF | 1264 |
| NS5B | IQRLHGLSAF | 1298 |
| NS5B | CLRKLGVPPL | 921 |
| NS5B | LRKLGVPPL | 1455 |
| NS5B | RARSVRAKL | 1632 |
| NS5B | RARSVRAKLL | 1633 |
| NS5B | ARSVRAKLL | 867 |
| NS5B | NWAVKTKLKL | 1540 |
| NS5B | NWAVRTKLKL | 1541 |
| NS5B | RTKLKLTPI | 1705 |

Algonomics
10-mer

| | | | | | |
|---|---|---|---|---|---|
| Core | 65 | 74 | IPKARRPEGR | S | 1287 |
| Core | 4 | 13 | NPKPQRKTKR | M | 1531 |
| Ns3 | 1395 | 1403 | HSKKKCDELA | M | 1243 |
| Core | 111 | 120 | DPRRRSRNLG | M | 975 |
| Core | 37 | 46 | LPRRGPRLGV | M | 450 |
| Core | 8 | 17 | QRKTKRNTNR | M | 1618 |
| Core | 21 | 30 | DVKFPGGGQI | M | 990 |
| Ns5b | 2696 | 1655 | YRRCRASGVL | M | 2066 |
| Ns5b | 2626 | 1655 | NAWKSKKCPM | M | 1514 |
| Core | 57 | 66 | QPRGRRQPIP | M | 1616 |
| Ns3 | 1491 | 1499 | TGRGRRGIYR | M | 1825 |
| Ns3 | 1605 | 1613 | DQMWKCLIRL | M | 982 |
| Ns3 | 1291 | 1299 | ITYSTYGKFL | M | 1310 |
| Ns3 | 1234 | 1242 | SGKSTKVPAA | M | 1734 |
| Ns3 | 1376 | 1384 | YGKAIPIEVI | M | 2035 |
| Core | 35 | 44 | YLLPRRGPRL | M | 2047 |
| Ns5b | 2797 | 1655 | SGKRVYYLTR | M | 1733 |
| Ns3 | 1493 | 1501 | RGRRGIYRFV | W | 1650 |
| Ns4b | 1844 | 1655 | LGKVLVDILA | W | 1383 |
| Core | 89 | 98 | EGMGWAGWLL | W | 1012 |
| Ns3 | 1237 | 1245 | STKVPAAYAA | W | 1790 |
| Ns3 | 1189 | 1197 | VAKAVDFIPV | W | 1893 |
| Ns3 | 1609 | 1617 | KCLIRLKPTL | W | 1319 |
| Ns3 | 1494 | 1502 | GRRGIYRFVT | W | 1183 |
| Ns3 | 1393 | 1401 | FCHSKKKCDE | W | 1051 |
| Ns3 | 1637 | 1645 | LTHPITKYIM | W | 1469 |
| Ns3 | 1482 | 1490 | VSRSQRRGRT | W | 1972 |
| Ns5b | 2677 | 1655 | TERLYIGGPL | W | 1820 |
| Ns3 | 1340 | 1348 | GARLVVLATA | W | 1106 |
| Ns5b | 2761 | 1655 | EAMTRYSAPP | W | 999 |
| Ns5b | 2627 | 1655 | AWKSKKCPMG | W | 899 |
| Ns5b | 2573 | 1655 | GRKPARLIVF | W | 1182 |
| Ns5b | 2572 | 1655 | GGRKPARLIV | W | 1139 |
| Ns3 | 1622 | 1630 | TPLLYRLGAV | W | 1851 |
| Core | 99 | 108 | SPRGSRPSWG | W | 1773 |
| Ns3 | 1611 | 1619 | LIRLKPTLHG | W | 1387 |
| Core | 41 | 50 | GPRLGVRATR | W | 1170 |
| Ns5b | 2671 | 1655 | QAIRSLTERL | W | 1597 |
| Core | 132 | 141 | DLMGYIPLVG | W | 965 |
| Ns5b | 2586 | 1655 | GVRVCEKMAL | W | 1203 |
| Core | 59 | 68 | RGRRQPIPKA | W | 1651 |
| Ns3 | 1488 | 1496 | RGRTGRGRRG | W | 1652 |
| Ns3 | 1294 | 1302 | STYGKFLADG | W | 1795 |
| Ns3 | 1486 | 1494 | QRRGRTGRGR | W | 1619 |
| Ns3 | 1384 | 1392 | VIKGGRHLIF | W | 1917 |
| Ns3 | 1536 | 1544 | SVRLRAYLNT | W | 1802 |
| Ns3 | 1373 | 1381 | IPFYGKAIPI | W | 1284 |
| Ns5b | 2795 | 1655 | DASGKRVYYL | W | 955 |
| Ns3 | 1485 | 1493 | SQRRGRTGRG | W | 1783 |
| Ns3 | 1552 | 1560 | QDHLEFWESV | W | 1600 |
| Core | 45 | 54 | GVRATRKTSE | W | 1200 |
| Ns5b | 2716 | 1655 | YLKASAACRA | W | 2045 |
| Ns5b | 2725 | 1655 | AAKLQDCTML | W | 775 |
| Ns3 | 1160 | 1169 | YLKGSSGGPL | W | 2046 |
| Core | 113 | 122 | RRRSRNLGKV | W | 1698 |
| Ns3 | 1242 | 1250 | AAYAAQGYKV | W | 783 |
| Ns3 | 1606 | 1614 | QMWKCLIRLK | W | 1610 |
| Core | 68 | 77 | ARRPEGRAWA | W | 866 |
| Ns5b | 2694 | 1655 | CGYRRCRASG | W | 917 |
| Ns3 | 1639 | 1647 | HPITKYIMAC | W | 1236 |
| Ns5b | 2840 | 1655 | WARMILMTHF | W | 1997 |

TABLE 7

Predicted HLA-B*3501 binding peptides

| | | | | | |
|---|---|---|---|---|---|
| | | | | | SEQ ID |
| Protein | CS_fr | CS_to | pep_seq | Score | NO |

Algonomics
9-mer

| | | | | | | |
|---|---|---|---|---|---|---|
| 1 | C | 169 | 177 | LPGCSFSIF | S | 515 |
| 2 | NS3 | 1464 | 1472 | FSLDPTFTI | M | 516 |
| 3 | NS5B | 2605 | 2613 | AVMGSSYGF | M | 517 |
| 4 | NS3 | 1583 | 1591 | EPYLVAYQA | M | 518 |
| 5 | C | 24 | 32 | FPGGGQIVG | M | 519 |
| 6 | NS5B | 2607 | 2615 | MGSSYGFQY | M | 520 |
| 7 | NS3 | 1531 | 1539 | TPAETSVRL | M | 521 |
| 8 | C | 156 | 164 | RVLEDGVNY | M | 522 |
| 9 | NS3 | 1359 | 1367 | HPNIEEIGL | M | 523 |
| 10 | NS3 | 1581 | 1589 | DNFPYLVAY | W | 524 |
| 11 | NS5B | 2795 | 2803 | DASGKRVYY | W | 525 |
| 12 | NS3 | 1456 | 1464 | TCVTQTVDF | W | 526 |
| 13 | NS3 | 1175 | 1183 | HVVGVFRAA | W | 527 |
| 14 | NS3 | 1522 | 1530 | DAGCAWYEL | W | 528 |
| 15 | NS5B | 2826 | 2834 | NSWLGNIIM | W | 529 |
| 16 | NS3 | 1438 | 1446 | DALMTGYTG | W | 530 |
| 17 | NS3 | 1367 | 1375 | LSNNGEIPF | W | 531 |
| 18 | NS5B | 2615 | 2623 | YSPGQRVEF | W | 532 |
| 19 | NS5B | 2840 | 2848 | WARMILMTH | W | 533 |
| 20 | NS3 | 1357 | 1365 | VPHPNIEEI | W | 534 |
| 21 | C | 78 | 86 | QPGYPWPLY | W | 535 |
| 22 | NS5B | 2720 | 2728 | SAACRAAKL | W | 536 |
| 23 | NS3 | 1289 | 1297 | APITYSTYG | W | 537 |
| 24 | C | 83 | 91 | WPLYGNEGL | W | 538 |
| 25 | NS3 | 1620 | 1628 | GPTPLLYRL | W | 539 |
| 26 | C | 174 | 182 | FSIFLLALL | W | 540 |
| 27 | NS3 | 1171 | 1179 | CPSGHVVGV | W | 541 |
| 28 | NS5B | 2633 | 2641 | CPMGFSYDT | W | 542 |
| 29 | NS5B | 2772 | 2780 | DPPQPEYDL | W | 543 |
| 30 | NS3 | 1219 | 1227 | VPQTFQVAH | W | 544 |
| 31 | C | 149 | 157 | RALAHGVRV | W | 545 |
| 32 | NS3 | 1433 | 1441 | VVVATDALM | W | 546 |
| 33 | C | 137 | 145 | IPLVGAPLG | W | 547 |
| 34 | NS3 | 1622 | 1630 | TPLLYRLGA | W | 548 |
| 35 | NS3 | 1240 | 1248 | VPAAYAAQG | W | 549 |
| 36 | NS3 | 1410 | 1418 | LGLNAVAYY | W | 550 |
| 37 | NS3 | 1578 | 1586 | QAGDNFPYL | W | 551 |
| 38 | C | 18 | 26 | RPQDVKFPG | W | 552 |
| 39 | NS5B | 2588 | 2596 | RVCEKMALY | W | 553 |
| 40 | NS5B | 2740 | 2748 | LVVICESAG | W | 554 |
| 41 | C | 142 | 150 | APLGGAARA | W | 555 |
| 42 | C | 172 | 180 | CSFSIFLLA | W | 556 |
| 43 | NS3 | 1259 | 1267 | AATLGFGAY | W | 557 |
| 44 | C | 128 | 136 | CGFADLMGY | W | 558 |
| 45 | NS5B | 2794 | 2802 | HDASGKRVY | W | 559 |
| 46 | NS3 | 1260 | 1268 | ATLGFGAYM | W | 560 |
| 47 | NS3 | 1380b | 1388b | IPIEVIKGG | W | 561 |
| 48 | NS5B | 2751 | 2759 | EDAASLRVF | W | 562 |

Algonomics
10-mer

| | | | | | | |
|---|---|---|---|---|---|---|
| 1 | Ns3 | 1548 | 1556 | LPVCQDHLEF | S | 1444 |
| 2 | Core | 169 | 178 | LPGCSFSIFL | M | 1426 |
| 3 | Ns3 | 1196 | 1204 | IPVESMETTM | M | 1296 |
| 4 | Ns3 | 1408 | 1416 | SALGLNAVAY | M | 1717 |
| 5 | Ns5b | 2604 | 1655 | QAVMGSSYGF | M | 1599 |
| 6 | Ns4b | 1873 | 1655 | MPSTEDLVNL | M | 1505 |
| 7 | Core | 24 | 33 | FPGGGQIVGG | M | 1077 |
| 8 | Ns3 | 1373 | 1381 | IPFYGKAIPI | M | 1284 |
| 9 | Ns3 | 1255 | 1263 | NPSVAATLGF | M | 1532 |
| 10 | Ns3 | 1521 | 1529 | YDAGCAWYEL | M | 2030 |
| 11 | Ns3 | 1171 | 1180 | CPSGHAVGIF | W | 932 |
| 12 | Ns5b | 2602 | 1655 | LPQAVMGSSY | W | 1437 |
| 13 | Ns5b | 2840 | 1655 | WARMILMTHF | W | 1997 |
| 14 | Ns5b | 2826 | 1655 | NSWLGNIIMY | W | 1534 |
| 15 | Ns3 | 1240 | 1248 | VPAAYAAQGY | W | 1943 |
| 16 | Core | 142 | 151 | APLGGAARAL | W | 836 |
| 17 | Core | 76 | 85 | WAQPGYPWPL | W | 1996 |
| 18 | Ns5b | 2795 | 1655 | DASGKRVYYL | W | 955 |
| 19 | Core | 74 | 83 | RAWAQPGYPW | W | 1634 |
| 20 | Ns3 | 1637 | 1645 | LTHPITKYIM | W | 1469 |
| 21 | Ns3 | 1175 | 1184 | HAVGIFRAAV | W | 1215 |
| 22 | Core | 81 | 90 | YPWPLYGNEG | W | 2064 |
| 23 | Ns5b | 2794 | 1655 | HDASGKRVYY | W | 1216 |
| 24 | Ns3 | 1622 | 1630 | TPLLYRLGAV | W | 1851 |
| 25 | Ns5b | 2836 | 1655 | APTLWARMIL | W | 853 |
| 26 | Ns4b | 1846 | 1655 | KVLVDILAGY | W | 1350 |
| 27 | Ns5b | 2757 | 1655 | RVFTEAMTRY | W | 1712 |
| 28 | Ns3 | 1258 | 1266 | VAATLGFGAY | W | 1887 |
| 29 | Ns5b | 2651 | 1655 | NDIRVEESIY | W | 1515 |
| 30 | Core | 83 | 92 | WPLYGNEGMG | W | 2020 |
| 31 | Ns5b | 2769 | 1655 | PPGDPPQPEY | W | 1566 |
| 32 | Core | 172 | 181 | CSFSIFLLAL | W | 935 |
| 33 | Core | 89 | 98 | EGMGWAGWLL | W | 1012 |
| 34 | Core | 137 | 146 | IPLVGAPLGG | W | 1290 |
| 35 | Ns3 | 1367 | 1375 | LSNTGEIPFY | W | 1459 |
| 36 | Ns5b | 2823 | 1655 | TPVNSWLGNI | W | 1862 |
| 37 | Ns5b | 2734 | 1655 | LVNGDDLVVI | W | 1480 |
| 38 | Ns3 | 1639 | 1647 | HPITKYIMAC | W | 1236 |
| 39 | Core | 18 | 27 | RPQDVKFPGG | W | 1681 |
| 40 | Ns5b | 2580 | 1655 | IVFPDLGVRV | W | 1312 |
| 41 | Ns5b | 2674 | 1655 | RSLTERLYIG | W | 1702 |
| 42 | Ns3 | 1219 | 1227 | VPQTFQVAHL | W | 1954 |
| 43 | Ns3 | 1216 | 1224 | PPAVPQTFQV | W | 1564 |
| 44 | Ns3 | 1242 | 1250 | AAYAAQGYKV | W | 783 |
| 45 | Ns5b | 2616 | 1655 | SPGQRVEFLV | W | 1765 |
| 46 | Ns5b | 2810 | 1655 | TPLARAAWET | W | 1850 |
| 47 | Ns3 | 1357 | 1365 | VPHPNIEEVA | W | 1951 |
| 48 | Ns3 | 1426 | 1434 | IPTSGDVVVV | W | 1295 |
| 49 | Core | 37 | 46 | LPRRGPRLGV | W | 450 |
| 50 | Ns5b | 2563 | 1655 | EVFCVQPEKG | W | 1038 |
| 51 | Ns3 | 1337 | 1345 | ETAGARLVVL | W | 1032 |
| 52 | Ns3 | 1245 | 1253 | AAQGYKVLVL | W | 777 |
| 53 | Ns5b | 2754 | 1655 | ASLRVFTEAM | W | 871 |
| 54 | Ns5b | 2593 | 1655 | MALYDVVSTL | W | 1492 |
| 55 | Ns5b | 2773 | 1655 | PPQPEYDLEL | W | 1575 |
| 56 | Ns4b | 1857 | 1655 | AGVAGALVAF | W | 808 |

TABLE 8

Predicted HLA-B*4403 and HLA-B*4002 binding
peptides

| | | | | | |
|---|---|---|---|---|---|
| | | | | | SEQ ID |
| Protein | CS_fr | CS_to | pep_seq | Score | NO |

Algonomics
9-mer

| | | | | | | |
|---|---|---|---|---|---|---|
| 1 | C | 77 | 85 | AQPGYPWPL | S | 563 |
| 2 | NS3 | 1555 | 1563 | LEFWESVFT | M | 564 |
| 3 | C | 88 | 96 | NEGLGWAGW | M | 565 |
| 4 | NS5B | 2828 | 2836 | WLGNIIMYA | M | 566 |
| 5 | NS5B | 2842 | 2850 | RMILMTHFF | M | 567 |
| 6 | NS5B | 2838 | 2846 | TLWARMILM | M | 568 |
| 7 | NS3 | 1401 | 1409 | DELAAKLSA | M | 569 |
| 8 | C | 28 | 36 | GQIVGGVYL | M | 570 |
| 9 | NS3 | 1558 | 1566 | WESVFTGLT | W | 571 |
| 10 | NS3 | 1633 | 1641 | NEVTLTHPI | W | 572 |
| 11 | NS3 | 1585 | 1593 | YLVAYQATV | W | 573 |
| 12 | NS3 | 1382 | 1390 | IEAIKGGRH | W | 574 |
| 13 | NS3 | 1382b | 1390b | IEVIKGGRH | W | 575 |
| 14 | NS5B | 2621 | 2629 | VEFLVNAWK | W | 576 |
| 15 | NS5B | 2817 | 2825 | WETARHTPV | W | 577 |
| 16 | NS3 | 1606 | 1614 | QMWKCLIRL | W | 578 |
| 17 | C | 90 | 98 | GLGWAGWLL | W | 579 |
| 18 | NS5B | 2677 | 2685 | TERLYIGGP | W | 580 |
| 19 | NS5B | 2590 | 2598 | CEKMALYDV | W | 581 |
| 20 | NS3 | 1336 | 1344 | AETAGARLV | W | 582 |
| 21 | NS3 | 1362 | 1370 | IEEIGLSNN | W | 583 |
| 22 | NS5B | 2679 | 2687 | RLYIGGPLT | W | 584 |
| 23 | NS3 | 1409 | 1417 | ALGLNAVAY | W | 585 |
| 24 | NS5B | 2760 | 2768 | TEAMTRYSA | W | 586 |
| 25 | NS5B | 2776 | 2784 | PEYDLELIT | W | 587 |
| 26 | NS3 | 1440 | 1448 | LMTGYTGDF | W | 588 |
| 27 | NS3 | 1518 | 1526 | CECYDAGCA | W | 589 |
| 28 | NS3 | 1201 | 1209 | METTMRSPV | W | 590 |
| 29 | NS5B | 2833 | 2841 | IMYAPTLWA | W | 591 |
| 30 | NS3 | 1260 | 1268 | ATLGFGAYM | W | 592 |

| | | | | | |
|---|---|---|---|---|---|
| | | | | Score | SEQ ID |
| Protein | CS_fr | CS_to | pep_seq | PIC | NO |

Epimmune

| | | | |
|---|---|---|---|
| | PEGRSWAQPG | 21 | 1548 |
| | PEGRTWAQPG | 62 | 1549 |
| | NEGLGWAGW | 2452 | 565 |
| | NEGLGWAGWL | 58 | 1516 |
| E2 | SELSPLLLST | 8.1 | 1726 |
| | TEWAILPCSY | 30 | 1822 |
| | WEWVILLFL | 1.1 | 2007 |
| | WEWVVLLFL | 2.1 | 2009 |
| | WEYVVLLFL | 2.1 | 2011 |
| | WEWVILLFLL | 1.2 | 2008 |
| | WEWVVLLFLL | 0.67 | 2010 |
| | WEYVVLLFLL | 0.75 | 2012 |
| | AEAALENLV | 47 | 790 |
| | AEAALEKLV | 83 | 788 |
| | AEAALENLVI | 55 | 791 |
| | AEAALEKLVI | 74 | 789 |
| | LENLVILNA | 72 | 1373 |
| NS2 | REMAASCGG | 53 | 1641 |
| | TEPVIFSPM | 1.8 | 1819 |
| | REILLGPADG | 72 | 1638 |
| NS3 | GEIQVLSTV | 11 | 1120 |
| NS3 | GEVQVLSTA | 35 | 1129 |
| NS3 | GEVQVVSTA | 29 | 1131 |
| NS3 | GEIQVLSTVT | 52 | 1121 |
| NS3 | GEVQVLSTAT | 69 | 1130 |
| NS3 | METTMRSPV | 57 | 590 |
| NS3 | LETTMRSPV | 51 | 1375 |
| NS3 | AETAGVRLT | 177 | 797 |
| NS3 | AETAGARLVV | 36 | 796 |
| NS3 | AETAGVRLTV | 70 | 798 |
| NS3 | GEIPFYGKA | 11 | 1116 |
| NS3 | GEIPFYGRA | 7.1 | 1118 |
| NS3 | GEIPFYGKAI | 6.5 | 1117 |
| NS3 | GEIPFYGRAI | 2.7 | 1119 |
| NS3 | DELAAALRG | 284 | 956 |
| NS3 | YELTPAETT | 94 | 2031 |
| NS3 | AETTVRLRA | 120 | 800 |
| NS3 | LEFWEGVFT | 72 | 1369 |
| NS3 | LEFWEGVFTG | 91 | 1370 |
| NS3 | WEAVFTGLT | 12 | 2000 |
| NS3 | WESVFTGLT | 11 | 571 |
| NS3 | WEGVFTGLT | 19 | 2001 |
| NS3 | GENFAYLTA | 31 | 1122 |
| NS3 | GENLPYLVA | 1.2 | 1126 |
| NS3 | GENFPYLVA | 2.4 | 1124 |
| NS3 | GENFAYLTAY | 35 | 1123 |
| NS3 | GENLPYLVAY | 37 | 1127 |
| NS3 | GENFPYLVAY | 12 | 1125 |
| NS3 | NEVVLTHPI | 29 | 1520 |
| NS3 | NEITLTHPV | 49 | 1518 |
| NS3 | NEVTLTHPI | 76 | 572 |
| | LEVVTSTWVL | 31 | 1378 |
| | IELGGKPAL | 7.7 | 1257 |
| | LEQFWAKHMW | 100 | 1374 |
| | LEVFWAKHMW | 84 | 1376 |
| | VEDVVNLLPA | 87 | 1898 |
| | PEFFSWVDGV | 20 | 1547 |
| | TEVLASMLT | 39 | 1821 |
| | AEAAARRLA | 281 | 787 |
| | SEASSSASQL | 10 | 1723 |
| NS5A | VESENKVVV | 97 | 1903 |
| NS5A | VESENKVVI | 67 | 1901 |
| NS5A | VESETKVVIL | 94 | 1905 |
| NS5A | VESENKVVVL | 95 | 1904 |
| NS5A | VESENKVVIL | 58 | 1902 |
| NS5A | REPSIPSEY | 50 | 1642 |
| NS5A | REVSVAAEI | 55 | 1644 |
| NS5A | REISVPAEI | 17 | 1639 |
| NS5A | REVSVPAEI | 38 | 1646 |
| NS5A | REPSIPSEYL | 38 | 1643 |
| NS5A | REVSVAAEIL | 2.6 | 1645 |
| NS5A | REISVPAEIL | 1.1 | 1640 |
| NS5A | REVSVPAEIL | 1.8 | 1647 |
| NS5A | SEYLLPKSRF | 6.2 | 1727 |
| NS5A | AEILRKSRRF | 18 | 792 |
| NS5A | AELATKTFG | 152 | 793 |
| | EEQSVVCCSM | 15 | 1006 |
| | SEDVVCCSM | 35 | 1724 |
| | GEDVVCCSM | 14 | 1113 |
| | EEKLPISPL | 5.6 | 1005 |
| | EEKLPINPL | 7.9 | 1004 |
| | KEVRSLSRRA | 3.2 | 1320 |
| | CEKMALYDI | 99 | 911 |
| | EESIYQACSL | 63 | 1007 |
| | EEARTAIHSL | 91 | 1003 |
| NS5B | PEYDLELIT | 72 | 587 |
| NS5B | WETVRHSPV | 7.0 | 2005 |
| NS5B | WETVRHTPV | 12 | 2006 |
| NS5B | WETARHTPI | 20 | 2003 |
| NS5B | WETARHTPV | 29 | 577 |
| NS5B | FEMYGATYSV | 25 | 1054 |
| NS5B | FEMYGAVYSV | 13 | 1055 |
| NS5B | IEPLDLPQI | 97 | 1258 |
| NS5B | IERLHGLEA | 19 | 1261 |
| NS5B | IERLHGLDA | 20 | 1259 |
| NS5B | IERLHGLSA | 15 | 1263 |
| NS5B | IERLHGLEAF | 33 | 1262 |
| NS5B | IERLHGLDAF | 40 | 1260 |
| NS5B | IERLHGLSAF | 20 | 1264 |
| NS5B | HELTRVAAA | 41 | 1217 |
| NS5B | HELTRVAAAL | 5.8 | 1218 |
| NS5B | GEINRVASCL | 16 | 1115 |

| | | | | | |
|---|---|---|---|---|---|
| | | | | | SEQ ID |
| Protein | CS_fr | CS_to | pep_seq | Score | NO |

Algonomics
10-mer

| | | | | | |
|---|---|---|---|---|---|
| Ns3 | 1382 | 1391 | IEVIKGGRHL | S | 1265 |
| Ns3 | 1555 | 1564 | LEFWESVFTG | M | 1371 |
| Ns5b | 2621 | 2630 | VEFLVNAWKS | M | 1900 |
| Ns5b | 2606 | 2615 | VMGSSYGFQY | M | 1939 |
| Ns3 | 1558 | 1567 | WESVFTGLTH | M | 2002 |
| Core | 88 | 97 | NEGMGWAGWL | M | 1517 |
| Ns5b | 2677 | 2686 | TERLYIGGPL | M | 1820 |
| Ns3 | 1533 | 1542 | AETSVRLRAY | M | 799 |
| Ns3 | 1518 | 1527 | CECYDAGCAW | M | 910 |
| Ns3 | 1201 | 1210 | METTMRSPVF | M | 1493 |
| Ns3 | 1371 | 1380 | GEIPFYGKAI | M | 1117 |
| Ns3 | 1409 | 1418 | ALGLNAVAYY | W | 822 |
| Ns4b | 1913 | 1922 | VQWMNRLIAF | W | 1963 |
| Ns5b | 2750 | 2759 | QEDAASLRVF | W | 1601 |
| Ns3 | 1633 | 1642 | NEVTLTHPIT | W | 1519 |
| Core | 77 | 86 | AQPGYPWPLY | W | 861 |
| Ns5b | 2656 | 2665 | EESIYQCCDL | W | 1008 |
| Ns5b | 2679 | 2688 | RLYIGGPLTN | W | 1670 |
| Ns5b | 2667 | 2676 | PEARQAIRSL | W | 1546 |
| Ns5b | 2838 | 2847 | TLWARMILMT | W | 1837 |
| Ns4b | 1876 | 1885 | TEDLVNLLPA | W | 1818 |
| Ns3 | 1605 | 1614 | DQMWKCLIRL | W | 982 |
| Ns3 | 1401 | 1410 | DELAAKLSAL | W | 957 |
| Ns3 | 1223 | 1232 | FQVAHLHAPT | W | 1080 |
| Ns5b | 2817 | 2826 | WETARHTPVN | W | 2004 |
| Ns4b | 1909 | 1918 | GEGAVQWMNR | W | 1114 |
| Ns5b | 2655 | 2664 | VEESIYQCCD | W | 1899 |
| Ns3 | 1577 | 1586 | KQAGDNFPYL | W | 1346 |
| Ns5b | 2776 | 2785 | PEYDLELITS | W | 1552 |
| Core | 28 | 37 | GQIVGGVYLL | W | 1178 |
| Ns4b | 1847 | 1856 | VLVDILAGYG | W | 1932 |

TABLE 9

Predicted HLA-Cw0401 binding peptides

| | | | | | |
|---|---|---|---|---|---|
| | | | | | SEQ ID |
| Protein | CS_fr | CS_to | pep_seq | Score | NO |

Algonomics
9-mer

| | | | | | | |
|---|---|---|---|---|---|---|
| 1 | Ns3 | 1603 | 1611 | SWDQMWKCL | S | 593 |
| 2 | Core | 173 | 181 | SFSIFLLAL | M | 594 |
| 3 | Ns3 | 1557 | 1565 | FWESVFTGL | M | 595 |
| 4 | Ns3 | 1292 | 1300 | TYSTYGKFL | M | 596 |
| 5 | Ns5b | 2777 | 2785 | EYDLELITS | M | 597 |
| 6 | Ns3 | 1243 | 1251 | AYAAQGYKV | M | 598 |
| 7 | Ns5b | 2581 | 2589 | VFPDLGVRV | M | 599 |
| 8 | Core | 129 | 137 | GFADLMGYI | M | 600 |
| 9 | Ns3 | 1554 | 1562 | HLEFWESVF | W | 601 |
| 10 | Ns3 | 1520 | 1528 | CYDAGGAWY | W | 602 |
| 11 | Core | 85 | 93 | LYGNEGLGW | W | 603 |
| 12 | Ns5b | 2842 | 2850 | RMILMTHFF | W | 604 |
| 13 | Ns3 | 1266 | 1274 | AYMSKAHGV | W | 605 |
| 14 | Ns3 | 1645 | 1653 | IMACMSADL | W | 606 |
| 15 | Ns3 | 1527 | 1535 | WYELTPAET | W | 607 |
| 16 | Core | 23 | 31 | KFPGGGQIV | W | 608 |
| 17 | Ns5b | 2636 | 2644 | GFSYDTRCF | W | 609 |
| 18 | Ns3 | 1417 | 1425 | YYRGLDVSV | W | 610 |
| 19 | Ns3 | 1469 | 1477 | TFTIETTTV | W | 611 |
| 20 | Ns5b | 2758 | 2766 | VFTEAMTRY | W | 612 |
| 21 | Ns3 | 1359 | 1367 | HPNIEEIGL | W | 613 |
| 22 | Core | 122 | 130 | VIDTLTCGF | W | 614 |
| 23 | Ns5b | 2638 | 2646 | SYDTRCFDS | W | 615 |
| 24 | Core | 29 | 37 | QIVGGVYLL | N | 616 |
| 25 | Core | 90 | 98 | GLGWAGWLL | N | 617 |
| 26 | Core | 125 | 133 | TLTCGFADL | N | 618 |
| 27 | Core | 135 | 143 | GYIPLVGAP | N | 619 |
| 28 | Core | 168 | 176 | NLPGCSFSI | N | 620 |

Algonomics
10-mer

| | | | | | |
|---|---|---|---|---|---|
| Ns3 | 1603 | 1611 | SWDQMWKCLI | S | 1804 |
| Ns3 | 1556 | 1564 | EFWESVFTGL | M | 1010 |
| Core | 173 | 182 | SFSIFLLALL | M | 1730 |
| Core | 130 | 139 | FADLMGYIPL | M | 1048 |
| Ns5b | 2834 | 1655 | MYAPTLWARM | M | 1511 |
| Ns3 | 1463 | 1471 | DFSLDPTFTI | M | 958 |
| Ns5b | 2614 | 1655 | QYSPGQRVEF | M | 1626 |
| Core | 82 | 91 | PWPLYGNEGM | M | 1590 |
| Ns3 | 1243 | 1251 | AYAAQGYKVL | M | 900 |
| Ns5b | 2816 | 1655 | AWETARHTPV | M | 897 |
| Ns5b | 2777 | 1655 | EYDLELITSC | W | 1046 |
| Ns3 | 1584 | 1592 | PYLVAYQATV | W | 1594 |
| Core | 135 | 144 | GYIPLVGAPL | W | 1211 |
| Ns3 | 1192 | 1200 | AVDFIPVESM | W | 882 |
| Ns4b | 1854 | 1655 | GYGAGVAGAL | W | 1210 |
| Ns3 | 1292 | 1300 | TYSTYGKFLA | W | 1886 |
| Core | 176 | 185 | IFLLALLSCL | W | 1266 |
| Ns3 | 1375 | 1383 | FYGKAIPIEV | W | 1100 |
| Ns5b | 2638 | 1655 | SYDTRCFDST | W | 1808 |
| Core | 85 | 94 | LYGNEGMGWA | W | 1488 |
| Ns3 | 1582 | 1590 | NFPYLVAYQA | W | 1522 |
| Ns3 | 1541 | 1549 | AYLNTPGLPV | W | 903 |
| Ns4b | 1914 | 1655 | QWMNRLIAFA | W | 1624 |

TABLE 10

Predicted HLA-Cw0602 binding peptides

| | | | | | | |
|---|---|---|---|---|---|---|
| | | | | | | SEQ ID |
| # | Protein | CS_fr | CS_to | pep_seq | Score | NO |

Algonomics
9-mer

| | | | | | | |
|---|---|---|---|---|---|---|
| 1 | Ns3 | 1292 | 1300 | TYSTYGKFL | S | 621 |
| 2 | Ns3 | 1244 | 1252 | YAAQGYKVL | S | 622 |
| 3 | Ns5b | 2696 | 2704 | YRRCRASGV | S | 623 |
| 4 | Ns5b | 2673 | 2681 | IRSLTERLY | S | 624 |
| 5 | Ns3 | 1494 | 1502 | GRRGIYRFV | S | 625 |
| 6 | Ns5b | 2573 | 2581 | GRKPARLIV | S | 626 |
| 7 | Ns5b | 2842 | 2850 | RMILMTHFF | S | 627 |
| 8 | Ns3 | 1417 | 1425 | YYRGLDVSV | S | 628 |
| 9 | Ns3 | 1606 | 1614 | QMWKCLIRL | S | 629 |
| 10 | Core | 173 | 181 | SFSIFLLAL | M | 630 |
| 11 | Ns3 | 1603 | 1611 | SWDQMWKCL | M | 631 |
| 12 | Ns5b | 2587 | 2595 | VRVCEKMAL | M | 632 |
| 13 | Ns3 | 1385 | 1393 | IKGGRHLIF | M | 633 |
| 14 | Ns5b | 2668 | 2676 | EARQAIRSL | M | 634 |
| 15 | Ns3 | 1413 | 1421 | NAVAYYRGL | M | 635 |
| 16 | Ns3 | 1383 | 1391 | EAIKGGRHL | M | 636 |
| 17 | Ns3 | 1415 | 1423 | VAYYRGLDV | M | 637 |
| 18 | Ns5b | 2678 | 2686 | ERLYIGGPL | M | 638 |
| 19 | Ns3 | 1383b | 1391b | EVIKGGRHL | M | 639 |
| 20 | Ns3 | 1266 | 1274 | AYMSKAHGV | M | 640 |
| 21 | Ns5b | 2841 | 2849 | ARMILMTHF | M | 641 |
| 22 | Ns3 | 1645 | 1653 | IMACMSADL | M | 642 |
| 23 | Ns5b | 2577 | 2585 | ARLIVFPDL | M | 643 |
| 24 | Ns3 | 1243 | 1251 | AYAAQGYKV | M | 644 |
| 25 | Ns3 | 1534 | 1542 | ETSVRLRAY | M | 645 |
| 26 | Ns5b | 2574 | 2582 | RKPARLIVF | M | 646 |
| 27 | Ns3 | 1245 | 1253 | AAQGYKVLV | M | 647 |
| 28 | Ns5b | 2613 | 2621 | FQYSPGQRV | M | 648 |
| 29 | Core | 29 | 37 | QIVGGVYLL | W | 649 |
| 30 | Core | 174 | 182 | FSIFLLALL | W | 650 |
| 31 | Ns3 | 1557 | 1565 | FWESVFTGL | W | 651 |
| 32 | Ns3 | 1553 | 1561 | DHLEFWESV | W | 652 |
| 33 | Core | 177 | 185 | FLLALLSCL | W | 653 |
| 34 | Core | 38 | 46 | PRRGPRLGV | W | 654 |
| 35 | Ns5b | 2631 | 2639 | KKCPMGFSY | W | 655 |
| 36 | Ns5b | 2607 | 2615 | MGSSYGFQY | W | 656 |
| 37 | Ns5b | 2835 | 2843 | YAPTLWARM | W | 657 |
| 38 | Core | 83 | 91 | WPLYGNEGL | W | 658 |
| 39 | Ns3 | 1522 | 1530 | DAGCAWYEL | W | 659 |
| 40 | Ns5b | 2796 | 2804 | ASGKRVYYL | W | 660 |
| 41 | Ns3 | 1338 | 1346 | TAGARLVVL | W | 661 |
| 42 | Ns5b | 2795 | 2803 | DASGKRVYY | W | 662 |
| 43 | Ns3 | 1376b | 1384b | YGKAIPIEV | W | 663 |
| 44 | Ns5b | 2833 | 2841 | IMYAPTLWA | W | 664 |
| 45 | Ns5b | 2827 | 2835 | SWLGNIIMY | W | 665 |
| 46 | Core | 77 | 85 | AQPGYPWPL | W | 666 |
| 47 | Ns5b | 2840 | 2848 | WARMILMTH | W | 667 |
| 48 | Ns5b | 2838 | 2846 | TLWARMILM | W | 668 |
| 49 | Ns3 | 1618 | 1626 | LHGPTPLLY | W | 669 |
| 50 | Ns3 | 1637 | 1645 | LTHPITKYI | W | 670 |
| 51 | Ns3 | 1638 | 1646 | THPITKYIM | W | 671 |
| 52 | Ns3 | 1440 | 1448 | LMTGYTGDF | W | 672 |
| 53 | Core | 111 | 119 | DPRRRSRNL | W | 673 |
| 54 | Core | 171 | 179 | GCSFSIFLL | W | 674 |
| 55 | Core | 150 | 158 | ALAHGVRVL | W | 675 |
| 56 | Ns3 | 1554 | 1562 | HLEFWESVF | W | 676 |
| 57 | Ns3 | 1404 | 1412 | AAKLSALGL | W | 677 |
| 58 | Ns3 | 1585 | 1593 | YLVAYQATV | W | 678 |
| 59 | Ns5b | 2636 | 2644 | GFSYDTRCF | W | 679 |
| 60 | Ns3 | 1583 | 1591 | FPYLVAYQA | W | 680 |
| 61 | Ns3 | 1540 | 1548 | RAYLNTPGL | W | 681 |
| 62 | Ns3 | 1418 | 1426 | YRGLDVSVI | W | 682 |
| 63 | Core | 23 | 31 | KFPGGGQIV | W | 683 |
| 64 | Ns3 | 1581 | 1589 | DNFPYLVAY | W | 684 |
| 65 | Core | 16 | 24 | NRRPQDVKF | W | 685 |
| 66 | Ns5b | 2720 | 2728 | SAACRAAKL | W | 686 |
| 67 | Ns3 | 1620 | 1628 | GPTPLLYRL | W | 687 |
| 68 | Core | 136 | 144 | YIPLVGAPL | W | 688 |
| 69 | Core | 28 | 36 | GQIVGGVYL | W | 689 |
| 70 | Ns3 | 1176 | 1184 | VVGVFRAAV | W | 690 |
| 71 | Ns5b | 2758 | 2766 | VFTEAMTRY | W | 691 |
| 72 | Core | 132 | 140 | DLMGYIPLV | W | 692 |
| 73 | Ns3 | 1181 | 1189 | RAAVCTRGV | W | 693 |
| 74 | Ns5b | 2616 | 2624 | SPGQRVEFL | W | 694 |
| 75 | Ns5b | 2605 | 2613 | AVMGSSYGF | W | 695 |
| 76 | Ns3 | 1402 | 1410 | ELAAKLSAL | W | 696 |
| 77 | Core | 149 | 157 | RALAHGVRV | W | 697 |
| 78 | Ns3 | 1246 | 1254 | AQGYKVLVL | W | 698 |
| 79 | Core | 114 | 122 | RRSRNLGKV | W | 699 |
| 80 | Ns5b | 2821 | 2829 | RHTPVNSWL | W | 700 |
| 81 | Ns5b | 2598 | 2606 | VVSTLPQAV | W | 701 |
| 82 | Ns5b | 2831 | 2839 | NIIMYAPTL | W | 702 |
| 83 | Ns5b | 2834 | 2842 | MYAPTLWAR | W | 703 |
| 84 | Ns5b | 2615 | 2623 | YSPGQRVEF | W | 704 |
| 85 | Ns5b | 2619 | 2627 | QRVEFLVNA | W | 705 |
| 86 | Ns5b | 2594 | 2602 | ALYDVVSTL | W | 706 |
| 87 | Core | 73 | 81 | GRTWAQPGY | W | 707 |
| 88 | Ns5b | 2581 | 2589 | VFPDLGVRV | W | 708 |
| 89 | Ns5b | 2579 | 2587 | LIVFPDLGV | W | 709 |
| 90 | Ns5b | 2697 | 2705 | RRCRASGVL | W | 710 |
| 91 | Ns3 | 1571 | 1579 | HFLSQTKQA | W | 711 |
| 92 | Ns3 | 1458 | 1466 | VTQTVDFSL | W | 712 |
| 93 | Ns5b | 2837 | 2845 | PTLWARMIL | W | 713 |
| 94 | Ns5b | 2794 | 2802 | HDASGKRVY | W | 714 |
| 95 | Core | 89 | 97 | EGLGWAGWL | W | 715 |

Algonomics
10-mer

| | | | | | |
|---|---|---|---|---|---|
| Ns3 | 1180 | 1188 | FRAAVCTRGV | S | 1081 |
| Ns5b | 2696 | 1655 | YRRCRASGVL | S | 2066 |
| Ns3 | 1243 | 1251 | AYAAQGYKVL | S | 900 |
| Ns5b | 2573 | 1655 | GRKPARLIVF | S | 1182 |
| Ns5b | 2841 | 1655 | ARMILMTHFF | S | 864 |
| Ns5b | 2820 | 1655 | ARHTPVNSWL | S | 863 |
| Ns5b | 2628 | 1655 | WKSKKCPMGF | S | 2013 |
| Ns3 | 1539 | 1547 | LRAYLNIPGL | M | 1454 |
| Ns4b | 1942 | 1655 | ARVTQILSSL | M | 868 |
| Core | 76 | 85 | WAQPGYPWPL | M | 1996 |
| Ns3 | 1492 | 1500 | GRGRRGIYRF | M | 1181 |
| Ns5b | 2834 | 1655 | MYAPTLWARM | M | 1511 |
| Ns5b | 2673 | 1655 | IRSLTERLYI | M | 1301 |
| Ns3 | 1626 | 1634 | YRLGAVQNEV | M | 2065 |
| Ns5b | 2614 | 1655 | QYSPGQRVEF | M | 1626 |
| Core | 149 | 158 | RALAHGVRVL | M | 1629 |
| Ns5b | 2587 | 1655 | VRVCEKMALY | M | 1967 |
| Core | 148 | 157 | ARALAHGVRV | M | 862 |
| Core | 22 | 31 | VKFPGGGQIV | M | 1920 |
| Ns3 | 1384 | 1392 | VIKGGRHLIF | M | 1917 |
| Ns5b | 2840 | 1655 | WARMILMTHF | M | 1997 |
| Ns3 | 1244 | 1252 | YAAQGYKVLV | M | 2024 |
| Core | 28 | 37 | GQIVGGVYLL | M | 1178 |
| Core | 35 | 44 | YLLPRRGPRL | M | 2047 |
| Ns5b | 2630 | 1655 | SKKCPMGFSY | M | 1739 |
| Ns3 | 1416 | 1424 | AYYRGLDVSV | M | 907 |
| Ns3 | 1375 | 1383 | FYGKAIPIEV | M | 1100 |
| Ns5b | 2593 | 1655 | MALYDVVSTL | M | 1492 |
| Core | 130 | 139 | FADLMGYIPL | M | 1048 |
| Core | 176 | 185 | IFLLALLSCL | M | 1266 |
| Ns3 | 1417 | 1425 | YYRGLDVSVI | M | 2083 |
| Ns3 | 1242 | 1250 | AAYAAQGYKV | W | 783 |
| Ns5b | 2836 | 1655 | APTLWARMIL | W | 853 |
| Ns5b | 2792 | 1655 | VAHDASGKRV | W | 1892 |
| Ns3 | 1556 | 1564 | EFWESVFTGL | W | 1010 |
| Core | 172 | 181 | CSFSIFLLAL | W | 935 |
| Ns3 | 1291 | 1299 | ITYSTYGKFL | W | 1310 |
| Ns5b | 2795 | 1655 | DASGKRVYYL | W | 955 |
| Core | 113 | 122 | RRRSRNLGKV | W | 1698 |
| Core | 173 | 182 | SFSIFLLALL | W | 1730 |
| Ns3 | 1249 | 1257 | YKVLVLNPSV | W | 2041 |
| Core | 155 | 164 | VRVLEDGVNY | W | 1968 |
| Core | 77 | 86 | AQPGYPWPLY | W | 861 |
| Ns5b | 2833 | 1655 | IMYAPTLWAR | W | 1279 |
| Ns4b | 1913 | 1655 | VQWMNRLIAF | W | 1963 |
| Ns3 | 1403 | 1411 | LAAKLSALGL | W | 1356 |
| Ns3 | 1382 | 1390 | IEVIKGGRHL | W | 1265 |
| Ns3 | 1553 | 1561 | DHLEFWESVF | W | 960 |
| Core | 135 | 144 | GYIPLVGAPL | W | 1211 |
| Core | 169 | 178 | LPGCSFSIFL | W | 1426 |
| Ns5b | 2835 | 1655 | YAPTLWARMI | W | 2028 |
| Ns3 | 1292 | 1300 | TYSTYGKFLA | W | 1886 |
| Ns4b | 1839 | 1655 | VGSIGLGKVL | W | 1911 |
| Ns3 | 1335 | 1343 | QAETAGARLV | W | 1595 |
| Ns5b | 2726 | 1655 | AKLQDCTMLV | W | 819 |
| Ns3 | 1605 | 1613 | DQMWKCLIRL | W | 982 |
| Ns4b | 1897 | 1655 | VCAAILRRHV | W | 1895 |
| Ns3 | 1189 | 1197 | VAKAVDFIPV | W | 1893 |
| Ns3 | 1541 | 1549 | AYLNTPGLPV | W | 903 |
| Ns3 | 1489 | 1497 | GRTGRGRRGI | W | 1184 |
| Ns3 | 1175 | 1184 | HAVGIFRAAV | W | 1215 |
| Ns4b | 1939 | 1655 | DAAARVTQIL | W | 952 |
| Ns5b | 2604 | 1655 | QAVMGSSYGF | W | 1599 |
| Ns5b | 2615 | 1655 | YSPGQRVEFL | W | 2068 |
| Ns5b | 2695 | 1655 | GYRRCRASGV | W | 1212 |
| Ns5b | 2606 | 1655 | VMGSSYGFQY | W | 1939 |
| Ns3 | 1602 | 1610 | PSWDQMWKCL | W | 1585 |
| Ns3 | 1644 | 1652 | YIMACMSADL | W | 2037 |
| Core | 142 | 151 | APLGGAARAL | W | 836 |
| Ns5b | 2580 | 1655 | IVFPDLGVRV | W | 1312 |
| Ns5b | 2635 | 1655 | MGFSYDTRCF | W | 1494 |
| Core | 37 | 46 | LPRRGPRLGV | W | 450 |
| Ns5b | 2793 | 1655 | AHDASGKRVY | W | 810 |
| Ns5b | 2586 | 1655 | GVRVCEKMAL | W | 1203 |
| Ns3 | 1265 | 1273 | GAYMSKAHGV | W | 1110 |
| Ns3 | 1619 | 1627 | HGPTPLLYRL | W | 1219 |
| Ns3 | 1200 | 1208 | SMETTMRSPV | W | 1750 |
| Ns3 | 1609 | 1617 | KCLIRLKPTL | W | 1319 |
| Ns4b | 1873 | 1655 | MPSTEDLVNL | W | 1505 |
| Core | 114 | 123 | RRSRNLGKVI | W | 1699 |
| Ns5b | 2570 | 1655 | EKGGRKPARL | W | 1015 |
| Ns3 | 1337 | 1345 | ETAGARLVVL | W | 1032 |
| Ns3 | 1161 | 1170 | LKGSSGGPLL | W | 1388 |
| Ns3 | 1584 | 1592 | PYLVAYQATV | W | 1594 |
| Ns3 | 1534 | 1542 | ETSVRLRAYL | W | 1033 |
| Ns3 | 1412 | 1420 | LNAVAYYRGL | W | 1416 |
| Ns3 | 1637 | 1645 | LTHPITKYIM | W | 1469 |
| Ns3 | 1186 | 1194 | TRGVAKAVDF | W | 1866 |
| Ns5b | 2589 | 1655 | VCEKMALYDV | W | 1897 |
| Ns3 | 1578 | 1586 | QAGDNFPYLV | W | 1596 |
| Ns3 | 1646 | 1654 | MACMSADLEV | W | 1490 |
| Core | 89 | 98 | EGMGWAGWLL | W | 1012 |
| Ns5b | 2602 | 1655 | LPQAVMGSSY | W | 1437 |
| Ns3 | 1219 | 1227 | VPQTFQVAHL | W | 1954 |
| Ns4b | 1859 | 1655 | VAGALVAFKV | W | 1891 |
| Ns3 | 1245 | 1253 | AAQGYKVLVL | W | 777 |
| Ns3 | 1622 | 1630 | TPLLYRLGAV | W | 1851 |
| Ns4b | 1920 | 1655 | IAFASRGNHV | W | 1255 |
| Ns5b | 2616 | 1655 | SPGQRVEFLV | W | 1765 |
| Ns4b | 1864 | 1655 | VAFKVMSGEM | W | 1888 |
| Ns3 | 1521 | 1529 | YDAGCAWYEL | W | 2030 |
| Ns3 | 1240 | 1248 | VPAAYAAQGY | W | 1943 |
| Ns5b | 2672 | 1655 | AIRSLTERLY | W | 817 |
| Ns4b | 1840 | 1655 | GSIGLGKVLV | W | 1187 |
| Ns3 | 1617 | 1625 | TLHGPTPLLY | W | 1833 |
| Ns3 | 1507 | 1515 | RPSGMFDSSV | W | 1687 |
| Ns3 | 1235 | 1243 | GKSTKVPAAY | W | 1146 |
| Ns3 | 1373 | 1381 | IPFYGKAIPI | W | 1284 |
| Ns3 | 1408 | 1416 | SALGLNAVAY | W | 1717 |
| Ns3 | 1414 | 1422 | AVAYYRGLDV | W | 880 |
| Core | 46 | 55 | VRATRKTSER | W | 1964 |

TABLE 11

Predicted HLA-Cw0702 binding peptides

| | | | | | | |
|---|---|---|---|---|---|---|
| | | | | | | SEQ ID |
| # | Protein | CS_fr | CS_to | pep_seq | Score | NO |

Algonomics
9-mer

| | | | | | | |
|---|---|---|---|---|---|---|
| 1 | Ns3 | 1292 | 1300 | TYSTYGKFL | S | 716 |
| 2 | Ns3 | 1243 | 1251 | AYAAQGYKV | S | 717 |
| 3 | Ns3 | 1417 | 1425 | YYRGLDVSV | M | 718 |
| 4 | Ns5b | 2517 | 2585 | ARLIVFPDL | M | 719 |
| 5 | Core | 173 | 181 | SFSIFLLAL | M | 720 |
| 6 | Ns5b | 2673 | 2681 | IRSLTERLY | M | 721 |
| 7 | Core | 85 | 93 | LYGNEGLGW | M | 722 |
| 8 | Core | 129 | 137 | GFADLMGYI | M | 723 |
| 9 | Core | 73 | 81 | GRTWAQPGY | M | 724 |
| 10 | Ns5b | 2636 | 2644 | GFSYDTRCF | M | 725 |
| 11 | Ns5b | 2827 | 2835 | SWLGNIIMY | M | 726 |
| 12 | Ns3 | 1643 | 1651 | KYIMACMSA | M | 727 |
| 13 | Ns5b | 2841 | 2849 | ARMILMTHF | M | 728 |
| 14 | Ns5b | 2587 | 2595 | VRVCEKMAL | M | 729 |
| 15 | Ns5b | 2835 | 2843 | YAPTLWARM | M | 730 |
| 16 | Core | 75 | 83 | TWAQPGYPW | M | 731 |
| 17 | Ns5b | 2802 | 2810 | YYLTRDPTT | W | 732 |
| 18 | Ns5b | 2834 | 2842 | MYAPTLWAR | W | 733 |
| 19 | Ns5b | 2821 | 2829 | RHTPVNSWL | W | 734 |
| 20 | Ns3 | 1244 | 1252 | YAAQGYKVL | W | 735 |
| 21 | Core | 83 | 91 | WPLYGNEGL | W | 736 |
| 22 | Ns3 | 1571 | 1579 | HFLSQTKQA | W | 737 |
| 23 | Ns3 | 1266 | 1274 | AYMSKAHGV | W | 738 |
| 24 | Ns3 | 1557 | 1565 | FWESVFTGL | W | 739 |
| 25 | Ns5b | 2574 | 2582 | RKPARLIVF | W | 740 |
| 26 | Ns5b | 2697 | 2705 | RRCRASGVL | W | 741 |
| 27 | Ns5b | 2842 | 2850 | RMILMTHFF | W | 742 |
| 28 | Ns5b | 2610 | 2618 | SYGFQYSPG | W | 743 |
| 29 | Ns3 | 1618 | 1626 | LHGPTPLLY | W | 744 |
| 30 | Ns5b | 2678 | 2686 | ERLYIGGPL | W | 745 |
| 31 | Ns3 | 1603 | 1611 | SWDQMWKCL | W | 746 |
| 32 | Ns3 | 1583 | 1591 | FPYLVAYQA | W | 747 |
| 33 | Core | 16 | 24 | NRRPQDVKF | W | 748 |
| 34 | Ns5b | 2581 | 2589 | VFPDLGVRV | W | 749 |
| 35 | Core | 17 | 25 | RRPQDVKFP | W | 750 |
| 36 | Core | 136 | 144 | YIPLVGAPL | W | 751 |
| 37 | Ns3 | 1248 | 1256 | GYKVLVLNP | W | 752 |
| 38 | Ns3 | 1638 | 1646 | THPITKYIM | W | 753 |
| 39 | Ns5b | 2765 | 2773 | RYSAPPGDP | W | 754 |
| 40 | Ns3 | 1494 | 1502 | GRRGIYRFV | W | 755 |
| 41 | Ns3 | 1520 | 1528 | CYDAGCAWY | W | 756 |
| 42 | Ns3 | 1492 | 1500 | GRGRRGIYR | W | 757 |
| 43 | Ns3 | 1499 | 1507 | YRFVTPGER | W | 758 |
| 44 | Ns5b | 2695 | 2703 | GYRRCRASG | W | 759 |
| 45 | Core | 68 | 76 | ARRPEGRTW | W | 760 |
| 46 | Ns5b | 2631 | 2639 | KKCPMGFSY | W | 761 |
| 47 | Core | 69 | 77 | RRPEGRTWA | W | 762 |
| 48 | Core | 34 | 42 | VYLLPRRGP | W | 763 |
| 49 | Ns5b | 2833 | 2841 | IMYAPTLWA | W | 764 |
| 50 | Core | 114 | 122 | RRSRNLGKV | W | 765 |
| 51 | Ns3 | 1418 | 1426 | YRGLDVSVI | W | 766 |
| 52 | Ns3 | 1341 | 1349 | ARLVVLATA | W | 767 |
| 53 | Ns5b | 2796 | 2804 | ASGKRVYYL | W | 768 |

Algonomics
10-mer

| | | | | | |
|---|---|---|---|---|---|
| Ns3 | 1243 | 1251 | AYAAQGYKVL | S | 900 |
| Core | 135 | 144 | GYIPLVGAPL | S | 1211 |
| Ns5b | 2834 | 1655 | MYAPTLWARM | S | 1511 |
| Ns5b | 2587 | 1655 | VRVCEKMALY | M | 1967 |
| Ns5b | 2696 | 1655 | YRRCRASGVL | M | 2066 |
| Core | 173 | 182 | SESIELLALL | M | 1730 |
| Ns3 | 1398 | 1406 | KKCDELAAKL | M | 1327 |
| Ns3 | 1417 | 1425 | YYRGLDVSVI | M | 2083 |
| Core | 85 | 94 | LYGNEGMGWA | M | 1488 |
| Ns5b | 2841 | 1655 | ARMILMTHFF | M | 864 |
| Ns3 | 1416 | 1424 | AYYRGLDVSV | M | 907 |
| Ns3 | 1375 | 1383 | FYGKAIPIEV | M | 1100 |
| Ns3 | 1539 | 1547 | LRAYLNTPGL | M | 1454 |
| Ns5b | 2820 | 1655 | ARHTPVNSWL | M | 863 |
| Ns4b | 1933 | 1655 | HYVPESDAAA | M | 1254 |
| Ns3 | 1582 | 1590 | NEPYLVAYQA | M | 1522 |
| Ns3 | 1541 | 1549 | AYLNTPGLPV | W | 903 |
| Ns5b | 2573 | 1655 | GRKPARLIVF | W | 1182 |
| Core | 114 | 123 | RRSRNLGKVI | W | 1699 |
| Ns5b | 2673 | 1655 | IRSLTERLYI | W | 1301 |
| Core | 176 | 185 | IFLLALLSCL | W | 1266 |
| Ns3 | 1292 | 1300 | TYSTYGKFLA | W | 1886 |
| Core | 129 | 138 | GFADLMGYIP | W | 1132 |
| Core | 155 | 164 | VRVLEDGVNY | W | 1968 |
| Ns5b | 2827 | 1655 | SWLGNIIMYA | W | 1806 |
| Core | 73 | 82 | GRAWAQPGYP | W | 1180 |
| Ns4b | 1854 | 1655 | GYGAGVAGAL | W | 1210 |
| Core | 76 | 85 | WAQPGYPWPL | W | 1996 |
| Ns3 | 1492 | 1500 | GRGRRGIYRF | W | 1181 |
| Ns4b | 1942 | 1655 | ARVTQILSSL | W | 868 |
| Ns5b | 2628 | 1655 | WKSKKCPMGF | W | 2013 |
| Core | 74 | 83 | RAWAQPGYPW | W | 1634 |
| Ns5b | 2593 | 1655 | MALYDVVSTL | W | 1492 |
| Ns3 | 1584 | 1592 | PYLVAYQATV | W | 1594 |
| Ns5b | 2802 | 1655 | YYLTRDPTTP | W | 2081 |
| Ns3 | 1235 | 1243 | GKSTKVPAAY | W | 1146 |
| Ns5b | 2801 | 1655 | VYYLTRDPTI | W | 1991 |
| Core | 34 | 43 | VYLLPRRGPR | W | 1990 |
| Ns3 | 1358 | 1366 | PHPNIEEVAL | W | 1555 |
| Core | 149 | 158 | RALAHGVRVL | W | 1629 |
| Ns4b | 1873 | 1655 | MPSTEDLVNL | W | 1505 |
| Ns3 | 1498 | 1506 | IYRFVTPGER | W | 1314 |
| Ns5b | 2840 | 1655 | WARMILMTHF | W | 1997 |
| Ns3 | 1443 | 1451 | GYTGDFDSVI | W | 1213 |
| Ns5b | 2614 | 1655 | QYSPGQRVEF | W | 1626 |
| Ns5b | 2695 | 1655 | GYRRCRASGV | W | 1212 |
| Ns5b | 2757 | 1655 | RVFTEAMTRY | W | 1712 |
| Ns3 | 1626 | 1634 | YRLGAVQNEV | W | 2065 |
| Core | 23 | 32 | KFPGGGQIVG | W | 1321 |
| Core | 17 | 26 | RRPQDVKFPG | W | 1696 |
| Ns5b | 2835 | 1655 | YAPTLWARMI | W | 2028 |
| Ns3 | 1644 | 1652 | YIMACMSADL | W | 2037 |
| Ns4b | 1914 | 1655 | QWMNRLIAFA | W | 1624 |
| Core | 68 | 77 | ARRPEGRAWA | W | 866 |

TABLE 12

Predicted HLA-DRB1*0101/0401/0701 and
-DRB1*0301 binding peptides

| | | | |
|---|---|---|---|
| Protein | Full Sequence | Score (PIC) | SEQ ID NO |

| | | | |
|---|---|---|---|
| NS4B | AAQLAPPSAASAFVG | 0.074 | 2102 |
| NS5B | ACKLTPPHSAKSKFG | 1.07 | 2103 |
| NS5A | ADLIEANLLWRQEMG | 5.63 | 2104 |
| C | ADLMGYIPLVGAPLG | 0.043 | 2105 |
| NS5B | APTLWARMILMTHFF | 6.67 | 2106 |
| NS3 | AQGYKVLVLNPSVAA | 0.5 | 2107 |
| NS5B | ARAAWETARHTPVNS | | 2108 |
| NS3 | ARLVVLATATPPGSV | 0.12 | 2109 |
| NS5B | ASCLRKLGVPPLRVW | 0.47 | 2110 |
| NS5A | ASQLSAPSLKATCTT | 0.41 | 2111 |
| NS3 | AVGIFRAAVCTRGVA | 5.96 | 2112 |
| NS4B | AVQWMNRLIAFASRG | 9.61 | 2113 |
| E1 | AWDMMMNWSPTTALV | 2.56 | 2114 |
| NS4A | AYCLTTGSVVIVGRI | 0.74 | 2115 |
| NS5B | CQIYGACYSIEPLDL | 2.04 | 2116 |
| NS5A | DADLIEANLLWRQEM | 7.67 | 2117 |
| NS3 | DAHFLSQTKQAGDNF | | 2118 |
| NS3 | DIIICDECHSTDSTT | | 2119 |
| NS2 | DLAVAVEPVVFSDME | 2.42 | 2120 |
| NS2 | DLAVAVEPVVFSDME | | 2120 |
| NS4B | DLVNLLPAILSPGA | 0.17 | 2121 |
| NS3 | DPTFTIETTTVPQDA | | 2122 |
| NS3 | DSSVLCECYDAGCAW | | 2123 |
| | DVVVVATDALMTGFT | 3.12 | 2124 |
| NS3 | DVVVVATDALMTGYI | 3.12 | 2125 |
| NS4B | EDLVNLLPAILSPG | 0.72 | 2126 |
| NS5A | EPDVAVLTSMLTDPS | 0.027 | 2127 |
| NS4B | FNILGGWVAAQLAPP | 0.16 | 2128 |
| NS3 | FPYLVAYQATVCARA | 4.76 | 2129 |
| C | FSIFLLALLSCLTIP | 5.47 | 2130 |
| | FSIFLLALLSCLTVP | 5.47 | 2131 |
| E2 | FTTLPALSTGLIHLH | 7.05 | 2132 |
| NS5B | GACYSIEPLDLPQII | | 2133 |
| | GAGVAGALVAFKIMS | 0.29 | 2134 |
| NS4B | GAGVAGALVAFKVMS | 0.29 | 2135 |
| NS4B | GALVVGVVCAAILRR | 3.59 | 2136 |
| NS3 | GARLVVLATATPPGS | 0.11 | 2137 |
| | GCGWAGWLLSPRGSR | 6.86 | 2138 |
| C | GCSFSIFLLALLSCL | 4.62 | 2139 |
| NS3 | GDNFPYLVAYQATVC | 0.05 | 2140 |
| NS4A | GGVLAALAAYCLTTG | 0.44 | 2141 |
| E1 | GHRMAWDMMMNWSPT | 6.3 | 2142 |
| E1 | GHRMAWDMMMNWSPT | | 2142 |
| NS4B | GIQYLAGLSTLPGNP | 0.1 | 2143 |
| NS5B | GKYLFNWAVRTKLKL | 9.61 | 2144 |
| | GLGWAGWLLSPRGSR | 6.86 | 2145 |
| NS3 | GLPVCQDHLEFWESV | | 2146 |
| C | GMGWAGWLLSPRGSR | 6.86 | 2147 |
| NS4B | GMQLAEQFKQKALGL | | 2148 |
| C | GPRLGVRAIRKTSER | 2.87 | 2149 |
| | GQGWRLLAPITAYSQ | 1.34 | 2150 |
| C | GQIVGGVYLLPRRGP | 1.3 | 2151 |
| NS5A | GSQLPCEPEPDVAVL | | 2152 |
| NS5B | GSSYGFQYSPGQRVE | 0.52 | 2153 |
| NS3 | GTVLDQAETAGARLV | 0.32 | 2154 |
| C | GVNYATGNLPGCSFS | 4.02 | 2155 |
| C | GVRVLEDGVNYATGN | | 2156 |
| NS3 | GYKVLVLNPSVAATL | 6.3 | 2157 |
| NS3 | HLIFCHSKKKCDELA | | 2158 |
| | HQWINEDCSTPCSGS | | 2159 |
| NS5B | IERLHGLSAFSLHSY | 2.56 | 2160 |
| | IQRLHGLSAFSLHSY | 2.56 | 2161 |
| NS4B | IQYLAGLSTLPGNPA | 0.66 | 2162 |
| NS5A | ITRVESENKVVILDS | | 2163 |
| NS3 | KPTLHGPTPLLYRLG | 0.56 | 2164 |
| NS3 | KVLVLNPSVAATLGF | 0.52 | 2165 |
| NS4B | LAGYGAGVAGALVAF | 1.63 | 2166 |
| E2 | LFLLLADARVCACLW | | 2167 |
| NS4B | LFNILGGWVAAQLAP | 3.69 | 2168 |
| NS4B | LGKVLVDILAGYGAG | | 2169 |
| NS5B | LHSYSPGEINRVASC | 2.87 | 2170 |
| NS3 | LIRLKPTLHGPTPLL | | 2171 |
| NS4B | LPAILSPGALVVGVV | 0.11 | 2172 |
| E2 | LPALSTGLIHLHQNI | 0.68 | 2173 |
| NS4B | LSTLPGNPAIASLMA | 0.24 | 2174 |
| NS3 | LTHIDAHFLSQTKQA | 3.03 | 2175 |
| | LTHIDAHFLSQTKQS | 3.03 | 2176 |
| NS5A | LTSMLTDPSHITAET | 2.29 | 2177 |
| NS5A | LTSMLTDPSHITAET | | 2177 |
| NS3 | LVAYQATVCARAQAP | 4.76 | 2178 |
| NS4B | LVNLLPAILSPGALV | 5.63 | 2179 |
| NS3 | LVVLATATPPGSVIV | 0.24 | 2180 |
| | MACMSADLEVVTSTW | | 2181 |
| NS4B | MNRLIAFASRGNHVS | 2.79 | 2182 |
| NS5A | MPPLEGEPGDPDL | | 2183 |
| C | MSTNPKPQRKTK | | 2184 |
| | MTGFTGDFDSVIDCN | | 2185 |
| NS4B | NPAIASLMAFTASIT | 0.29 | 2186 |
| NS5B | NSWLGNIIMYAPTLW | 1.2 | 2187 |
| NS5B | NVSVAHDASGKRVYY | | 2188 |
| E2 | PCSFTILPALSTGLI | 0.04 | 2189 |
| NS4B | PGALVVGVVCAAILR | 0.55 | 2190 |
| NS5B | PMGFSYDTRCFDSTV | | 2191 |
| NS3 | PQTFQVAHLHAPTGS | 0.28 | 2192 |
| NS4B | PTHYVPESDAAARVT | | 2193 |
| NS5B | PTLWARMILMTHFFS | 0.85 | 2194 |
| NS3 | PYLVAYQATVCARAQ | 0.072 | 2195 |
| NS3 | QDAVSRSQRRGRTGR | | 2196 |
| NS5B | QKKVTFDRLQVLDDH | | 2197 |
| NS5B | QPEYDLELITSCSSN | | 2198 |
| NS3 | RAAVCTRGVAKAVDF | 3.8 | 2199 |
| NS3 | RGLLGCIITSLTGRD | 8.59 | 2200 |
| C | RLGVRATRKTSERSQ | | 2201 |
| NS5B | RLIVFPDLGVRVCEK | | 2202 |
| NS3 | RLVVLATATPPGSVT | 9.34 | 2203 |
| | RPEYDLELITSCSSN | | 2204 |
| NS5A | RQEMGGNITRVESEN | 5.03 | 2205 |
| E2 | RSELSPLLLSTTEWQ | 0.72 | 2206 |
| NS3 | RSPVFTDNSSPPAVP | | 2207 |
| E1 | SAMYVGDLCGSVFLV | | 2208 |
| NS3 | SDLYLVTRHADVIPV | | 2209 |
| C | SFSIFLLALLSCLTI | 4.02 | 2210 |
| | SFSIFLLALLSCLTV | 4.02 | 2211 |
| C | SIFLLALLSCLTIPA | 0.23 | 2212 |
| | SKGWRLLAPITAYAQ | 1.34 | 2213 |
| NS5B | SLRVFTEAMTRYSAP | 2.49 | 2214 |
| NS5B | SLRVFTEAMTRYSAP | | 2214 |
| E1 | SRCWVALTPTLAARN | 0.047 | 2215 |
| NS3 | STKVPAAYAAQGYKV | 0.15 | 2216 |
| NS3 | STIILGIGTVLDQAE | 0.37 | 2217 |
| NS4A | STWVLVGGVLAALAA | 0.28 | 2218 |
| NS5B | SYTWTGALITPCAAE | 5.63 | 2219 |
| NS3 | TFQVAHLHAPTGSGK | 8.35 | 2220 |
| | TMLVCGDDLVVICES | | 2221 |
| NS5B | TPCAAEESKLPINAL | | 2222 |
| | TPCAAEESKLPINPL | | 2223 |
| NS3 | TPLLYRLGAVQNEVT | 0.32 | 2224 |
| NS3 | TRGLLGCIITSLTGR | 7.05 | 2225 |
| NS5B | TTIMAKNEVFCVQPE | 0.55 | 2226 |
| NS3 | TTTVPQDAVSRSQRR | | 2227 |
| NS3 | TVDFSLDPTFTIETT | | 2228 |
| NS4A | TWVLVGGVLAALAAY | 0.15 | 2229 |
| NS4B | VDILAGYGAGVAGAL | 3.69 | 2230 |
| NS3 | VESMETTMRSPVFTD | | 2231 |
| NS5B | VFCVQPEKGGRKPAR | | 2232 |
| NS4A | VGGVLAALAAYCLTT | 1.2 | 2233 |
| NS3 | VGIFRAAVCTRGVAK | 3.8 | 2234 |
| NS3 | VLVLNPSVAATLGFG | 9.61 | 2235 |
| NS4B | VNLLPAILSPGALVV | 0.4 | 2236 |
| NS5B | VNSWLGNIIMYAPTL | 1.42 | 2237 |
| NS4B | VQWMNRLIAFASRGN | 1.59 | 2238 |
| NS4B | VVGVVCAAILRRHVG | 5.03 | 2239 |
| | VVVVATDALMTGFTG | 4.37 | 2240 |
| | VVVVATDALMTGFTG | | 2240 |
| NS3 | VVVVATDALMTGYTG | 4.37 | 2241 |
| NS3 | VVVVATDALMTGYTG | | 2241 |
| P7 | VWPLLLLLLALPPRA | 0.29 | 2242 |
| NS5B | VYYLTRDPTTPLARA | | 2243 |
| NS3 | WDQMWKCLIRLKPTL | 3.69 | 2244 |
| NS3 | WESVFTGLTHIDAHF | 1.73 | 2245 |
| NS3 | WKCLIRLKPTLHGPT | 2.95 | 2246 |
| NS4A | WVLVGGVLAALAAYC | 0.021 | 2247 |
| NS3 | YDIIICDECHSTDST | | 2248 |
| NS3 | YGKFLADGGCSGGAY | | 2249 |
| P7 | YGVWPLLLLLLALPP | 1.2 | 2250 |
| C | YIPLVGAPLGGAARA | 0.072 | 2251 |
| NS3 | YKVLVLNPSVAATLG | 0.18 | 2252 |
| E1 | YYSMVGNWAKVLIVM | 2.56 | 2253 |

TABLE 13

Selection of predicted CTL epitopes
Immun mice = immunogenicity in transgenic or
surrogate mice
Immun recall = immunoreactivity in human recall
assay
High = Ki > 20.000 nM
Tg = transgenic mice

| | | | | |
|---|---|---|---|---|
| | | Immun | Immun | SEQ ID |
| Genotype | Ki (nM) | mice | recall | NO |

| | | | | | |
|---|---|---|---|---|---|
| HLA-A0101 | | | | | |
| AATLGFGAY | 1b/1a | 694 | + | | 557 |
| AGDNFPYLV | 1b | high | | | 24 |
| ALGLNAVAYY | 1b | 14286 | | | 822 |
| ATDALMTGY | 1b | 4 | | + | 1 |
| ATDALMTGYT | 1b | 227 | | + | 877 |
| AVMGSSYGF | 1b/1a | 16100 | | | 16 |
| CTCGSSDLY | 1b/1a | 14 | | | 940 |
| CYDAGGAWY | 1b/1a | 3072 | | | 13 |
| DASGKRVYY | 1b | 5625 | | | 10 |
| DNFPYLVAY | 1b | 1111 | | | 8 |
| DSSVLCECY | 1b/1a | 454 | | + | 17 |
| ECYDAGCAWY | 1b/1a | 20000 | | | 1002 |
| EPEPDVAVL | 1b/1a | high | | | 1024 |
| EVDGVRLHRY | | 167 | | | 1037 |
| FADLMGYIP | 1b/1a/3a | high | | | 4 |
| FTDNSSPPA | 1b/1a | 10 | | + | 7 |
| FTDNSSPPAV | 1b/1a | 45 | | + | 1086 |
| FTEAMTRYS | 1b/1a/3a | 1803 | | | 9 |
| FTEAMTRYSA | 1b/1a/3a | 1857 | | + | 1087 |
| GAPITYSTY | 1b | high | | | 11 |
| GLDVSVIPT | 1b/1a/3a | high | | | 29 |
| GLSAFSLHSY | | 61 | | | 1154 |
| HIDAHFLSQ | 1b/1a/3a | high | | | 1221 |
| HSAKSKFGY | 1b/1a | 615 | + | | 1241 |
| ITTGAPITY | 1b | 403 | | | 6 |
| ITYSTYGKF | 1b/1a | high | | | 26 |
| IVDVQYLYG | 1b/1a/3a | 6146 | | | 1311 |
| KCDELAAKL | 1b/1a | 20000 | | | 1318 |
| KSTKVPAAY | 1b/1a/3a | 858 | | | 25 |
| LADGGCSGGAY | | 60 | | | 1359 |
| LCECYDAGC | 1b/1a/3a | high | | | 1366 |
| LDPTFTIET | 1b/1a | high | | | 30 |
| LGLNAVAYY | 1b | high | | | 18 |
| LHGPTPLLY | 1b/1a/3a | 20000 | | | 219 |
| LSAFSLHSY | 1b/1a | 28 | + | | 1456 |
| LTCGFADLM | 1b/1a/3a | 759 | | | 2 |
| LTCGFADLMGY | 1b/1a/3a | 11 | | | 1465 |
| LTDPSHITA | 1b/1a | 15 | | | 1467 |
| LTDPSHITAE | 1b/1a | 237 | | + | 1468 |
| LTHIDAHFL | 1b/1a/3a | high | | | 5 |
| LTHPITKYI | 1b | high | | | 23 |
| LTHPITKYIM | 1b | 20000 | | | 1469 |
| LVDILAGYGA | 1b/1a | 258.5 | + | + | 1478 |
| MGSSYGFQY | 1b/1a | 917 | | | 22 |
| NSWLGNIIMY | 1b/3a | 1857 | | | 1534 |
| PAAYAAQGY | 1b/1a | 1457 | | | 12 |
| PAETSVRLR | 1b | high | | | 27 |
| PGDPPQPEY | 1b/1a | 14188 | | | 19 |
| PTDPRRRSR | 1b/1a | high | | | 55 |
| PTDPRRRSRN | 1b/1a | 17816 | | | 1586 |
| PTLHGPTPLLY | | 452 | | | 1587 |
| PVESMETTM | 1b | high | | | 15 |
| QAETAGARL | 1b/1a | high | | | 60 |
| RSELSPLLL | 1b/1a | 106 | | + | 1700 |
| RSELSPLLLS | 1b/1a | 1853 | | | 1701 |
| RVCEKMALY | 1b/1a | 2384 | | | 3 |
| RVFTEAMTRY | 1b | 3490 | | | 1712 |
| SLDPTFTIET | 1b/1a | high | | | 1741 |
| STEDLVNLL | 1b/1a | 8223 | | | 1787 |
| STEDLVNLLP | 1b/1a | high | | | 1788 |
| TLHGPTPLLY | 1b/1a/3a | 343 | + | | 1833 |
| TRDPTTPLAR | 1b/1a | high | | | 1865 |
| TSCGNTLTCY | 1b/1a | 246 | | | 1867 |
| VAATLGFGAY | 1b/1a | 122 | + | | 1887 |
| VATDALMTGY | 1b | 452 | | + | 1894 |
| VIDTLTCGF | 1b/1a/3a | 1017 | | | 28 |
| VIDTLTCGFA | 1b/1a/3a | 110.5 | | | 1914 |
| VPAAYAAQGY | 1b/1a | 20000 | | | 1943 |
| VTLTHPITK | 1b | high | | | 21 |
| VTLTHPITKY | 1b | 183 | | | 1976 |
| YAPTLWARM | 1b | high | | | 14 |
| HLA-A0201 | | | | | |
| ALAHGVRVL | 1b/1a | 627 | | | 72 |
| ALSTGLIHL | 1b/1a/3a | 329 | | | 825 |
| ALYDVVSTL | 1b | 19 | + | | 67 |
| AQPGYPWPL | 1b/1a/3a | 382 | | | 65 |
| CLVDYPYRL | 1b/1a | 437 | + | | 922 |
| DLCGSVFLV | 1b/1a | 789 | | | 963 |
| DLMGYIPLV | 1b/1a/3a | 36 | + | | 66 |
| FIPVESMET | | 934 | | | 1059 |
| FLLALLSCL | 1b/1a | 136 | + | | 361 |
| FLLALLSCLT | 1b/1a | 132 | | + | 1070 |
| FLLLADARV | 1b/1a/3a | 20 | + | | 1072 |
| GLGWAGWLL | | 27 | | | 1150 |
| GLLGCIITSL | 1b/1a | 26 | + | + | 1151 |
| GMFDSSVLC | 1b/1a | 71 | + | | 71 |
| GTQEDAASL | 1b | 1295 | | | 88 |
| HLHQNIVDV | 1b/1a/3a | 500 | | | 1227 |
| HMWNFISGI | 1b/1a | 12 | + | | 1233 |
| ILAGYGAGV | 1b/1a/3a | 88 | + | | 1269 |
| ILSPGALVV | 1b/1a/3a | 238 | | | 1275 |
| IMACMSADL | 1b | 66 | | | 90 |
| IMAKNEVFCV | 1b/1a/3a | 199 | | | 1278 |
| IMYAPTLWA | 1b | 46 | | | 84 |
| KLQDCTMLV | 1b | 4.6 | + | | 75 |
| KVLVLNPSV | 1b/1a | 50 | + | | 73 |
| LLFLLLADA | 1b/1a | 16 | | | 1395 |
| LLFNILGGWV | 1b/1a | 4.1 | + | | 1396 |
| LLGCIITSL | 1b/1a | 56 | | | 1397 |
| LLSCLTIPA | 1b | 12 | | | 70 |
| LTHIDAHFL | 1b/1a/3a | 181 | | + | 5 |
| LVLNPSVAA | 1b/1a/3a | 1679 | | | 82 |
| MALYDVVST | 1b | 1142 | | | 85 |
| NIIMYAPTL | 1b | 70 | + | + | 87 |
| NLPGCSFSI | 1b/1a/3a | 70 | | | 93 |
| PLLLLLLAL | 1b/1a | 6554 | | | 1557 |
| QIVGGVYLL | 1b/1a | 219 | + | | 91 |
| QMWKCLIRL | 1b/1a | 153 | + | | 238 |
| RLGAVQNEV | 1b/1a | 221 | + | + | 265 |
| RLHGLSAFSL | 1b/1a | 179 | | | 1659 |
| RLIVFPDLGV | 1b/1a | 89 | | + | 1661 |
| RLVVLATAT | 1b/1a | 16737 | | | 86 |
| RLYIGGPLT | 1b | 536 | | | 79 |
| SMVGNWAKV | 1b/1a | 158 | + | | 1753 |
| SVFTGLTHI | 1b/3a | 84 | + | | 76 |
| TILGIGTVL | 1b | 207 | | | 89 |
| TLHGPTPLL | 1b/1a/3a | 68 | + | | 81 |
| TLWARMILM | 1b/1a | 8 | + | + | 92 |
| VLVGGVLAA | 1b/1a | 219 | | + | 1933 |
| VLVGGVLAAL | 1b/1a | 26 | + | + | 1934 |
| VVATDALMT | 1b/1a | high | | | 44 |
| VVSTLPQAV | 1b | 884 | | | 68 |
| WLGNIIMYA | 1b/3a | 14.5 | | | 62 |
| WMNRLIAFA | 1b/1a/3a | 122 | | | 2015 |
| YIPLVGAPL | 1b/1a | 77 | + | | 69 |
| YLFNWAVRT | 1b/1a/3a | 29 | + | | 2043 |
| YLLPRRGPRL | 1b/1a | 140 | + | + | 2047 |
| YLNTPGLPV | 1b | 6.2 | + | | 74 |
| YLVAYQATV | 1b/1a | 19.5 | + | | 63 |
| YLVTRHADV | 1b/1a | 292 | + | | 2053 |
| YQATVCARA | 1b/1a/3a | 20 | + | | 83 |
| HLA-A1101 | | | | | |
| AAYAAQGYK | 1b/1a | 13* | + | | 56 |
| ALGLNAVAY | 1b | | | | 51 |
| ALYDVVSTL | 1b | 16468 | | | 67 |
| ASAACRAAK | 1b | 15* | + | | 155 |
| AVCTRGVAK | 1b/1a/3a | 48* | + | | 156 |
| DLGVRVCEK | 1b/1a/3a | | | | 154 |
| FLVNAWKSK | 1b | 1778 | | | 150 |
| GIFRAAVCTR | 1b/1a/3a | 129 | | | 1141 |
| GLNAVAYYR | 1b/3a | 44* | | | 145 |
| GMFDSSVLC | 1b/1a | | | | 71 |
| GNTLTCYLK | 1b | 160 | | | 40 |
| GVAGALVAFK | | | | | 1193 |
| GVLAALAAY | 1b/1a/3a | 545 | | | 1196 |
| GVVCAAILR | 1b/1a | 38 | + | | 1205 |
| GVVCAAILRR | 1b/1a | 215 | + | | 1206 |
| HLHAPTGSGK | 1b/1a | 501* | | | 1226 |
| HLIFCHSKK | 1b/1a/3a | 1531* | | | 148 |
| HLIFCHSKKK | 1b/1a/3a | 423 | | | 1228 |
| HMWNFISGI | 1b/1a | 7293 | | | 1233 |
| IVFPDLGVR | 1b/1a | 770 | | | 168 |
| KTKRNTNRR | 1b/1a | 646* | | | 164 |
| KTSERSQPR | 1b/1a/3a | 147* | + | | 167 |
| KVLVDILAGY | 1b/1a | 163* | + | | 1350 |
| LFNWAVRTK | 1b/1a/3a | 7567 | | | 1380 |
| LGFGAYMSK | 1b/1a | 22* | | | 50 |
| LIFCHSKKK | 1b/1a/3a | 104* | + | | 47 |
| LLYRLGAVQ | 1b/1a | | | | 200 |
| LVNAWKSKK | 1b | 50* | + | | 146 |
| QLFTFSPRR | 1b/1a | 197* | + | | 1609 |
| RLGVRATRK | 1b/1a/3a | 221* | + | | 144 |
| RLLAPITAY | 1b/1a/3a | 222* | | | 1662 |
| RMYVGGVEHR | 1b/1a | 15 | | | 1672 |
| RQPIPKARR | 1b/1a/3a | * | | | 159 |
| RVCEKMALY | 1b/1a | 160* | + | | 3 |
| RVFTEAMTR | 1b | 21* | + | | 39 |
| RVLEDGVNY | 1b/1a | 893 | | | 45 |
| SQLSAPSLK | 1b/1a/3a | 14* | | | 1781 |
| STNPKPQRK | 1b/1a | 14* | + | | 158 |
| TLGFGAYMSK | 1b/1a | 44* | | | 1831 |
| VAGALVAFK | 1b/1a | 46 | | | 1890 |
| VQPEKGGRK | 1b/1a | 1460 | | | 157 |
| VTLTHPITK | 1b | 7.7* | | | 21 |
| WLLSPRGSR | 1b/1a/3a | | | | 163 |
| WMNSTGFTK | 1b/1a/3a | 138* | | | 2016 |
| YLFNWAVRTK | 1b/1a/3a | 164* | | | 2044 |
| YLKASAACR | 1b | | | | 161 |
| YLLPRRGPR | 1b/1a | * | | | 149 |

* = binds A0301 with Ki < 1000 nM
HLA-A2402

| | | | | |
|---|---|---|---|---|
| AIKGGRHLI | | 336 | | 813 |
| ALYDVVSTL | 1b | 340 | | 67 |
| AQGYKVLVL | 1b/1a | 2164 | | 130 |
| AVMGSSYGF | 1b/1a | 8 | + | 16 |
| AWKSKKCPM | | 6675 | | 898 |
| AYAAQGYKV | 1b/1a | 30 | | 277 |
| AYAAQGYKVL | 1b/1a | 2102 | | 900 |
| AYMSKAHGV | 1b | 30 | | 244 |
| CLIRLKPTL | 1b/1a | 113 | + | 122 |
| CYSIEPLDL | 1b/1a | 786 | | 951 |
| ELAAKLSAL | | 932 | | 1016 |
| EPEPDVAVL | 1b/1a | high | | 1024 |
| ETTMRSPVF | | 219 | + | 1034 |
| FSLDPTFTI | 1b/1a | 74 | | 106 |
| FWAKHMWNF | 1b/1a | 2 | + | 1095 |
| FWAKHMWNFI | 1b/1a | 69.5 | + | 1096 |
| FWESVFTGL | 1b/3a | 15 | | 234 |
| GFADLMGYI | 1b/1a/3a | 75 | | 236 |
| GFSYDTRCF | 1b/1a/3a | 40 | | 281 |
| GLGWAGWLL | | 46 | + | 1150 |
| GLTHIDAHF | 1b/1a/3a | 3 | | 258 |
| GQIVGGVYL | 1b/1a | 17682 | | 127 |
| GYGAGVAGAL | 1b/1a | high | | 1210 |
| GYIPLVGAPL | 1b/1a | high | | 1211 |
| IFLLALLSCL | 1b/1a | high | | 1266 |
| IIMYAPTLW | 1b | 0.8 | + | 246 |
| KAHGVDPNI | 1b | 17123 | | 242 |
| KCDELAAKL | 1b/1a | high | | 1318 |
| KFPGGGQIV | 1b/1a/3a | 164 | | 284 |
| KGSSGGPLL | | 625 | | 1325 |
| KLQDCTMLV | | 2776 | | 1333 |
| KYIMACMSA | 1b | 6475 | | 239 |
| LFNWAVRTKL | 1b/1a | 1699 | | 1381 |
| LLPRRGPRL | | 226 | + | 1406 |
| LTHPITKYI | 1b | 403 | + | 23 |
| LWARMILMTHF | | 177 | + | 1483 |
| LYGNEGLGW | | 3.45 | | 1487 |
| MGSSYGFQY | | 9808 | | 1496 |
| MYTNVDQDL | 1b/1a/3a | 31 | + | 1512 |
| MYVGGVEHRL | 1b/1a | 291 | | 1513 |
| NFISGIQYL | 1b/1a | 293 | | 1521 |
| NIIMYAPTL | 1b | 249 | + | 87 |
| NLGKVIDTL | 1b/1a/3a | 93 | | 283 |
| NLPGCSFSI | 1b/1a/3a | 8 | + | 93 |
| PAVPQIFQV | 1b | 991 | | 282 |
| PFYGKAIPI | | 2 | | 1553 |
| PLLYRLGAV | | high | | 1558 |
| PVNSWLGNI | 1b/1a/3a | 319 | | 256 |
| QFKQKALGL | 1b/1a | high | | 1602 |
| QFKQKALGLL | 1b/1a | 4208 | | 1603 |
| QMWKCLIRL | 1b/1a | 439 | | 238 |
| QWMNRLIAF | 1b/1a/3a | 177 | | 1623 |
| QYLAGLSTL | 1b/1a/3a | 35 | + | 1625 |
| QYSPGQRVEF | 1b/1a | 298 | + | 1626 |
| RALAHGVRV | | high | | 1628 |
| RLGAVQNEV | | 3062 | | 1656 |
| RMILMTHFF | 1b/1a | 6 | + | 59 |
| RPDYNPPLL | 1b/1a/3a | high | | 1677 |
| RSELSPLLL | 1b/1a | high | | 1700 |
| RVEFLVNAW | | 261 | + | 1710 |
| SFSIFLLAL | 1b/1a/3a | 70 | + | 250 |
| SFSIFLLALL | 1b/1a | 9635 | | 1730 |
| SWDQMWKCL | 1b/1a | 550 | | 279 |
| TAGARLVVL | 1b/1a | 3194 | | 249 |
| TILGIGTVL | | 2070 | | 1826 |
| TLHGPTPLL | 1b/1a/3a | 217 | + | 81 |
| TLWARMILM | 1b/1a | 2101 | | 92 |
| IWAQPGYPW | | 8 | | 1883 |
| TYSTYGKF | | 147.5 | + | 1885 |
| TYSTYGKFL | 1b/1a/3a | 540 | | 241 |
| VIKGGRHLI | | 53 | + | 1916 |
| VILDSFDPL | 1b/1a | high | | 1918 |
| VMGSSYGF | | 23 | + | 1938 |
| WLGNIIMYA | 1b/3a | 15318 | | 62 |
| YAAQGYKVL | 1b/1a | 1636 | | 95 |
| YGKAIPIEV | | high | | 2034 |
| YIPLVGAPL | | 512 | | 2038 |
| YLNTPGLPV | | 372 | + | 2048 |
| YLVAYQATV | | 1844 | | 2052 |
| YYRGLDVSV | 1b/1a/3a | 31 | + | 271 |
| YYRGLDVSVI | 1b/1a/3a | 2 | + | 2083 |
| HLA-B0702 | | | | |
| AAKLSALGL | 1b | 277 | + | 402 |
| AAQGYKVLVL | 1b/1a | 5524 | | 777 |
| AARALAHGV | 1b/1a | 209 | | 403 |
| ALAHGVRVL | 1b/1a | 7950 | | 72 |
| APLGGAARA | 1b/1a | 115 | | 384 |
| APLGGAARAL | 1b/1a | 1 | + | 836 |
| APPPSWDQM | 1b/1a | 281 | + | 381 |
| APTGSGKST | 1b/1a/3a | 370 | | 397 |
| APTLWARM | 1b/1a | 13 | | 852 |
| APTLWARMI | 1b/1a | 11 | + | 371 |
| APTLWARMIL | 1b/1a | 1 | + | 853 |
| APTLWARMILM | 1b/1a | 423 | | 854 |
| ATLGFGAYM | 1b/1a | 12973 | | 32 |
| AVMGSSYGF | 1b/1a | 4400 | | 16 |
| DPPQPEYDL | 1b/1a | HIGH | | 543 |
| DPRRRSRNL | 1b/1a/3a | 18 | + | 370 |
| DPTTPLARA | 1b/1a | 13058 | | 980 |
| EARQAIRSL | 1b | 291 | | 388 |
| EPDVAVLTSM | 1b/1a | 454* | | 1023 |
| EPEPDVAVL | 1b/1a | HIGH* | | 1024 |
| EVIKGGRHL | 1b/1a | HIGH | | 376 |
| GPGEGAVQWM | 1b/1a/3a | 4747 | | 1163 |
| GPRLGVRAT | 1b/1a/3a | 128 | + | 387 |
| GPTPLLYRL | 1b/1a/3a | 209 | + | 307 |
| HPITKYIMA | 1b | 1106* | | 396 |
| HPNIEEVAL | 1b/1a | 230* | + | 1237 |
| IPFYGKAI | | 458 | | 1283 |
| IPLVGAPL | | 25* | + | 1289 |
| IPTSGDVVV | 1b/1a | 3152* | | 415 |
| KPARLIVF | | 367 | | 1336 |
| KPTLHGPTPL | 1b/1a/3a | 6 | + | 1343 |
| LPAILSPGAL | 1b/1a/3a | 255* | + | 1418 |
| LPALSTGLI | 1b/1a | 233 | + | 1419 |
| LPCEPEPDV | 1b/1a/3a | HIGH | | 1421 |
| LPCSFTTLPA | 1b/1a | 423 | | 1424 |
| LPGCSFSI | | 500 | | 1425 |
| LPGCSFSIF | 1b/1a/3a | 29* | + | 375 |
| LPGCSFSIFL | 1b/1a/3a | 558 | | 1426 |
| LPGNPAIASL | 1b/1a | 266 | | 1428 |
| LPINALSNSL | 1b/1a | 12* | + | 1430 |
| LPRRGPRL | | 1 | | 1442 |
| LPRRGPRLG | 1b/1a/3a | 124 | + | 380 |
| LPRRGPRLGV | 1b/1a/3a | 3 | + | 450 |
| LPVCQDHLEF | 1b/1a | 1564* | | 1444 |
| LPYIEQGM | | 423 | | 1445 |
| NAVAYYRGL | 1b/1a/3a | HIGH | | 400 |
| NPAIASLMA | 1b/1a | 676 | | 1527 |
| NPAIASLMAF | 1b/1a | 121* | | 1528 |
| NPSVAATLGF | 1b/1a/3a | 1197 | | 1532 |
| PPHSAKSKF | 1b/1a | HIGH | | 1568 |
| PPQPEYDLEL | 1b/1a | HIGH | | 1575 |
| PPVVHGCPL | 1b/1a | 433 | + | 1582 |
| QPRGRRQPI | 1b/1a/3a | 1 | + | 390 |
| RPDYNPPLL | 1b/1a/3a | 143 | + | 1677 |
| RPSGMFDSSV | 1b/1a | 14 | + | 1687 |
| SAACRAAKL | 1b | 106 | + | 121 |
| SPGALVVGV | 1b/1a/3a | 627 | | 1759 |
| SPGQRVEFL | 1b/1a | 38 | | 373 |
| SPRGSRPSW | 1b/1a/3a | 11 | + | 386 |
| SVFTGLTHI | 1b/3a | HIGH | | 76 |
| TPAETSVRL | 1b | 375 | | 383 |
| TPCTCGSSDL | 1b/1a | 168 | | 1843 |
| TPGERPSGM | 1b | 199 | + | 372 |
| TPLLYRLGA | 1b/1a | 74 | | 389 |
| TPLLYRLGAV | 1b/1a | 458 | | 1851 |
| VAYYRGLDV | 1b/1a/3a | 1417 | | 394 |
| WPLLLLLLAL | 1b/1a | 1474 | | 2018 |
| YAAQGYKVL | 1b/1a | 313 | + | 95 |

* = binds B3501 with Ki < 1000 nM

| | | | | | |
|---|---|---|---|---|---|
| HLA-B0801 | | | | | |
| AIRSLTERL | 1b | 3621 | | | 473 |
| AQGYKVLVL | 1b/1a | 1920 | | | 130 |
| ARRGREILL | 1b/1a | 3039 | | | 865 |
| CLRKLGVPPL | 1b/1a | 549 | | | 921 |
| DLCGSVFL | 1b/1a | 11347 | | | 962 |
| DLMGYIPLV | 1b/1a/3a | 1966 | | | 66 |
| DPRRRSRN | 1b/1a/3a | 12066 | | | 974 |
| DPRRRSRNL | 1b/1a/3a | 111 | | | 370 |
| DPRRRSRNLG | 1b/1a/3a | | | | 975 |
| DQMWKCLIRL | 1b/1a | | | | 982 |
| DTRCFDSTV | 1b/1a/3a | 13145 | | | 466 |
| DVKFPGGGQI | 1b/1a/3a | 14129 | | | 990 |
| EARQAIRSL | 1b | 9 | | | 388 |
| ESENKVVIL | 1b/1a | HIGH | | | 1030 |
| FPYLVAYQA | 1b/1a | 12 | | + | 443 |
| GCSFSIFL | 1b/1a/3a | 6708 | | | 1111 |
| GKRVYYLTR | 1b/1a | HIGH | | | 172 |
| GRRGIYRFV | 1b | 4984 | | | 464 |
| HPITKYIMA | 1b | 59 | | + | 396 |
| HSKKKCDEL | 1b/1a | 76 | | + | 455 |
| HSKKKCDELA | 1b/1a | | | | 1243 |
| IPKARRPE | 1b/1a | 1214 | | | 1286 |
| IPKARRPEG | 1b/1a | 874 | | | 409 |
| IPKARRPEGR | 1b/1a | | | | 1287 |
| IPLVGAPL | 1b/1a | 20 | | | 1288 |
| ITKYIMACM | 1b | 2610 | | | 305 |
| ITYSTYGKFL | 1b/1a | | | | 1310 |
| LIRLKPTLH | 1b/1a | 62 | | | 205 |
| LLPRRGPRL | 1b/1a | 183 | | | 132 |
| LPRRGPRL | 1b/1a/3a | 6 | | + | 1441 |
| LPRRGPRLGV | 1b/1a/3a | | | | 450 |
| LTHPITKYI | 1b | HIGH | | | 23 |
| LTRDPTTPL | 1b/1a | 4322 | | | 1475 |
| NAVAYYRGL | 1b/1a/3a | 11365 | | | 400 |
| NAWKSKKCPM | 1b | | | | 1514 |
| NIRTGVRTI | 1b/1a | 619 | | + | 436 |
| NPKPQRKTK | 1b/1a | | | | 404 |
| NPKPQRKTKR | 1b/1a | | | | 1531 |
| PARLIVFPDL | 1b/1a | 5747 | | | 1545 |
| QFKQKALGL | 1b/1a | 65 | | | 1602 |
| QMWKCLIRL | 1b/1a | 8712 | | | 238 |
| QPRGRRQP | 1b/1a/3a | HIGH | | | 1615 |
| QPRGRRQPI | 1b/1a/3a | 3 | | | 390 |
| QPRGRRQPIP | 1b/1a/3a | | | | 1616 |
| QRKTKRNTNR | 1b/1a | | | | 1618 |
| QRRGRTGRG | 1b/1a/3a | 314 | | | 456 |
| QTRGLLGCI | 1b/1a | HIGH | | | 1621 |
| QTRGLLGCII | 1b/1a | 17793 | | | 1622 |
| RSRNLGKVI | 1b/1a/3a | 17415 | | | 318 |
| RTKLKLTPI | 1b/1a | 2 | | | 1705 |
| SARRGREIL | 1b/1a | 6454 | | | 1718 |
| SARRGREILL | 1b/1a | 133 | | + | 1719 |
| SGKRVYYLTR | 1b | | | | 1733 |
| SGKSTKVPAA | 1b/1a/3a | | | | 1734 |
| SPGQRVEFL | 1b/1a | 120 | | | 373 |
| SVRLRAYLN | 1b | 3314 | | | 459 |
| TAGARLVVL | 1b/1a | 16 | | | 249 |
| TGRGRRGIYR | 1b | | | | 1825 |
| TIMAKNEVF | 1b/1a/3a | 6 | | | 1827 |
| TLWARMILM | 1b/1a | 59 | | + | 92 |
| VAYYRGLDV | 1b/1a/3a | 278 | | + | 394 |
| VSARRGREI | 1b/1a | 192 | | + | 1969 |
| VTFDRLQVL | 1b/1a/3a | 4358 | | | 1975 |
| WAKHMWNFI | 1b/1a | 104 | | | 1993 |
| WARMILMTH | 1b/1a | 166 | | + | 287 |
| WARPDYNPPL | 1b/1a/3a | 1030 | | | 1998 |
| YGKAIPIEVI | 1b | | | | 2035 |
| YLKGSSGGPL | 1b/1a | 10 | | | 2046 |
| YLLPRRGPRL | 1b/1a | 142 | | | 2047 |
| YRRCRASGV | 1b/1a/3a | 11 | | | 475 |
| YRRCRASGVL | 1b/1a/3a | | | | 2066 |
| HLA-B3501 | | | | | |
| APPPSWDQM | 1b/1a | 17771* | | | 381 |
| APTLWARMI | 1b/1a | HIGH* | | | 371 |
| AVMGSSYGF | 1b/1a | HIGH | | | 16 |
| DPPQPEYDL | 1b/1a | HIGH | | | 543 |
| EPEPDVAVL | 1b/1a | HIGH | | | 1024 |
| FPGGGQIVG | 1b/1a/3a | 2596 | | | 407 |
| FPGGGQIVGG | 1b/1a/3a | | | | 1077 |
| FPYLVAYQA | 1b/1a | 18 | | | 443 |
| FSLDPTFTI | 1b/1a | | | | 106 |
| GPGEGAVQW | 1b/1a/3a | HIGH | | | 1162 |
| GPTPLLYRL | 1b/1a/3a | 17916* | | | 307 |
| HPNIEEVAL | 1b/1a | 7* | + | | 1237 |
| IPFYGKAIPI | 1b/3a | | | | 1284 |
| IPLVGAPL | | 295* | + | | 1289 |
| IPVESMETTM | | | | | 1296 |
| LPALSTGLI | 1b/1a | HIGH* | | | 1419 |
| LPCEPEPDV | 1b/1a/3a | HIGH | | | 1421 |
| LPGCSFSIF | 1b/1a/3a | 90* | + | | 375 |
| LPGCSFSIFL | 1b/1a/3a | 1494* | | | 1426 |
| LPINALSNSL | 1b/1a | 137* | + | | 1430 |
| LPVCQDHLEF | 1b/1a | 104 | + | | 1444 |
| MGSSYGFQY | 1b/1a | 2607 | | | 22 |
| MPSTEDLVNL | 1b | | | | 1505 |
| NPSVAATLGF | 1b/1a/3a | 1401 | | | 1532 |
| QAVMGSSYGF | 1b | | | | 1599 |
| QPRGRRQPI | 1b/1a/3a | HIGH* | | | 390 |
| RPDYNPPLL | 1b/1a/3a | HIGH* | | | 1677 |
| RVLEDGVNY | 1b/1a | HIGH | | | 45 |
| SALGLNAVAY | 1b | | | | 1717 |
| SPGQRVEFL | 1b/1a | HIGH* | | | 373 |
| TPAETSVRL | 1b | 1643* | | | 383 |
| YDAGCAWYEL | 1b/1a | | | | 2030 |

* = binds B0702 with Ki < 1000 nM

| | | | | | |
|---|---|---|---|---|---|
| HLA-B4403 | | | | | |
| AATLGFGAY | 1b/1a | 11055 | | | 557 |
| ADLEVVTST | 1b/1a | HIGH | | | 786 |
| AEAALENLV | 1b/1a/3a | 126 | | | 790 |
| AEQFKQKAL | 1b/1a | 67 | | | 794 |
| AEQFKQKALG | 1b/1a | 3058 | | | 795 |
| AETAGARLV | 1b/1a | 122 | + | | 582 |
| AETAGARLVV | 1b/1a | 2704 | | | 796 |
| AETSVRLRAY | 1b | | | | 799 |
| AGYSGGDIY | 1b/1a | HIGH | | | 809 |
| AQPGYPWPL | 1b/1a/3a | HIGH | | | 65 |
| CECYDAGGA | 1b/1a | 13219 | | | 589 |
| CECYDAGCAW | 1b/1a | 1056 | | | 910 |
| CEKMALYDV | 1b/1a | 7759 | | | 581 |
| CEPEPDVAV | 1b/1a | high | | | 912 |
| CEPEPDVAVL | 1b/1a | HIGH | | | 913 |
| DELAAKLSA | 1b | 12653 | | | 569 |
| DGGCSGGAY | 1b/1a/3a | HIGH | | | 959 |
| DQRPYCWHY | 1b/1a | HIGH | | | 984 |
| DSSVLCECY | 1b/1a | HIGH | | | 17 |
| FDITKLLLA | 1b/1a | HIGH | | | 1053 |
| GEIPFYGKA | 1b/1a/3a | HIGH | | | 1116 |
| GEIPFYGKAI | 1b/1a/3a | 354 | + | | 1117 |
| GEPGDPDLS | 1b/1a/3a | HIGH | | | 1128 |
| GGQIVGGVY | 1b/1a/3a | HIGH | | | 1137 |
| GQIVGGVYL | 1b/1a | HIGH | | | 127 |
| HSAKSKFGY | 1b/1a | HIGH | | | 1241 |
| IEVIKGGRHL | 1b | | | | 1265 |
| LEDGVNYAT | 1b/1a | HIGH | | | 31 |
| LEDRDRSEL | 1b/1a | high | | | 1368 |
| LEFWESVFT | 1b | | | | 129 |
| LEFWESVFTG | 1b | | | | 1371 |
| LELITSCSS | 1b/1a/3a | HIGH | | | 1372 |
| LEVVTSTWV | 1b/1a | HIGH | | | 1377 |
| LEVVTSTWVL | 1b/1a | 5346 | | | 1378 |
| LSAFSLHSY | 1b/1a | 3145 | | | 1456 |
| METTMRSPVF | 1b | | | | 1493 |
| NEGMGWAGWL | 1b | | | | 1517 |
| PEKGGRKPA | 1b/1a | high | | | 1550 |
| PESDAAARVT | 1b/1a/3a | high | | | 1551 |
| PEYDLELIT | 1b/1a | high | | | 587 |
| RGVAKAVDF | 1b/1a | HIGH | | | 317 |
| RMILMTHFF | 1b/1a | 389 | + | | 59 |
| SCGNTLTCY | 1b/1a | 11574 | | | 1722 |
| SELSPLLLS | 1b/1a | 10368 | | | 1725 |
| SELSPLLLST | 1b/1a | 2177 | | | 1726 |
| TEAMTRYSA | 1b/1a/3a | 4934 | | | 586 |
| TEDLVNLLPA | 1b/1a | high | | | 1818 |
| TERLYIGGPL | 1b | | | | 1820 |
| TLWARMILM | 1b/1a | 10410 | | | 92 |
| VDFSLDPTF | 1b/1a/3a | 1462 | | | 350 |
| VEFLVNAWKS | 1b | | | | 1900 |
| VESENKVVI | 1b/1a | 1702 | | | 1901 |
| VESENKVVIL | 1b/1a | 10100 | | | 1902 |
| VMGSSYGFQY | 1b/1a | | | | 1939 |
| WESVFTGLTH | 1b/3a | | | | 2002 |
| WETARHTPV | 1b/1a/3a | HIGH | | | 577 |
| WLGNIIMYA | 1b/3a | 1949 | | | 62 |
| HLA-Cw0301 | | | | | |
| AALENLVVL | 1b | | | | 776 |
| ADLMGYIPL | 1b/1a/3a | | | | 2085 |
| AILGPLMVL | 1b | | | | 816 |
| AKVLIVMLL | 1b | | | | 2097 |
| ALYDVVSTL | 1b | 54.38 | | | 67 |
| ARLIVFPDL | 1b/1a | 18558 | | | 643 |
| AVIPDREVL | 1b | | | | 891 |
| CLIRLKPTL | 1b/1a | 2108 | | | 122 |
| CQVPAPEFF | 1b | | | | 2094 |
| CWVALTPTL | 1b | | | | 950 |
| ERLYIGGPL | 1b | 17523 | | | 428 |
| ESYSSMPPL | 1b/1a | | | | 2089 |
| EVIKGGRHL | 1b/1a | 8355 | | | 376 |
| FLLALLSCL | 1b/1a | 1229 | | | 361 |
| FQVGLNQYL | 1b | | | | 2100 |
| FWESVFTGL | 1b/3a | 10265 | | | 234 |
| GALVAFKVM | 1b | | | | 2086 |
| GAVFVGLAL | 1b | | | | 1107 |
| GAVQNEVTL | 1b/1a | | | | 347 |
| GAVQWMNRL | 1b/1a/3a | | | | 1109 |
| GEIPFYGKA | 1b/1a/3a | | | | 1116 |
| GEMPSTEDL | 1b | | | | 2084 |
| GQIVGGVYL | 1b/1a | 70.23 | | | 127 |
| GSIGLGKVL | 1b | | | | 1186 |
| GSVFLVSQL | 1b | | | | 1189 |
| HGILSFLVF | 1b | | | | 2095 |
| ILMTHFFSI | 1b | | | | 1273 |
| ITYSTYGKF | 1b/1a | 4273 | | | 26 |
| LSVEEACKL | 1b | | | | 2092 |
| NAVAYYRGL | 1b/1a/3a | 1814 | | | 400 |
| NFISGIQYL | 1b/1a | 1070 | | | 1521 |
| NIIMYAPTL | 1b | 32.21 | | | 87 |
| NQYLVGSQL | 1b | | | | 2099 |
| QAGDNFPYL | 1b | | | | 551 |
| QIIERLHGL | 1b | | | | 2091 |
| QIVGGVYLL | 1b/1a | 124 | | | 91 |
| QNIVDVQYL | 1b/1a/3a | | | | 2098 |
| QYLAGLSTL | 1b/1a/3a | | | | 1625 |
| RAYLNTPGL | 1b | 512.8 | | | 434 |
| SDVESYSSM | 1b | | | | 2088 |
| SGMFDSSVL | 1b/1a | | | | 135 |
| SPLTTQHTL | 1b | | | | 1767 |
| SSLTITQLL | 1b | | | | 1785 |
| STLPGNPAI | 1b/1a | | | | 2096 |
| TALNCNDSL | 1b | | | | 1812 |
| TALVVSQLL | 1b | | | | 1813 |
| TILGIGTVL | 1b | 57.24 | | | 89 |
| TMLVNGDDL | 1b | | | | 2101 |
| TPIPAASQL | 1b | | | | 1848 |
| TRVPYFVRA | 1b | | | | 2087 |
| TTIRRHVDL | 1b | | | | 1874 |
| VILDSFDPL | 1b/1a | 49.21 | | | 1918 |
| VLYREFDEM | 1b/1a | | | | 2090 |
| VRVCEKMAL | 1b/1a | high | | | 632 |
| WAVRIKLKL | 1b/1a | | | | 1999 |
| WHYPCTVNF | 1b/3a | | | | 2093 |
| YAAQGYKVL | 1b/1a | 2155 | | | 95 |
| YALYGVWPL | 1b | | | | 2025 |
| YVLLLFLLL | 1b | | | | 2073 |
| HLA-Cw0401 | | | | | |
| AWETARHTPV | 1b/1a/3a | 2297 | | | 897 |
| AYAAQGYKV | 1b/1a | high | | | 277 |
| AYAAQGYKVL | 1b/1a | high | | | 900 |
| CYSIEPLDL | 1b/1a | 702 | | | 951 |
| DFSLDPTFTI | 1b/1a | high | | | 958 |
| DPPQPEYDL | 1b/1a | high | | | 543 |
| EFWESVFTGL | 1b | 46.5 | | | 1010 |
| EPEPDVAVL | 1b/1a | high | | | 1024 |
| EYDLELITS | 1b/1a | high | | | 597 |
| FADLMGYIPL | 1b/1a/3a | 613 | | + | 1048 |
| FWAKHMWNF | 1b/1a | 124 | | + | 1095 |
| FWESVFTGL | 1b/3a | 2 | | | 234 |
| GFADLMGYI | 1b/1a/3a | 569 | | | 236 |
| GPTPLLYRL | 1b/1a/3a | high | | | 307 |
| HPNIEEVAL | 1b/1a | high | | | 1237 |
| MYAPTLWARM | 1b | high | | | 1511 |
| NFISGIQYL | 1b/1a | 37.5 | | | 1521 |
| PWPLYGNEGM | 1b | 1083 | | | 1590 |
| QFKQKALGL | 1b/1a | high | | | 1602 |
| QYLAGLSTL | 1b/1a/3a | 8058 | | | 1625 |
| QYSPGQRVEF | 1b/1a | high | | | 1626 |
| RPDYNPPLL | 1b/1a/3a | 356 | | | 1677 |
| SFSIFLLAL | 1b/la/3a | 396 | | | 250 |
| SFSIFLLALL | 1b/1a | 10188 | | + | 1730 |
| SPGQRVEFL | 1b/1a | high | | | 373 |
| SWDQMWKCL | 1b/1a | 66 | | | 279 |
| SWDQMWKCLI | 1b/1a | 989 | | | 1804 |
| TYSTYGKFL | 1b/1a/3a | 18449 | | | 241 |
| VFPDLGVRV | 1b/1a | 787 | | + | 349 |
| HLA-Cw0602 | | | | | |
| AAQGYKVLV | 1b/1a | 6319 | | | 115 |
| AEQFKQKAL | 1b/1a | high | | | 794 |
| ALAHGVRVL | 1b/1a | 3308 | | | 72 |
| AQGYKVLVL | 1b/1a | high | | | 130 |
| ARALAHGVRV | 1b/1a | 9879 | | | 862 |
| ARHTPVNSWL | 1b/1a/3a | high | | | 863 |
| ARLIVFPDL | 1b/1a | high | | | 643 |
| ARMILMTHF | 1b/1a | high | | | 641 |
| ARMILMTHFF | 1b/1a | high | | | 864 |
| ARVTQILSSL | 1b | high | | | 868 |
| AYAAQGYKV | 1b/1a | high | | | 277 |
| AYAAQGYKVL | 1b/1a | high | | | 900 |
| AYMSKAHGV | 1b | high | | | 244 |
| AYYRGLDVSV | 1b/1a/3a | 1074 | | + | 907 |
| CLIRLKPTL | 1b/1a | high | | | 122 |
| DLVNLLPAI | 1b/1a | high | | | 968 |
| DRSELSPLL | 1b/1a | high | | | 986 |
| DVAVLTSML | 1b/1a | high | | | 989 |
| EARQAIRSL | 1b | high | | | 388 |
| EPEPDVAVL | 1b/1a | high | | | 1024 |
| ERLYIGGPL | 1b | high | | | 428 |
| ESENKVVIL | 1b/1a | high | | | 1030 |
| ETSVRLRAY | 1b | high | | | 46 |
| EVIKGGRHL | 1b/1a | 12838 | | | 376 |
| FADLMGYIPL | 1b/1a/3a | high | | | 1048 |
| FKQKALGLL | 1b/1a | high | | | 1062 |
| FLLALLSCL | 1b/1a | high | | | 361 |
| FQYSPGQRV | 1b/1a | 387 | | + | 111 |
| FRAAVCTRGV | 1b/1a/3a | 486 | | | 1081 |
| FSIFLLALL | 1b/1a | high | | | 362 |
| FYGKAIPIEV | 1b | 5690 | | | 1100 |
| GGGQIVGGV | 1b/1a/3a | high | | | 1136 |
| GPTPLLYRL | 1b/1a/3a | high | | | 307 |
| GQIVGGVYLL | 1b/1a | high | | | 1178 |
| GRGRRGIYRF | 1b | high | | | 1181 |
| GRKPARLIV | 1b/1a/3a | 2037 | | | 507 |
| GRKPARLIVF | 1b/1a | 8955 | | | 1182 |
| GRRGIYRFV | 1b | 575.5 | | | 464 |
| HMWNFISGI | 1b/1a | high | | | 1233 |
| IFLLALLSCL | 1b/1a | high | | | 1266 |
| IKGGRHLIF | 1b/1a | high | | | 311 |
| IMACMSADL | 1b | high | | | 90 |
| IRSLTERLY | 1b | 318 | | | 624 |
| IRSLTERLYI | 1b | 6225 | | | 1301 |
| KCDELAAKL | 1b/1a | high | | | 1318 |
| LGKVLVDIL | 1b/1a | high | | | 1382 |
| LLGCIITSL | 1b/1a | high | | | 1397 |
| LRAYLNTPGL | 1b | high | | | 1454 |
| LVNLLPAIL | 1b/1a | high | | | 1481 |
| MALYDVVSTL | 1b | high | | | 1492 |
| MYAPTLWARM | 1b | high | | | 1511 |
| NAVAYYRGL | 1b/1a/3a | 10829 | | | 400 |
| NFISGIQYL | 1b/1a | 6642 | | | 1521 |
| QMWKCLIRL | 1b/1a | high | | | 238 |
| QTRGLLGCI | 1b/1a | high | | | 1621 |
| QYSPGQRVEF | 1b/1a | high | | | 1626 |
| RALAHGVRVL | 1b/1a | 7337 | | | 1629 |
| RKPARLIVF | 1b/1a | 8281 | | | 646 |
| RMILMTHFF | 1b/1a | high | | | 59 |
| SFSIFLLAL | 1b/1a/3a | high | | | 250 |
| SKKCPMGFSY | 1b | high | | | 1739 |
| SPGALVVGV | 1b/1a/3a | high | | | 1759 |
| STEDLVNLL | 1b/1a | high | | | 1787 |
| STWVLVGGV | 1b/1a | high | | | 1793 |
| SWDQMWKCL | 1b/1a | high | | | 279 |
| TLPALSTGL | 1b/1a | high | | | 1835 |
| TYSTYGKFL | 1b/1a/3a | 4859 | | | 241 |
| VAYYRGLDV | 1b/1a/3a | 231 | | | 394 |
| VIKGGRHLIF | 1b/1a | 17503 | | | 1917 |
| VKFPGGGQIV | 1b/1a/3a | 417.5 | | | 1920 |
| VLVDILAGY | 1b/1a | high | | | 1931 |
| VRVCEKMAL | 1b/1a | high | | | 632 |
| VRVCEKMALY | 1b/1a | high | | | 1967 |
| VTFDRLQVL | 1b/1a/3a | 1131.5 | | | 1975 |
| WAQPGYPWPL | 1b/1a/3a | high | | | 1996 |
| WARMILMTHF | 1b/1a | high | | | 1997 |
| WKSKKCPMGF | 1b | high | | | 2013 |
| YAAQGYKVL | 1b/1a | 1784 | | | 95 |
| YAAQGYKVLV | 1b/1a | 9209 | | | 2024 |
| YLLPRRGPRL | 1b/1a | high | | | 2047 |
| YRLGAVQNEV | 1b/1a | 216.5 | | | 2065 |
| YRRCRASGV | 1b/1a/3a | 634 | | + | 475 |
| YRRCRASGVL | 1b/1a/3a | | | | 2066 |
| YYRGLDVSV | 1b/1a/3a | | | | 271 |
| YYRGLDVSVI | 1b/1a/3a | | | | 2083 |
| HLA-Cw0702 | | | | | |
| AATLGFGAY | 1b/1a | high | | | 557 |
| ARHTPVNSWL | 1b/1a/3a | 122 | | | 863 |
| ARLIVFPDL | 1b/1a | 298 | | | 643 |
| ARMILMTHF | 1b/1a | 155 | | | 641 |
| ARMILMTHFF | 1b/1a | 272 | | | 864 |
| ARVTQILSSL | 1b | 488 | | | 868 |
| AYAAQGYKV | 1b/1a | 12544 | | | 277 |
| AYAAQGYKVL | 1b/1a | 4955 | | | 900 |
| AYYRGLDVSV | 1b/1a/3a | 23 | | + | 907 |
| CYDAGCAWY | 1b/1a | 1091.3 | | | 13 |
| DGGCSGGAY | 1b/1a/3a | high | | | 959 |
| DPPQPEYDL | 1b/1a | high | | | 543 |
| DQRPYCWHY | 1b/1a | 709.5 | | | 984 |
| DREVLYREF | 1b/1a | high | | | 985 |
| DSSVLCECY | 1b/1a | high | | | 17 |
| DYPYRLWHY | 1b/1a/3a | 0.2 | | | 997 |
| FRKHPEATY | 1b/1a/3a | 0.2 | | | 1082 |
| FYGKAIPIEV | 1b | 31 | | + | 1100 |
| GFADLMGYI | 1b/1a/3a | high | | | 236 |
| GFSYDTRCF | 1b/1a/3a | high | | | 281 |
| GGQIVGGVY | 1b/1a/3a | high | | | 1137 |
| GPTPLLYRL | 1b/1a/3a | 11601 | | | 307 |
| GRKPARLIVF | 1b/1a | 753 | | | 1182 |
| GVAGALVAF | 1b/1a | 6162 | | | 1191 |
| GVLAALAAY | 1b/1a/3a | high | | | 1196 |
| GYIPLVGAPL | 1b/1a | 2403 | | | 1211 |
| HQNIVDVQY | 1b/1a/3a | 14194 | | | 1239 |
| HSAKSKFGY | 1b/1a | high | | | 1241 |
| HYVPESDAAA | 1b/1a/3a | high | | | 1254 |
| IRSLTERLY | 1b | 25 | | | 624 |
| KKCDELAAKL | 1b/1a | high | | | 1327 |
| KSTKVPAAY | 1b/1a/3a | high | | | 25 |
| KYIMACMSA | 1b | 7210 | | | 239 |
| LHGPTPLLY | 1b/1a/3a | 31 | | | 219 |
| LLGCIITSL | 1b/1a | 9762 | | | 1397 |
| LPGCSFSIF | 1b/1a/3a | 10440 | | | 375 |
| LRAYLNTPGL | 1b | 236 | | + | 1454 |
| LSAFSLHSY | 1b/1a | high | | | 1456 |
| LVGGVLAAL | 1b/1a | high | | | 1479 |
| LYGNEGMGWA | 1b | 5108 | | | 1488 |
| MYAPTLWARM | 1b | 548 | | + | 1511 |
| MYTNVDQDL | 1b/1a/3a | 81 | | | 1512 |
| NEPYLVAYQA | 1b/1a | 5300 | | | 1522 |
| NIVDVQYLY | 1b/1a/3a | 56 | | | 1523 |
| NLGKVIDTL | 1b/1a/3a | high | | | 283 |
| QPGYPWPLY | 1b/1a/3a | high | | | 216 |
| RLLAPITAY | 1b/1a/3a | 4821 | | | 1662 |
| SCGNTLTCY | 1b/1a | high | | | 1722 |
| SFSIFLLAL | 1b/1a/3a | 312 | | | 250 |
| SFSIFLLALL | 1b/1a | high | | | 1730 |
| SKKCPMGFSY | 1b | 901 | | | 1739 |
| SPGALVVGV | 1b/1a/3a | high | | | 1759 |
| SWLGNIIMY | 1b/3a | high | | | 665 |
| TYSTYGKFL | 1b/1a/3a | 239 | | | 241 |
| VLVDILAGY | 1b/1a | high | | | 1931 |
| VRVCEKMAL | 1b/1a | 279 | | | 632 |
| VRVCEKMALY | 1b/1a | 2586 | | | 1967 |
| WARMILMTHF | 1b/1a | 855 | | | 1997 |
| YAPTLWARM | 1b | high | | | 14 |
| YRRCRASGVL | 1b/1a/3a | 83 | | + | 2066 |
| YYRGLDVSV | 1b/1a/3a | 12 | | | 271 |
| YYRGLDVSVI | 1b/1a/3a | 87 | | | 2083 |

TABLE 14

Selection of HLA-DRB1*0101 and -DRB1*0301 predicted peptides
Immun = Immunogenicity
Cons. = presence of the “core” in the indicated consensus sequence

| | | | | | | | | |
|---|---|---|---|---|---|---|---|---|
| | | | | 0101 | 0301 | 0401 | | SEQ |
| | | Cons | Cons | IC₅₀ | IC₅₀ | IC₅₀ | | ID |
| Sequence | Cons 3a | 1b/1a | 1b/1a/3a | nM | nM | nM | Immun | NO |

| | | | | | | | | |
|---|---|---|---|---|---|---|---|---|
| ADLMGYIPLVGAPLG | | X | X | 8333 | | | | 2105 |
| GHRMAWDMMMNWSPT | | X | X | 183 | | | | 2142 |
| SKGWRLLAPITAYAQ | | X | X | 0.40 | | | | 2213 |
| RAAVCTRGVAKAVDF | | X | X | 619 | | | | 2199 |
| GYKVLVLNPSVAATL | | X | X | 8.4 | | | | 2157 |
| VLVLNPSVAATLGFG | | X | X | 3.0 | 1431 | 6.5 | + | 2235 |
| WESVFTGLTHIDAHF | | X | X | 122 | | | | 2245 |
| KPTLHGPTPLLYRLG | | X | X | 156 | 1510 | 4861 | + | 2164 |
| IQYLAGLSTLPGNPA | | X | X | 3.4 | | 2.6 | + | 2162 |
| AVQWMNRLIAFASRG | | X | X | 2 | 17313 | 1009 | + | 2113 |
| MNRLIAFASRGNHVS | | X | X | 57 | 9529 | 813 | + | 2182 |
| ASQLSAPSLKATCTT | | X | X | 329 | | | | 2111 |
| TTIMAKNEVFCVQPE | | X | X | 3727 | | | | 2226 |
| GIQYLAGLSTLPGNP | | X | X | | | | | 2143 |
| LPAILSPGALVVGVV | | X | X | | | | | 2172 |
| DLVNLLPAILSPGA | | X | X | | | | | 2121 |
| YKVLVLNPSVAATLG | | X | X | | | | | 2252 |
| LVVLATATPPGSVTV | | X | X | 73 | 4230 | 4 | + | 2180 |
| STTILGIGTVLDQAE | | X | X | | | | | 2217 |
| VNLLPAILSPGALVV | | X | X | 2.3 | 12603 | 1558 | + | 2236 |
| GGVLAALAAYCLTTG | | X | X | | | | | 2141 |
| KVLVLNPSVAATLGF | | X | X | | | | | 2165 |
| LPALSTGLIHLHQNI | | X | X | | | | | 2173 |
| VGGVLAALAAYCLTT | | X | X | | | | | 2233 |
| GQGWRLLAPITAYSQ | | X | X | | | | | 2150 |
| VQWMNRLIAFASRGN | | X | X | | | | | 2238 |
| GPRLGVRATRKTSER | | X | X | | 18887 | | + | 2149 |
| LTHIDAHFLSQTKQA | | X | X | | | | | 2175 |
| LTHIDAHFLSQTKQS | | X | X | | | | | 2176 |
| VGIFRAAVCTRGVAK | | X | X | | | | | 2234 |
| LVAYQATVCARAQAP | | X | X | | | | | 2178 |
| LVNLLPAILSPGALV | | X | X | | | | | 2179 |
| AVGIFRAAVCTRGVA | | X | X | 29 | 10143 | 31 | + | 2112 |
| GLGWAGWLLSPRGSR | | X | X | | | | | 2145 |
| GCGWAGWLLSPRGSR | | X | X | | | | | 2138 |
| GMGWAGWLLSPRGSR | | X | X | | | | | 2147 |
| RLVVLATATPPGSVT | | X | X | | | | | 2203 |
| GKYLFNWAVRTKLKL | | X | X | | | | | 2144 |
| GHRMAWDMMMNWSPT | | X | X | | 919 | | | 2142 |
| DSSVLCECYDAGCAW | | X | X | | | | | 2123 |
| PTHYVPESDAAARVT | | X | X | | 4954 | | | 2193 |
| GSQLPCEPEPDVAVL | | X | X | | | | | 2152 |
| QPEYDLELITSCSSN | | X | X | | | | | 2198 |
| RLGVRATRKTSERSQ | | X | X | | 16443 | 5868 | + | 2201 |
| YGKFLADGGCSGGAY | | X | X | 5082 | 2565 | 21 | + | 2249 |
| HLIFCHSKKKCDELA | | X | X | | | | + | 2158 |
| LIRLKPTLHGPTPLL | | X | X | | | | | 2171 |
| MPPLEGEPGDPDL | | X | X | | | | | 2183 |
| QKKVTFDRLQVLDDH | | X | X | | | | | 2197 |
| PMGFSYDTRCFDSTV | | X | X | | | | | 2191 |
| RPEYDLELITSCSSN | | X | X | | | | | 2204 |
| ARAAWETARHTPVNS | | X | X | 1402 | | 14756 | + | 2108 |
| GVNYATGNLPGCSFS | | X | | 4564 | | | | 2155 |
| GCSFSIFLLALLSCL | | X | | 1634 | | | | 2139 |
| FTTLPALSTGLIHLH | | X | | 1.3 | | 2080 | | 2132 |
| PQTFQVAHLHAPTGS | | X | | 22 | | | | 2192 |
| AQGYKVLVLNPSVAA | | X | | 4.6 | 5169 | 1.6 | + | 2107 |
| GTVLDQAETAGARLV | | X | | | | | | 2154 |
| GARLVVLATATPPGS | | X | | 173 | | | | 2137 |
| DVVVVATDALMTGYT | | X | | 456 | | | | 2125 |
| VVVVATDALMTGYTG | | X | | 1082 | | | | 2241 |
| TWVLVGGVLAALAAY | | X | | 6.9 | | 369 | + | 2229 |
| LAGYGAGVAGALVAF | | X | | 137 | | | | 2166 |
| GALVVGVVCAAILRR | | X | | 314 | | | | 2136 |
| LTSMLTDPSHITAET | | X | | 12.50 | | | | 2177 |
| DADLIEANLLWRQEM | | X | | 574 | | | | 2117 |
| RQEMGGNITRVESEN | | X | | | | | | 2205 |
| GSSYGFQYSPGQRVE | | X | | 11 | | | | 2153 |
| PTLWARMILMTHFFS | | X | | 788 | 3424 | 178 | + | 2194 |
| ASCLRKLGVPPLRVW | | X | | 4.9 | | | | 2110 |
| WVLVGGVLAALAAYC | | X | | | | | | 2247 |
| EPDVAVLTSMLTDPS | | X | | | | | | 2127 |
| PCSFTTLPALSTGLI | | X | | | | | | 2189 |
| GDNFPYLVAYQATVC | | X | | | | | | 2140 |
| YIPLVGAPLGGAARA | | X | | | | | | 2251 |
| PYLVAYQATVCARAQ | | X | | | | | | 2195 |
| ARLVVLATATPPGSV | | X | | | | | | 2109 |
| STKVPAAYAAQGYKV | | X | | | | | | 2216 |
| FNILGGWVAAQLAPP | | X | | | | | | 2128 |
| SIFLLALLSCLTIPA | | X | | | | | | 2212 |
| LSTLPGNPAIASLMA | | X | | | | | | 2174 |
| STWVLVGGVLAALAA | | X | | | | | | 2218 |
| NPAIASLMAFTASIT | | X | | | | | | 2186 |
| VWPLLLLLLALPPRA | | X | | | | | | 2242 |
| GAGVAGALVAFKVMS | | X | | | | | | 2135 |
| GAGVAGALVAFKIMS | | X | | | | | | 2134 |
| TPLLYRLGAVQNEVT | | X | | | | | | 2224 |
| PGALVVGVVCAAILR | | X | | | | | | 2190 |
| RSELSPLLLSTTEWQ | | X | | | | | | 2206 |
| EDLVNLLPAILSPG | | X | | | | | | 2126 |
| ACKLTPPHSAKSKFG | | X | | | | | | 2103 |
| YGVWPLLLLLLALPP | | X | | | | | | 2250 |
| GQIVGGVYLLPRRGP | | X | | | | | | 2151 |
| CQIYGACYSIEPLDL | | X | | | | | | 2116 |
| DLAVAVEPVVFSDME | | X | | | | | | 2120 |
| YYSMVGNWAKVLIVM | | X | | | | | | 2253 |
| IQRLHGLSAFSLHSY | | X | | | | | | 2161 |
| IERLHGLSAFSLHSY | | X | | | | | | 2160 |
| LHSYSPGEINRVASC | | X | | | | | | 2170 |
| WKCLIRLKPTLHGPT | | X | | | | | | 2246 |
| DVVVVATDALMTGFT | | X | | | | | | 2124 |
| WDQMWKCLIRLKPTL | | X | | | | | | 2244 |
| LFNILGGWVAAQLAP | | X | | | | | | 2168 |
| VDILAGYGAGVAGAL | | X | | | | | | 2230 |
| SFSIFLLALLSCLTI | | X | | | | | | 2210 |
| SFSIFLLALLSCLTV | | X | | | | | | 2211 |
| VVVVATDALMTGFTG | | X | | | | | | 2240 |
| FPYLVAYQATVCARA | | X | | | | | | 2129 |
| VVGVVCAAILRRHVG | | X | | | | | | 2239 |
| FSIFLLALLSCLTIP | | X | | | | | | 2130 |
| FSIFLLALLSCLTVP | | X | | | | | | 2131 |
| ADLIEANLLWRQEMG | | X | | | | | | 2104 |
| APTLWARMILMTHFF | | X | | | | | | 2106 |
| TRGLLGCIITSLTGR | | X | | | | | | 2225 |
| TFQVAHLHAPTGSGK | | X | | | | | | 2220 |
| RGLLGCIITSLTGRD | | X | | | | | | 2200 |
| GVRVLEDGVNYATGN | | X | | 8713 | 266 | 132 | + | 2156 |
| SAMYVGDLCGSVFLV | | X | | | | | | 2208 |
| SDLYLVTRHADVIPV | | X | | | | | | 2209 |
| VVVVATDALMTGYTG | | X | | | 93 | | | 2241 |
| TVDFSLDPTFTIETT | | X | | 10395 | 46 | 59 | + | 2228 |
| GLPVCQDHLEFWESV | | X | | | 14286 | | | 2146 |
| GMQLAEQFKQKALGL | | X | | | 4992 | | | 2148 |
| LTSMLTDPSHITAET | | X | | | 567 | | | 2177 |
| MSTNPKPQRKTK | | X | | | | | | 2184 |
| DLAVAVEPVVFSDME | | X | | | | | | 2120 |
| RSPVFTDNSSPPAVP | | X | | 1676 | 1711 | 10 | + | 2207 |
| YDIIICDECHSTDST | | X | | | | | | 2248 |
| DIIICDECHSTDSTT | | X | | | | | | 2119 |
| VVVVATDALMTGFTG | | X | | | | | | 2240 |
| MACMSADLEVVTSTW | | X | | | | | | 2181 |
| LGKVLVDILAGYGAG | | X | | | | | | 2169 |
| ITRVESENKVVILDS | | X | | | | | | 2163 |
| VFCVQPEKGGRKPAR | | X | | | | | + | 2232 |
| RLIVFPDLGVRVCEK | | X | | | | | | 2202 |
| VYYLTRDPTTPLARA | | X | | | | | | 2243 |
| GACYSIEPLDLPQII | | X | | | | | | 2133 |
| SYTWTGALITPCAAE | X | | | 5.63 | | | | 2219 |
| NSWLGNIIMYAPTLW | X | | | 1.2 | | | | 2187 |
| VNSWLGNIIMYAPTL | X | | | 1.42 | | | | 2237 |
| QDAVSRSQRRGRTGR | X | | | | | | | 2196 |
| MTGFTGDFDSVIDCN | X | | | | | | | 2185 |

### EXAMPLES

### Example 1

### Identification of CTL Specific HCV Peptides peptides Using the Algonomics Algorithm

HLA Class I protein subclasses that should be targeted are defined: HLA-A01, 02, 03 and 24; HLA-B07, 08, 35 and 44; HLA-Cw04, -Cw06 and Cw07.

These HLA-Class I subclasses are modeled based on known homologue structures.

Based on X-ray data, an in depth analysis is performed of the main chain conformational changes in a given HLA-class I subclass for different peptides bound to said HLA-class I. This analysis results in rules that will be applied when generating backbone variability. On the average 8 to 10 different HLA-class I peptide complexes for each of the HLA-class I subclasses are built based on a series of epitopes and using Algonomics flexible peptide docking tools (wherein the peptide main and side chains are considered flexible, as well as the side chains of the HLA molecules). This yields in total 88 to 110 different three-dimensional models.

By using the above rules for main chain flexibility and/or by using molecular dynamics techniques or main chain perturbation/relaxation approaches, about five different versions differing in main chain conformation in the neighborhood of the bound peptide of the above models are derived. Hence, about 500 different three-dimensional models of HLA-class I peptide complexes are generated.

For each of the HLA-class I peptide models a prediction of the sequence variability of the peptide moieties in the context with surrounding HLA molecules is made: thread through the peptide backbones all HCV protein sequences of interest for all known HCV genotypes and asses for each “threaded” peptide the likelihood that it can form a stable complex with the underlying HLA-class I.

This is done using Algonomics' advanced inverse folding tools which have been developed within the Extended Dead-End Elimination framework. The end-point of this analysis is a list of binding peptides for each of the 11 HLA-Class I subclasses.

### Example 2

### Identification of CTL Specific B07-Restricted Peptides Using 4 Different Algorithms

For the HLA B07, a selection of the best scoring peptides is retrieved from the 3 on-line prediction servers (BIMAS, Syfpeithi and nHLAPred) using HCV consensus sequence 1b, and from the PIC-algorithm described by Epimmune using 57 HCV sequences. These peptides can either be 8-mers, 9-mers, 10-mers and in some cases 11-mers. Four hundred peptides were retrieved from BIMAS, 250 peptide from Syfpeithi, 100 from nHLAPred and 58 from the PIC algorithm from Epimmune. Said peptides are given in Table 15.

TABLE 15

Predicted CTL specific B07-restricted peptides
Prot: protein
GT = genotype

| | | | | | |
|---|---|---|---|---|---|
| | | | | | SEQ |
| | Peptide | | | | ID |
| Prot | sequence | Score | GT | rank | NO |

| | | | | | |
|---|---|---|---|---|---|
| BIMAS | | | | | |
| NS5B | RPRWFMLCL | 800 | 1b | 1 | 1684 |
| C | DPRRRSRNL | 800 | 1b/1a/3a | 2 | 370 |
| NS5B | RPRWFMLCLL | 800 | 1b | 1 | 1685 |
| NS5B | APTLWARMIL | 360 | 1b/1a | 2 | 853 |
| C | APLGGAARAL | 240 | 1b/1a | 3 | 836 |
| NS5B | GVRVCEKMAL | 200 | 1b/1a | 4 | 1203 |
| NS5B | RARSVRAKL | 180 | 1b | 3 | 1632 |
| NS2 | SARRGREIL | 180 | 1b/1a | 4 | 1718 |
| C | QPRGRRQPI | 120 | 1b/1a/3a | 5 | 390 |
| NS5B | RARPRWFML | 120 | 1b | 6 | 1631 |
| NS5B | DPPQPEYDL | 120 | 1b/1a | 7 | 543 |
| NS5A | LARGSPPSL | 120 | 1b | 8 | 1363 |
| NS5B | AIRSLTERL | 120 | 1b | 9 | 473 |
| NS5B | EARQAIRSL | 120 | 1b | 10 | 388 |
| NS2 | SARRGREILL | 120 | 1b/1a | 5 | 1719 |
| NS5A | WARPDYNPPL | 120 | 1b/1a/3a | 6 | 1998 |
| NS5B | RARSVRAKLL | 120 | 1b | 7 | 1633 |
| NS4A | AVIPDREVL | 90 | 1b | 11 | 891 |
| C | LPRRGPRLGV | 90 | 1b/1a/3a | 8 | 450 |
| NS3 | HPNIEEVAL | 80 | 1b/1a | 12 | 1237 |
| NS3 | TPAETSVRL | 80 | 1b | 13 | 383 |
| NS5B | SPGQRVEFL | 80 | 1b/1a | 14 | 373 |
| NS4B | SPLTTQHTL | 80 | 1b | 15 | 1767 |
| NS3 | GPTPLLYRL | 80 | 1b/1a/3a | 16 | 307 |
| E2 | GPWLTPRCL | 80 | 1b | 17 | 1173 |
| NS5B | TPIPAASQL | 80 | 1b | 18 | 1848 |
| NS5B | IPAASQLDL | 80 | 1b | 19 | 1280 |
| NS4B | MPSTEDLVNL | 80 | 1b | 9 | 1505 |
| NS2 | VPYFVRAQGL | 80 | 1b | 10 | 1960 |
| E2 | SPGPSQKIQL | 80 | 1b | 11 | 1764 |
| NS5A | SPAPNYSRAL | 80 | 1b | 12 | 1756 |
| P7 | WPLLLLLLAL | 80 | 1b/1a | 13 | 2018 |
| NS3 | KPTLHGPTPL | 80 | 1b/1a/3a | 14 | 1343 |
| NS4B | LPAILSPGAL | 80 | 1b/1a/3a | 15 | 1418 |
| NS4B | LPGNPAIASL | 80 | 1b/1a | 16 | 1428 |
| C | LPGCSFSIFL | 80 | 1b/1a/3a | 17 | 1426 |
| NS4B | SPLTTQHTLL | 80 | 1b | 18 | 1768 |
| NS5B | TPCAAEESKL | 80 | 1b | 19 | 1840 |
| NS3 | TPCTCGSSDL | 80 | 1b/1a | 20 | 1843 |
| NS5A | PPRRKRTVVL | 80 | 1b | 21 | 1579 |
| NS3 | VPQTFQVAHL | 80 | 1b | 22 | 1954 |
| NS4B | LPYIEQGMQL | 80 | 1b | 23 | 1447 |
| NS5B | LPINALSNSL | 80 | 1b/1a | 24 | 1430 |
| NS5A | VPPVVHGCPL | 80 | 1b | 25 | 1953 |
| E2 | WTRGERCDL | 60 | 1b/1a | 20 | 2022 |
| NS2 | AVFVGLALL | 60 | 1b | 21 | 886 |
| NS3 | APPPSWDQM | 60 | 1b/1a | 22 | 381 |
| NS5B | LTRDPTTPL | 60 | 1b/1a | 23 | 1475 |
| E2 | APRPCGIVPA | 60 | 1b | 26 | 847 |
| E1 | TIRRHVDLL | 40 | 1b | 24 | 1828 |
| NS5B | HIRSVWKDL | 40 | 1b | 25 | 1223 |
| NS5A | KSRKFPPAL | 40 | 1b | 26 | 1348 |
| NS2 | GGRDAIILL | 40 | 1b | 27 | 1138 |
| NS5B | HIRSVWKDLL | 40 | 1b | 27 | 1224 |
| NS5B | CLRKLGVPPL | 40 | 1b/1a | 28 | 921 |
| P7 | AAWYIKGRL | 36 | 1b | 28 | 782 |
| E2/P7 | AALENLVVL | 36 | 1b | 29 | 776 |
| NS4B | AAARVTQIL | 36 | 1b | 30 | 773 |
| NS3 | AAKLSALGL | 36 | 1b | 31 | 402 |
| NS2 | AACGDIILGL | 36 | 1b | 29 | 774 |
| NS3 | AAQGYKVLVL | 36 | 1b/1a | 30 | 777 |
| NS5B | AAKLQDCTML | 36 | 1b | 31 | 775 |
| NS4A | VVIVGRIIL | 30 | 1b | 32 | 1982 |
| NS2 | NVRGGRDAI | 30 | 1b | 33 | 1538 |
| NS3 | GPKGPITQM | 30 | 1b | 34 | 1165 |
| E2 | DARVCACLWM | 30 | 1b | 32 | 954 |
| NS4A | SVVIVGRIIL | 30 | 1b | 33 | 1803 |
| NS5A | EPEPDVAVL | 24 | 1b/1a | 35 | 1024 |
| NS5A | RPDYNPPLL | 24 | 1b/1a/3a | 36 | 1677 |
| NS5B | APTLWARMI | 24 | 1b/1a | 37 | 371 |
| NS5B | LSRARPRWFM | 22.5 | 1b | 34 | 1462 |
| P7 | LVPGAAYAL | 20 | 1b | 38 | 1482 |
| NS3 | VVVVATDAL | 20 | 1b/1a | 39 | 1988 |
| E2 | YVLLLFLLL | 20 | 1b | 40 | 2073 |
| NS3/NS4A | EVVISTWVL | 20 | 1b/1a | 41 | 1042 |
| NS4A | LVGGVLAAL | 20 | 1b/1a | 42 | 1479 |
| P7 | GVWPLLLLL | 20 | 1b | 43 | 1207 |
| C | GVNYATGNL | 20 | 1b/1a | 44 | 339 |
| NS4B | RVTQILSSL | 20 | 1b | 45 | 1714 |
| E2 | YVGGVEHRL | 20 | 1b/1a | 46 | 2072 |
| NS3 | EVIKGGRHL | 20 | 1b/1a | 47 | 376 |
| NS5A | TVSSALAEL | 20 | 1b | 48 | 1881 |
| NS5B | SVGVGIYLL | 20 | 1b | 49 | 1799 |
| NS5A | EVSVAAEIL | 20 | 1b | 50 | 1040 |
| NS2 | YVYDHLTPL | 20 | 1b | 51 | 2077 |
| NS5A | DVAVLTSML | 20 | 1b/1a | 52 | 989 |
| P7 | SVAGAHGIL | 20 | 1b | 53 | 1796 |
| NS2 | FVGLALLTL | 20 | 1b | 54 | 1090 |
| C | GPRLGVRAT | 20 | 1b/1a/3a | 55 | 387 |
| E1 | MVGNWAKVL | 20 | 1b/1a | 56 | 1510 |
| NS4B | LVNLLPAIL | 20 | 1b/1a | 57 | 1481 |
| NS2 | YVQMAFMKL | 20 | 1b | 58 | 2074 |
| NS3 | TPGERPSGM | 20 | 1b | 59 | 372 |
| C | WPLYGNEGM | 20 | 1b | 60 | 2019 |
| NS5B | RVASCLRKL | 20 | 1b | 61 | 1707 |
| E2 | RVCACLWMML | 20 | 1b | 35 | 1709 |
| NS4B | GPGEGAVQWM | 20 | 1b/1a/3a | 36 | 1163 |
| NS5B | KVTFDRLQVL | 20 | 1b/1a/3a | 37 | 1352 |
| NS5A | DVWDWICTVL | 20 | 1b | 38 | 995 |
| NS3 | DVVVVATDAL | 20 | 1b/1a | 39 | 994 |
| NS5A | VVILDSFDPL | 20 | 1b/1a | 40 | 1981 |
| E1 | YPGHVSGHRM | 20 | 1b | 41 | 2063 |
| E1 | MVAGAHWGVL | 20 | 1b | 42 | 1509 |
| P7 | GVWPLLLLLL | 20 | 1b | 43 | 1208 |
| NS4B | VVGVVCAAIL | 20 | 1b/1a | 44 | 1980 |
| NS3 | VPVESMETIM | 20 | 1b | 45 | 1958 |
| NS5A | TVLTDFKTWL | 20 | 1b | 46 | 1880 |
| NS2 | NVRGGRDAII | 20 | 1b | 47 | 1539 |
| NS3 | CVTQTVDFSL | 20 | 1b/1a | 48 | 949 |
| NS3 | VVSTATQSFL | 20 | 1b | 49 | 1985 |
| NS2 | AILGPLMVL | 18 | 1b | 62 | 816 |
| C | AARALAHGV | 18 | 1b/1a | 63 | 403 |
| NS2 | LAILGPLMVL | 18 | 1b | 50 | 1361 |
| NS5B | AVRTKLKLT | 15 | 1b/1a | 64 | 895 |
| NS2 | ARRGREILL | 12 | 1b/1a | 65 | 865 |
| NS3 | NAVAYYRGL | 12 | 1b/1a/3a | 66 | 400 |
| NS4B | GAVQWMNRL | 12 | 1b/1a/3a | 67 | 1109 |
| NS3 | QAGDNFPYL | 12 | 1b | 68 | 551 |
| NS5B | ATTSRSASL | 12 | 1b | 69 | 879 |
| NS5B | ALYDVVSIL | 12 | 1b | 70 | 67 |
| NS5B | WAVRTKLKL | 12 | 1b/1a | 71 | 1999 |
| NS2 | TAACGDIIL | 12 | 1b | 72 | 1811 |
| NS4A | LAALAAYCL | 12 | 1b/1a/3a | 73 | 1357 |
| E2 | ALSTGLIHL | 12 | 1b/1a/3a | 74 | 825 |
| NS3 | DAGCAWYEL | 12 | 1b/1a | 75 | 528 |
| E2 | CACLWMMLL | 12 | 1b | 76 | 909 |
| NS5A | LASSSASQL | 12 | 1b | 77 | 1365 |
| NS2 | GAVFVGLAL | 12 | 1b | 78 | 1107 |
| NS3 | SGMFDSSVL | 12 | 1b/1a | 79 | 135 |
| NS5B | ASGKRVYYL | 12 | 1b | 80 | 334 |
| NS3 | YAAQGYKVL | 12 | 1b/1a | 81 | 95 |
| NS5B | GACYSIEPL | 12 | 1b/1a | 82 | 1103 |
| NS2 | ACGDIILGL | 12 | 1b | 83 | 784 |
| E2 | FAIKWEYVL | 12 | 1b | 84 | 1049 |
| NS3 | QAPPGARSL | 12 | 1b | 85 | 1598 |
| C | AQPGYPWPL | 12 | 1b/1a/3a | 86 | 65 |
| NS3 | AQGYKVLVL | 12 | 1b/1a | 87 | 130 |
| NS3 | RAYLNTPGL | 12 | 1b | 88 | 434 |
| NS2 | WAHAGLRDL | 12 | 1b | 89 | 1992 |
| NS4B | GAAVGSIGL | 12 | 1b | 90 | 1102 |
| NS5A | ASQLSAPSL | 12 | 1b/1a/3a | 91 | 872 |
| P7 | GAHGILSFL | 12 | 1b | 92 | 1104 |
| NS5B | SAACRAAKL | 12 | 1b | 93 | 121 |
| NS3 | GAVQNEVIL | 12 | 1b/1a | 94 | 347 |
| E2 | AIKWEYVLL | 12 | 1b | 95 | 814 |
| NS3 | TAGARLVVL | 12 | 1b/1a | 96 | 249 |
| E1 | WAKVLIVML | 12 | 1b | 97 | 1994 |
| NS5B | ASTVKAKLL | 12 | 1b | 98 | 875 |
| NS5A | YAPACKPLL | 12 | 1b | 99 | 2027 |
| NS5B | RAATCGKYL | 12 | 1b | 100 | 1627 |
| NS2 | MAFMKLAAL | 12 | 1b | 101 | 1491 |
| C | ALAHGVRVL | 12 | 1b/1a | 102 | 72 |
| P7 | ALYGVWPLL | 12 | 1b | 103 | 830 |
| E1 | TALVVSQLL | 12 | 1b | 104 | 1813 |
| C | EGMGWAGWL | 12 | 1b | 105 | 1011 |
| E2 | TALNCNDSL | 12 | 1b | 106 | 1812 |
| NS5B | KASTVKAKL | 12 | 1b | 107 | 1316 |
| E1 | VAGAHWGVL | 12 | 1b | 108 | 1889 |
| P7 | YALYGVWPL | 12 | 1b | 109 | 2025 |
| NS5B | PARLIVFPDL | 12 | 1b/1a | 51 | 1545 |
| P7 | YALYGVWPLL | 12 | 1b | 52 | 2026 |
| NS5B | ILMTHFFSIL | 12 | 1b | 53 | 1274 |
| NS5B | LAQEQLEKAL | 12 | 1b | 54 | 1362 |
| NS5B | ACYSIEPLDL | 12 | 1b/1a | 55 | 785 |
| NS2 | GAVFVGLALL | 12 | 1b | 56 | 1108 |
| E2 | AIKWEYVLLL | 12 | 1b | 57 | 815 |
| NS5B | YATTSRSASL | 12 | 1b | 58 | 2029 |
| NS3 | YIMACMSADL | 12 | 1b | 59 | 2037 |
| NS5B | QAIRSLTERL | 12 | 1b | 60 | 1597 |
| P7 | CAAWYIKGRL | 12 | 1b | 61 | 908 |
| E2/P7 | AQAEAALENL | 12 | 1b | 62 | 858 |
| E1 | WAKVLIVMLL | 12 | 1b | 63 | 1995 |
| NS3 | LAAKLSALGL | 12 | 1b | 64 | 1356 |
| P7 | ALYGVWPLLL | 12 | 1b | 65 | 831 |
| NS5A | SASQLSAPSL | 12 | 1b/1a/3a | 66 | 1720 |
| NS3 | TAYSQQIRGL | 12 | 1b | 67 | 1814 |
| E2/P7 | EAALENLWL | 12 | 1b | 68 | 998 |
| NS5B | MALYDVVSTL | 12 | 1b | 69 | 1492 |
| NS5B | CTMLVNGDDL | 12 | 1b | 70 | 942 |
| C | RALAHGVRVL | 12 | 1b/1a | 71 | 1629 |
| E2 | FAIKVVEYVLL | 12 | 1b | 72 | 1050 |
| NS5B | DASGKRVYYL | 12 | 1b | 73 | 955 |
| E1 | GAHWGVLAGL | 12 | 1b | 74 | 1105 |
| NS5B | KASTVKAKLL | 12 | 1b | 75 | 1317 |
| C | EGMGWAGWLL | 12 | 1b | 76 | 1012 |
| NS2 | AQGLIRACML | 12 | 1b | 77 | 860 |
| P7 | AGAHGILSFL | 12 | 1b | 78 | 805 |
| NS4B | AGAAVGSIGL | 12 | 1b | 79 | 804 |
| P7 | ASVAGAHGIL | 12 | 1b | 80 | 876 |
| NS5B | ASAACRAAKL | 12 | 1b | 81 | 869 |
| NS3 | DQMWKCLIRL | 12 | 1b/1a | 82 | 982 |
| NS4B | APPSAASAFV | 12 | 1b | 83 | 845 |
| NS4B | DAAARVTQIL | 12 | 1b | 84 | 952 |
| NS5B | AGTQEDAASL | 12 | 1b | 85 | 807 |
| C | WAQPGYPWPL | 12 | 1b/1a/3a | 86 | 1996 |
| NS5B | SLRVFTEAM | 10 | 1b | 110 | 495 |
| C | GVRVLEDGV | 10 | 1b/1a | 111 | 447 |
| E2 | KVRMYVGGV | 10 | 1b/1a | 112 | 1351 |
| NS5B | DVRNLSSKAV | 10 | 1b | 87 | 993 |
| E1 | AARNSSVPT | 9 | 1b | 113 | 778 |
| NS5B | AAKLQDCTM | 9 | 1b | 114 | 440 |
| E2 | AARTTSGFT | 9 | 1b | 115 | 780 |
| NS3 | APPGARSLT | 9 | 1b | 116 | 839 |
| NS3 | AATLGFGAYM | 9 | 1b/1a | 88 | 781 |
| E1 | AARNSSVPTT | 9 | 1b | 89 | 779 |
| E2 | GPWLTPRCLV | 9 | 1b | 90 | 1174 |
| NS5A | PPVVHGCPL | 8 | 1b/1a | 117 | 1582 |
| E2 | YPCTVNFTI | 8 | 1b | 118 | 2059 |
| NS3 | CPSGHAVGI | 8 | 1b | 119 | 931 |
| NS5B | EPLDLPQII | 8 | 1b | 120 | 1027 |
| E2 | LPALSTGLI | 8 | 1b/1a | 121 | 1419 |
| E2 | GPSQKIQLI | 8 | 1b | 122 | 1171 |
| NS5A | LPPTKAPPI | 8 | 1b | 123 | 1435 |
| NS2 | GPLMVLQAGI | 8 | 1b | 91 | 1168 |
| NS3 | IPFYGKAIPI | 8 | 1b | 92 | 1284 |
| NS5B | TPVNSWLGNI | 8 | 1b/1a/3a | 93 | 1862 |
| NS5B | PPQPEYDLEL | 8 | 1b/1a | 94 | 1575 |
| NS5B | VVSTLPQAVM | 7.5 | 1b | 95 | 1986 |
| NS3 | AVDFVPVESM | 6.75 | 1b | 96 | 883 |
| NS3 | TLHGPTPLL | 6 | 1b/1a/3a | 124 | 81 |
| C | APLGGAARA | 6 | 1b/1a | 125 | 384 |
| NS5B | ESKLPINAL | 6 | 1b | 126 | 1031 |
| C | SPRGSRPSW | 6 | 1b/1a/3a | 127 | 386 |
| NS3 | YSQQTRGLL | 6 | 1b | 128 | 2069 |
| NS3 | LSPRPVSYL | 6 | 1b | 129 | 1460 |
| NS5B | CPMGFSYDT | 6 | 1b | 130 | 421 |
| NS5A | LPCEPEPDV | 6 | 1b/1a/3a | 131 | 1421 |
| NS2 | ILLGPADSL | 6 | 1b | 132 | 1271 |
| NS5A | APSLKATCT | 6 | 1b/1a | 133 | 848 |
| NS5B | FNWAVRTKL | 6 | 1b/1a | 134 | 1076 |
| NS3 | TSVRLRAYL | 6 | 1b | 135 | 1870 |
| NS3 | APTGSGKST | 6 | 1b/1a/3a | 136 | 397 |
| NS5A | LPRLPGVPF | 6 | 1b | 137 | 1439 |
| NS4B | VVESKWRAL | 6 | 1b | 138 | 1979 |
| E2 | APRPCGIVP | 6 | 1b | 139 | 846 |
| P7 | YGVWPLLLL | 6 | 1b | 140 | 2036 |
| NS5B | GGRKPARLI | 6 | 1b/1a/3a | 141 | 435 |
| NS4B | AVQWMNRLI | 6 | 1b/1a/3a | 142 | 893 |
| E1 | MNWSPTTAL | 6 | 1b | 143 | 1503 |
| NS5A | PPRRKRTVV | 6 | 1b | 144 | 1578 |
| NS4B | APVVESKWRA | 6 | 1b | 97 | 856 |
| NS3 | APITAYSQQT | 6 | 1b | 98 | 835 |
| NS2 | VSARRGREIL | 6 | 1b/1a | 99 | 1970 |
| NS5B | YLTRDPTTPL | 6 | 1b/1a | 100 | 2051 |
| NS2 | EILLGPADSL | 6 | 1b | 101 | 1013 |
| NS2 | AVHPELIFDI | 6 | 1b | 102 | 890 |
| E1 | MMNWSPTTAL | 6 | 1b | 103 | 1502 |
| NS3 | LLSPRPVSYL | 6 | 1b | 104 | 1409 |
| E1 | VPTTTIRRHV | 6 | 1b | 105 | 1956 |
| NS5A | EPDVAVLTSM | 6 | 1b/1a | 106 | 1023 |
| NS3 | RRRGDSRGSL | 6 | 1b/1a | 107 | 1697 |
| E2 | GSWHINRTAL | 6 | 1b | 108 | 1190 |
| NS5A | APSLKATCTT | 6 | 1b | 109 | 849 |
| NS3 | ETSVRLRAYL | 6 | 1b | 110 | 1033 |
| NS4A | RPAVIPDREV | 6 | 1b | 111 | 1674 |
| NS3 | VVVATDALM | 5 | 1b/1a | 145 | 343 |
| NS5B | GVRVCEKMA | 5 | 1b/1a | 146 | 498 |
| NS3 | GVRTITTGA | 5 | 1b | 147 | 1202 |
| NS5B | DVRNLSSKA | 5 | 1b | 148 | 992 |
| NS5A | GVWRGDGIM | 5 | 1b/1a | 149 | 1209 |
| E2 | RVCACLWMM | 5 | 1b | 150 | 1708 |
| NS4A | EVLYREFDEM | 5 | 1b/1a | 112 | 1039 |
| NS3 | VVVVATDALM | 5 | 1b/1a | 113 | 1989 |
| NS2 | KVAGGHYVQM | 5 | 1b | 114 | 1349 |
| NS3 | SVRLRAYLNT | 5 | 1b | 115 | 1802 |
| NS2 | FVRAQGLIRA | 5 | 1b | 116 | 1092 |
| E1 | CVRENNSSRC | 5 | 1b | 117 | 948 |
| E1 | IVYEAADMIM | 5 | 1b | 118 | 1313 |
| NS5A | GVRLHRYAPA | 5 | 1b | 119 | 1201 |
| NS5A | ANLLWRQEM | 4.5 | 1b/1a/3a | 151 | 832 |
| C | KARRPEGRA | 4.5 | 1b | 152 | 1315 |
| NS3 | AVGIFRAAV | 4.5 | 1b/1a | 153 | 887 |
| NS2 | AQGLIRACM | 4.5 | 1b | 154 | 859 |
| NS5A | LARGSPPSLA | 4.5 | 1b | 120 | 1364 |
| NS5B | EARQAIRSLT | 4.5 | 1b | 121 | 1001 |
| NS5A | EANLLWRQEM | 4.5 | 1b/1a | 122 | 1000 |
| NS2 | RAQGLIRACM | 4.5 | 1b | 123 | 1630 |
| NS3 | AGPKGPITQM | 4.5 | 1b | 124 | 806 |
| NS2 | AVEPVVFSDM | 4.5 | 1b | 125 | 885 |
| NS5A | LQSKLLPRL | 4 | 1b | 155 | 1449 |
| E1 | NSSRCWVAL | 4 | 1b | 156 | 1533 |
| NS3 | NIRTGVRTI | 4 | 1b/1a | 157 | 436 |
| NS5B | SGGDIYHSL | 4 | 1b | 158 | 1731 |
| E1 | TTIRRHVDL | 4 | 1b | 159 | 1874 |
| NS4B | SSLTITQLL | 4 | 1b | 160 | 1785 |
| NS5A | VILDSFDPL | 4 | 1b/1a | 161 | 1918 |
| NS2 | LTCAVHPEL | 4 | 1b | 162 | 1464 |
| NS5B | DLPQIIERL | 4 | 1b | 163 | 967 |
| E2 | CSFTTLPAL | 4 | 1b/1a | 164 | 936 |
| NS5B | EINRVASCL | 4 | 1b | 165 | 1014 |
| E1 | GSVFLVSQL | 4 | 1b | 166 | 1189 |
| C | TLTCGFADL | 4 | 1b/1a/3a | 167 | 363 |
| NS5B | LTTSCGNTL | 4 | 1b/1a | 168 | 304 |
| NS4B | FTASITSPL | 4 | 1b | 169 | 1085 |
| NS2 | CGGAVFVGL | 4 | 1b | 170 | 915 |
| E2 | TLPALSTGL | 4 | 1b/1a | 171 | 1835 |
| NS5B | LSVGVGIYL | 4 | 1b | 172 | 1463 |
| NS3 | VTQTVDFSL | 4 | 1b/1a | 173 | 712 |
| C | FSIFLLALL | 4 | 1b/1a | 174 | 362 |
| C | RSRNLGKVI | 4 | 1b/1a/3a | 175 | 318 |
| C | GQIVGGVYL | 4 | 1b/1a | 176 | 127 |
| NS2 | HLQVWVPPL | 4 | 1b | 177 | 1232 |
| NS3 | TCGSSDLYL | 4 | 1b/1a | 178 | 1816 |
| C | GCSFSIFLL | 4 | 1b/1a/3a | 179 | 294 |
| NS3 | HSKKKCDEL | 4 | 1b/1a | 180 | 455 |
| NS5B | NIIMYAPTL | 4 | 1b | 181 | 87 |
| NS2 | ITKLLLAIL | 4 | 1b | 182 | 1308 |
| E2 | RTTSGFTSL | 4 | 1b | 183 | 1706 |
| NS3 | QMWKCLIRL | 4 | 1b/1a | 184 | 238 |
| NS5B | GIQEDAASL | 4 | 1b | 185 | 88 |
| NS3 | HSTDSTTIL | 4 | 1b | 186 | 1244 |
| NS2 | LSPYYKVFL | 4 | 1b | 187 | 1461 |
| NS3 | QTRGLLGCI | 4 | 1b/1a | 188 | 1621 |
| C | NLGKVIDTL | 4 | 1b/1a/3a | 189 | 283 |
| NS3 | VSTATQSFL | 4 | 1b | 190 | 1973 |
| NS3 | KGSSGGPLL | 4 | 1b/1a | 191 | 260 |
| NS3 | LLGCIITSL | 4 | 1b/1a | 192 | 1397 |
| C | YIPLVGAPL | 4 | 1b/1a | 193 | 69 |
| NS3 | IPTSGDVVV | 4 | 1b/1a | 194 | 415 |
| E2 | LQTGFLAAL | 4 | 1b | 195 | 1450 |
| NS4B | VGVVCAAIL | 4 | 1b/1a | 196 | 1912 |
| NS2 | LIFDITKLL | 4 | 1b | 197 | 1386 |
| NS2 | FITRAEAHL | 4 | 1b | 198 | 1061 |
| NS4B | SGIQYLAGL | 4 | 1b/1a/3a | 199 | 1732 |
| NS4B | GSIGLGKVL | 4 | 1b | 200 | 1186 |
| P7 | RLVPGAAYAL | 4 | 1b | 126 | 1666 |
| E2 | VCACLWMMLL | 4 | 1b | 127 | 1896 |
| NS5A | WLQSKLLPRL | 4 | 1b | 128 | 2014 |
| NS5B | LLSVEEACKL | 4 | 1b | 129 | 1410 |
| C | RNLGKVIDTL | 4 | 1b/1a/3a | 130 | 1673 |
| E1 | TTALVVSQLL | 4 | 1b | 131 | 1871 |
| NS4A | VLAALAAYCL | 4 | 1b/1a/3a | 132 | 1921 |
| NS3 | YLKGSSGGPL | 4 | 1b/1a | 133 | 2046 |
| NS4B | DLVNLLPAIL | 4 | 1b/1a | 134 | 969 |
| NS3 | CTCGSSDLYL | 4 | 1b/1a | 135 | 941 |
| E1 | TTIRRHVDLL | 4 | 1b | 136 | 1875 |
| NS5B | LMTHFFSILL | 4 | 1b | 137 | 1415 |
| NS5B | YSGGDIYHSL | 4 | 1b | 138 | 2067 |
| NS3 | GLLGCIITSL | 4 | 1b/1a | 139 | 1151 |
| NS5B | RQKKVTFDRL | 4 | 1b/1a/3a | 140 | 1695 |
| NS3 | IPTSGDVVVV | 4 | 1b/1a | 141 | 1295 |
| E2 | VPASQVCGPV | 4 | 1b | 142 | 1946 |
| C | GQIVGGVYLL | 4 | 1b/1a | 143 | 1178 |
| NS2 | ELIFDITKLL | 4 | 1b | 144 | 1018 |
| NS5A | SLASSSASQL | 4 | 1b | 145 | 1740 |
| NS2 | TLSPYYKVFL | 4 | 1b | 146 | 1836 |
| E2 | QILPCSFTTL | 4 | 1b | 147 | 1606 |
| NS4B | GLGKVLVDIL | 4 | 1b/1a | 148 | 1149 |
| NS5B | YGACYSIEPL | 4 | 1b/1a | 149 | 2033 |
| NS4B | EQFKQKALGL | 4 | 1b/1a | 150 | 1029 |
| E1 | CGSVFLVSQL | 4 | 1b | 151 | 916 |
| NS3 | WQAPPGARSL | 4 | 1b | 152 | 2021 |
| E2 | RCLVDYPYRL | 4 | 1b/1a | 153 | 1635 |
| NS2 | KLLLAILGPL | 4 | 1b | 154 | 1331 |
| NS5B | LTPIPAASQL | 4 | 1b | 155 | 1472 |
| C | YLLPRRGPRL | 4 | 1b/1a | 156 | 2047 |
| NS5A | IPPPRRKRTV | 4 | 1b | 157 | 1292 |
| NS5B | GNIIMYAPTL | 4 | 1b | 158 | 1160 |
| NS2 | DITKLLLAIL | 4 | 1b | 159 | 961 |
| NS2 | PLRDWAHAGL | 4 | 1b | 160 | 1559 |
| NS2 | LLTCAVHPEL | 4 | 1b | 161 | 1413 |
| NS5A | ITAETAKRRL | 4 | 1b | 162 | 1306 |
| E2 | SGPWLTPRCL | 4 | 1b | 163 | 1735 |
| NS3 | QMYTNVDQDL | 4 | 1b/1a/3a | 164 | 1611 |
| NS5B | ESILLAQEQL | 4 | 1b | 165 | 1083 |
| E2 | RDRSELSPLL | 4 | 1b/1a | 166 | 1637 |
| E2 | TTLPALSTGL | 4 | 1b/1a | 167 | 1876 |
| NS3 | GPITQMYTNV | 4 | 1b | 168 | 1164 |
| NS2 | GGAVFVGLAL | 4 | 1b | 169 | 1135 |
| NS3 | QTRGLLGCII | 4 | 1b/1a | 170 | 1622 |
| NS5A | STVSSALAEL | 4 | 1b | 171 | 1792 |
| E2 | RTALNCNDSL | 4 | 1b | 172 | 1703 |
| NS3 | LNAVAYYRGL | 4 | 1b | 173 | 1416 |
| NS5B | VLTTSCGNTL | 4 | 1b/1a | 174 | 1930 |
| E2 | HQNIVDVQYL | 4 | 1b/1a/3a | 175 | 1240 |
| NS4B | LTITQLLKRL | 4 | 1b | 176 | 1470 |
| NS4B | ILSSLTITQL | 4 | 1b | 177 | 1276 |
| NS5B | SPGQRVEFLV | 4 | 1b/1a | 178 | 1765 |
| NS2 | SCGGAVFVGL | 4 | 1b | 179 | 1721 |
| NS3 | LTPAETSVRL | 4 | 1b | 180 | 1471 |
| NS5B | YSPGQRVEFL | 4 | 1b/1a | 181 | 2068 |
| NS3 | RPSGMFDSSV | 4 | 1b/1a | 182 | 1687 |
| NS4A | STWVLVGGVL | 4 | 1b/1a | 183 | 1794 |
| NS2 | RGGRDAIILL | 4 | 1b | 184 | 1649 |
| NS3 | HGPTPLLYRL | 4 | 1b/1a/3a | 185 | 1219 |
| NS5B | LLSVGVGIYL | 4 | 1b | 186 | 1412 |
| C | CSFSIFLLAL | 4 | 1b/1a/3a | 187 | 935 |
| C | DTLTCGFADL | 4 | 1b/1a/3a | 188 | 988 |
| NS5B | YRRCRASGVL | 4 | 1b/1a/3a | 189 | 2066 |
| NS4B | ISGIQYLAGL | 4 | 1b/1a | 190 | 1303 |
| NS5B | TERLYIGGPL | 4 | 1b | 191 | 1820 |
| NS4B/NS5A | CSTPCSGSWL | 4 | 1b | 192 | 937 |
| E2 | YTKCGSGPWL | 4 | 1b | 193 | 2071 |
| E2 | TPRCLVDYPY | 4 | 1b/1a | 194 | 1857 |
| NS4B | SPGALVVGVV | 4 | 1b/1a | 195 | 1760 |
| E1 | SMVGNWAKVL | 4 | 1b/1a | 196 | 1754 |
| NS5A | LPRLPGVPFF | 4 | 1b | 197 | 1440 |
| E1 | FCSAMYVGDL | 4 | 1b | 198 | 1052 |
| E1 | NNSSRCWVAL | 4 | 1b | 199 | 1526 |
| NS4B | TSPLTTQHTL | 4 | 1b | 200 | 1869 |
| Syfpeithi | | | | | |
| NS5A | PPRRKRTVVL | 26 | 1b | 1 | 1579 |
| C | APLGGAARAL | 25 | 1b/1a | 2 | 836 |
| NS5A | LPRLPGVPF | 25 | 1b | 3 | 1439 |
| NS5A | EPEPDVAVL | 25 | 1b/1a | 4 | 1024 |
| NS5B | IPAASQLDL | 25 | 1b | 5 | 1280 |
| NS5B | RPRWFMLCL | 25 | 1b | 6 | 1684 |
| E2 | APRPCGIVPA | 24 | 1b | 7 | 847 |
| NS4B | MPSTEDLVNL | 24 | 1b | 8 | 1505 |
| P7 | WPLLLLLLAL | 23 | 1b/1a | 9 | 2018 |
| NS3 | KPTLHGPTPL | 23 | 1b/1a/3a | 10 | 1343 |
| NS5A | SPAPNYSRAL | 23 | 1b | 11 | 1756 |
| NS5B | PPQPEYDLEL | 23 | 1b/1a | 12 | 1575 |
| NS5B | APTLWARMIL | 23 | 1b/1a | 13 | 853 |
| NS5B | RPRWFMLCLL | 23 | 1b | 14 | 1685 |
| NS3 | HPNIEEVAL | 23 | 1b/1a | 15 | 1237 |
| NS3 | TPAETSVRL | 23 | 1b | 16 | 383 |
| NS5A | RPDYNPPLL | 23 | 1b/1a/3a | 17 | 1677 |
| NS5B | SPGQRVEFL | 23 | 1b/1a | 18 | 313 |
| NS5B | DPPQPEYDL | 23 | 1b/1a | 19 | 543 |
| C | LPGCSFSIFL | 22 | 1b/1a/3a | 20 | 1426 |
| E2 | SPGPSQKIQL | 22 | 1b | 21 | 1764 |
| NS3 | VPQTFQVAHL | 22 | 1b | 22 | 1954 |
| NS4B | LPGNPAIASL | 22 | 1b/1a | 23 | 1428 |
| NS4B | LPAILSPGAL | 22 | 1b/1a/3a | 24 | 1418 |
| C | QPRGRRQPI | 22 | 1b/1a/3a | 25 | 390 |
| C | DPRRRSRNL | 22 | 1b/1a/3a | 26 | 370 |
| E2 | TPSPVVVGT | 22 | 1b/1a/3a | 27 | 1860 |
| NS3 | GPKGPITQM | 22 | 1b | 28 | 1165 |
| NS3 | CPSGHAVGI | 22 | 1b | 29 | 931 |
| NS5A | PPVVHGCPL | 22 | 1b/1a | 30 | 1582 |
| C | LPRRGPRLGV | 21 | 1b/1a/3a | 31 | 450 |
| NS3 | CPSGHAVGIF | 21 | 1b | 32 | 932 |
| NS3 | NPSVAATLGF | 21 | 1b/1a/3a | 33 | 1532 |
| NS3 | IPTSGDVVVV | 21 | 1b/1a | 34 | 1295 |
| NS3 | RPSGMFDSSV | 21 | 1b/1a | 35 | 1687 |
| NS4B | SPLTTQHTLL | 21 | 1b | 36 | 1768 |
| NS5A | LPRLPGVPFF | 21 | 1b | 37 | 1440 |
| NS5A | APSLKATCTT | 21 | 1b | 38 | 849 |
| NS5A | VPPVVHGCPL | 21 | 1b | 39 | 1953 |
| NS5B | TPCAAEESKL | 21 | 1b | 40 | 1840 |
| C | APLGGAARA | 21 | 1b/1a | 41 | 384 |
| NS3 | APPGARSLT | 21 | 1b | 42 | 839 |
| NS3 | APTGSGKST | 21 | 1b/1a/3a | 43 | 397 |
| NS3 | GPTPLLYRL | 21 | 1b/1a/3a | 44 | 307 |
| NS4B | PPSAASAFV | 21 | 1b | 45 | 1580 |
| NS4B | SPGALVVGV | 21 | 1b/1a/3a | 46 | 1759 |
| NS5A | APSLKATCT | 21 | 1b/1a | 47 | 848 |
| NS5A | PPRRKRTVV | 21 | 1b | 48 | 1578 |
| NS5B | TPIPAASQL | 21 | 1b | 49 | 1848 |
| E2 | TPSPVVVGTI | 20 | 1b/1a/3a | 50 | 1861 |
| E2 | LPCSFTTLPA | 20 | 1b/1a | 51 | 1424 |
| NS2 | VPYFVRAQGL | 20 | 1b | 52 | 1960 |
| NS3 | TPCTCGSSDL | 20 | 1b/1a | 53 | 1843 |
| NS4B | LPYIEQGMQL | 20 | 1b | 54 | 1447 |
| NS4B | APPSAASAFV | 20 | 1b | 55 | 845 |
| NS4B | SPGALVVGVV | 20 | 1b/1a | 56 | 1760 |
| NS5A | VPAPEFFIEV | 20 | 1b | 57 | 1944 |
| NS5A | EPEPDVAVLT | 20 | 1b/1a | 58 | 1025 |
| NS5B | LPINALSNSL | 20 | 1b/1a | 59 | 1430 |
| C | GPRLGVRAT | 20 | 1b/1a/3a | 60 | 387 |
| E2 | GPWLTPRCL | 20 | 1b | 61 | 1173 |
| NS3 | IPTSGDVVV | 20 | 1b/1a | 62 | 415 |
| NS4B | SPLTTQHTL | 20 | 1b | 63 | 1767 |
| NS5A | LPCEPEPDV | 20 | 1b/1a/3a | 64 | 1421 |
| NS5A | DPSHITAET | 20 | 1b | 65 | 976 |
| NS5B | DPTTPLARA | 20 | 1b/1a | 66 | 980 |
| E1 | IPQAWDMVA | 19 | 1b | 67 | 1294 |
| E2 | PPQGNWFGCT | 19 | 1b | 68 | 1574 |
| NS5A | KPLLREEVTF | 19 | 1b | 69 | 1339 |
| NS5A | EPDVAVLTSM | 19 | 1b/1a | 70 | 1023 |
| NS5A | PPPRRKRTVV | 19 | 1b | 71 | 1573 |
| NS5B | QPEKGGRKPA | 19 | 1b/1a | 72 | 1612 |
| E1 | IPQAVVDMV | 19 | 1b | 73 | 1293 |
| NS3 | APPPSWDQM | 19 | 1b/1a | 74 | 381 |
| NS3 | TPLLYRLGA | 19 | 1b/1a | 75 | 389 |
| NS4B | NPAIASLMA | 19 | 1b/1a | 76 | 1527 |
| NS4B | APPSAASAF | 19 | 1b | 77 | 844 |
| NS5A | DPDYVPPVV | 19 | 1b | 78 | 971 |
| NS5A | IPPPRRKRT | 19 | 1b | 79 | 1291 |
| NS5B | CPMGFSYDT | 19 | 1b | 80 | 421 |
| E2 | VPASQVCGPV | 18 | 1b | 81 | 1946 |
| E2 | YPCTVNFTIF | 18 | 1b | 82 | 2060 |
| NS3 | APITAYSQQT | 18 | 1b | 83 | 835 |
| NS3 | PPAVPQTFQV | 18 | 1b | 84 | 1564 |
| NS3 | AAQGYKVLVL | 18 | 1b/1a | 85 | 777 |
| NS3 | DPNIRTGVRT | 18 | 1b/1a | 86 | 973 |
| NS3 | VPHPNIEEVA | 18 | 1b/1a | 87 | 1951 |
| NS3 | IPFYGKAIPI | 18 | 1b | 88 | 1284 |
| NS3 | LPVCQDHLEF | 18 | 1b/1a | 89 | 1444 |
| NS4A | RPAVIPDREV | 18 | 1b | 90 | 1674 |
| NS4B | APVVESKWRA | 18 | 1b | 91 | 856 |
| NS4B | NPAIASLMAF | 18 | 1b/1a | 92 | 1528 |
| NS4B | GPGEGAVQWM | 18 | 1b/1a/3a | 93 | 1163 |
| NS5A | DPSHITAETA | 18 | 1b | 94 | 977 |
| NS5A | IPPPRRKRTV | 18 | 1b | 95 | 1292 |
| NS5B | QPEYDLELIT | 18 | 1b/1a | 96 | 1614 |
| C | LPGCSFSIF | 18 | 1b/1a/3a | 97 | 375 |
| E2 | GPSQKIQLI | 18 | 1b | 98 | 1171 |
| E2 | LPALSTGLI | 18 | 1b/1a | 99 | 1419 |
| P7 | WPLLLLLLA | 18 | 1b/1a | 100 | 2017 |
| NS2 | AILGPLMVL | 18 | 1b | 101 | 816 |
| NS4B | LPAILSPGA | 18 | 1b/1a/3a | 102 | 1417 |
| NS5A | SPAPNYSRA | 18 | 1b | 103 | 1755 |
| NS5A | KPLLREEVT | 18 | 1b | 104 | 1338 |
| NS5A | PPSLASSSA | 18 | 1b | 105 | 1581 |
| NS5A | SPDADLIEA | 18 | 1b | 106 | 1758 |
| NS5A | LPPTKAPPI | 18 | 1b | 107 | 1435 |
| NS5B | LTRDPTIPL | 18 | 1b/1a | 108 | 1475 |
| NS5B | APTLWARMI | 18 | 1b/1a | 109 | 371 |
| NS5B | SPGEINRVA | 18 | 1b/1a | 110 | 1761 |
| C | KPQRKTKRNT | 17 | 1b/1a/3a | 111 | 1341 |
| E1 | VPTTTIRRHV | 17 | 1b | 112 | 1956 |
| E1 | YPGHVSGHRM | 17 | 1b | 113 | 2063 |
| E2 | GPWLTPRCLV | 17 | 1b | 114 | 1174 |
| NS2 | GPLMVLQAGI | 17 | 1b | 115 | 1168 |
| NS2 | EPVVFSDMET | 17 | 1b | 116 | 1028 |
| NS3 | GPITQMYTNV | 17 | 1b | 117 | 1164 |
| NS3 | VPVESMETTM | 17 | 1b | 118 | 1958 |
| NS3 | ETAGARLVVL | 17 | 1b/1a | 119 | 1032 |
| NS3 | DPTFTIETTT | 17 | 1b/1a | 120 | 979 |
| NS3 | TPGERPSGMF | 17 | 1b | 121 | 1845 |
| NS3 | EPYLVAYQAT | 17 | 1b/1a | 122 | 1079 |
| NS3 | TPLLYRLGAV | 17 | 1b/1a | 123 | 1851 |
| NS4B | VPESDAAARV | 17 | 1b/1a/3a | 124 | 1948 |
| NS5A | CPCQVPAPEF | 17 | 1b | 125 | 927 |
| NS5A | LPCEPEPDVA | 17 | 1b/1a | 126 | 1422 |
| NS5B | SPGQRVEFLV | 17 | 1b/1a | 127 | 1765 |
| NS5B | DPTTPLARAA | 17 | 1b/1a | 128 | 981 |
| NS5B | TPLARAAWET | 17 | 1b/1a/3a | 129 | 1850 |
| NS5B | PPLRVWRHRA | 17 | 1b | 130 | 1571 |
| E2 | APRPCGIVP | 17 | 1b | 131 | 846 |
| NS2 | SPYYKVFLA | 17 | 1b | 132 | 1780 |
| NS2 | HPELIFDIT | 17 | 1b | 133 | 1235 |
| NS2 | TPLRDWAHA | 17 | 1b | 134 | 1852 |
| NS3 | SPPAVPQTF | 17 | 1b | 135 | 1770 |
| NS3 | AQGYKVLVL | 17 | 1b/1a | 136 | 130 |
| NS3 | VPHPNIEEV | 17 | 1b/1a | 137 | 1950 |
| NS3 | TPGERPSGM | 17 | 1b | 138 | 372 |
| NS3 | TLHGPTPLL | 17 | 1b/1a/3a | 139 | 81 |
| NS5A | EPPALPIWA | 17 | 1b | 140 | 1078 |
| NS5A | PRRKRTVVL | 17 | 1b | 141 | 1583 |
| NS5A | EPGDPDLSD | 17 | 1b/1a | 142 | 1026 |
| NS5B | TPIDTTIMA | 17 | 1b/1a | 143 | 1847 |
| NS5B | EPLDLPQII | 17 | 1b | 144 | 1027 |
| P7 | ALYGVWPLLL | 16 | 1b | 145 | 831 |
| NS2 | SCGGAVFVGL | 16 | 1b | 146 | 1721 |
| NS2 | TLSPYYKVFL | 16 | 1b | 147 | 1836 |
| NS2 | AACGDIILGL | 16 | 1b | 148 | 774 |
| NS3 | APPGARSLTP | 16 | 1b | 149 | 840 |
| NS3 | RRRGDSRGSL | 16 | 1b/1a | 150 | 1697 |
| NS3 | GPLLCPSGHA | 16 | 1b | 151 | 1167 |
| NS3 | IPPGSVTVPH | 16 | 1b/1a | 152 | 1854 |
| NS3 | KQAGDNFPYL | 16 | 1b | 153 | 1346 |
| NS5A | SPPSLASSSA | 16 | 1b | 154 | 1771 |
| NS5B | TPPHSAKSKF | 16 | 1b/1a | 155 | 1856 |
| NS5B | TPVNSWLGNI | 16 | 1b/1a/3a | 156 | 1862 |
| NS5B | CLRKLGVPPL | 16 | 1b/1a | 157 | 921 |
| C | PRRGPRLGV | 16 | 1b/1a/3a | 158 | 449 |
| C | WPLYGNEGM | 16 | 1b | 159 | 2019 |
| C | SPRGSRPSW | 16 | 1b/1a/3a | 160 | 386 |
| E1 | MNWSPTTAL | 16 | 1b | 161 | 1503 |
| E2 | RPCGIVPAS | 16 | 1b | 162 | 1676 |
| E2 | GPPCNIGGV | 16 | 1b | 163 | 1169 |
| E2 | YPCTVNFTI | 16 | 1b | 164 | 2059 |
| NS2 | ARRGREILL | 16 | 1b/1a | 165 | 865 |
| NS2 | ILLGPADSL | 16 | 1b | 166 | 1271 |
| NS3 | VPVESMETT | 16 | 1b | 167 | 1957 |
| NS3 | PPAVPQTFQ | 16 | 1b | 168 | 1563 |
| NS3 | DPTFTIETT | 16 | 1b/1a | 169 | 978 |
| NS3 | RPSGMFDSS | 16 | 1b/1a | 170 | 1686 |
| NS3 | FPYLVAYQA | 16 | 1b/1a | 171 | 443 |
| NS3 | HPITKYIMA | 16 | 1b | 172 | 396 |
| NS5A | KSRKFPPAL | 16 | 1b | 173 | 1348 |
| NS5A | CPLPPTKAP | 16 | 1b | 174 | 928 |
| NS5A | APPIPPPRR | 16 | 1b | 175 | 843 |
| NS5A | PPPRRKRTV | 16 | 1b | 176 | 1572 |
| NS5B | PPHSAKSKF | 16 | 1b/1a | 177 | 1568 |
| NS5B | QPEYDLELI | 16 | 1b/1a | 178 | 1613 |
| C | FPGGGQIVGG | 15 | 1b/1a/3a | 179 | 1077 |
| C | QPRGRRQPIP | 15 | 1b/1a/3a | 180 | 1616 |
| C | RPSWGPTDPR | 15 | 1b/1a | 181 | 1690 |
| E1 | NNSSRCWVAL | 15 | 1b | 182 | 1526 |
| E1 | SPRRHETVQD | 15 | 1b | 183 | 1778 |
| E2 | AIKWEYVLLL | 15 | 1b | 184 | 815 |
| E21P7 | EAALENLVVL | 15 | 1b | 185 | 998 |
| P7 | AYALYGVWPL | 15 | 1b | 186 | 901 |
| NS2 | AHLQVWVPPL | 15 | 1b | 187 | 811 |
| NS3 | AYSQQTRGLL | 15 | 1b | 188 | 906 |
| NS3 | SPRPVSYLKG | 15 | 1b | 189 | 1776 |
| NS3 | AYAAQGYKVL | 15 | 1b/1a | 190 | 900 |
| NS3 | ETSVRLRAYL | 15 | 1b | 191 | 1033 |
| NS4B | AFTASITSPL | 15 | 1b | 192 | 802 |
| NS4B | ILGGWVAAQL | 15 | 1b/1a | 193 | 1270 |
| NS5A | APACKPLLRE | 15 | 1b | 194 | 834 |
| NS5A | VESENKVVIL | 15 | 1b/1a | 195 | 1902 |
| NS5A | RKSRKFPPAL | 15 | 1b | 196 | 1655 |
| NS5A | WARPDYNPPL | 15 | 1b/1a/3a | 197 | 1998 |
| NS5B | EESKLPINAL | 15 | 1b | 198 | 1009 |
| NS5B | EKGGRKPARL | 15 | 1b/1a | 199 | 1015 |
| NS5B | ASAACRAAKL | 15 | 1b | 200 | 869 |
| NS5B | APPGDPPQPE | 15 | 1b/1a | 201 | 842 |
| NS5B | DASGKRVYYL | 15 | 1b | 202 | 955 |
| NS5B | SPGEINRVAS | 15 | 1b | 203 | 1762 |
| C | AQPGYPWPL | 15 | 1b/1a/3a | 204 | 65 |
| C | QPGYPWPLY | 15 | 1b/1a/3a | 205 | 216 |
| C | ALAHGVRVL | 15 | 1b/1a | 206 | 72 |
| C | SFSIFLLAL | 15 | 1b/1a/3a | 207 | 250 |
| E1 | NSSRCWVAL | 15 | 1b | 208 | 1533 |
| E1 | AHWGVLAGL | 15 | 1b | 209 | 812 |
| E2 | WTRGERCDL | 15 | 1b/1a | 210 | 2022 |
| E2/P7 | AALENLVVL | 15 | 1b | 211 | 776 |
| P7 | ALYGVWPLL | 15 | 1b | 212 | 830 |
| P7 | YGVWPLLLL | 15 | 1b | 213 | 2036 |
| NS2 | CGGAVFVGL | 15 | 1b | 214 | 915 |
| NS2 | ACGDIILGL | 15 | 1b | 215 | 784 |
| NS3 | AYSQQTRGL | 15 | 1b | 216 | 905 |
| NS3 | KGSSGGPLL | 15 | 1b/1a | 217 | 260 |
| NS3 | TILGIGTVL | 15 | 1b | 218 | 89 |
| NS3 | TAGARLVVL | 15 | 1b/1a | 219 | 249 |
| NS3 | TPPGSVTVP | 15 | 1b/1a | 220 | 1853 |
| NS3 | PPGSVTVPH | 15 | 1b/1a | 221 | 1567 |
| NS3 | TPGLPVCQD | 15 | 1b/1a/3a | 222 | 1846 |
| NS4A | LVGGVLAAL | 15 | 1b/1a | 223 | 1479 |
| NS4A | AVIPDREVL | 15 | 1b | 224 | 891 |
| NS4A | IPDREVLYR | 15 | 1b/1a | 225 | 1282 |
| NS4B | LPGNPAIAS | 15 | 1b/1a | 226 | 1427 |
| NS4B | MPSTEDLVN | 15 | 1b | 227 | 1504 |
| NS5A | LPGVPFFSC | 15 | 1b | 228 | 1429 |
| NS5A | GPCTPSPAP | 15 | 1b | 229 | 1161 |
| NS5A | APACKPLLR | 15 | 1b | 230 | 833 |
| NS5A | EPDVAVLTS | 15 | 1b/1a | 231 | 1022 |
| NS5A | LARGSPPSL | 15 | 1b | 232 | 1363 |
| NS5A | HHDSPDADL | 15 | 1b | 233 | 1220 |
| NS5A | PPALPIWAR | 15 | 1b | 234 | 1562 |
| NS5B | ESKLPINAL | 15 | 1b | 235 | 1031 |
| NS5B | KPARLIVFP | 15 | 1b/1a | 236 | 1337 |
| NS5B | AIRSLTERL | 15 | 1b | 237 | 473 |
| NS5B | APPGDPPQP | 15 | 1b/1a | 238 | 841 |
| NS5B | PPGDPPQPE | 15 | 1b/1a | 239 | 1565 |
| NS5B | ASGKRVYYL | 15 | 1b | 240 | 334 |
| NS5B | RARSVRAKL | 15 | 1b | 241 | 1632 |
| C | GPRLGVRATR | 14 | 1b/1a/3a | 242 | 1170 |
| C | RPEGRAWAQP | 14 | 1b | 243 | 1678 |
| C | EGMGWAGWLL | 14 | 1b | 244 | 1012 |
| C | SPRGSRPSWG | 14 | 1b/1a/3a | 245 | 1773 |
| C | RALAHGVRVL | 14 | 1b/1a | 246 | 1629 |
| C/E1 | IPASAYEVRN | 14 | 1b | 247 | 1281 |
| E1 | FCSAMYVGDL | 14 | 1b | 248 | 1052 |
| E1 | VGDLCGSVFL | 14 | 1b/1a | 249 | 1910 |
| E1 | MVAGAHWGVL | 14 | 1b | 250 | 1509 |
| nHLAPred | | | | | |
| NS4B | LPAILSPGA | 1.000 | 1b/1a/3a | 1 | 1417 |
| NS4B | PPSAASAFV | 1.000 | 1b | 2 | 1580 |
| NS5B | DPTTPLARA | 1.000 | 1b/1a | 3 | 980 |
| NS3 | PPGSVTVPH | 1.000 | 1b/1a | 4 | 1567 |
| NS5B | DPPQPEYDL | 1.000 | 1b/1a | 5 | 543 |
| NS5B | SPGQRVEFL | 1.000 | 1b/1a | 6 | 373 |
| C | SPRGSRPSW | 1.000 | 1b/1a/3a | 7 | 386 |
| NS3 | IPTSGDVVV | 1.000 | 1b/1a | 8 | 415 |
| NS5A | RPDYNPPLL | 1.000 | 1b/1a/3a | 9 | 1677 |
| NS5B | MTHFFSILL | 1.000 | 1b | 10 | 1508 |
| NS2 | FLARLIWWL | 1.000 | 1b | 11 | 1063 |
| NS5B | TPPHSAKSK | 1.000 | 1b/1a | 12 | 1855 |
| E1 | VPTTTIRRH | 1.000 | 1b | 13 | 1955 |
| NS5A | APACKPLLR | 1.000 | 1b | 14 | 833 |
| NS3 | SPPAVPQTF | 1.000 | 1b | 15 | 1770 |
| C | DPRRRSRNL | 1.000 | 1b/1a/3a | 16 | 370 |
| NS3 | VPQTFQVAH | 1.000 | 1b | 17 | 410 |
| E2 | SPGPSQKIQ | 1.000 | 1b | 18 | 1763 |
| C | RPQDVKFPG | 1.000 | 1b/1a/3a | 19 | 552 |
| NS5B | RHTPVNSWL | 1.000 | 1b/1a/3a | 20 | 298 |
| NS5B | LMTHFFSIL | 1.000 | 1b | 21 | 1414 |
| NS5A | SPAPNYSRA | 1.000 | 1b | 22 | 1755 |
| C | LPGCSFSIF | 1.000 | 1b/1a/3a | 23 | 375 |
| NS3 | TPAETSVRL | 1.000 | 1b | 24 | 383 |
| C | FPGGGQIVG | 1.000 | 1b/1a/3a | 25 | 407 |
| NS3 | IMACMSADL | 1.000 | 1b | 26 | 90 |
| NS4B | SPLTTQHTL | 1.000 | 1b | 27 | 1767 |
| NS5A | PPVVHGCPL | 1.000 | 1b/1a | 28 | 1582 |
| NS5A | FPPALPIWA | 1.000 | 1b | 29 | 1078 |
| NS5B | IPAASQLDL | 1.000 | 1b | 30 | 1280 |
| NS3 | HPNIEEVAL | 1.000 | 1b/1a | 31 | 1237 |
| NS5B | TPIPAASQL | 1.000 | 1b | 32 | 1848 |
| NS5B | RPRWFMLCL | 1.000 | 1b | 33 | 1684 |
| NS3 | VPHPNIEEV | 1.000 | 1b/1a | 34 | 1950 |
| NS5A | LPPTKAPPI | 1.000 | 1b | 35 | 1435 |
| NS5A | PPPRRKRTV | 1.000 | 1b | 36 | 1572 |
| NS5A | PPRRKRTVV | 1.000 | 1b | 37 | 1578 |
| E2 | YPCTVNFTI | 1.000 | 1b | 38 | 2059 |
| E2 | GPWLTPRCL | 1.000 | 1b | 39 | 1173 |
| NS3 | GPTPLLYRL | 1.000 | 1b/1a/3a | 40 | 307 |
| NS5A | LPRLPGVPF | 1.000 | 1b | 41 | 1439 |
| C | QPIPKARRP | 0.990 | 1b/1a | 42 | 479 |
| NS5A | PPALPIWAR | 0.990 | 1b | 43 | 1562 |
| E2 | RPIDKFAQG | 0.990 | 1b | 44 | 1679 |
| C | LPRRGPRLG | 0.990 | 1b/1a/3a | 45 | 380 |
| NS5A | PPIPPPRRK | 0.990 | 1b | 46 | 1570 |
| NS5B | LPQIIERLH | 0.990 | 1b | 47 | 1438 |
| E1 | SPRRHETVQ | 0.990 | 1b | 48 | 1777 |
| NS5A | APPIPPPRR | 0.990 | 1b | 49 | 843 |
| NS3 | PPAVPQTFQ | 0.990 | 1b | 50 | 1563 |
| P7/NS2 | PPRAYAMDR | 0.990 | 1b | 51 | 1576 |
| E2 | CPTDCFRKH | 0.990 | 1b/1a/3a | 52 | 934 |
| E2 | GPPCNIGGV | 0.990 | 1b | 53 | 1169 |
| NS4B | NPAIASLMA | 0.990 | 1b/1a | 54 | 1527 |
| NS3 | HPITKYIMA | 0.990 | 1b | 55 | 396 |
| NS2 | SPYYKVFLA | 0.990 | 1b | 56 | 1780 |
| NS5B | KPARLIVFP | 0.990 | 1b/1a | 57 | 1337 |
| NS5A | LPCEPEPDV | 0.990 | 1b/1a/3a | 58 | 1421 |
| NS3 | IPVRRRGDS | 0.990 | 1b/1a | 59 | 1297 |
| NS4B/NS5 | ATPCSGSWLR | 0.990 | 1b/1a | 60 | 1841 |
| C | GPTDPRRRS | 0.990 | 1b/1a | 61 | 1172 |
| E2 | LPALSTGLI | 0.990 | 1b/1a | 62 | 1419 |
| NS4B | LPYIEQGMQ | 0.990 | 1b | 63 | 1446 |
| E1 | TPGCVPCVR | 0.980 | 1b/1a | 64 | 1844 |
| E1 | IPQAVVDMV | 0.980 | 1b | 65 | 1293 |
| C | IPLVGAPLG | 0.980 | 1b/1a | 66 | 442 |
| NS5A | CPCGAQITG | 0.980 | 1b | 67 | 926 |
| E2 | RPYCWHYAP | 0.980 | 1b | 68 | 1691 |
| NS5A | IPPPRRKRT | 0.980 | 1b | 69 | 1291 |
| C | QPRGRRQPI | 0.980 | 1b/1a/3a | 70 | 390 |
| NS4B | LPGNPAIAS | 0.980 | 1b/1a | 71 | 1427 |
| NS5B | TPIDTTIMA | 0.980 | 1b/1a | 72 | 1847 |
| E2 | RPPQGNWFG | 0.980 | 1b | 73 | 1680 |
| P7/NS2 | LPPRAYAMD | 0.980 | 1b | 74 | 1434 |
| NS5A | DPDYVPPVV | 0.970 | 1b | 75 | 971 |
| NS3 | IPIEVIKGG | 0.970 | 1b | 76 | 561 |
| C | KPQRKTKRN | 0.970 | 1b/1a/3a | 77 | 1340 |
| NS5A | VPPVVHGCP | 0.970 | 1b | 78 | 1952 |
| NS5A | APNYSRALW | 0.970 | 1b | 79 | 838 |
| NS5B | TPLARAAWE | 0.970 | 1b/1a/3a | 80 | 1849 |
| NS3 | SPRPVSYLK | 0.970 | 1b | 81 | 1775 |
| NS3 | TPLLYRLGA | 0.970 | 1b/1a | 82 | 389 |
| E2 | GPSQKIQLI | 0.970 | 1b | 83 | 1171 |
| E1 | YPGHVSGHR | 0.970 | 1b | 84 | 2062 |
| NS4B | SPTHYVPES | 0.960 | 1b/1a/3a | 85 | 1779 |
| NS5B | LPQAVMGSS | 0.960 | 1b | 86 | 1436 |
| NS5A | LPGVPFFSC | 0.960 | 1b | 87 | 1429 |
| C | IPKARRPEG | 0.960 | 1b/1a | 88 | 409 |
| NS4B | SPGALVVGV | 0.960 | 1b/1a/3a | 89 | 1759 |
| NS3 | KPTLHGPTP | 0.960 | 1b/1a/3a | 90 | 1342 |
| NS5A | EPGDPDLSD | 0.960 | 1b/1a | 91 | 1026 |
| NS5A | LPIWARPDY | 0.960 | 1b | 92 | 1431 |
| NS5B | PPHSAKSKF | 0.960 | 1b/1a | 93 | 1568 |
| NS3 | TPCTCGSSD | 0.960 | 1b/1a | 94 | 1842 |
| NS4A | IPDREVLYR | 0.960 | 1b/1a | 95 | 1282 |
| NS5B | TPCAAEESK | 0.960 | 1b | 96 | 1839 |
| NS3 | CPSGHAVGI | 0.950 | 1b | 97 | 931 |
| NS3 | DPNIRTGVR | 0.950 | 1b/1a | 98 | 972 |
| NS5B | CPMGFSYDT | 0.950 | 1b | 99 | 421 |
| NS5A | TPSPAPNYS | 0.950 | 1b | 100 | 1859 |
| Epimmune | | | | | |
| NS5B | APTLWARMIL | 1.24 | 1b/1a | 1 | 853 |
| C | SPRGSRPSW | 1.64 | 1b/1a/3a | 2 | 386 |
| E2 | RPCGIVPAL | 1.89 | — | 3 | 1675 |
| C | QPRGRRQPI | 2.95 | 1b/1a/3a | 4 | 390 |
| C | APLGGAARAL | 3.46 | 1b/1a | 5 | 836 |
| NS4B | LPAILSPGAL | 4.39 | 1b/1a/3a | 6 | 1418 |
| C | LPRRGPRLGV | 4.88 | 1b/1a/3a | 7 | 450 |
| C | APLGGVARAL | 5.53 | — | 8 | 837 |
| NS4B | NPAIASLMAF | 7.2 | 1b/1a | 9 | 1528 |
| P7 | WPLLLLLLAL | 7.45 | 1b/1a | 10 | 2018 |
| NS5B | SPAQRVEFL | 7.57 | — | 11 | 1757 |
| NS3 | KPTLHGPTPL | 7.91 | 1b/1a/3a | 12 | 1343 |
| NS3 | IPFYGKAIPL | 7.94 | 1a | 13 | 1285 |
| C | SPRGSRPNW | 10.81 | — | 14 | 1772 |
| C | DPRRRSRNL | 12.09 | 1b/1a/3a | 15 | 370 |
| NS5B | APTLWARMI | 13.88 | 1b/1a | 16 | 371 |
| E2 | YPCTVNFTL | 16.54 | 3a | 17 | 2061 |
| NS5B | SPGQRVEFL | 21.27 | 1b/1a | 18 | 373 |
| NS5A | VPPVVHGCPL | 26.04 | 1b | 19 | 1953 |
| NS3 | IPFYGKAIPI | 29.35 | 1b/3a | 20 | 1284 |
| | PPRKKRTVV | 30.59 | 1a | 21 | 1577 |
| C | LPGCSFSIFL | 31.72 | 1b/1a/3a | 22 | 1426 |
| NS3 | RPSGMFDSSV | 37.43 | 1b/1a | 23 | 1687 |
| E2 | APRPCGIVPA | 38.12 | 1b | 24 | 847 |
| NS3 | HPITKYIMA | 38.64 | 1b | 25 | 396 |
| E2 | YPCTVNFSI | 41.7 | — | 26 | 2058 |
| NS4B | LPYIEQGMQL | 43.26 | 1b | 27 | 1447 |
| NS5B | LPINALSNSL | 45.73 | 1b/1a | 28 | 1430 |
| NS3 | KPTLQGPTPL | 47.48 | — | 29 | 1344 |
| NS3 | HPVTKYIMA | 47.89 | — | 30 | 1238 |
| | CPAGHAVGIF | 56.36 | 1a | 31 | 925 |
| | CPSGHVVGI | 61.68 | — | 32 | 933 |
| E2 | GPWLTPRCL | 64.48 | 1b | 33 | 1173 |
| E2 | YPCTVNFTI | 70.75 | 1b | 34 | 2059 |
| NS5A | RPDYNPPLL | 72.65 | 1b/1a/3a | 35 | 1677 |
| NS4B | APPSAASAFV | 75.67 | 1b | 36 | 845 |
| | GPKGPVTQM | 86.45 | — | 37 | 1166 |
| C | LPGCSFSIF | 101.3 | 1b/1a/3a | 38 | 375 |
| E2 | GPWLTPRCM | 104.59 | 3a | 39 | 1175 |
| E2 | TPRCLVDYPY | 237.55 | 1b/1a | 40 | 1857 |
| E1 | YPGHVSGHRM | 264.06 | 1b | 41 | 2063 |
| E2 | YPCTVNFTIF | 307.31 | 1b | 42 | 2060 |
| E2 | TPRCMVDYPY | 445.13 | 3a | 43 | 1858 |
| NS5A | EPDVAVLTSM | 597.05 | 1b/1a | 44 | 1023 |
| NS5B | IPPHSAKSKF | 699.16 | 1b/1a | 45 | 1856 |
| NS3 | TPGERPSGMF | 699.46 | 1b | 46 | 1845 |
| NS3 | IPGERPSGM | 833.63 | 1b | 47 | 372 |
| NS3 | APPPSWDQM | 933.01 | 1b/1a | 48 | 381 |
| NS4B | GPGEGAVQWM | 976.39 | 1b/1a/3a | 49 | 1163 |
| NS3 | NPSVAATLGF | 1610.36 | 1b/1a/3a | 50 | 1532 |
| NS3 | VPAAYAAQGY | 2733.82 | 1b/1a | 51 | 1943 |
| NS5B | PPHSARSKF | 4228.63 | 3a | 52 | 1569 |
| NS5A | LPIWARPDY | 4289.5 | 1b/3a | 53 | 1431 |
| NS3 | LPVCQDHLEF | 5715.31 | 1b/1a | 54 | 1444 |
| NS5B | PPHSAKSKF | 9169.56 | 1b/1a | 55 | 1568 |
| P7 | VPGAAYALY | 27777.1 | 1b | 56 | 1949 |
| C | QPGYPWPLY | 39918.4 | 1b/1a/3a | 57 | 216 |
| NS5B | PPGDPPQPEY | 633519.2 | 1b/1a | 58 | 1566 |

Those peptides that are present in at least the consensus sequence of genotype 1a and 1b, are selected. Table 15 contains all these peptides, with their score, and designated ranknumber, of each of the prediction servers in separate columns, and their occurrence in the different genotypes.

A selection according to genotype and ranknumber results in 232 different peptide sequences, i.e. 150+113+45+28=336. The table 16 contains the selection of peptides for which min. 2 out of 4 prediction servers give a rank =<100. This renders 40 different sequences. Said peptides are finally incorporated in Table 13.

The selection of potential HLA B07 peptide binders is summarized as follows: BIMAS (B7):

| | |
|---|---|
| output | 200 9-mers |
| prediction | 200 10-mers |
| server: | |
| BIMAS | paste 9-mers + 10-mers, sort on BIMAS score |
| results: | → 400 peptides, ranknumber for 9- and 10-mers |
| | separately (2× 1-200) |
| | → BIMAS ranking for peptides with same score unknown |
| BIMAS | selection on genotype (at least in 1b + 1a consensus): |
| selection: | → 150 peptides |

Syfpeithi (B0702):

| | |
|---|---|
| output | 3002 9-mers |
| prediction | 3001 10-mers |
| server: | |
| Syfpeithi | paste 9-mers + 10-mers, sort on Syfpeithi score |
| results: | → select 250 peptides, 1 ranking 1-250 |
| | (126 9-mers + 124 10-mers) |
| | → Syfpeithi ranking for peptides with same score unknown |
| Syfpeithi | selection on genotype (at least in 1b + 1a consensus): |
| selection: | → 113 peptides |

nHLAPred (B0702):

| | |
|---|---|
| output | 200 9-mers |
| prediction | no 10-mers |
| server: | |
| nHLAPred | → select 100 peptides, ranking 1-100 |
| results: | → nHLAPred ranking for peptides with same score unknown |
| nHLAPred | selection on genotype (at least in 1b + 1a consensus): |
| selection: | → 45 peptides |

EPMN (B07):

| | |
|---|---|
| EPMN | 85 peptides (38 9-mers + 47 10-mers) with motif OK |
| results: | PIC between 0.17 and 633519; 64 with PIC =< 100 |
| EPMN | → selection on genotype: select 58 peptides, that |
| selection: | are present in at least 1/32 |
| | 1b sequences EPMN used for predictions |
| EPMN 2^{nd} | selection on genotype (at least in 1b + 1a consensus): |
| selection: | → 28 peptides (16 with PIC =< 100) |

TABLE 16

Selected B07 predicted peptides

| | | | | | |
|---|---|---|---|---|---|
| | Peptide | Number | | | SEQ ID |
| Protein | sequence | of pred. | Ki | Genotype | NO |

| | | | | | |
|---|---|---|---|---|---|
| C | DPRRRSRNL | 4 | 18 | 1b/1a/3a | 370 |
| C | QPRGRRQPI | 4 | 1 | 1b/1a/3a | 390 |
| NS5A | RPDYNPPLL | 4 | 143 | 1b/1a/3a | 1677 |
| NS5B | SPGQRVEFL | 4 | 38 | 1b/1a | 373 |
| C | LPRRGPRLGV | 3 | 3 | 1b/1a/3a | 450 |
| NS3 | GPTPLLYRL | 3 | 209 | 1b/1a/3a | 307 |
| NS3 | KPTLHGPTPL | 3 | 6 | 1b/1a/3a | 1343 |
| NS4B | LPAILSPGAL | 3 | 255 | 1b/1a/3a | 1418 |
| C | LPGCSFSIFL | 3 | 558 | 1b/1a/3a | 1426 |
| NS4B | GPGEGAVQWM | 3 | 4747 | 1b/1a/3a | 1163 |
| NS5B | APTLWARMIL | 3 | 1 | 1b/1a | 853 |
| C | APLGGAARAL | 3 | 1 | 1b/1a | 836 |
| NS5B | DPPQPEYDL | 3 | high | 1b/1a | 543 |
| NS3 | HPNIEEVAL | 3 | 230 | 1b/1a | 1237 |
| P7 | WPLLLLLLAL | 3 | 1474 | 1b/1a | 2018 |
| NS5B | LPINALSNSL | 3 | 12 | 1b/1a | 1430 |
| NS3 | APPPSWDQM | 3 | 281 | 1b/1a | 381 |
| C | LPGCSFSIF | 3 | high | 1b/1a/3a | 375 |
| C | GPRLGVRAT | 2 | 128 | 1b/1a/3a | 387 |
| C | SPRGSRPSW | 2 | 11 | 1b/1a/3a | 386 |
| NS5A | LPCEPEPDV | 2 | high | 1b/1a/3a | 1421 |
| NS4B | LPGNPAIASL | 2 | 266 | 1b/1a | 1428 |
| NS3 | TPCTCGSSDL | 2 | 168 | 1b/1a | 1843 |
| NS3 | AAQGYKVLVL | 2 | 5524 | 1b/1a | 777 |
| NS5A | EPEPDVAVL | 2 | high | 1b/1a | 1024 |
| NS5B | APTLWARMI | 2 | 11 | 1b/1a | 371 |
| NS5A | PPVVHGCPL | 2 | 433 | 1b/1a | 1582 |
| E2 | LPALSTGLI | 2 | 233 | 1b/1a | 1419 |
| NS5B | PPQPEYDLEL | 2 | high | 1b/1a | 1575 |
| NS5A | EPDVAVLTSM | 2 | 454 | 1b/1a | 1023 |
| NS3 | IPTSGDVVV | 2 | 3152 | 1b/1a | 415 |
| NS3 | RPSGMFDSSV | 2 | 14 | 1b/1a | 1687 |
| NS4B | SPGALVVGV | 2 | 627 | 1b/1a/3a | 1759 |
| NS5B | DPTTPLARA | 2 | 13058 | 1b/1a | 980 |
| NS4B | NPAIASLMA | 2 | 676 | 1b/1a | 1527 |
| NS3 | TPLLYRLGA | 2 | 74 | 1b/1a | 389 |
| NS5B | PPHSAKSKF | 2 | high | 1b/1a | 1568 |
| NS3 | NPSVAATLGF | 2 | 1197 | 1b/1a/3a | 1532 |
| NS4B | NPAIASLMAF | 2 | 121 | 1b/1a | 1528 |
| NS3 | LPVCQDHLEF | 2 | 1564 | 1b/1a | 1444 |

### Example 3

### HLA Class I Competition Cell-Based Binding Assays

The interaction of the peptides with the binding groove of the HLA molecules is studied using competition-based cellular peptide binding assays as described by Kessler et al. (2003). Briefly, Epstein-Barr virus (EBV)-transformed B cell lines (B-LCLs) expressing the class I allele of interest are used. EBV transformation is done according to standard procedures (Current Protocols in Immunology, 1991, Wiley Interscience). Naturally bound class I peptide are eluted from the B-LCLs by acid-treatment to obtain free class I molecules. Subsequently, B-LCLs are incubated with a mixture of fluorescein (F1)-labelled reference peptide, and titrating amounts of the competing test peptide. The reference peptide should have a known, high affinity for the HLA-molecule. Cell-bound fluorescence is determined by flow cytometry. The inhibition of binding of the F1-labelled reference peptide is determined and IC50-values are calculated (IC50=concentration of competing peptide that is able to occupy 50% of the HLA molecules). The affinity (Kd) of the reference peptide is determined in a separate experiment in which the direct binding of different concentrations of reference peptide is monitored and data are analysed using a model for one-site binding interactions. The inhibition constant (Kᵢ) of the competing peptides (reflecting their affinity) is calculated as:

$${K}_{i} = \frac{IC50}{1 + [ F1 \text{-} pep ] / Kd}$$

[F1-pep]: concentration of the F1-labeled peptide used in the competition experiment.

The predicted peptides were synthesized using standard technology and tested for binding to B-LCLs with the corresponding HLA-allele. F1-labelled reference peptides are synthesized as Cys-derivatives and labelling is performed with 5-(iodoacetamido) fluorescein at pH 8,3 (50 mM Bicarbonate/1 mM EDTA buffer). The labelled peptides were desalted and purified by C18 RP-HPLC. Labelled peptides were analysed by mass spectrometry.

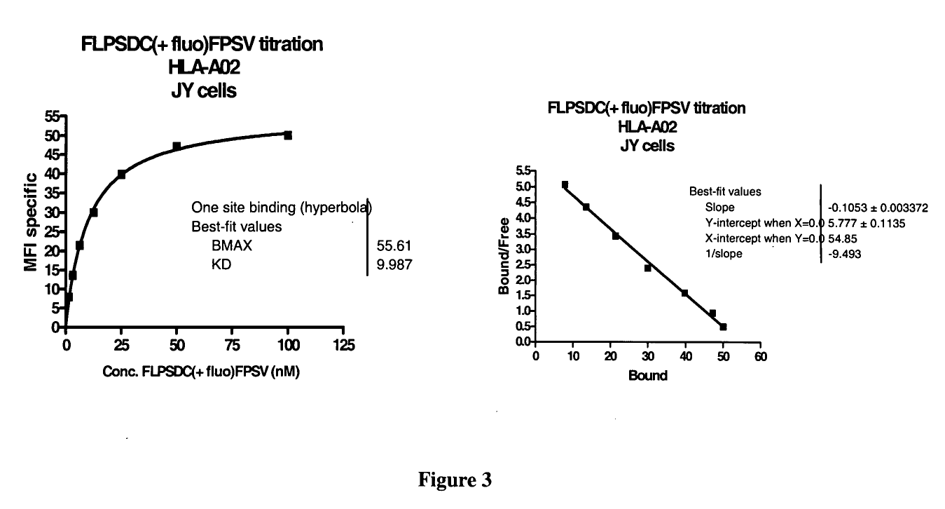

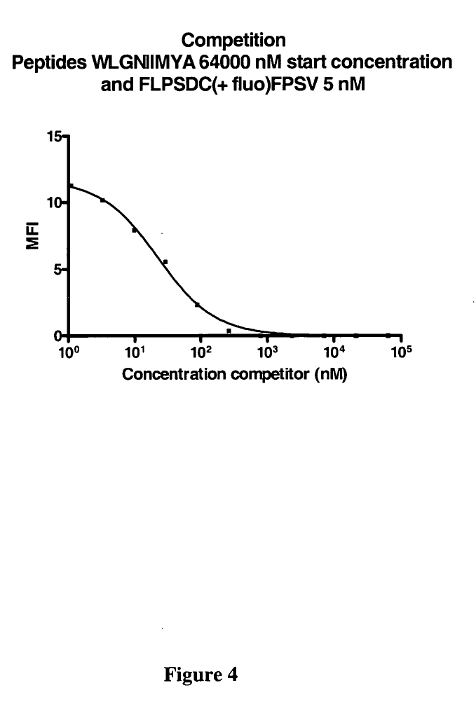

As an example, the interaction of a predicted strong binding peptide with HLA-A02 is shown. An HLA-A02 positive B-LCL (J Y, Kessler et al., 2003) is used for analysing the competition of the F1-labelled reference peptide FLPSDC(F1)FPSV and the predicted peptides (SEQ ID NO 62 to SEQ ID NO 93). The binding of the reference peptide to HLA A02 is shown in FIG. 3. Analysing the data according to a one-site binding model reveals an affinity of the reference peptide of about 10 nM. A typical competition experiment is shown in FIG. 4. This particular set up was used for all class C binding peptides as well as part of the HLA A24 binding peptides. Table 13 contains the calculated inhibition constants (Ki).

### Example 4

### HLA Class I and II Competition Binding Assays Using Soluble HLA

The following example of peptide binding to soluble HLA molecules demonstrates quantification of binding affinities of HLA class I and class II peptides.

Epstein-Barr virus (EBV)-transformed homozygous cell lines, fibroblasts or transfectants were used as sources of HLA class I molecules. Cell lysates were prepared and HLA molecules purified in accordance with disclosed protocols (Sidney et al., 1998; Sidney et al., 1995; Sette, et al., 1994).

HLA molecules were purified from lysates by affinity chromatography. The lysate was passed over a column of Sepharose CL-4B beads coupled to an appropriate antibody. The antibodies used for the extraction of HLA from cell lysates are W6/32 (for HLA-A, -B and -C), B123.2 (for HLA-B and -C) and LB3.1 (for HLA-DR).

The anti-HLA column was then washed with 10 mM Tris-HCL, pH8, in 1% NP-40, PBS, and PBS containing 0,4% n-octylglucoside and HLA molecules were eluted with 50 mM diethylamine in 0,15M NaCl containing 0,4% n-octylglucoside, pH 11,5. A 1/25 volume of 2M Tris, pH6,8, was added to the eluate to reduce the pH to +/−pH8. Eluates were then concentrated by centrifugation in Centriprep 30 concentrators (Amicon, Beverly, Mass.). Protein content was evaluated by a BCA protein assay (Pierce Chemical Co., Rockford, Ill.) and confirmed by SDS-PAGE.

A detailed description of the protocol utilized to measure the binding of peptides to Class I and Class II MHC has been published (Sette et al., 1994; Sidney et al., 1998). Briefly, purified MHC molecules (5 to 500 nM) were incubated with various unlabeled peptide inhibitors and 1-10 nM ¹²⁵I-radiolabeled probe peptides for 48 h in PBS containing 0,05% Nonidet P-40 (NP40) in the presence of a protease inhibitor cocktail. All assays were at pH7 with the exception of DRB1*0301, which was performed at pH 4,5, and DRB1*1601 (DR2w21 1) and DRB4*0101 (DRw53), which were performed at pH5.

Following incubation, MHC-peptide complexes were separated from free peptide by gel filtration on 7,8 mm×15 cm TSK200 columns (TosoHaas 16215, Montgomeryville, Pa.). The eluate from the TSK columns was passed through a Beckman 170 radioisotope detector, and radioactivity was plotted and integrated using a Hewlett-Packard 3396A integrator, and the fraction of peptide bound was determined. Alternatively, MHC-peptide complexes were separated from free peptide by capturing onto ELISA plates coated with anti-HLA antibodies. After free peptide has been washed away, remaining reactivities were measured using the same method as above.

Radiolabeled peptides were iodinated using the chloramine-T method.

Typically, in preliminary experiments, each MHC preparation was titered in the presence of fixed amounts of radiolabeled peptides to determine the concentration of HLA molecules necessary to bind 10-20% of the total radioactivity. All subsequent inhibition and direct binding assays were performed using these HLA concentrations.

Since under these conditions [label]<[HLA] and IC50≧[HLA], the measured IC50 values are reasonable approximations of the true KD values. Peptide inhibitors are typically tested at concentrations ranging from 120 μg/ml to 1,2 ng/ml, and are tested in two to four completely independent experiments. To allow comparison of the data obtained in different experiments, a relative binding figure is calculated for each peptide by dividing the IC50 of a positive control for inhibition by the IC50 for each tested peptide (typically unlabeled versions of the radiolabeled probe peptide). For database purposes, and inter-experiment comparisons, relative binding values are compiled. These values can subsequently be converted back into IC50 nM values by dividing the IC50 nM of the positive controls for inhibition by the relative binding of the peptide of interest. This method of data compilation has proven to be the most accurate and consistent for comparing peptides that have been tested on different days, or with different lots of purified MHC.

This particular set up was used for all class A and B binding peptides (except for some HLA A24 binding peptides, where the cell-based binding assay was used). Table 13 contains the IC 50 values.

Because the antibody used for HLA-DR purification (LB3.1) is alpha-chain specific, beta-1 molecules are not separated from beta-3 (and/or beta-4 and beta-5) molecules. The beta-1 specificity of the binding assay is obvious in the cases of DRB1*0101 (DR1), DRB1*0802 (DR8w2), and DRB1*0803 (DR8w3), where no beta-3 is expressed. It has also been demonstrated for DRB1*0301 (DR3) and DRB3*0101 (DR52a), DRB1*0401 (DR4w4), DRB1*0404 (DR4w14), DRB1*0405 (DR4w15), DRB*1L101 (DR5), DRB1*1201 (DR5w12), DRB1*1302 (DR6w19) and DRB1*0701 (DR7). The problem of beta chain specificity for DRB1*1501 (DR2w2beta-1), DRB5*0101 (DR2w2beta-2), DRB1*1601 (DR2w21beta-1), DRB5*0201 (DR51Dw21), and DRB4*0101 (DRw53) assays is circumvented by the use of fibroblasts. Development and validation of assays with regard to DRbeta molecule specificity have been described previously (see, e.g., Southwood et al., 1998). Table 14 contains the IC50 values.

### Example 5

### Use of Peptides to Evaluate Human Recall Responses for CD8 Epitopes

The peptide epitopes of the invention are used as reagents to evaluate T cell responses, such as acute or recall responses, in patients. Such an analysis may be performed on patients who have recovered from infection, who are chronically infected with HCV, or who have been vaccinated with an HCV vaccine.

For example, PBMC are collected from patients recovered from infection and HLA typed. Appropriate peptide epitopes of the invention that are preferably binding with strong or intermediate affinity (more preferably below the threshold affinity) are then used for analysis of samples derived from patients who bear that HLA type. PBMC from these patients are separated on density gradients and plated. PBMC are stimulated with peptide on different time points. Subsequently, the cultures are tested for cytotoxic activity.

Cytotoxicity assays are performed in the following manner. Target cells (either autologous or allogeneic EBV-transformed B-LCL that are established from human volunteers or patients; Current Protocols in Immunology, 1991) are incubated overnight with the synthetic peptide epitope, and labelled with ⁵¹Cr (Amersham Corp., Arlington Heights, Ill.) after which they are washed and radioactivity is counted. Percent cytotoxicity is determined from the formula: 100×[(experimental release-spontaneous release)/maximum release-spontaneous release)]. Maximum release is determined by lysis of targets.

The results of such an analysis indicate the extent to which HLA-restricted CTL populations have been stimulated by previous exposure to HCV or an HCV vaccine.

Alternatively, human in vitro CTL recall responses in chronic and resolved HCV patients towards HLA-restricted HCV-specific CTL-epitopes may be evaluated in the human IFNγ ELISPOT assay. As an example, in vitro recall responses of cells from HLA-A02 donors (homozygous or heterozygous) to a selected set of HLA-A02 restricted peptides are described. Basically, in vitro CTL recall responses are visualized in the IFN-gamma ELISPOT assay after overnight incubation of human PBMC with HLA-restricted peptides. The same has been done for HLA-A*01, HLA-B*08 and HLA-Cw04, Cw06 and Cw07.

Materials and Methods

Human PBMC

PBMC from healthy donors that are used to determine the cut off value for each individual peptide, are isolated according to the standard procedures.

PBMC from chronically infected HCV patients and (therapy) resolved HCV patients are used to determine the HCV-specific responses. All donors are HLA-A02 positive.

For use in the IFNγ ELISPOT assay, PBMC are thawed following standard procedures, washed twice with RPMI medium supplemented with 10% inactivate Fetal Calf Serum (iFCS) and counted with Trypan Blue in a Bürker Counting Chambre. Cells are resuspended in complete RPMI medium (=RPMI medium+NEAA+NaPy+Gentamycin+beta-MeOH) supplemented with 10% iFCS to the appropriate cell density.

HLA-A02 Restricted CTL Peptides

A selection of HLA-A02-restricted HCV peptides was made based on their affinity (IC50). The tested peptides are indicated in Table B. GILGFVFTL is a HLA-A02-restricted immunodominant Influenza-specific epitope that is used as a control peptide. All peptides are dissolved in 100% DMSO at 5 or 10 mg/ml and stored in aliquots at −20° C.

Shortly before use, peptides are further diluted in complete RPMI medium supplemented with 10% iFCS and used in the IFNγ ELISPOT assay at a final concentration of 10 μg/ml.

Cytokines

Lyophilized human IL-7 (R&D 207-IL) and human IL-15 (R&D 215-IL) is reconstituted in RPMI medium supplemented with 10% iFCS at 5 μg/ml and stored in aliquots at −70° C. Both cytokines are used in the IFNγ ELISPOT assay at final concentrations of 0.5 ng/ml per cytokine.

Human IFNγ ELISPOT

To pre-wet the membrane of the ELISPOT plates, 50 μl ethanol 99% p.a. is added to each well. After 10 minutes at room temperature, the ethanol is removed by washing all wells twice with purified water and once with PBS.

Pre-wetted 96-well ELISPOT plates are coated overnight with an anti-human IFNγ antibody (Mabtech Mab-1-D1K) and blocked for 2 hours with RPMI medium supplemented with 10% iFCS.

PBMC are resuspended in complete RPMI medium supplemented with 10% iFCS and seeded in triplicate in the coated ELISPOT plates at the required cell density between 3×10⁵ cells/well and 4×10⁵ cells/well. Cells are incubated with HLA-A02-restricted (CTL) peptides at 10 μg peptide/ml or with a polyclonal stimulus phytohemagglutinin (PHA) at 2 μg/ml as positive control, with and without cytokines.

After 23 hours incubation, all cells are lysed, washed away and the plates are further developed with biotinylated anti-human IFNγ antibody (Mabtech Mab 7-B6-1-bio) and streptavidin-HRP (BD 557630). Spots are visualized using AEC (BD 551951) as substrate. Rinsing the plates with tap water stops the color reaction. After drying the plates, the number of spots/well is determined using an A.EL.VIS ELISPOT reader. Every spot represents one IFNγ-producing CD8+ cell.

Method for Data-Analysis

A peptide is considered positive in human recall if at least one patient shows an active response (=response above cut-off level P80) to that peptide and whereby this active response is seen both with and without the addition of the cytokine coctail (IL-7+IL-15).

Cut-off values are determined by measuring the immune response in healthy individuals (n=20) and are based on statistical p80 and p90 values (=80%, resp. 90% of the back-ground immune responses are below this cut-off value after ranking the back-ground immune response for each individual peptide). Overall, higher cut-offs are measured after addition of cytokines.

Results

Table B contains the results for a set of HLA-A02 binding peptides. The result “+” is also indicated in Table 13.

TABLE B

| | # Subj | # Subj | # Subj | # Subj | | |
|---|---|---|---|---|---|---|
| | >P80 − | >P80 + | >P90 − | >P90 + | # | Immune |
| Sequence | Cyt | Cyt | Cyt | Cyt | Match | recall |
| SMVGNWAKV | 1 | 1 | 1 | 0 | 0 | |
| YLLPRRGPRL | 4 | 8 | 4 | 5 | 4 | + |
| DLMGYIPLV | 6 | 0 | 2 | 0 | 0 | |
| QIVGGVYLL | 0 | 0 | 0 | 0 | 0 | |
| YIPLVGAPL | 1 | 0 | 1 | 0 | 0 | |
| NLPGCSFSI | 3 | 0 | 3 | 0 | 0 | |
| FLLALLSCL | 4 | 0 | 1 | 0 | 0 | |
| LLSCLTIPA | 4 | 2 | 2 | 2 | 0 | |
| WLGNIIMYA | 2 | 1 | 1 | 1 | 0 | |
| YLVAYQATV | 1 | 0 | 0 | 0 | 0 | |
| LTHIDAHFL | 3 | 1 | 2 | 0 | 1 | + |
| ALYDVVSTL | 0 | 0 | 0 | 1 | 0 | |
| GMFDSSVLC | 2 | 0 | 2 | 0 | 0 | |
| KVLVLNPSV | 1 | 1 | 0 | 0 | 0 | |
| YLNTPGLPV | 0 | 2 | 0 | 2 | 0 | |
| KLQDCTMLV | 0 | 2 | 0 | 1 | 0 | |
| SVFTGLTHI | 2 | 0 | 1 | 0 | 0 | |
| TLHGPTPLL | 0 | 0 | 0 | 0 | 0 | |
| YQATVCARA | 1 | 1 | 0 | 0 | 0 | |
| IMYAPTLWA | 0 | 1 | 0 | 1 | 0 | |
| NIIMYAPTL | 1 | 4 | 1 | 3 | 1 | + |
| IMACMSADL | 0 | 5 | 0 | 0 | 0 | |
| TLWARMILM | 2 | 1 | 2 | 1 | 1 | + |
| QMWKCLIRL | 0 | 0 | 0 | 0 | 0 | |
| RLGAVQNEV | 3 | 3 | 1 | 3 | 1 | + |
| LLGCIITSL | 0 | 0 | 0 | 0 | 0 | |
| HMWNFISGI | 4 | 5 | 0 | 1 | 3 | + |
| CLVDYPYRL | 2 | 1 | 1 | 1 | 0 | |
| VLVGGVLAA | 3 | 7 | 3 | 2 | 3 | + |
| YLFNWAVRT | 0 | 3 | 0 | 0 | 0 | |
| GLLGCIITSL | 3 | 2 | 3 | 0 | 2 | + |
| VLVGGVLAAL | 2 | 7 | 2 | 5 | 2 | + |
| IMAKNEVFCV | 1 | 2 | 0 | 1 | 0 | |
| RLIVFPDLGV | 2 | 5 | 1 | 3 | 2 | + |
| LLFLLLADA | 2 | 2 | 1 | 2 | 0 | |
| FLLALLSCLT | 5 | 5 | 4 | 3 | 1 | + |

The class II restricted HTL responses may also be analyzed in a comparable way.

### Example 6

### Activity of CTL Epitopes in Transgenic (Tg) or Surrogate Mice

This example illustrates the induction of CTLs in transgenic mice by use of one ore more HCV CTL eitopes. The epitope composition can comprise any combination of CTL epitopes as described in the current invention, and more specific as given in Table 13. Similarly, a surrogate mouse can be used when no transgenic animals are available. Surrogate mice are non-transgenic animals that express MHC molecules resembling specific human HLA molecules and as such are useful for the evaluation of human CTL and/or HTL epitopes. Examples of surrogate mice are: CB6F1 for HLA-A24, CBA for HLA-B44, PLJ for HLA-A01 and Balb/c for HLA-DR.

HLA-B07 and B35 Epitopes

For this specific example, the experiment is performed to evaluate the immunogenicity of the peptides with Ki <1000 nM disclosed in Table 13, section B07 and B35.

The HLA-B7 restricted CTL response induced by peptides which bind to B7 or B35 emulsified in IFA in HLA-B7 Tg mice (F1, crossed with Balb/c) is evaluated. As a comparison, a group of naïve mice were included. The magnitude of CTL responses to the HLA-B7 and -B35 restricted epitopes in immunized HLA-B7/K^{b} transgenic mice are compared to the response in naïve animals.

Experimental Set-Up

HLA-B7/K^{b} transgenic mice (BALB/c×HLA-B7/K^{b}.C57BL/6 F1 mice; H₂^{bxd}), both male and female, were utilized. Mice were used between 8 and 14 weeks of age. Each group consisted of 3 mice and the naïve group consisted of 4 mice. Each set up was repeated in two independent experiments.

The immunization and testing scheme is shown in Table 17. In general, HLA-B7/K^{b} mice were immunized with a pool of B7-restricted CTL peptides emulsified in Incomplete Freund's Adjuvant (IFA). Nine peptide pools, each consisting of 4 to 6 CTL peptides, of similar binding affinity at a dose of 25 μg/peptide and 120 μg of the HTL epitope, HBV Core 128 (TPPAYRPPNAPIL) (known HTL epitope in these animals), were tested. Each experiment tested three of the pools, and each pool was tested in two independent experiments. Naïve animals (non-immunized HLA-B7/K^{b} transgenic mice) were included in each experiment as a control group. The mice were immunized with 100 μl of the emulsion sub-cutaneously at the base of the tail. Eleven to 14 days after immunization, the mice were euthanized, and the spleens were removed.

TABLE 17
Immunization and testing schedule for peptide
immunogenicity experiments using experiment 6 as an example.

In vivo
11-14 days

| Group | Week −2 | Week −1 | In vitro |
|---|---|---|---|
| 1 | Peptide Pool | | ELISPOT |
| 2 | Peptide Pool | | Assay |
| 3 | Peptide Pool | | |
| 4 | Näive | | |

Spleens were disrupted with a 15-ml tissue grinder and the resulting single cell suspension was treated with DNAse solution (10 μl/spleen of 30 mg/ml DNAse in PBS), washed in RPMI-1640 with 2% FCS, and counted. Splenocytes were then incubated at 4° C. for 15-20 minutes in 300 μl MACS buffer (PBS with 0.5% BSA and 2 mM EDTA) with 35 μl of MACS CD8a(Ly-2) Microbeads/10⁸ cells according to the manufacturer's specifications. The cells were then applied to a MACS column (Milltenyi) and washed four times. The cells were removed from the column in culture medium consisting of RPMI 1640 medium with HEPES (Gibco Life Technologies) supplemented with 10% FBS, 4 mM L-glutamine, 50 μM 2-ME, 0,5 mM sodium pyruvate, 100 μg/ml streptomycin and 100 U/ml penicillin. (RPMI-10), washed, and counted again.

The responses to CTL epitopes were evaluated using an IFN-γ ELISPOT assay. Briefly, IP membrane-based 96-well plates (Millipore, Bedford Mass.) were coated overnight at 4° C. with α-mouse IFN-γ monoclonal antibody (Mabtech MabAN18) at a concentration of 10 μg/ml in PBS. After washing 3 times with PBS, RPMI-10 was added to each well, and the plates were incubated at 37° C. for 1 hour to block the plates. The purified CD8+ cells were applied to the blocked membrane plates at a cell concentration of 4×10⁵ cells/well.

The peptides were dissolved in RPMI-10 (final peptide concentration 10 μg/ml), and mixed with target cells (10⁵ HLA-B7/K^{b} transfected Jurkat cells/well). Controls of media only and Con A (10 μg/ml) were also utilized. The target cell/peptide mixture was layered over the effector cells in the membrane plates, which were incubated for 20 hours at 37° C. with 5% CO₂.

Media and cells were then washed off the ELISPOT plates with PBS+0,05% Tween-20, and the plates were incubated with α-mouse biotinylated α-IFN-γ antibody (Mabtech MabR4-6A2-Biotin) at a final concentration of 1 μg/ml for 4 hours at 37° C. After washing, the plates were incubated with Avidin-Peroxidase Complex (Vectastain), prepared according to the manufacturer's instructions, and incubated at room temperature for 1 hour. Finally, the plates were developed with AEC (1 tablet 3-Amino-9-ethylcarbazole dissolved in 2,5 ml dimethylformamid, and adjusted to 50 ml with acetate buffer; 25 μl of 30% H₂O₂ was added to the AEC solution), washed, and dried. Spots were counted using AID plate reader.

Data-Analysis

Each peptide was tested for recognition in both the immunized group and the naïve group. Data was collected in triplicate for each experimental condition.

The raw data for the media control were averaged for each group (both naïve and immunized). Net spots were calculated by subtracting the average media control for each group from the raw data points within the group. The average and standard error were then calculated for each peptide, and the average and standard error were normalized to 10⁶ cells (by multiplying by a factor of 2,5). Finally, a type 1, one-tailed T test was performed to compare the data from immunized groups to that from naïve controls. Data was considered to be significantly different from the naïve controls if p≦0,1. The data are reported as the number of peptide-specific IFNγ producing cells/10⁶ CD8+ cells.

Data from two replicate experiments are compared. Peptides with discordant data (i.e. positive in one experiment and negative in the other) are repeated in a third experiment. The data from two or more experiments may be averaged as described above.

Peptide Immunogenicity Results for B7 and B35-Restricted Peptides.

The data are shown in Tables 18 (B7) and 19 (B35), and represent responses in 2-4 independent experiments. Twenty-six peptides showed a positive response when compared with the response in naïve mice (p≦0,1).

Ten of the peptides that were tested bound both B7 and B35 (6 peptides) or B35 only (4 peptides). Of the 6 peptides that bound both B7 and B35, four were immunogenic in the HLA-B7/K^{b} transgenic mice (Table 2). The 4 peptides that bound B35 only were all negative in the B7 transgenic mice.

TABLE 18

Immunogenicity data for HCV-derived peptides binding to
HLA-B7.
The peptides are sorted by peptide position, and the data are
reported in IFN-γ SFC/10⁶ CD8⁺ splenocytes. Responses that are
significant (p ≦ 0.1) are bolded. These are indicated in Table
13 as “+”.

| | |
|---|---|
| Immunized | Naïve |

| | | | | | |
|---|---|---|---|---|---|
| nM IC₅₀ | SFC/ | St | SFC/ | St | # of |

| | | | | | | | | | | |
|---|---|---|---|---|---|---|---|---|---|---|
| Sequence | B*0702 | B*3501 | 10⁶ | ± | Error | 10⁶ | ± | Error | Ttest | Exp. |

| | | | | | | | | | | |
|---|---|---|---|---|---|---|---|---|---|---|
| LPRRGPRLG | 124 | — | 138.1 | ± | 25.1 | 8.1 | ± | 5.9 | 0.00 | 4 |
| LPRRGPRLGV | 2.6 | — | 499.2 | ± | 28.5 | 11.3 | ± | 1.6 | 0.00 | 2 |
| GPRLGVRAT | 128 | — | 161.3 | ± | 71.9 | 8.8 | ± | 7.5 | 0.03 | 2 |
| QPRGRRQPI | 1.2 | — | 96.7 | ± | 20.3 | 2.1 | ± | 1.2 | 0.00 | 2 |
| SPRGSRPSW | 11 | — | 266.3 | ± | 9.5 | 2.9 | ± | 1.3 | 0.00 | 2 |
| DPRRRSRNL | 18 | — | 16.5 | ± | 8.1 | 3.5 | ± | 7.9 | 0.10 | 4 |
| IPLVGAPL | 25 | 295 | 206.7 | ± | 39.1 | 2.1 | ± | 2.6 | 0.00 | 2 |
| APLGGAARA | 115 | — | 10.4 | ± | 4.5 | 2.9 | ± | 2.8 | 0.11 | 2 |
| APLGGAARAL | 0.80 | 1048 | 116.3 | ± | 26.4 | 4.2 | ± | 2.8 | 0.00 | 2 |
| LPGCSFSIF | 29 | 90 | 15.4 | ± | 5.0 | 2.1 | ± | 0.8 | 0.02 | 2 |
| LPALSTGLI | 233 | — | 82.9 | ± | 21.3 | 3.3 | ± | 3.5 | 0.00 | 2 |
| TPCTCGSSDL | 168 | 7976 | 7.5 | ± | 7.4 | 7.9 | ± | 5.4 | 0.46 | 4 |
| APTGSGKST | 370 | — | 4.6 | ± | 2.6 | 9.2 | ± | 6.0 | 0.20 | 2 |
| YAAQGYKVL | 313 | 5836 | 68.3 | ± | 29.1 | 1.3 | ± | 5.7 | 0.03 | 4 |
| HPNIEEVAL | 230 | 7.4 | 17.5 | ± | 5.2 | 3.3 | ± | 4.2 | 0.01 | 2 |
| AAKLSALGL | 277 | — | 15.0 | ± | 4.0 | 0.4 | ± | 3.1 | 0.00 | 2 |
| TPGERPSGM | 199 | — | 45.0 | ± | 22.0 | 7.5 | ± | 5.7 | 0.06 | 4 |
| RPSGMFDSSV | 14 | — | 104.2 | ± | 27.7 | 1.7 | ± | 7.5 | 0.00 | 4 |
| TPAETSVRL | 375 | 1643 | 10.8 | ± | 6.7 | 7.5 | ± | 4.4 | 0.17 | 2 |
| APPPSWDQM | 281 | 17.771 | 489.6 | ± | 15.8 | 4.6 | ± | 3.3 | 0.00 | 2 |
| KPTLHGPTPL | 5.8 | 14.102 | 291.3 | ± | 67.4 | 7.1 | ± | 3.4 | 0.00 | 2 |
| GPTPLLYRL | 209 | 17.916 | 59.6 | ± | 6.3 | 7.1 | ± | 5.9 | 0.00 | 2 |
| TPLLYRLGA | 74 | — | 1.5 | ± | 6.9 | 6.9 | ± | 7.8 | 0.19 | 4 |
| LPGNPAIASL | 266 | 3539 | 17.1 | ± | 4.4 | 9.2 | ± | 6.1 | 0.22 | 2 |
| NPAIASLMAF | 121 | 312 | 5.4 | ± | 1.7 | 4.6 | ± | 3.0 | 0.34 | 2 |
| LPAILSPGAL | 255 | 550 | 14.6 | ± | 3.9 | 3.3 | ± | 4.1 | 0.04 | 2 |
| EPDVAVLTSM | 454 | 150 | 8.8 | ± | 3.6 | 6.3 | ± | 5.7 | 0.32 | 2 |
| RPDYNPPLL | 143 | — | 163.3 | ± | 47.2 | 6.7 | ± | 7.3 | 0.01 | 2 |
| PPVVHGCPL | 433 | — | 30.8 | ± | 9.6 | 7.9 | ± | 5.9 | 0.07 | 2 |
| LPINALSNSL | 12 | 137 | 223.8 | ± | 50.3 | 1.7 | ± | 1.8 | 0.00 | 2 |
| SPGQRVEFL | 38 | — | 12.7 | ± | 8.3 | 11.5 | ± | 6.8 | 0.43 | 4 |
| SAACRAAKL | 106 | — | 286.3 | ± | 32.4 | 8.8 | ± | 4.4 | 0.00 | 2 |
| APTLWARMI | 11 | — | 302.1 | ± | 48.8 | 25.0 | ± | 5.4 | 0.00 | 4 |
| APTLWARMIL | 1.2 | — | 859.2 | ± | 25.5 | 5.0 | ± | 3.8 | 0.00 | 2 |

TABLE 19

Immunogenicity data for HCV-derived peptides binding to
HLA-B35.
The peptides are sorted by peptide position, and the data
are reported in IFN-γ SFC/10⁶ CD8⁺ splenocytes. Responses
that are significant (p ≦ 0.1) are bolded. These are in-
dicated in Table 13 as “+”.

| | |
|---|---|
| Immunized | Naïve |

| | | | | | |
|---|---|---|---|---|---|
| nM IC₅₀ | SFC/ | St | SFC/ | St | # of |

| | | | | | | | | | | |
|---|---|---|---|---|---|---|---|---|---|---|
| Sequence | B*0702 | B*3501 | 10⁶ | ± | Error | 10⁶ | ± | Error | Ttest | Exp |

| | | | | | | | | | | |
|---|---|---|---|---|---|---|---|---|---|---|
| IPLVGAPL | 25 | 295 | 206.7 | ± | 39.1 | 2.1 | ± | 2.6 | 0.00 | 2 |
| LPGCSFSIF | 29 | 90 | 15.4 | ± | 5.0 | 2.1 | ± | 0.8 | 0.02 | 2 |
| HPNIEEVAL | 230 | 7.4 | 17.5 | ± | 5.2 | 3.3 | ± | 4.2 | 0.01 | 2 |
| IPTSGDVVV | 3152 | 380 | 7.5 | ± | 3.8 | 14.2 | ± | 3.6 | 0.18 | 2 |
| LPVCQDHLEF | 1564 | 104 | 13.3 | ± | 5.2 | 2.5 | ± | 3.1 | 0.08 | 2 |
| FPYLVAYQA | 1336 | 18 | −0.8 | ± | 4.9 | 4.6 | ± | 3.9 | 0.20 | 2 |
| NPAIASLMAF | 121 | 312 | 5.4 | ± | 1.7 | 4.6 | ± | 3.0 | 0.34 | 2 |
| EPEPDVAVL | — | 194 | 8.3 | ± | 4.9 | 8.8 | ± | 6.3 | 0.47 | 2 |
| EPDVAVLTSM | 454 | 150 | 8.8 | ± | 3.6 | 6.3 | ± | 5.7 | 0.32 | 2 |
| LPINALSNSL | 12 | 137 | 223.8 | ± | 50.3 | 1.7 | ± | 1.8 | 0.00 | 2 |

HLA-A01, A02, A03/A11, A24 and B44 Epitopes

Comparable experiments in the respective Tg or surrogate animals were performed for all the peptides with Ki <1000 nM disclosed in Table 13. The results are indicated in Tables 20-25.

TABLE 20

Immunogenicity data for HCV-derived peptides binding to
HLA-A01 in surrogate mice (PLJ)

| | |
|---|---|
| Immu- | |
| nized | Naïve |

| | | | | | | | | | |
|---|---|---|---|---|---|---|---|---|---|
| | IC₅₀ nM | SFC/ | | St | SFC/ | | St | | # of |
| Sequence | A*0101 | 10⁶ | ± | Error | 10⁶ | ± | Error | Ttest | Exp. |

| | | | | | | | | | |
|---|---|---|---|---|---|---|---|---|---|
| VIDTLTCGFA | 38 | −5.0 | ± | 10.0 | 8.8 | ± | 10.3 | 0.06 | 2 |
| RSELSPLLL | 106 | −5.0 | ± | 8.2 | −3.8 | ± | 9.5 | 0.41 | 2 |
| CTCGSSDLY | 14 | 4.2 | ± | 7.1 | −7.9 | ± | 1.5 | 0.07 | 2 |
| FTDNSSPPA | 10 | −4.2 | ± | 9.9 | 0.8 | ± | 4.4 | 0.26 | 2 |
| FTDNSSPPAV | 45 | 5.8 | ± | 12.9 | −12.1 | ± | 2.2 | 0.10 | 2 |
| VAATLGFGAY | 48 | 477.9 | ± | 30.2 | 4.2 | ± | 6.8 | 0.00 | 2 |
| AATLGFGAY | 694 | 725.8 | ± | 105.8 | 17.1 | ± | 6.7 | 0.00 | 2 |
| ITIGAPITY | 910 | 13.3 | ± | 9.4 | 7.9 | ± | 8.0 | 0.24 | 2 |
| VATDALMTGY | 452 | 13.3 | ± | 18.0 | −3.3 | ± | 8.7 | 0.17 | 2 |
| ATDALMTGY | 4.0 | 0.4 | ± | 8.6 | −6.3 | ± | 7.2 | 0.16 | 2 |
| ATDALMTGYT | 227 | −20.8 | ± | 11.7 | 9.6 | ± | 8.6 | 0.00 | 2 |
| DSSVLCECY | 719 | 17.5 | ± | 14.7 | 10.0 | ± | 3.6 | 0.27 | 2 |
| TLHGPTPLLY | 343 | 260.0 | ± | 33.7 | 22.5 | ± | 10.6 | 0.00 | 2 |
| LVDILAGYGA | 98 | 81.3 | ± | 31.1 | 20.8 | ± | 6.5 | 0.03 | 2 |
| LTDPSHITA | 15 | −8.3 | ± | 6.3 | −2.5 | ± | 7.7 | 0.26 | 2 |
| LTDPSHITAE | 237 | 7.5 | ± | 5.7 | 12.9 | ± | 8.5 | 0.26 | 2 |
| HSAKSKFGY | 615 | 37.1 | ± | 6.0 | 4.2 | ± | 6.4 | 0.00 | 2 |
| TSCGNTLTCY | 246 | −3.8 | ± | 6.9 | −7.5 | ± | 7.9 | 0.27 | 2 |
| FTEAMTRYSA | 464 | 15.4 | ± | 14.4 | 5.4 | ± | 3.8 | 0.28 | 2 |
| LSAFSLHSY | 28 | 387.9 | ± | 15.9 | −1.7 | ± | 7.0 | 0.00 | 2 |

TABLE 21

Immunogenicity data for HCV-derived peptides binding to
HLA-A02 in HLA-A02 Tg mice

| | |
|---|---|
| Immunized | Naïve |

| | | | | | |
|---|---|---|---|---|---|
| nM IC50 | SFC/ | St | SFC/ | St | # of |

| | | | | | | | | | |
|---|---|---|---|---|---|---|---|---|---|
| Sequence | A*0201 | 106 | ± | Error | 106 | ± | Error | Ttest | Exp. |

| | | | | | | | | | |
|---|---|---|---|---|---|---|---|---|---|
| QIVGGVYLL | 228 | 3.8 | ± | 1.4 | 0.0 | ± | 0.4 | 0.01 | 2 |
| YLLPRRGPRL | 140 | 73.8 | ± | 27.6 | 0.4 | ± | 0.5 | 0.02 | 2 |
| DLMGYIPLV | 83 | 3.8 | ± | 1.8 | 0.8 | ± | 0.6 | 0.07 | 2 |
| YIPLVGAPL | 337 | 19.2 | ± | 6.7 | 3.9 | ± | 3.4 | 0.01 | 3 |
| NLPGCSFSI | 83 | 0.8 | ± | 1.7 | 0.0 | ± | 0.6 | 0.30 | 2 |
| FLLALLSCLT | 132 | −0.8 | ± | 0.0 | 0.0 | ± | 0.9 | 0.19 | 2 |
| FLLALLSCL | 136 | 270.3 | ± | 72.9 | −3.1 | ± | 1.8 | 0.00 | 3 |
| LLSCLTIPA | 12 | −4.2 | ± | 4.4 | −1.4 | ± | 2.6 | 0.10 | 3 |
| SMVGNWAKV | 158 | 30.0 | ± | 2.4 | 2.1 | ± | 1.1 | 0.00 | 2 |
| CLVDYPYRL | 437 | 271.4 | ± | 87.5 | −0.6 | ± | 2.2 | 0.01 | 3 |
| ALSTGLIHL | 329 | 1.3 | ± | 0.8 | 0.8 | ± | 0.6 | 0.35 | 2 |
| LLFLLLADA | 16 | 3.3 | ± | 5.9 | −0.6 | ± | 2.4 | 0.18 | 3 |
| FLLLADARV | 20 | 25.8 | ± | 7.1 | 0.0 | ± | 0.4 | 0.01 | 2 |
| GLLGCIITSL | 26 | 241.4 | ± | 66.5 | −2.2 | ± | 2.2 | 0.00 | 3 |
| LLGCIITSL | 56 | −3.3 | ± | 2.4 | 2.2 | ± | 3.3 | 0.05 | 3 |
| YLVTRHADV | 292 | 34.2 | ± | 2.7 | 0.8 | ± | 0.6 | 0.00 | 2 |
| KVLVLNPSV | 50 | 10.4 | ± | 5.6 | −2.1 | ± | 3.1 | 0.04 | 2 |
| GMFDSSVLC | 114 | 211.9 | ± | 95.1 | −2.8 | ± | 1.3 | 0.03 | 3 |
| YLNTPGLPV | 6.2 | 419.4 | ± | 102.3 | 0.8 | ± | 3.0 | 0.00 | 3 |
| SVFTGLIHI | 101 | 674.2 | ± | 161.2 | −4.2 | ± | 3.7 | 0.00 | 2 |
| LTHIDAHFL | 1937 | −3.3 | ± | 2.9 | 0.3 | ± | 2.3 | 0.00 | 3 |
| YLVAYQATV | 29 | 22.5 | ± | 5.8 | 0.8 | ± | 0.9 | 0.01 | 2 |
| YQATVCARA | 20 | 187.1 | ± | 68.8 | −8.8 | ± | 1.4 | 0.02 | 2 |
| QMWKCLIRL | 153 | 418.1 | ± | 107.4 | −1.1 | ± | 2.4 | 0.00 | 3 |
| TLHGPTPLL | 68 | 99.4 | ± | 32.9 | −0.3 | ± | 2.8 | 0.01 | 3 |
| RLGAVQNEV | 221 | 96.9 | ± | 34.8 | 0.6 | ± | 3.5 | 0.01 | 3 |
| IMACMSADL | 66 | 38.1 | ± | 22.3 | 0.8 | ± | 3.4 | 0.07 | 3 |
| VLVGGVLAA | 219 | 7.9 | ± | 3.3 | 1.3 | ± | 2.0 | 0.10 | 2 |
| VLVGGVLAAL | 26 | 243.9 | ± | 65.3 | 1.9 | ± | 3.4 | 0.00 | 3 |
| HMWNFISGI | 12 | 374.2 | ± | 91.6 | −1.1 | ± | 3.2 | 0.00 | 3 |
| LLFNILGGWV | 4.1 | 17.9 | ± | 3.7 | 0.4 | ± | 0.5 | 0.00 | 2 |
| ILAGYGAGV | 88 | 5.4 | | 1.5 | 0.0 | ± | 0.4 | 0.01 | 2 |
| IMAKNEVFCV | 199 | 3.6 | ± | 4.0 | −0.6 | ± | 1.9 | 0.17 | 3 |
| RLIVFPDLGV | 89 | −1.9 | ± | 5.2 | 3.6 | ± | 3.5 | 0.02 | 3 |
| ALYDVVSTL | 19 | 88.6 | ± | 25.7 | −1.4 | ± | 2.4 | 0.00 | 3 |
| KLQDCTMLV | 4.6 | 218.1 | ± | 53.4 | −1.9 | ± | 2.5 | 0.00 | 3 |
| NIIMYAPTL | 70 | 335.8 | ± | 152.5 | −0.8 | ± | 3.3 | 0.03 | 3 |
| IMYAPTLWA | 46 | −0.8 | ± | 2.6 | 0.8 | ± | 3.2 | 0.24 | 3 |
| TLWARMILM | 11 | 180.0 | ± | 51.3 | 0.0 | ± | 0.4 | 0.01 | 2 |
| YLFNWAVRT | 29 | 196.1 | ± | 54.9 | −1.4 | ± | 2.3 | 0.00 | 3 |

TABLE 22

Immunogenicity data for HCV-derived peptides binding to
HLA-A03 and/or A11 in HLA-A11 Tg mice

| | |
|---|---|
| Immunized | Naïve |

| | | | | | |
|---|---|---|---|---|---|
| nM IC50 | SFC/ | St | SFC/ | St | # of |

| | | | | | | | | | | |
|---|---|---|---|---|---|---|---|---|---|---|
| Sequence | A0301 | A1101 | 106 | ± | Error | 106 | ± | Error | Ttest | Exp. |

| | | | | | | | | | | |
|---|---|---|---|---|---|---|---|---|---|---|
| STNPKPQRK | 7.2 | 14 | 428.8 | ± | 76.0 | 4.2 | ± | 6.3 | 0.00 | 2 |
| KTKRNTNRR | 283 | 646 | 6.7 | ± | 3.9 | 5.4 | ± | 4.6 | 0.43 | 2 |
| RLGVRATRK | 12 | 221 | 5.0 | ± | 8.2 | 2.1 | ± | 3.3 | 0.40 | 2 |
| KTSERSQPR | 41 | 147 | 1041.7 | ± | 170.9 | 6.7 | ± | 6.1 | 0.00 | 2 |
| QLFTFSPRR | 15 | 197 | 17.1 | ± | 6.3 | 3.3 | ± | 6.7 | 0.03 | 2 |
| WMNSTGFTK | 277 | 138 | 1.3 | ± | 2.47 | 0.4 | ± | 3.5 | 0.41 | 2 |
| RLLAPITAY | 4.6 | 222 | 4.2 | ± | 3.5 | 0.0 | ± | 3.0 | 0.23 | 2 |
| GIFRAAVCTR | 3382 | 129 | 0.0 | ± | 2.45 | 1.3 | ± | 3.6 | 0.32 | 2 |
| AVCTRGVAK | 136 | 48 | 437.9 | ± | 93.67 | 1.7 | ± | 4.8 | 0.00 | 2 |
| HLHAPTGSGK | 5.3 | 501 | 7.5 | ± | 4.9 | 5.0 | ± | 3.9 | 0.39 | 2 |
| AAYAAQGYK | 13 | 13 | 301.3 | ± | 36.7 | −0.4 | ± | 2.7 | 0.00 | 2 |
| TLGFGAYMSK | 134 | 44 | 10.8 | ± | 3.95 | 7.5 | ± | 4.5 | 0.15 | 2 |
| LGFGAYMSK | 113 | 22 | 0.8 | ± | 5.1 | −0.4 | ± | 4.2 | 0.41 | 2 |
| HLIFCHSKK | 30 | 1531 | −5.8 | ± | 6.6 | −1.3 | ± | 4.4 | 0.33 | 2 |
| LIFCHSKKK | 27 | 104 | 120.8 | ± | 41.70 | 7.5 | ± | 3.1 | 0.02 | 2 |
| GLNAVAYYR | 9.2 | 44 | 7.5 | ± | 2.27 | 7.5 | ± | 6.0 | 0.50 | 2 |
| KVLVDILAGY | 72 | 163 | 6.3 | ± | 4.1 | −3.8 | ± | 3.7 | 0.02 | 2 |
| GVVCAAILR | 5875 | 38 | 162.9 | ± | 26.7 | −2.5 | ± | 3.0 | 0.00 | 2 |
| GVVCAAILRR | 1066 | 215 | 598.8 | ± | 43.0 | 2.1 | ± | 6.0 | 0.00 | 2 |
| SQLSAPSLK | 81 | 14 | 4.2 | ± | 5.2 | 0.0 | ± | 4.2 | 0.16 | 2 |
| RVCEKMALY | 53 | 160 | 131.7 | ± | 32.8 | 3.3 | ± | 5.0 | 0.01 | 2 |
| LVNAWKSKK | 68 | 50 | 23.8 | ± | 5.90 | 2.5 | ± | 4.3 | 0.04 | 2 |
| GNTLTCYLK | 16.809 | 160 | 5.4 | ± | 4.6 | 5.0 | ± | 5.3 | 0.48 | 2 |
| ASAACRAAK | 51 | 15 | 223.8 | ± | 17.8 | 10.4 | ± | 8.2 | 0.00 | 2 |
| RVFTEAMTR | 45 | 21 | 173.3 | ± | 12.6 | 4.2 | ± | 3.9 | 0.00 | 2 |
| YLFNWAVRTK | 65 | 164 | 0.4 | ± | 6.2 | −0.4 | ± | 3.9 | 0.45 | 2 |

TABLE 23

Immunogenicity data for HCV-derived peptides binding to
HLA-B44 in surrogate mice (CBA)

| | |
|---|---|
| Immunized | Naïve |

| | | | | | |
|---|---|---|---|---|---|
| nM IC₅₀ | SFC/ | St | SFC/ | St | # of |

| | | | | | | | | | |
|---|---|---|---|---|---|---|---|---|---|
| Sequence | B*4402 | 10⁶ | ± | Error | 10⁶ | ± | Error | Ttest | Exp. |

| | | | | | | | | | |
|---|---|---|---|---|---|---|---|---|---|
| AEAALENLV | 126 | −5.4 | ± | 6.2 | −3.75 | ± | 4.1 | 0.39 | 2 |
| AETAGARLV | 176 | 1295.4 | ± | 114.1 | −6.3 | ± | 7.2 | 0.00 | 2 |
| AETAGARLV | 68 | 697.5 | ± | 36.8 | 27.5 | ± | 14.2 | 0.00 | 2 |
| GEIPFYGKAI | 354 | 799.6 | ± | 116.4 | 15.4 | ± | 8.0 | 0.00 | 2 |
| AEQFKQKAL | 67 | 7.9 | ± | 6.7 | 13.8 | ± | 5.5 | 0.29 | 2 |
| AEQFKQKAL | 201 | 10.0 | ± | 8.3 | 35.0 | ± | 16.8 | 0.06 | 2 |
| TEAMTRYSA | 2302 | 12.9 | ± | 20.8 | 10.8 | ± | 6.5 | 0.46 | 2 |
| RMILMTHFF | 389 | 11.3 | ± | 5.0 | −17.9 | ± | 3.8 | 0.00 | 2 |

TABLE 24

Immunogenicity data for HCV-derived peptides
binding to HLA-A24 in surrogate mice (Balb/c)

| | | | |
|---|---|---|---|
| Sequence | SFC/10⁶ | ± | SE |

| | | | |
|---|---|---|---|
| LLPRRGPRL | 64.2 | ± | 12.5 |
| YIPLVGAPL | 64.6 | ± | 10.7 |
| SFSIFLLAL | 185 | ± | 69.3 |
| PFYGKAIPI | 168.8 | ± | 60.1 |
| VIKGGRHLI | 191.3 | ± | 73.5 |
| YYRGLDVSVI | 41.7 | ± | 10.2 |
| FSLDPTFTI | 134.2 | ± | 19.2 |
| YLNTPGLPV | 544.2 | ± | 48.3 |
| CLIRLKPTL | 60 | ± | 28.2 |
| FWAKHMWNF | 45.4 | ± | 6.1 |
| FWAKHMWNFI | 293.3 | ± | 48.8 |
| QYLAGLSTL | 865.4 | ± | 183.5 |
| GFSYDTRCF | 56.3 | ± | 22.4 |
| RMILMTHFF | 128.3 | ± | 24.8 |

HLA-A24 Epitopes

In this experiment, a slightly different approach is used for the evaluation of the immunogenicity of the HLA-A24 binding epitopes in that the analysis of the peptide responses is performed in individual mice. ELISPOT results are reported as number of peptide-specific IFN-gamma producing cells per million (CD8 selected) spleen cells per mouse and the average delta values of triplicates (by subtracting the negative control conditions without stimulus) of the responses in the reacting animals are calculated. A peptide is considered to be immunogenic in the mouse model if at least one animal shows a significant positive response to that peptide.

TABLE 25

Immunogenicity data for HCV-derived peptides
binding to HLA-A24 in HLA A24 Tg mice

ELISPOT

| | | | | |
|---|---|---|---|---|
| | | | average pos. | |
| | | | result | Immun |
| Sequence | # subjects | # pos | (SFC/10⁶) | mice |

| | | | | |
|---|---|---|---|---|
| MYTNVDQDL | 5 | 5 | 659 | + |
| SFSIFLLAL | 4 | 4 | 150 | + |
| LLPRRGPRL | 5 | 5 | 63 | + |
| RMILMTHFF | 5 | 4 | 169 | + |
| CLIRLKPTL | 5 | 2 | 121 | + |
| FWAKHMWNF | 5 | 2 | 244 | + |
| TLHGPTPLL | 5 | 4 | 73 | + |
| RVEFLVNAW | 5 | 4 | 70 | + |
| QYLAGLSTL | 5 | 1 | 243 | + |
| LWARMILMTHF | 5 | 3 | 68 | + |
| VIKGGRHLI | 4 | 1 | 276 | + |
| AVMGSSYGF | 5 | 1 | 240 | + |
| IIMYAPTLW | 5 | 3 | 48 | + |
| GLGWAGWLL | 2 | 4 | 47 | + |
| YLNTPGLPV | 5 | 2 | 40 | + |
| ETTMRSPVF | 5 | 2 | 36 | + |
| NIIMYAPTL | 5 | 2 | 35 | + |
| TYSTYGKF | 5 | 1 | 53 | + |
| FWAKHMWNFI | 5 | 1 | 49 | + |
| NLPGCSFSI | 5 | 1 | 42 | + |
| VMGSSYGF | 5 | 1 | 36 | + |
| QYSPGQRVEF | 5 | 1 | 33 | + |
| LTHPITKYI | 5 | 1 | 31 | + |
| YYRGLDVSVI | 5 | 0 | neg | 0 |
| GLTHIDAHF | 5 | 0 | neg | 0 |
| FWESVFTGL | 5 | 0 | neg | 0 |
| AYMSKAHGV | 5 | 0 | neg | 0 |
| YYRGLDVSV | 5 | 0 | neg | 0 |
| GFSYDTRCF | 5 | 0 | neg | 0 |
| AYAAQGYKV | 5 | 0 | neg | 0 |
| NLGKVIDTL | 5 | 0 | neg | 0 |
| KFPGGGQIV | 5 | 0 | neg | 0 |
| QWMNRLIAF | 5 | 0 | neg | 0 |
| MYVGGVEHRL | 5 | 0 | neg | 0 |
| NFISGIQYL | 5 | 0 | neg | 0 |
| AIKGGRHLI | 5 | 0 | neg | 0 |
| ALYDWSTL | 5 | 0 | neg | 0 |
| QMWKCLIRL | 5 | 0 | neg | 0 |
| FSLDPTFTI | 4 | 0 | neg | 0 |
| GFADLMGYI | 4 | 0 | neg | 0 |

### Example 7

### Activity of HTL Epitopes in Transgenic (Tg) and Surrogate Mice

The experiments to test the immunogenicity of HLA-DR peptides differs slightly from example 6 in that complete Freund's is used as the adjuvant. Peptides are tested in either DRB1*0401-Tg mice or surrogate mice such as Balb/c and CBA. In this particular example, HLA-restricted peptide responses are analyzed in pooled samples.

The data for the DR4 transgenic mice are shown in table 26 and represent responses in 2 independent experiments. Seventeen of the peptides gave positive responses (defined as >10 SFC/10⁶ CD4+ cells and p≦0.05) in these mice.

The data for the H2^{bxd} background (Balb/c) are shown in table 27 and represent responses in 2 independent experiments. Seven of the peptides give positive responses (defined as >10 SFC/10⁶ CD4+ cells and p≦0.05) in these mice.

The data for the CBA mice (H2^{k}) are shown in table 28 and represent responses in 2 independent experiments. Twelve of the peptides give positive responses (defined as >10 SFC/10⁶ CD4+ cells and p≦0.05) in these mice.

TABLE 26

Immunogenicity in DR4 Tg mice

| | |
|---|---|
| Immunized | Naïve |

| | | | | | | | | |
|---|---|---|---|---|---|---|---|---|
| | DRB1 | SFC/ | | St | SFC/ | | St | |
| Sequence | *0401 | 10⁶ | ± | Error | 10⁶ | ± | Error | Ttest |

| | | | | | | | | |
|---|---|---|---|---|---|---|---|---|
| GPRLGVRATRKTSER | — | 2.1 | ± | 3.0 | 3.3 | ± | 2.4 | 0.38 |
| RLGVRATRKTSERSQ | 5868 | 15.0 | ± | 4.9 | 0.0 | ± | 1.2 | 0.02 |
| GVRVLEDGVNYATGN | 132 | 147.1 | ± | 61.9 | 2.5 | ± | 1.3 | 0.03 |
| FTTLPALSTGLIHLH | 2080 | 142.9 | ± | 43.3 | 4.6 | ± | 2.4 | 0.01 |
| AVGIFRAAVCTRGVA | 31 | 631.3 | ± | 132.1 | 0.0 | ± | 0.5 | 0.00 |
| RSPVFTDNSSPPAVP | 10 | 423.3 | ± | 93.0 | 0.4 | ± | 1.0 | 0.00 |
| AQGYKVLVLNPSVAA | 1.6 | 69.2 | ± | 15.9 | −0.4 | ± | 1.0 | 0.00 |
| VLVLNPSVAATLGFG | 6.5 | 67.5 | ± | 11.9 | 2.5 | ± | 3.6 | 0.00 |
| YGKFLADGGCSGGAY | 21 | 989.6 | ± | 19.5 | 1.7 | ± | 1.2 | 0.00 |
| LVVLAIATPPGSVTV | 4.0 | 111.7 | ± | 42.3 | 2.5 | ± | 1.1 | 0.03 |
| HLIFCHSKKKCDELA | — | 22.1 | ± | 8.5 | 2.9 | ± | 1.5 | 0.04 |
| TVDFSLDPTFTIETT | 59 | 130.0 | ± | 36.9 | 5.8 | ± | 2.4 | 0.01 |
| KPTLHGPTPLLYRLG | 4861 | 23.3 | ± | 8.0 | 1.3 | ± | 1.1 | 0.01 |
| TWVLVGGVLAALAAY | 369 | 623.3 | ± | 98.9 | −0.8 | ± | 0.5 | 0.00 |
| IQYLAGLSTLPGNPA | 2.6 | 1435.8 | ± | 111.5 | 2.9 | ± | 3.1 | 0.00 |
| VNLLPAILSPGALVV | 1558 | 613.3 | ± | 59.4 | −0.8 | ± | 1.6 | 0.00 |
| AVQWMNRLIAFASRG | 1009 | 1006.7 | ± | 70.1 | 4.2 | ± | 5.6 | 0.00 |
| MNRLIAFASRGNHVS | 813 | 1.3 | ± | 3.9 | 0.0 | ± | 1.4 | 0.40 |
| VFCVQPEKGGRKPAR | — | −0.4 | ± | 1.7 | 2.1 | ± | 1.1 | 0.09 |
| ARAAWETARHTPVNS | 14.766 | 2.9 | ± | 3.4 | 0.8 | ± | 0.9 | 0.28 |
| PTLWARMILMTHFFS | 178 | 1442.9 | ± | 107.2 | 2.5 | ± | 2.0 | 0.00 |

TABLE 27

immunogenicity in Balb/c (H2^{bxd})

| | |
|---|---|
| Immunized | Naïve |

| | | | | | | | |
|---|---|---|---|---|---|---|---|
| | SFC/ | | St | SFC/ | | St | T |
| Sequence | 10⁶ | ± | Error | 10⁶ | ± | Error | test |

| | | | | | | | |
|---|---|---|---|---|---|---|---|
| GPRLGVRATRKTSER | 0.8 | ± | 1.2 | −0.8 | ± | 0.0 | 0.12 |
| RLGVRATRKTSERSQ | 2.1 | ± | 2.1 | −0.4 | ± | 0.4 | 0.10 |
| GVRVLEDGVNYATGN | 1.3 | ± | 1.1 | −0.8 | ± | 0.4 | 0.02 |
| FTTLPALSTGLIHLH | 5.8 | ± | 2.2 | −0.4 | ± | 0.5 | 0.02 |
| AVGIFRAAVCIRGVA | 1330.4 | ± | 111.9 | 0.8 | ± | 0.5 | 0.00 |
| RSPVFTDNSSPPAVP | −5.0 | ± | 0.6 | 0.4 | ± | 0.4 | 0.00 |
| AQGYKVLVLNPSVAA | 68.8 | ± | 17.5 | −1.3 | ± | 0.0 | 0.01 |
| VLVLNPSVAATLGFG | 238.8 | ± | 84.6 | 0.8 | ± | 0.8 | 0.02 |
| YGKFLADGGCSGGAY | −1.7 | ± | 3.5 | 1.7 | ± | 0.8 | 0.15 |
| LVVLATATPPGSVTV | −5.8 | ± | 0.8 | 1.3 | ± | 0.9 | 0.00 |
| HLIFCHSKKKCDELA | 5.8 | ± | 3.8 | 0.4 | ± | 0.4 | 0.11 |
| TVDFSLDPTFTIETT | −0.4 | ± | 0.5 | −0.4 | ± | 0.5 | 0.50 |
| KPTLHGPTPLLYRLG | 43.3 | ± | 12.0 | −0.8 | ± | 0.4 | 0.01 |
| TWVLVGGVLAALAAY | 263.8 | ± | 35.0 | −0.8 | ± | 0.4 | 0.00 |
| IQYLAGLSTLPGNPA | 0.0 | ± | 0.5 | 0.0 | ± | 0.5 | 0.50 |
| VNLLPAILSPGALVV | 6.7 | ± | 2.4 | −0.4 | ± | 0.4 | 0.01 |
| AVQWMNRLIAFASRG | 286.3 | ± | 69.0 | −0.4 | ± | 0.4 | 0.00 |
| MNRLIAFASRGNHVS | 95.0 | ± | 31.9 | 0.4 | ± | 0.6 | 0.02 |
| VFCVQPEKGGRKPAR | 9.6 | ± | 6.8 | 1.3 | ± | 0.9 | 0.11 |
| ARAAWETARHTPVNS | 3.3 | ± | 2.4 | 0.4 | ± | 0.4 | 0.15 |
| PTLWARMILMTHFFS | 2.5 | ± | 1.5 | 0.8 | ± | 1.1 | 0.12 |

TABLE 28

immunogenicity in CBA (H2^{k}) mice

| | |
|---|---|
| Immunized | Naïve |

| | | | | | | | |
|---|---|---|---|---|---|---|---|
| | | | St | | | St | |
| | SFC/ | | Er- | SFC/ | | Er- | |
| Sequence | 10⁶ | ± | ror | 10⁶ | ± | ror | Ttest |

| | | | | | | | |
|---|---|---|---|---|---|---|---|
| GPRLGVRATRKTSER | 175.4 | ± | 27.9 | −4.6 | ± | 9.7 | 0.00 |
| RLGVRATRKTSERSQ | 189.6 | ± | 57.2 | 3.3 | ± | 12.8 | 0.02 |
| GVRVLEDGVNYATGN | −67.92 | ± | 63.7 | −20.00 | ± | 9.6 | 0.35 |
| FTTLPALSTGLIHLH | −106.67 | ± | 28.3 | −24.58 | ± | 4.9 | 0.03 |
| AVGIFRAAVCTRGVA | 148.3 | ± | 76.3 | 17.1 | ± | 17.5 | 0.06 |
| RSPVFTDNSSPPAVP | 90.8 | ± | 35.9 | −0.4 | ± | 10.7 | 0.02 |
| AQGYKVLVLNPSVAA | −90.83 | ± | 46.9 | −29.17 | ± | 6.3 | 0.11 |
| VLVLNPSVAATLGFG | −40.83 | ± | 24.9 | −28.33 | ± | 2.5 | 0.40 |
| YGKFLADGGCSGGAY | 138.3 | ± | 42.6 | 4.2 | ± | 16.4 | 0.02 |
| LVVLATATPPGSVTV | 27.9 | ± | 23.4 | 33.3 | ± | 39.6 | 0.44 |
| HLIFCHSKKKCDELA | 167.1 | ± | 37.9 | −9.2 | ± | 7.5 | 0.00 |
| TVDFSLDPTFTIETT | −95.00 | ± | 78.0 | −7.50 | ± | 25.7 | 0.32 |
| KPTLHGPTPLLYRLG | −52.50 | ± | 48.7 | −24.58 | ± | 8.6 | 0.48 |
| TWVLVGGVLAALAAY | 593.33 | ± | 26.5 | −32.08 | ± | 5.0 | 0.00 |
| IQYLAGLSTLPGNPA | −22.5 | ± | 10.5 | −0.4 | ± | 5.0 | 0.06 |
| VNLLPAILSPGALVV | 10.0 | ± | 37.6 | 33.3 | ± | 45.3 | 0.36 |
| AVQWMNRLIAFASRG | 450.0 | ± | 94.2 | 12.5 | ± | 17.6 | 0.00 |
| MNRLIAFASRGNHVS | 255.0 | ± | 27.9 | 25.0 | ± | 26.5 | 0.00 |
| VFCVQPEKGGRKPAR | 247.5 | ± | 59.4 | −7.1 | ± | 11.4 | 0.00 |
| ARAAWETARHTPVNS | 93.3 | ± | 30.8 | −8.3 | ± | 5.3 | 0.01 |
| PTLWARMILMTHFFS | 114.6 | ± | 43.6 | −11.3 | ± | 4.1 | 0.01 |

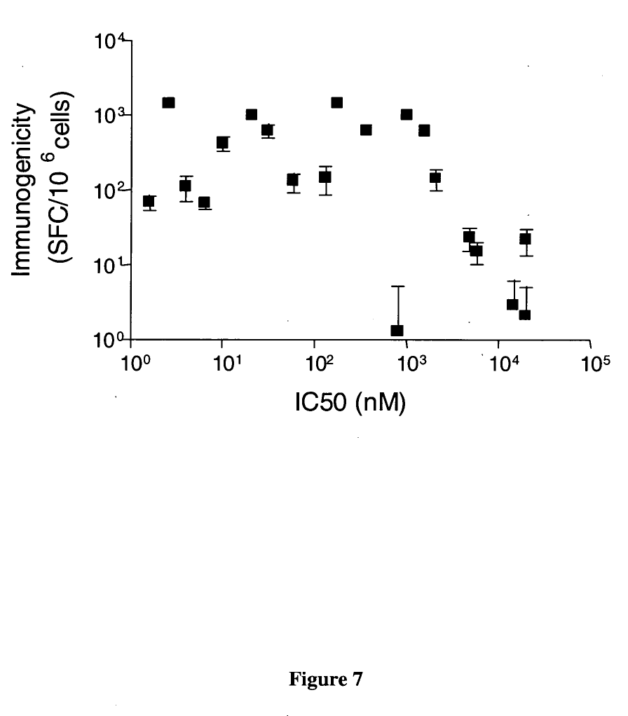

As shown in FIG. 7, a close relationship between binding and immunogenicity is detected. It can be concluded that all the peptides with binding affinity of less than 500 nM are immunogenic. Hence, the threshold affinity for DRB1 is 500 nM.

### Example 8

### Immunogenicity of CTL Epitopes Embedded in a Nested Epitope

This example illustrates the induction of CTL responses to a selection of epitopes embedded in a nested epitope, when injected into susceptible mice. Similar experiments can be performed to illustrate the induction of HTL responses to epitopes embedded in a nested epitope.

For this example, the A24 specific T cell responses in HLA A24 Tg mice injected with nested epitopes containing A24 restricted epitopes is measured. The magnitude of the CTL response to the individual HLA-A24 restricted epitopes is determined and compared with the response measured towards these epitopes in cells from mice immunized with a buffer/adjuvant (CFA) control. All HLA-A24 epitopes binding with an affinity (Ki) of less than 500 nM were tested.

The immunogenicity of epitopes embedded in these nested epitopes and restricted to other HLA-class I types can be evaluated in a comparable way in susceptible mice.

In Vivo Experimental Set-Up

Two groups of 5 mice (age 8 to 10 weeks, randomized females and males) are included of which animals from each group receive either a single injection with a nested epitope emulsified in CFA or—as a negative control—the buffer without peptide and emulsified in CFA. All injections were performed subcutaneously at the base of the tail. In this particular experiment, the nested epitope FWAKHMWNFISGIQYLAGLSTLPGNPA (SEQ ID NO 2278) was evaluated (table 29).

TABLE 29

nested epitope evaluated in A24 Tg mice

| | | Dose/ | |
|---|---|---|---|
| Mice | Sequence | adjuvant | group |
| HLA | FWAKHMWNFISGIQYLAGLSTLPGNPA | 50 μg/CFA | 05 |
| A24 Tg | | | 040/3 |
| HLA | PBS | −/CFA | 05 |
| A24 Tg | | | 040/5 |

In Vitro Experimental Set-Up

Spleen cells from all individual animals are isolated 11 to 14 days after injection. A direct ex vivo IFN-γ ELISPOT assay is used as a surrogate CTL readout. To this, CD8 spleen cells from each individual mouse are purified by positive magnetic bead selection on (part of) the spleen cells.
For the group 05 040/3, the response in the purified CD8 spleen cells (2.10⁵ cells/well) from each individual mouse is evaluated by presenting the HLA-A24-specific peptides (10 μg/ml) on antigen presenting cells expressing the HLA-A24/Kb molecule (10⁴ cells/well) and on gamma-irradiated syngeneic spleen cells (2.10⁵ cells/well). After loading, the excess of peptide is removed by washing.

For the group 05 040/5, the spleen cells from each mouse are pooled prior to CD8 purification. An IFN-γ ELISPOT using the same conditions as mentioned above is performed to determine the baseline response against all peptides tested.

TABLE 30

overview read out

| | HLA-A24 restricted CTL | |
|---|---|---|
| | epitopes tested for immune | |
| group | response | SEQ ID NO |
| 05 040/3 | FWAKHMWNF | 1095 |
| | FWAKHMWNFI | 1096 |
| | NFISGIQYL | 1521 |
| | QYLAGLSTL | 1625 |
| 05 040/5 | FWAKHMWNF | 1095 |
| | FWAKHMWNIFI | 1096 |
| | NFISGIQYL | 1521 |
| | QYLAGLSTL | 1625 |

Methods for Data-Analysis

ELISPOT results are reported as number of peptide-specific IFN-γ producing cells per million (CD8/CD4 selected) spleen cells per mouse or pooled group. Based on the average/median delta values of triplicates (by subtracting the negative control conditions without stimulus), a descriptive comparison between different groups/experimental set-ups for each epitope tested is made.

In addition, non-specific background responses in control-immunized mice are used as an additional negative control to determine the immunogenicity of the individual epitopes.

Acceptation Criteria

For the in vivo part of the experiment, all mice are evaluated (general welfare document) and weighted at the beginning and end of the study.

The acceptance of the in vitro-generated experimental results are based on well-documented viability and positive response after polyclonal stimulation of the cells. Results are shown for the 4 tested HLA-A24 epitopes in the individual mice.

TABLE 31

immunoreactivity of the embedded epitopes in
the 5 animals injected with the nested epitope

| | HLA-A24 restricted | | | | | |
|---|---|---|---|---|---|---|
| | CTL epitopes | Sub- | Sub- | Sub- | Sub- | Sub- |
| | tested for immune | ject | ject | ject | ject | ject |
| group | response | 1 | 2 | 3 | 4 | 5 |
| 05 040/3 | FWAKHMWNF | +++ | ++ | +++ | ++ | +++ |
| | FWAKHMWNH | +++ | ++ | +++ | ++ | +++ |
| | NFISGIQYL | ++ | | | | ++ |
| | QYLAGISTL | | | | | ++ |

+ 0-10 SFC/10⁶ CD8 cells
++ 10-100 SFC/10⁶ CD8 cells
+++ >100 SFC/10⁶ CD8 cells

### REFERENCES

Alexander, J. et al., J Immunol. 159:4753, 1997
Alexander J. et al., Hum Immunol 64(2): 211-223, 2003
Altman et al., Science 174:94-96, 1996
An, L. and Whitton, J. L., J. Virol. 71:2292, 1997
Arndt et al., Immuno.l Res., 16: 261-72, 1997
Barton, G. M. & Medzhitov, R. Toll-like receptors and their ligands. Curr. Top. Microbiol. Immunol. 270, 81-92, 2002
Bertoni, R. et al., J. Clin. Invest. 100:503, 1997
Beaucage & Caruthers, Tetrahedron Letts. 22:1859-1862, 1981
Blum et al., Grit. Rev. Imnunol., 17: 411-17, 1997
Brooks et al., J Immunol, 161: 5252-5259, 1998
Brown. J. et al., (1993) Nature 364, 33-39
Busch et al., Int. Immunol. 2:443, 1990
Buus et al., Science 242: 1065, 1988
Byl, B. et al. OM197-MP-AC induces the maturation of human dendritic cells and promotes a primary T cell response. Int Immunopharmacol. 3, 417-425, 2003
Celis, E. et al., Proc. Natl. Acad. Sci. USA 91:2105, 1994
Ceppellini et al., Nature 339:392, 1989
Cerundolo et al., J Immunol. 21:2069, 1991
Christnick et al., Nature 352:67, 1991
Collins et al., J. Immunol. 148:3336-3341, 1992
del Guercio et al., J Immunol. 154:685, 1995
Diepolder, H. M. et al., J. Virol. 71:6011, 1997
Donnelly J J, Ulmer J B, Shiver J W, Liu M A. DNA vaccines. Annu Rev Immunol. 1997;15:617-48.
Donnelly J J, Ulmer J B, Liu M A. DNA vaccines. Life Sci. 1997a;60(3):163-72.
Doolan, D. L. et al., Immunity 7:97, 1997
Falk, K. et al., (1991) Nature 351, 290-296)
Felgner, et al., Proc. Nat'l Acad. Sci. USA 84:7413, 1987
Fries et al., Proc. Natl. Acad. Sci. (USA) 89:358, 1992
Grakoui, A., Wychowski, C., Lin, C., Feinstone, S. M. & Rice, C. M. Expression and identification of hepatitis C virus polyprotein cleavage products. J. Virol. 67, 1385-1395, 1993
Gruters R A, van Baalen C A, Osterhaus A D. Vaccine. 2002 May 6;20(15):2011-5. The advantage of early recognition of HIV-infected cells by cytotoxic T-lymphocytes.
Hammer et al., J. Exp. Med. 180:2353, 1994
Hanke, R. et al., Vaccine 16:426, 1998
Henderson et al, Science 255: 1264, 1992
Hill et al., J. Immunol. 147:189, 1991
Hill et al., J. 1mmunol. 152, 2890, 1994
Hoffmann et al., Hepatology, 21(3):632-8, 1995
Hunt, et al., Science 225: 1261, 1992
Houssaint E, Saulquin X, Scotet E, Bonneville M Biomed Pharmacother. 2001 September; 55(7): 373-80. Immunodominant CD8 T cell response to Epstein—Barr virus.
Ishioka, et al., J. Immunol. (1999) 162(7):3915-3925
Jardetzky, et al., Nature 353: 326, 1991
Johnson, D. A. et al. Synthesis and biological evaluation of a new class of vaccine adjuvants: aminoalkyl glucosaminide 4-phosphates (AGPs). Bioorg. Med. Chem Lett 9, 2273-2278, 1999
Kessler et al. Human Immunol, 64: 245-255, 2003
Khilko et al., J. Biol. Chem. 268:15425, 1993
Kawashima, 1. et al., Human Immunol. 59:1, 1998
Knauf M J. et al, J Biol. Chem. Oct. 15;263(29):15064-70, 1988
Kriegler M. Gene transfer and expression: a laboratory manual. W.H. Freeman and Company, New York, 1991: 60-61 and 165-172.
Lamonaca et al., Hepatology, 30:1088-1098
Latron, F. et al., (1992) Science 257, 964-967
Lim, J. S. et al. (1996) Mol. Immunol. 33, 221-230
Lukacher A E, Braciale V L, Braciale T J. J Exp Med. 1984 Sep. 1;160(3):814-26. In vivo effector function of influenza virus-specific cytotoxic T lymphocyte clones is highly specific.
Ljunggren et al., Nature 346:476, 1990
Lauer, G. M. & Walker, B. D. Hepatitis C virus infection. N. Engl J. Med. 345, 41-52, 2001
Madden, D. R. et al., (1992) Cell 70, 1035-1048
Mannino & Gould-Fogerite, BioTechniques 6(7): 682, 1988
Marshall et al., J. Immunol. 152:4946, 1994
Matsumura, M. et al., (1992) Science 257, 927-934
Michelletti et al., Immunology, 96: 411-415, 1999
Murray N, McMichael A. Curr Opin Immunol. 1992 Aug.;4(4):401-7. Antigen presentation in virus infection.
Nabel et al., Proc. Natl. Acad. Sci. (USA) 89:5157, 1992
Niedermann et al., Immunity, 2: 289-99, 1995
Ogg et al., Science 279:2103-2106, 1998
Parker et al., J. Immunol. 149:1896, 1992
Pearson & Reanier, J. Chrom. 255:137-149, 1983
Perma et al., J. Exp. Med. 174:1565-1570, 1991
Persing, D. et al. Taking toll: lipid A mimetics as adjuvants and immunomodulators. Trends Microbiol. 10, S32, 2002
Reay et al., EMBO J. 11:2829, 1992
Rehermann, B. et al., J. Exp. Med. 181:1047, 1995
Reddy et al., J. Immunol. 148:1585, 1992
Riedl, P., Buschle, M., Reimann, J. & Schirmbeck, R. Binding immune-stimulating oligonucleotides to cationic peptides from viral core antigen enhances their potency as adjuvants. Eur. J. Immunol. 32, 1709-1716, 2002
Rognan, D. et al., (1999) J. Med. Chem. 42, 30 4650-4658
Rosenberg et al (1997), Science 278:1447-1450
Rotzschke and Falk, Immunol. Today 12: 447, 1991
Saper, M. A. et al., (1991) J. Mol. Biol. 219, 277-312
Schaeffer et al. Proc. Natl. Acad. Sci. USA 86:4649-4653, 1989
Schumacher et al., Cell 62:563, 1990
Sercarz, et al., Annu. Rev. Immunol 11: 729-766, 1993
Sette, et al, J Immunol 153:5586-5592, 1994
Sette, et al., Mol. Immunol. 31: 813 (1994).
Sette and Sidney (1990), Immunogenetics 50: 201-212
Sidney et al., Current Protocols in Immunology, Ed., John Wiley & Sons, NY, Section 18.3 (1998)
Sidney, et al., J. Immunol. 154: 247 (1995)
Shiffman, M. L. Improvement in liver histopathology associated with interferon therapy in patients with chronic hepatitis C. Viral Hepatitis Reviews 5, 27-43, 1999
Shimotohno, K. et al. Processing of the hepatitis C virus precursor protein. J. Hepatol. 22, 87-92, 1995
Southwood et al. J Immunology 160:3363-3373, 1998
Stemmer W P et al. Gene 164: 49-53, 1995
Stover et al., Nature 351:456-460, 1991
Szoka, et al., Ann. Rev. Biophys. Bioeng. 9:467, 1980
Threlkeld, S. C. et al., J. Immunol. 159:1648, 1997
Tigges M A, Koelle D, Hartog K, Sekulovich R E, Corey L, Burke R L J. Virol. 1992 March;66(3):1622-34. Human CD8+ herpes simplex virus-specific cytotoxic T-lymphocyte clones recognize diverse virion protein antigens.
Thomson, S. A. et al., J. Immunol. 157:822, 1996
Townsend et al., Cell 62:285, 1990
Tsai S L, Huang S N. J Gastroenterol Hepatol. 1997 Oct.;12(9-10):S227-35. T cell mechanisms in the immunopathogenesis of viral hepatitis B and C.
Tsai, V. et al., J. Immunol. 158:1796, 1997
Qi-Liang Cai et al. Vaccine 23: 267-277, 2004
van der Burg et al., J Immunol, 156: 3308-3314, 1996
Van Devanter et. al., Nucleic Acids Res. 12:6159-616S, 1984
Velders MP et al. J Immunol, 166(9): 5366-73, 2001
Yewdell et al., Aurz. Rev. Immunol., 17: 51-88, 1997
Walewski, J. L., Keller, T. R., Stump, D. D. & Branch, A. D. Evidence for a new hepatitis C virus antigen encoded in an overlapping reading frame. RNA. 7, 710-721, 2001
Wentworth, P. A. et al., Mol. Immunol. 32:603, 1995
Wentworth, P. A. et al., J. Immunol. 26:97, 1996
Wentworth, P. A. et al., Int. Immunol. 8:651, 1996
Wertheimer A M et al., Hepatology, 37: 577-589
Whitton, J. L. et al., J Prol. 67:348, 1993
Xu, Z. et al. Synthesis of a novel hepatitis C virus protein by ribosomal frameshift. EMBO J. 20, 3840-3848, 2001

1. An isolated polyepitopic peptide comprising at least two peptides derived from a HCV protein and capable of inducing a HLA class I and/or class II restricted T lymphocyte response, characterized in that at least one peptide is a HLA-C binding peptide.

2. The polyepitopic peptide according to claim 1 further characterized in that said at least two peptides are present in the HCV consensus sequence of genotype 1a, 1b and/or 3a.

3. The polyepitopic peptide according to claim 1, wherein the at least one HLA-C binding peptide is characterized in that it binds a HLA molecule, said molecule being selected from the HLA-C group HLA-Cw03, Cw04, Cw06 or Cw07.

4. The polyepitopic peptide according claim 1, wherein the at least two peptides consist of an HLA-C binding peptide and a peptide selected from the group consisting of:
a HLA-A binding peptide characterized in that it binds a HLA molecule, said molecule being selected from the HLA-A group HLA-A01, -A02, -A03, -A11 or -A24,
a HLA-B binding peptide characterized in that it binds a HLA molecule, said molecule being selected from the HLA-B group HLA-B07, -B08, -B35, -B40 or -B44,
a HLA-C binding peptide characterized in that it binds a HLA molecule, said molecule being selected from the HLA-C group HLA-Cw03, Cw04, Cw06 or Cw07, and
a HLA-DRB1 binding peptide characterized in that it binds a HLA molecule, said molecule being selected from the HLA-DRB1 group HLA-DRB1*01, -DRB1*03 or -DRB1*04.

5. The polyepitopic peptide according to claim 1, wherein the at least two peptides are selected from Tables 13 and/or 14.

6. The polyepitopic peptide according to claim 1, wherein the at least one HLA-C binding peptide is selected from the group consisting of: SEQ ID NO 1048, 1095, 1730, 349, 475, 111, 2066, 1511, 1454, 1100 and 907.

7. The polyepitopic peptide according to claim 6 further comprising a peptide selected from the group consisting of: SEQ ID NO 557, 1241, 1456, 1478, 1833, 1887, 67, 922, 66, 361, 1070, 1072, 1151, 71, 1233, 1269, 75, 73, 1396, 5, 87, 91, 238, 265, 1661, 1753, 76, 81, 92, 1933, 1934, 69, 2043, 2047, 74, 63, 2053, 83, 56, 155, 156, 1205, 1206, 167, 1350, 47, 146, 1609, 144, 3, 39, 158, 16, 122, 1034, 1095, 1096, 1150, 246, 1406, 23, 1483, 1512, 87, 93, 1625, 1626, 59, 1710, 250, 81, 1885, 1916, 1938, 2048, 271, 2083, 1, 877, 17, 7, 1086, 1087, 1468, 1700, 1894, 402, 836, 381, 371, 853, 370, 387, 307, 1237, 1289, 1343, 1418, 1419, 375, 1430, 380, 450, 1582, 390, 1677, 1687, 121, 386, 372, 95, 443, 396, 455, 1441, 436, 1719, 92, 394, 1969, 287, 1237, 1289, 375, 1430, 1444, 582, 1117 and 59.

8. An isolated polyepitopic peptide comprising at least three peptides selected from the HLA-A binding peptides selected from the group consisting of: SEQ ID NO 557, 1241, 1456, 1478, 1833, 1887, 67, 922, 66, 361, 1070, 1072, 1151, 71, 1233, 1269, 75, 73, 1396, 5, 87, 91, 238, 265, 1661, 1753, 76, 81, 92, 1933, 1934, 69, 2043, 2047, 74, 63, 2053, 83, 56, 155, 156, 1205, 1206, 167, 1350, 47, 146, 1609, 144, 3, 39, 158, 16, 122, 1034, 1095, 1096, 1150, 246, 1406, 23, 1483, 1512, 87, 93, 1625, 1626, 59, 1710, 250, 81, 1885, 1916, 1938, 2048, 271, 2083, 1, 877, 17, 7, 1086, 1087, 1468, 1700 and 1894,
whereby said peptides are characterized in that they are capable of inducing a CTL response.

9. An isolated polyepitopic peptide comprising at least three peptides selected from the HLA-B binding peptides selected from the group consisting of:
SEQ ID NO 402, 836, 381, 371, 853, 370, 387, 307, 1237, 1289, 1343, 1418, 1419, 375, 1430, 380, 450, 1582, 390, 1677, 1687, 121, 386, 372, 95, 443, 396, 455, 1441, 436, 1719, 92, 394, 1969, 287, 1237, 1289, 375, 1430, 1444, 582, 1117 and 59, whereby said peptides are characterized in that they are capable of inducing a CTL response.

10. An isolated polyepitopic peptide comprising at least three peptides selected from the HLA-C binding peptides selected from the group consisting of: SEQ ID NO 1048, 1095, 1730, 349, 475, 111, 2066, 1511, 1454, 1100 and 907, whereby said peptides are characterized in that they are capable of inducing a CTL response.

11. An isolated polyepitopic peptide comprising at least three peptides selected from the HLA-DRB1 binding peptides selected from the group consisting of: SEQ ID NO 2142, 2213, 2157, 2245, 2162, 2164, 2235, 2113, 2182, 2111, 2180, 2236, 2112, 2132, 2192, 2107, 2137, 2125, 2229, 2166, 2136, 2177, 2153, 2110, 2156, 2241, 2228, 2219, 2187, 2249, 2194, 2207, 2237, 2149, 2201, 2158, 2108 and 2232, whereby said peptides are characterized in that they are capable of inducing a HTL response.

12. The polyepitopic peptide according to claim 1 further comprising at least one HLA-DRB1 binding peptide selected from the group consisting of: SEQ ID NO 2142, 2213, 2157, 2245, 2162, 2164, 2235, 2113, 2182, 2111, 2180, 2236, 2112, 2132, 2192, 2107, 2137, 2125, 2229, 2166, 2136, 2177, 2153, 2110, 2156, 2241, 2228, 2219, 2187, 2249, 2194, 2207, 2237, 2149, 2201, 2158, 2108 and 2232.

13. The polyepitopic peptide according to claim 8 further characterized in that said at least three peptides are present in the HCV consensus sequence of genotype 1a, 1b and/or 3a.

14. The polyepitopic peptide according to claim 1 wherein at least one of said peptides is characterized in that it has cross-binding activity for HLA molecules derived from different HLA groups or loci.

15. The polyepitopic peptide according to claim 8 wherein the HLA-A binding peptide is characterized in that it binds a HLA molecule, said molecule being selected from the HLA-A group HLA-A01, -A02, -A03, -A11 or -A24.

16. The polyepitopic peptide according claim 9 wherein the HLA-B binding peptide is characterized in that it binds a HLA molecule, said molecule being selected from the HLA-B group HLA-B07, -B08, -B35, -B40 or -B44.

17. The polyepitopic peptide according to claim 10 wherein the HLA-C binding peptide is characterized in that it binds a HLA molecule, said molecule being selected from the HLA-C group HLA-Cw03, Cw04, Cw06 or Cw07.

18. The polyepitopic peptide according to claim 11 wherein the a HLA-DRB1 binding peptide characterized in that it binds a HLA molecule, said molecule being selected from the HLA-DRB1 group HLA-DRB1*01, -DRB1*03 or -DRB1*04.

19. The polyepitopic peptide according to claim 1 wherein the at least two peptides are selected from different HLA-loci.

20. An isolated peptide consisting of an amino acid sequence selected from the group consisting of: SEQ ID NO 557, 1241, 1456, 1478, 1833, 1887, 67, 922, 66, 361, 1070, 1072, 1151, 71, 1233, 1269, 75, 73, 1396, 5, 87, 91, 238, 265, 1661, 1753, 76, 81, 92, 1933, 1934, 69, 2043, 2047, 74, 63, 2053, 83, 56, 155, 156, 1205, 1206, 167, 1350, 47, 146, 1609, 144, 3, 39, 158, 16, 122, 1034, 1095, 1096, 1150, 246, 1406, 23, 1483, 1512, 87, 93, 1625, 1626, 59, 1710, 250, 81, 1885, 1916, 1938, 2048, 271, 2083, 1, 877, 17, 7, 1086, 1087, 1468, 1700, 1894, 402, 836, 381, 371, 853, 370, 387, 307, 1237, 1289, 1343, 1418, 1419, 375, 1430, 380, 450, 1582, 390, 1677, 1687, 121, 386, 372, 95, 443, 396, 455, 1441, 436, 1719, 92, 394, 1969, 287, 1237, 1289, 375, 1430, 1444, 582, 1117, 59, 1048, 1095, 1730, 349, 475, 111, 2066, 1511, 1454, 1100, 907, 2142, 2213, 2157, 2245, 2162, 2164, 2235, 2113, 2182, 2111, 2180, 2236, 2112, 2132, 2192, 2107, 2137, 2125, 2229, 2166, 2136, 2177, 2153, 2110, 2156, 2241, 2228, 2219, 2187, 2249, 2194, 2207, 2237, 2149, 2201, 2158, 2108 and 2232.

21. The peptide according to claim 20 comprised in an immunogenic peptide of less than 50 amino acid residues.

22. The peptide according to claim 20, wherein said peptide is capable of inducing a HLA class I and/or class II restricted T lymphocyte response.

23. An isolated peptide consisting of an amino acid sequence which is at least 70% identical to the amino acid sequence of the peptide according to claim 20, said peptide being capable of inducing a HLA class I and/or class II restricted T lymphocyte response.

24. An isolated nested epitope comprising two or more epitopes selected from Tables 13 and 14.

25. A nested epitope according to claim 24, wherein the two or more epitopes are selected from Table A.

26. A nested epitope according to claim 24, wherein the nested epitope consists of an amino acid sequence as identified by SEQ ID NO 2254 to 2278, or a part thereof.

27. A nested epitope according to claim 24 consisting of 9 to 35 amino acids.

28. An isolated polyepitopic peptide comprising at least one peptide or nested epitope according to claim 20.

29. An isolated polyepitopic peptide comprising at least two peptides or nested epitopes according to claim 20.

30. An isolated polyepitopic peptide comprising at least three peptides or nested epitopes according to claim 20.

31. The polyepitopic peptide according to claim 30 wherein the at least three peptides are at least two HLA-B binding peptides in combination with at least one HLA-A binding peptide or at least one HLA-C binding peptide.

32. The polyepitopic peptide according to claim 31 wherein the at least two HLA-B binding peptides are selected from a different HLA-group within the HLA-B locus.

33. The polyepitopic peptide according to claim 30 comprising at least one HLA-A binding peptide, at least one HLA-B binding peptide and at least one HLA-C binding peptide.

34. A polyepitopic peptide according to claim 29, wherein said at least two or three peptides are characterized in that they are present in the HCV consensus sequence of genotype 1a, 1b and/or 3a.

35. The polyepitopic peptide according to claim 28 further comprising a HTL epitope.

36. The polyepitopic peptide according to claim 35 wherein the HTL epitope is selected from Table 14.

37. The polyepitopic peptide according to claim 35, wherein the HTL epitope is a PanDR binding peptide.

38. The polyepitopic peptide according to claim 1 further comprising at least one HLA class I binding peptide, at least one HLA class II binding peptide or at least one HCV derived peptide.

39. The polyepitopic peptide according to claim 1, wherein the peptides are either contiguous or are separated by a linker or a spacer amino acid or spacer peptide.

40. The polyepitopic peptide according to claim 1, wherein the peptides are present as homopolymers and/or heteropolymers.

41. An isolated nucleic acid or polynucleotide encoding the peptide, nested epitope or polyepitopic peptide of claim 1.

42. The isolated nucleic or polynucleotide according to claim 41 further comprising at least one spacer nucleic acid.

43. The isolated nucleic or polynucleotide according to claim 41 further comprising a signal sequence and/or promotor sequence.

44. A vector comprising the nucleic acid or polynucleotide according to claim 41.

45. The vector according to claim 44 wherein said vector is a plasmid.

46. The vector according to claim 44 wherein said vector is viral vector.

47. A host cell comprising the vector according to claim 44.

48. A method for producing the vector comprising introducing the nucleic acid or polynucleotide according to claim 41 into a vector.

49. A composition comprising the peptide, nested epitope or polyepitopic peptide according to claim 1, or the nucleic acid or polynucleotide coding the same, or the vector including said nucleic acid or polynucleotide, or any combination thereof.

50. The composition according to claim 49 wherein the peptides or nucleic acids are present in an admixture.

51. The composition according to claim 49 wherein said composition is a pharmaceutical composition.

52. The composition according to claim 51 further comprising at least one of a pharmaceutically acceptable carrier, adjuvant or vehicle.

53. The composition according to claim 51 wherein said composition is a vaccine composition.

54. The peptide, nested epitope or polyepitopic peptide according to claim 1, or the nucleic acid or polynucleotide coding for the same, or the vector according including said nucleic acid or polynucleotide, or a composition including any of the same, or any combination thereof, for use as a medicament.

55. A method for inducing an immune response in a subject against HCV which comprises administration of the peptide, nested epitope or polyepitopic peptide according to claim 1, or the nucleic acid or polynucleotide encoding the same, or the vector including said nucleic acid or polynucleotide, or a composition including any of the same, or any combination thereof.

56. (canceled)

57. A method for producing the peptide, nested epitope or polyepitopic peptide according to claim 1 comprising the step of synthetic or recombinant production.

58. A method for producing the nucleic acid or polynucleotide according to claim 41 comprising the step of synthetic production.

59. A method of determining the outcome of infection for a subject exposed to HCV, comprising the steps of determining whether the subject has an immune response to one or more peptides, or the nucleic acids encoding them, according to claim 1.

60-63. (canceled)


SEQUENCE LISTING (2278 sequences)

SEQ ID NO: 1 - protein, 9 aa, hepatitis C virus
ATDALMTGY

SEQ ID NO: 2 - protein, 9 aa, hepatitis C virus
LTCGFADLM

SEQ ID NO: 3 - protein, 9 aa, hepatitis C virus
RVCEKMALY

SEQ ID NO: 4 - protein, 9 aa, hepatitis C virus
FADLMGYIP

SEQ ID NO: 5 - protein, 9 aa, hepatitis C virus
LTHIDAHFL

SEQ ID NO: 6 - protein, 9 aa, hepatitis C virus
ITTGAPITY

SEQ ID NO: 7 - protein, 9 aa, hepatitis C virus
FTDNSSPPA

SEQ ID NO: 8 - protein, 9 aa, hepatitis C virus
DNFPYLVAY

SEQ ID NO: 9 - protein, 9 aa, hepatitis C virus
FTEAMTRYS

SEQ ID NO: 10 - protein, 9 aa, hepatitis C virus
DASGKRVYY

SEQ ID NO: 11 - protein, 9 aa, hepatitis C virus
GAPITYSTY

SEQ ID NO: 12 - protein, 9 aa, hepatitis C virus
PAAYAAQGY

SEQ ID NO: 13 - protein, 9 aa, hepatitis C virus
CYDAGCAWY

SEQ ID NO: 14 - protein, 9 aa, hepatitis C virus
YAPTLWARM

SEQ ID NO: 15 - protein, 9 aa, hepatitis C virus
PVESMETTM

SEQ ID NO: 16 - protein, 9 aa, hepatitis C virus
AVMGSSYGF

SEQ ID NO: 17 - protein, 9 aa, hepatitis C virus
DSSVLCECY

SEQ ID NO: 18 - protein, 9 aa, hepatitis C virus
LGLNAVAYY

SEQ ID NO: 19 - protein, 9 aa, hepatitis C virus
PGDPPQPEY

SEQ ID NO: 20 - protein, 9 aa, hepatitis C virus
NGEIPFYGK

SEQ ID NO: 21 - protein, 9 aa, hepatitis C virus
VTLTHPITK

SEQ ID NO: 22 - protein, 9 aa, hepatitis C virus
MGSSYGFQY

SEQ ID NO: 23 - protein, 9 aa, hepatitis C virus
LTHPITKYI

SEQ ID NO: 24 - protein, 9 aa, hepatitis C virus
AGDNFPYLV

SEQ ID NO: 25 - protein, 9 aa, hepatitis C virus
KSTKVPAAY

SEQ ID NO: 26 - protein, 9 aa, hepatitis C virus
ITYSTYGKF

SEQ ID NO: 27 - protein, 9 aa, hepatitis C virus
PAETSVRLR

SEQ ID NO: 28 - protein, 9 aa, hepatitis C virus
VIDTLTCGF

SEQ ID NO: 29 - protein, 9 aa, hepatitis C virus
GLDVSVIPT

SEQ ID NO: 30 - protein, 9 aa, hepatitis C virus
LDPTFTIET

SEQ ID NO: 31 - protein, 9 aa, hepatitis C virus
LEDGVNYAT

SEQ ID NO: 32 - protein, 9 aa, hepatitis C virus
ATLGFGAYM

SEQ ID NO: 33 - protein, 9 aa, hepatitis C virus
PSWDQMWKC

SEQ ID NO: 34 - protein, 9 aa, hepatitis C virus
PTLWARMIL

SEQ ID NO: 35 - protein, 9 aa, hepatitis C virus
PTFTIETTT

SEQ ID NO: 36 - protein, 9 aa, hepatitis C virus
VFTEAMTRY

SEQ ID NO: 37 - protein, 9 aa, hepatitis C virus
PQAVMGSSY

SEQ ID NO: 38 - protein, 9 aa, hepatitis C virus
VAHDASGKR

SEQ ID NO: 39 - protein, 9 aa, hepatitis C virus
RVFTEAMTR

SEQ ID NO: 40 - protein, 9 aa, hepatitis C virus
GNTLTCYLK

SEQ ID NO: 41 - protein, 9 aa, hepatitis C virus
EVFCVQPEK

SEQ ID NO: 42 - protein, 9 aa, hepatitis C virus
CSFSIFLLA

SEQ ID NO: 43 - protein, 9 aa, hepatitis C virus
YSPGQRVEF

SEQ ID NO: 44 - protein, 9 aa, hepatitis C virus
VVATDALMT

SEQ ID NO: 45 - protein, 9 aa, hepatitis C virus
RVLEDGVNY

SEQ ID NO: 46 - protein, 9 aa, hepatitis C virus
ETSVRLRAY

SEQ ID NO: 47 - protein, 9 aa, hepatitis C virus
LIFCHSKKK

SEQ ID NO: 48 - protein, 9 aa, hepatitis C virus
CCDLAPEAR

SEQ ID NO: 49 - protein, 9 aa, hepatitis C virus
NSWLGNIIM

SEQ ID NO: 50 - protein, 9 aa, hepatitis C virus
LGFGAYMSK

SEQ ID NO: 51 - protein, 9 aa, hepatitis C virus
ALGLNAVAY

SEQ ID NO: 52 - protein, 9 aa, hepatitis C virus
ESMETTMRS

SEQ ID NO: 53 - protein, 9 aa, hepatitis C virus
TDALMTGYT

SEQ ID NO: 54 - protein, 9 aa, hepatitis C virus
FIPVESMET

SEQ ID NO: 55 - protein, 9 aa, hepatitis C virus
PTDPRRRSR

SEQ ID NO: 56 - protein, 9 aa, hepatitis C virus
AAYAAQGYK

SEQ ID NO: 57 - protein, 9 aa, hepatitis C virus
TTMRSPVFT

SEQ ID NO: 58 - protein, 9 aa, hepatitis C virus
DAHFLSQTK

SEQ ID NO: 59 - protein, 9 aa, hepatitis C virus
RMILMTHFF

SEQ ID NO: 60 - protein, 9 aa, hepatitis C virus
QAETAGARL

SEQ ID NO: 61 - protein, 9 aa, hepatitis C virus
MSADLEVVT

SEQ ID NO: 62 - protein, 9 aa, hepatitis C virus
WLGNIIMYA

SEQ ID NO: 63 - protein, 9 aa, hepatitis C virus
YLVAYQATV

SEQ ID NO: 64 - protein, 9 aa, hepatitis C virus
LTHIDAHFL

SEQ ID NO: 65 - protein, 9 aa, hepatitis C virus
AQPGYPWPL

SEQ ID NO: 66 - protein, 9 aa, hepatitis C virus
DLMGYIPLV

SEQ ID NO: 67 - protein, 9 aa, hepatitis C virus
ALYDVVSTL

SEQ ID NO: 68 - protein, 9 aa, hepatitis C virus
VVSTLPQAV

SEQ ID NO: 69 - protein, 9 aa, hepatitis C virus
YIPLVGAPL

SEQ ID NO: 70 - protein, 9 aa, hepatitis C virus
LLSCLTIPA

SEQ ID NO: 71 - protein, 9 aa, hepatitis C virus
GMFDSSVLC

SEQ ID NO: 72 - protein, 9 aa, hepatitis C virus
ALAHGVRVL

SEQ ID NO: 73 - protein, 9 aa, hepatitis C virus
KVLVLNPSV

SEQ ID NO: 74 - protein, 9 aa, hepatitis C virus
YLNTPGLPV

SEQ ID NO: 75 - protein, 9 aa, hepatitis C virus
KLQDCTMLV

SEQ ID NO: 76 - protein, 9 aa, hepatitis C virus
SVFTGLTHI

SEQ ID NO: 77 - protein, 9 aa, hepatitis C virus
VVATDALMT

SEQ ID NO: 78 - protein, 9 aa, hepatitis C virus
GLGWAGWLL

SEQ ID NO: 79 - protein, 9 aa, hepatitis C virus
RLYIGGPLT

SEQ ID NO: 80 - protein, 9 aa, hepatitis C virus
FIPVESMET

SEQ ID NO: 81 - protein, 9 aa, hepatitis C virus
TLHGPTPLL

SEQ ID NO: 82 - protein, 9 aa, hepatitis C virus
LVLNPSVAA

SEQ ID NO: 83 - protein, 9 aa, hepatitis C virus
YQATVCARA

SEQ ID NO: 84 - protein, 9 aa, hepatitis C virus
IMYAPTLWA

SEQ ID NO: 85 - protein, 9 aa, hepatitis C virus
MALYDVVST

SEQ ID NO: 86 - protein, 9 aa, hepatitis C virus
RLVVLATAT

SEQ ID NO: 87 - protein, 9 aa, hepatitis C virus
NIIMYAPTL

SEQ ID NO: 88 - protein, 9 aa, hepatitis C virus
GTQEDAASL

SEQ ID NO: 89 - protein, 9 aa, hepatitis C virus
TILGIGTVL

SEQ ID NO: 90 - protein, 9 aa, hepatitis C virus
IMACMSADL

SEQ ID NO: 91 - protein, 9 aa, hepatitis C virus
QIVGGVYLL

SEQ ID NO: 92 - protein, 9 aa, hepatitis C virus
TLWARMILM

SEQ ID NO: 93 - protein, 9 aa, hepatitis C virus
NLPGCSFSI

SEQ ID NO: 94 - protein, 9 aa, hepatitis C virus
MLVNGDDLV

SEQ ID NO: 95 - protein, 9 aa, hepatitis C virus
YAAQGYKVL

SEQ ID NO: 96 - protein, 9 aa, hepatitis C virus
GVAKAVDFI

SEQ ID NO: 97 - protein, 9 aa, hepatitis C virus
RMILMTHFF

SEQ ID NO: 98 - protein, 9 aa, hepatitis C virus
TVLDQAETA

SEQ ID NO: 99 - protein, 9 aa, hepatitis C virus
LTHPITKYI

SEQ ID NO: 100 - protein, 9 aa, hepatitis C virus
VLNPSVAAT

SEQ ID NO: 101 - protein, 9 aa, hepatitis C virus
FTDNSSPPA

SEQ ID NO: 102 - protein, 9 aa, hepatitis C virus
VLATATPPG

SEQ ID NO: 103 - protein, 9 aa, hepatitis C virus
VLVLNPSVA

SEQ ID NO: 104 - protein, 9 aa, hepatitis C virus
LLCPSGHVV

SEQ ID NO: 105 - protein, 9 aa, hepatitis C virus
GLDVSVIPT

SEQ ID NO: 106 - protein, 9 aa, hepatitis C virus
FSLDPTFTI

SEQ ID NO: 107 - protein, 9 aa, hepatitis C virus
ATLGFGAYM

SEQ ID NO: 108 - protein, 9 aa, hepatitis C virus
YAPTLWARM

SEQ ID NO: 109 - protein, 9 aa, hepatitis C virus
TITTGAPIT

SEQ ID NO: 110 - protein, 9 aa, hepatitis C virus
TTMRSPVFT

SEQ ID NO: 111 - protein, 9 aa, hepatitis C virus
FQYSPGQRV

SEQ ID NO: 112 - protein, 9 aa, hepatitis C virus
QVAHLHAPT

SEQ ID NO: 113 - protein, 9 aa, hepatitis C virus
AVPQTFQVA

SEQ ID NO: 114 - protein, 9 aa, hepatitis C virus
RTITTGAPI

SEQ ID NO: 115 - protein, 9 aa, hepatitis C virus
AAQGYKVLV

SEQ ID NO: 116 - protein, 9 aa, hepatitis C virus
LVAYQATVC

SEQ ID NO: 117 - protein, 9 aa, hepatitis C virus
GVFRAAVCT

SEQ ID NO: 118 - protein, 9 aa, hepatitis C virus
LMGYIPLVG

SEQ ID NO: 119 - protein, 9 aa, hepatitis C virus
AVQNEVTLT

SEQ ID NO: 120 - protein, 9 aa, hepatitis C virus
GIYRFVTPG

SEQ ID NO: 121 - protein, 9 aa, hepatitis C virus
SAACRAAKL

SEQ ID NO: 122 - protein, 9 aa, hepatitis C virus
CLIRLKPTL

SEQ ID NO: 123 - protein, 9 aa, hepatitis C virus
FLSQTKQAG

SEQ ID NO: 124 - protein, 9 aa, hepatitis C virus
SVIDCNTCV

SEQ ID NO: 125 - protein, 9 aa, hepatitis C virus
ATPPGSVTV

SEQ ID NO: 126 - protein, 9 aa, hepatitis C virus
AAWETARHT

SEQ ID NO: 127 - protein, 9 aa, hepatitis C virus
GQIVGGVYL

SEQ ID NO: 128 - protein, 9 aa, hepatitis C virus
VLEDGVNYA

SEQ ID NO: 129 - protein, 9 aa, hepatitis C virus
LEFWESVFT

SEQ ID NO: 130 - protein, 9 aa, hepatitis C virus
AQGYKVLVL

SEQ ID NO: 131 - protein, 9 aa, hepatitis C virus
LVNGDDLVV

SEQ ID NO: 132 - protein, 9 aa, hepatitis C virus
LLPRRGPRL

SEQ ID NO: 133 - protein, 9 aa, hepatitis C virus
STLPQAVMG

SEQ ID NO: 134 - protein, 9 aa, hepatitis C virus
VIPTSGDVV

SEQ ID NO: 135 - protein, 9 aa, hepatitis C virus
SGMFDSSVL

SEQ ID NO: 136 - protein, 9 aa, hepatitis C virus
CMSADLEVV

SEQ ID NO: 137 - protein, 9 aa, hepatitis C virus
YGKAIPIEV

SEQ ID NO: 138 - protein, 9 aa, hepatitis C virus
MSADLEVVT

SEQ ID NO: 139 - protein, 9 aa, hepatitis C virus
GNIIMYAPT

SEQ ID NO: 140 - protein, 9 aa, hepatitis C virus
GIGTVLDQA

SEQ ID NO: 141 - protein, 9 aa, hepatitis C virus
HVVGVFRAA

SEQ ID NO: 142 - protein, 9 aa, hepatitis C virus
KLSALGLNA

SEQ ID NO: 143 - protein, 9 aa, hepatitis C virus
AIPIEVIKG

SEQ ID NO: 144 - protein, 9 aa, hepatitis C virus
RLGVRATRK

SEQ ID NO: 145 - protein, 9 aa, hepatitis C virus
GLNAVAYYR

SEQ ID NO: 146 - protein, 9 aa, hepatitis C virus
LVNAWKSKK

SEQ ID NO: 147 - protein, 9 aa, hepatitis C virus
AAYAAQGYK

SEQ ID NO: 148 - protein, 9 aa, hepatitis C virus
HLIFCHSKK

SEQ ID NO: 149 - protein, 9 aa, hepatitis C virus
YLLPRRGPR

SEQ ID NO: 150 - protein, 9 aa, hepatitis C virus
FLVNAWKSK

SEQ ID NO: 151 - protein, 9 aa, hepatitis C virus
LIFCHSKKK

SEQ ID NO: 152 - protein, 9 aa, hepatitis C virus
VTLTHPITK

SEQ ID NO: 153 - protein, 9 aa, hepatitis C virus
ALGLNAVAY

SEQ ID NO: 154 - protein, 9 aa, hepatitis C virus
DLGVRVCEK

SEQ ID NO: 155 - protein, 9 aa, hepatitis C virus
ASAACRAAK

SEQ ID NO: 156 - protein, 9 aa, hepatitis C virus
AVCTRGVAK

SEQ ID NO: 157 - protein, 9 aa, hepatitis C virus
VQPEKGGRK

SEQ ID NO: 158 - protein, 9 aa, hepatitis C virus
STNPKPQRK

SEQ ID NO: 159 - protein, 9 aa, hepatitis C virus
RQPIPKARR

SEQ ID NO: 160 - protein, 9 aa, hepatitis C virus
RVFTEAMTR

SEQ ID NO: 161 - protein, 9 aa, hepatitis C virus
YLKASAACR

SEQ ID NO: 162 - protein, 9 aa, hepatitis C virus
GNTLTCYLK

SEQ ID NO: 163 - protein, 9 aa, hepatitis C virus
WLLSPRGSR

SEQ ID NO: 164 - protein, 9 aa, hepatitis C virus
KTKRNTNRR

SEQ ID NO: 165 - protein, 9 aa, hepatitis C virus
ALYDVVSTL

SEQ ID NO: 166 - protein, 9 aa, hepatitis C virus
LGFGAYMSK

SEQ ID NO: 167 - protein, 9 aa, hepatitis C virus
KTSERSQPR

SEQ ID NO: 168 - protein, 9 aa, hepatitis C virus
IVFPDLGVR

SEQ ID NO: 169 - protein, 9 aa, hepatitis C virus
RVLEDGVNY

SEQ ID NO: 170 - protein, 9 aa, hepatitis C virus
IMYAPTLWA

SEQ ID NO: 171 - protein, 9 aa, hepatitis C virus
GAPITYSTY

SEQ ID NO: 172 - protein, 9 aa, hepatitis C virus
GKRVYYLTR

SEQ ID NO: 173 - protein, 9 aa, hepatitis C virus
RHLIFCHSK

SEQ ID NO: 174 - protein, 9 aa, hepatitis C virus
GRGRRGIYR

SEQ ID NO: 175 - protein, 9 aa, hepatitis C virus
RLYIGGPLT

SEQ ID NO: 176 - protein, 9 aa, hepatitis C virus
PMGFSYDTR

SEQ ID NO: 177 - protein, 9 aa, hepatitis C virus
RGRRQPIPK

SEQ ID NO: 178 - protein, 9 aa, hepatitis C virus
VEFLVNAWK

SEQ ID NO: 179 - protein, 9 aa, hepatitis C virus
GMFDSSVLC

SEQ ID NO: 180 - protein, 9 aa, hepatitis C virus
DQMWKCLIR

SEQ ID NO: 181 - protein, 9 aa, hepatitis C virus
KAIPIEVIK

SEQ ID NO: 182 - protein, 9 aa, hepatitis C virus
YLNTPGLPV

SEQ ID NO: 183 - protein, 9 aa, hepatitis C virus
RVCEKMALY

SEQ ID NO: 184 - protein, 9 aa, hepatitis C virus
WAGWLLSPR

SEQ ID NO: 185 - protein, 9 aa, hepatitis C virus
YLVAYQATV

SEQ ID NO: 186 - protein, 9 aa, hepatitis C virus
GLGWAGWLL

SEQ ID NO: 187 - protein, 9 aa, hepatitis C virus
MWKCLIRLK

SEQ ID NO: 188 - protein, 9 aa, hepatitis C virus
SVAHDASGK

SEQ ID NO: 189 - protein, 9 aa, hepatitis C virus
RATRKTSER

SEQ ID NO: 190 - protein, 9 aa, hepatitis C virus
WLGNIIMYA

SEQ ID NO: 191 - protein, 9 aa, hepatitis C virus
KAIPIEAIK

SEQ ID NO: 192 - protein, 9 aa, hepatitis C virus
HGPTPLLYR

SEQ ID NO: 193 - protein, 9 aa, hepatitis C virus
EVFCVQPEK

SEQ ID NO: 194 - protein, 9 aa, hepatitis C virus
MSTNPKPQR

SEQ ID NO: 195 - protein, 9 aa, hepatitis C virus
LHAPTGSGK

SEQ ID NO: 196 - protein, 9 aa, hepatitis C virus
VSRSQRRGR

SEQ ID NO: 197 - protein, 9 aa, hepatitis C virus
VGGVYLLPR

SEQ ID NO: 198 - protein, 9 aa, hepatitis C virus
GVFRAAVCT

SEQ ID NO: 199 - protein, 9 aa, hepatitis C virus
TNRRPQDVK

SEQ ID NO: 200 - protein, 9 aa, hepatitis C virus
LLYRLGAVQ

SEQ ID NO: 201 - protein, 9 aa, hepatitis C virus
TLTHPITKY

SEQ ID NO: 202 - protein, 9 aa, hepatitis C virus
GLDVSVIPT

SEQ ID NO: 203 - protein, 9 aa, hepatitis C virus
PSWGPTDPR

SEQ ID NO: 204 - protein, 9 aa, hepatitis C virus
VVGVFRAAV

SEQ ID NO: 205 - protein, 9 aa, hepatitis C virus
LIRLKPTLH

SEQ ID NO: 206 - protein, 9 aa, hepatitis C virus
GVRATRKTS

SEQ ID NO: 207 - protein, 9 aa, hepatitis C virus
GAPLGGAAR

SEQ ID NO: 208 - protein, 9 aa, hepatitis C virus
DLMGYIPLV

SEQ ID NO: 209 - protein, 9 aa, hepatitis C virus
QTFQVAHLH

SEQ ID NO: 210 - protein, 9 aa, hepatitis C virus
ATDALMTGY

SEQ ID NO: 211 - protein, 9 aa, hepatitis C virus
KQAGDNFPY

SEQ ID NO: 212 - protein, 9 aa, hepatitis C virus
DNFPYLVAY

SEQ ID NO: 213 - protein, 9 aa, hepatitis C virus
LLPRRGPRL

SEQ ID NO: 214 - protein, 9 aa, hepatitis C virus
PTGSGKSTK

SEQ ID NO: 215 - protein, 9 aa, hepatitis C virus
ITYSTYGKF

SEQ ID NO: 216 - protein, 9 aa, hepatitis C virus
QPGYPWPLY

SEQ ID NO: 217 - protein, 9 aa, hepatitis C virus
AMTRYSAPP

SEQ ID NO: 218 - protein, 9 aa, hepatitis C virus
GIGTVLDQA

SEQ ID NO: 219 - protein, 9 aa, hepatitis C virus
LHGPTPLLY

SEQ ID NO: 220 - protein, 9 aa, hepatitis C virus
LTPAETSVR

SEQ ID NO: 221 - protein, 9 aa, hepatitis C virus
SQRRGRTGR

SEQ ID NO: 222 - protein, 9 aa, hepatitis C virus
KSTKVPAAY

SEQ ID NO: 223 - protein, 9 aa, hepatitis C virus
IVGGVYLLP

SEQ ID NO: 224 - protein, 9 aa, hepatitis C virus
RTGRGRRGI

SEQ ID NO: 225 - protein, 9 aa, hepatitis C virus
KLSALGLNA

SEQ ID NO: 226 - protein, 9 aa, hepatitis C virus
FQYSPGQRV

SEQ ID NO: 227 - protein, 9 aa, hepatitis C virus
RTWAQPGYP

SEQ ID NO: 228 - protein, 9 aa, hepatitis C virus
QNCGYRRCR

SEQ ID NO: 229 - protein, 9 aa, hepatitis C virus
DSSVLCECY

SEQ ID NO: 230 - protein, 9 aa, hepatitis C virus
RMILMTHFF

SEQ ID NO: 231 - protein, 9 aa, hepatitis C virus
TLWARMILM

SEQ ID NO: 232 - protein, 9 aa, hepatitis C virus
CLIRLKPTL

SEQ ID NO: 233 - protein, 9 aa, hepatitis C virus
TLHGPTPLL

SEQ ID NO: 234 - protein, 9 aa, hepatitis C virus
FWESVFTGL

SEQ ID NO: 235 - protein, 9 aa, hepatitis C virus
TWAQPGYPW

SEQ ID NO: 236 - protein, 9 aa, hepatitis C virus
GFADLMGYI

SEQ ID NO: 237 - protein, 9 aa, hepatitis C virus
NIIMYAPTL

SEQ ID NO: 238 - protein, 9 aa, hepatitis C virus
QMWKCLIRL

SEQ ID NO: 239 - protein, 9 aa, hepatitis C virus
KYIMACMSA

SEQ ID NO: 240 - protein, 9 aa, hepatitis C virus
AQGYKVLVL

SEQ ID NO: 241 - protein, 9 aa, hepatitis C virus
TYSTYGKFL

SEQ ID NO: 242 - protein, 9 aa, hepatitis C virus
KAHGVDPNI

SEQ ID NO: 243 - protein, 9 aa, hepatitis C virus
LYGNEGLGW

SEQ ID NO: 244 - protein, 9 aa, hepatitis C virus
AYMSKAHGV

SEQ ID NO: 245 - protein, 9 aa, hepatitis C virus
GLGWAGWLL

SEQ ID NO: 246 - protein, 9 aa, hepatitis C virus
IIMYAPTLW

SEQ ID NO: 247 - protein, 9 aa, hepatitis C virus
GQIVGGVYL

SEQ ID NO: 248 - protein, 9 aa, hepatitis C virus
WLGNIIMYA

SEQ ID NO: 249 - protein, 9 aa, hepatitis C virus
TAGARLVVL

SEQ ID NO: 250 - protein, 9 aa, hepatitis C virus
SFSIFLLAL

SEQ ID NO: 251 - protein, 9 aa, hepatitis C virus
FSLDPTFTI

SEQ ID NO: 252 - protein, 9 aa, hepatitis C virus
YLVAYQATV

SEQ ID NO: 253 - protein, 9 aa, hepatitis C virus
VIKGGRHLI

SEQ ID NO: 254 - protein, 9 aa, hepatitis C virus
PLLYRLGAV

SEQ ID NO: 255 - protein, 9 aa, hepatitis C virus
TILGIGTVL

SEQ ID NO: 256 - protein, 9 aa, hepatitis C virus
PVNSWLGNI

SEQ ID NO: 257 - protein, 9 aa, hepatitis C virus
ETTMRSPVF

SEQ ID NO: 258 - protein, 9 aa, hepatitis C virus
GLTHIDAHF

SEQ ID NO: 259 - protein, 9 aa, hepatitis C virus
AVMGSSYGF

SEQ ID NO: 260 - protein, 9 aa, hepatitis C virus
KGSSGGPLL

SEQ ID NO: 261 - protein, 9 aa, hepatitis C virus
KLQDCTMLV

SEQ ID NO: 262 - protein, 9 aa, hepatitis C virus
YAAQGYKVL

SEQ ID NO: 263 - protein, 9 aa, hepatitis C virus
LTHPITKYI

SEQ ID NO: 264 - protein, 9 aa, hepatitis C virus
PFYGKAIPI

SEQ ID NO: 265 - protein, 9 aa, hepatitis C virus
RLGAVQNEV

SEQ ID NO: 266 - protein, 9 aa, hepatitis C virus
AIKGGRHLI

SEQ ID NO: 267 - protein, 9 aa, hepatitis C virus
ALYDVVSTL

SEQ ID NO: 268 - protein, 9 aa, hepatitis C virus
RALAHGVRV

SEQ ID NO: 269 - protein, 9 aa, hepatitis C virus
YIPLVGAPL

SEQ ID NO: 270 - protein, 9 aa, hepatitis C virus
LLPRRGPRL

SEQ ID NO: 271 - protein, 9 aa, hepatitis C virus
YYRGLDVSV

SEQ ID NO: 272 - protein, 9 aa, hepatitis C virus
ELAAKLSAL

SEQ ID NO: 273 - protein, 9 aa, hepatitis C virus
YGKAIPIEV

SEQ ID NO: 274 - protein, 9 aa, hepatitis C virus
MGSSYGFQY

SEQ ID NO: 275 - protein, 9 aa, hepatitis C virus
LLCPSGHVV

SEQ ID NO: 276 - protein, 9 aa, hepatitis C virus
AWKSKKCPM

SEQ ID NO: 277 - protein, 9 aa, hepatitis C virus
AYAAQGYKV

SEQ ID NO: 278 - protein, 9 aa, hepatitis C virus
RVEFLVNAW

SEQ ID NO: 279 - protein, 9 aa, hepatitis C virus
SWDQMWKCL

SEQ ID NO: 280 - protein, 9 aa, hepatitis C virus
NLPGCSFSI

SEQ ID NO: 281 - protein, 9 aa, hepatitis C virus
GFSYDTRCF

SEQ ID NO: 282 - protein, 9 aa, hepatitis C virus
PAVPQTFQV

SEQ ID NO: 283 - protein, 9 aa, hepatitis C virus
NLGKVIDTL

SEQ ID NO: 284 - protein, 9 aa, hepatitis C virus
KFPGGGQIV

SEQ ID NO: 285 - protein, 9 aa, hepatitis C virus
YLNTPGLPV

SEQ ID NO: 286 - protein, 9 aa, hepatitis C virus
WDQMWKCLI

SEQ ID NO: 287 - protein, 9 aa, hepatitis C virus
WARMILMTH

SEQ ID NO: 288 - protein, 9 aa, hepatitis C virus
AQPGYPWPL

SEQ ID NO: 289 - protein, 9 aa, hepatitis C virus
QIVGGVYLL

SEQ ID NO: 290 - protein, 9 aa, hepatitis C virus
YSTYGKFLA

SEQ ID NO: 291 - protein, 9 aa, hepatitis C virus
GMFDSSVLC

SEQ ID NO: 292 - protein, 9 aa, hepatitis C virus
MYAPTLWAR

SEQ ID NO: 293 - protein, 9 aa, hepatitis C virus
CSFSIFLLA

SEQ ID NO: 294 - protein, 9 aa, hepatitis C virus
GCSFSIFLL

SEQ ID NO: 295 - protein, 9 aa, hepatitis C virus
GVAKAVDFI

SEQ ID NO: 296 - protein, 9 aa, hepatitis C virus
FQYSPGQRV

SEQ ID NO: 297 - protein, 9 aa, hepatitis C virus
ALAHGVRVL

SEQ ID NO: 298 - protein, 9 aa, hepatitis C virus
RHTPVNSWL

SEQ ID NO: 299 - protein, 9 aa, hepatitis C virus
PTLWARMIL

SEQ ID NO: 300 - protein, 9 aa, hepatitis C virus
RGRRGIYRF

SEQ ID NO: 301 - protein, 9 aa, hepatitis C virus
KSKKCPMGF

SEQ ID NO: 302 - protein, 9 aa, hepatitis C virus
LALLSCLTI

SEQ ID NO: 303 - protein, 9 aa, hepatitis C virus
SVTVPHPNI

SEQ ID NO: 304 - protein, 9 aa, hepatitis C virus
LTTSCGNTL

SEQ ID NO: 305 - protein, 9 aa, hepatitis C virus
ITKYIMACM

SEQ ID NO: 306 - protein, 9 aa, hepatitis C virus
FYGKAIPIE

SEQ ID NO: 307 - protein, 9 aa, hepatitis C virus
GPTPLLYRL

SEQ ID NO: 308 - protein, 9 aa, hepatitis C virus
LMTGYTGDF

SEQ ID NO: 309 - protein, 9 aa, hepatitis C virus
RVCEKMALY

SEQ ID NO: 310 - protein, 9 aa, hepatitis C virus
DLMGYIPLV

SEQ ID NO: 311 - protein, 9 aa, hepatitis C virus
IKGGRHLIF

SEQ ID NO: 312 - protein, 9 aa, hepatitis C virus
PQTFQVAHL

SEQ ID NO: 313 - protein, 9 aa, hepatitis C virus
YYLTRDPTT

SEQ ID NO: 314 - protein, 9 aa, hepatitis C virus
LWARMILMT

SEQ ID NO: 315 - protein, 9 aa, hepatitis C virus
KVLVLNPSV

SEQ ID NO: 316 - protein, 9 aa, hepatitis C virus
RTITTGAPI

SEQ ID NO: 317 - protein, 9 aa, hepatitis C virus
RGVAKAVDF

SEQ ID NO: 318 - protein, 9 aa, hepatitis C virus
RSRNLGKVI

SEQ ID NO: 319 - protein, 9 aa, hepatitis C virus
RLYIGGPLT

SEQ ID NO: 320 - protein, 9 aa, hepatitis C virus
CYLKASAAC

SEQ ID NO: 321 - protein, 9 aa, hepatitis C virus
WYELTPAET

SEQ ID NO: 322 - protein, 9 aa, hepatitis C virus
LTHIDAHFL

SEQ ID NO: 323 - protein, 9 aa, hepatitis C virus
PGQRVEFLV

SEQ ID NO: 324 - protein, 9 aa, hepatitis C virus
KLSALGLNA

SEQ ID NO: 325 - protein, 9 aa, hepatitis C virus
THIDAHFLS

SEQ ID NO: 326 - protein, 9 aa, hepatitis C virus
YSPGQRVEF

SEQ ID NO: 327 - protein, 9 aa, hepatitis C virus
AGDNFPYLV

SEQ ID NO: 328 - protein, 9 aa, hepatitis C virus
IMACMSADL

SEQ ID NO: 329 - protein, 9 aa, hepatitis C virus
PVCQDHLEF

SEQ ID NO: 330 - protein, 9 aa, hepatitis C virus
AAQGYKVLV

SEQ ID NO: 331 - protein, 9 aa, hepatitis C virus
IGLSNNGEI

SEQ ID NO: 332 - protein, 9 aa, hepatitis C virus
RSLTERLYI

SEQ ID NO: 333 - protein, 9 aa, hepatitis C virus
CMSADLEVV

SEQ ID NO: 334 - protein, 9 aa, hepatitis C virus
ASGKRVYYL

SEQ ID NO: 335 - protein, 9 aa, hepatitis C virus
YGKAIPIEA

SEQ ID NO: 336 - protein, 9 aa, hepatitis C virus
LITSCSSNV

SEQ ID NO: 337 - protein, 9 aa, hepatitis C virus
ATLGFGAYM

SEQ ID NO: 338 - protein, 9 aa, hepatitis C virus
LAPEARQAI

SEQ ID NO: 339 - protein, 9 aa, hepatitis C virus
GVNYATGNL

SEQ ID NO: 340 - protein, 9 aa, hepatitis C virus
RVLEDGVNY

SEQ ID NO: 341 - protein, 9 aa, hepatitis C virus
YTGDFDSVI

SEQ ID NO: 342 - protein, 9 aa, hepatitis C virus
PITKYIMAC

SEQ ID NO: 343 - protein, 9 aa, hepatitis C virus
VVVATDALM

SEQ ID NO: 344 - protein, 9 aa, hepatitis C virus
RAQAPPPSW

SEQ ID NO: 345 - protein, 9 aa, hepatitis C virus
PGCSFSIFL

SEQ ID NO: 346 - protein, 9 aa, hepatitis C virus
SVFTGLTHI

SEQ ID NO: 347 - protein, 9 aa, hepatitis C virus
GAVQNEVTL

SEQ ID NO: 348 - protein, 9 aa, hepatitis C virus
KQAGDNFPY

SEQ ID NO: 349 - protein, 9 aa, hepatitis C virus
VFPDLGVRV

SEQ ID NO: 350 - protein, 9 aa, hepatitis C virus
VDFSLDPTF

SEQ ID NO: 351 - protein, 9 aa, hepatitis C virus
GLPVCQDHL

SEQ ID NO: 352 - protein, 9 aa, hepatitis C virus
VNGDDLVVI

SEQ ID NO: 353 - protein, 9 aa, hepatitis C virus
GYTGDFDSV

SEQ ID NO: 354 - protein, 9 aa, hepatitis C virus
PSGHVVGVF

SEQ ID NO: 355 - protein, 9 aa, hepatitis C virus
VVSTLPQAV

SEQ ID NO: 356 - protein, 9 aa, hepatitis C virus
ITYSTYGKF

SEQ ID NO: 357 - protein, 9 aa, hepatitis C virus
PLGGAARAL

SEQ ID NO: 358 - protein, 9 aa, hepatitis C virus
PYLVAYQAT

SEQ ID NO: 359 - protein, 9 aa, hepatitis C virus
THPITKYIM

SEQ ID NO: 360 - protein, 9 aa, hepatitis C virus
FGAYMSKAH

SEQ ID NO: 361 - protein, 9 aa, hepatitis C virus
FLLALLSCL

SEQ ID NO: 362 - protein, 9 aa, hepatitis C virus
FSIFLLALL

SEQ ID NO: 363 - protein, 9 aa, hepatitis C virus
TLTCGFADL

SEQ ID NO: 364 - protein, 9 aa, hepatitis C virus
LMGYIPLVG

SEQ ID NO: 365 - protein, 9 aa, hepatitis C virus
WPLYGNEGL

SEQ ID NO: 366 - protein, 9 aa, hepatitis C virus
GYIPLVGAP

SEQ ID NO: 367 - protein, 9 aa, hepatitis C virus
EGLGWAGWL

SEQ ID NO: 368 - protein, 9 aa, hepatitis C virus
LLSCLTIPA

SEQ ID NO: 369 - protein, 9 aa, hepatitis C virus
GYPWPLYGN

SEQ ID NO: 370 - protein, 9 aa, hepatitis C virus
DPRRRSRNL

SEQ ID NO: 371 - protein, 9 aa, hepatitis C virus
APTLWARMI

SEQ ID NO: 372 - protein, 9 aa, hepatitis C virus
TPGERPSGM

SEQ ID NO: 373 - protein, 9 aa, hepatitis C virus
SPGQRVEFL

SEQ ID NO: 374 - protein, 9 aa, hepatitis C virus
DPRRRSRNL

SEQ ID NO: 375 - protein, 9 aa, hepatitis C virus
LPGCSFSIF

SEQ ID NO: 376 - protein, 9 aa, hepatitis C virus
EVIKGGRHL

SEQ ID NO: 377 - protein, 9 aa, hepatitis C virus
EAIKGGRHL

SEQ ID NO: 378 - protein, 9 aa, hepatitis C virus
WPLYGNEGL

SEQ ID NO: 379 - protein, 9 aa, hepatitis C virus
ALAHGVRVL

SEQ ID NO: 380 - protein, 9 aa, hepatitis C virus
LPRRGPRLG

SEQ ID NO: 381 - protein, 9 aa, hepatitis C virus
APPPSWDQM

SEQ ID NO: 382 - protein, 9 aa, hepatitis C virus
GPTPLLYRL

SEQ ID NO: 383 - protein, 9 aa, hepatitis C virus
TPAETSVRL

SEQ ID NO: 384 - protein, 9 aa, hepatitis C virus
APLGGAARA

SEQ ID NO: 385 - protein, 9 aa, hepatitis C virus
ATLGFGAYM

SEQ ID NO: 386 - protein, 9 aa, hepatitis C virus
SPRGSRPSW

SEQ ID NO: 387 - protein, 9 aa, hepatitis C virus
GPRLGVRAT

SEQ ID NO: 388 - protein, 9 aa, hepatitis C virus
EARQAIRSL

SEQ ID NO: 389 - protein, 9 aa, hepatitis C virus
TPLLYRLGA

SEQ ID NO: 390 - protein, 9 aa, hepatitis C virus
QPRGRRQPI

SEQ ID NO: 391 - protein, 9 aa, hepatitis C virus
YAAQGYKVL

SEQ ID NO: 392 - protein, 9 aa, hepatitis C virus
VPHPNIEEI

SEQ ID NO: 393 - protein, 9 aa, hepatitis C virus
AVMGSSYGF

SEQ ID NO: 394 - protein, 9 aa, hepatitis C virus
VAYYRGLDV

SEQ ID NO: 395 - protein, 9 aa, hepatitis C virus
HPNIEEIGL

SEQ ID NO: 396 - protein, 9 aa, hepatitis C virus
HPITKYIMA

SEQ ID NO: 397 - protein, 9 aa, hepatitis C virus
APTGSGKST

SEQ ID NO: 398 - protein, 9 aa, hepatitis C virus
SVFTGLTHI

SEQ ID NO: 399 - protein, 9 aa, hepatitis C virus
CPSGHVVGV

SEQ ID NO: 400 - protein, 9 aa, hepatitis C virus
NAVAYYRGL

SEQ ID NO: 401 - protein, 9 aa, hepatitis C virus
SAACRAAKL

SEQ ID NO: 402 - protein, 9 aa, hepatitis C virus
AAKLSALGL

SEQ ID NO: 403 - protein, 9 aa, hepatitis C virus
AARALAHGV

SEQ ID NO: 404 - protein, 9 aa, hepatitis C virus
NPKPQRKTK

SEQ ID NO: 405 - protein, 9 aa, hepatitis C virus
APEARQAIR

SEQ ID NO: 406 - protein, 9 aa, hepatitis C virus
RSRNLGKVI

SEQ ID NO: 407 - protein, 9 aa, hepatitis C virus
FPGGGQIVG

SEQ ID NO: 408 - protein, 9 aa, hepatitis C virus
APITYSTYG

SEQ ID NO: 409 - protein, 9 aa, hepatitis C virus
IPKARRPEG

SEQ ID NO: 410 - protein, 9 aa, hepatitis C virus
VPQTFQVAH

SEQ ID NO: 411 - protein, 9 aa, hepatitis C virus
NSWLGNIIM

SEQ ID NO: 412 - protein, 9 aa, hepatitis C virus
RALAHGVRV

SEQ ID NO: 413 - protein, 9 aa, hepatitis C virus
RTGRGRRGI

SEQ ID NO: 414 - protein, 9 aa, hepatitis C virus
ITKYIMACM

SEQ ID NO: 415 - protein, 9 aa, hepatitis C virus
IPTSGDVVV

SEQ ID NO: 416 - protein, 9 aa, hepatitis C virus
PTLHGPTPL

SEQ ID NO: 417 - protein, 9 aa, hepatitis C virus
IPFYGKAIP

SEQ ID NO: 418 - protein, 9 aa, hepatitis C virus
YAPTLWARM

SEQ ID NO: 419 - protein, 9 aa, hepatitis C virus
ASGKRVYYL

SEQ ID NO: 420 - protein, 9 aa, hepatitis C virus
TAGARLVVL

SEQ ID NO: 421 - protein, 9 aa, hepatitis C virus
CPMGFSYDT

SEQ ID NO: 422 - protein, 9 aa, hepatitis C virus
AIKGGRHLI

SEQ ID NO: 423 - protein, 9 aa, hepatitis C virus
NPSVAATLG

SEQ ID NO: 424 - protein, 9 aa, hepatitis C virus
TILGIGTVL

SEQ ID NO: 425 - protein, 9 aa, hepatitis C virus
IPIEAIKGG

SEQ ID NO: 426 - protein, 9 aa, hepatitis C virus
LTHPITKYI

SEQ ID NO: 427 - protein, 9 aa, hepatitis C virus
LVLNPSVAA

SEQ ID NO: 428 - protein, 9 aa, hepatitis C virus
ERLYIGGPL

SEQ ID NO: 429 - protein, 9 aa, hepatitis C virus
GVAKAVDFI

SEQ ID NO: 430 - protein, 9 aa, hepatitis C virus
QPEKGGRKP

SEQ ID NO: 431 - protein, 9 aa, hepatitis C virus
RPSWGPTDP

SEQ ID NO: 432 - protein, 9 aa, hepatitis C virus
GVNYATGNL

SEQ ID NO: 433 - protein, 9 aa, hepatitis C virus
ELAAKLSAL

SEQ ID NO: 434 - protein, 9 aa, hepatitis C virus
RAYLNTPGL

SEQ ID NO: 435 - protein, 9 aa, hepatitis C virus
GGRKPARLI

SEQ ID NO: 436 - protein, 9 aa, hepatitis C virus
NIRTGVRTI

SEQ ID NO: 437 - protein, 9 aa, hepatitis C virus
VVVATDALM

SEQ ID NO: 438 - protein, 9 aa, hepatitis C virus
AQGYKVLVL

SEQ ID NO: 439 - protein, 9 aa, hepatitis C virus
ETAGARLVV

SEQ ID NO: 440 - protein, 9 aa, hepatitis C virus
AAKLQDCTM

SEQ ID NO: 441 - protein, 9 aa, hepatitis C virus
KGSSGGPLL

SEQ ID NO: 442 - protein, 9 aa, hepatitis C virus
IPLVGAPLG

SEQ ID NO: 443 - protein, 9 aa, hepatitis C virus
FPYLVAYQA

SEQ ID NO: 444 - protein, 9 aa, hepatitis C virus
WAGWLLSPR

SEQ ID NO: 445 - protein, 9 aa, hepatitis C virus
QPGYPWPLY

SEQ ID NO: 446 - protein, 9 aa, hepatitis C virus
LALLSCLTI

SEQ ID NO: 447 - protein, 9 aa, hepatitis C virus
GVRVLEDGV

SEQ ID NO: 448 - protein, 9 aa, hepatitis C virus
AQPGYPWPL

SEQ ID NO: 449 - protein, 9 aa, hepatitis C virus
PRRGPRLGV

SEQ ID NO: 450 - protein, 10 aa, hepatitis C virus
LPRRGPRLGV

SEQ ID NO: 451 - protein, 9 aa, hepatitis C virus
DPRRRSRNL

SEQ ID NO: 452 - protein, 9 aa, hepatitis C virus
QPRGRRQPI

SEQ ID NO: 453 - protein, 9 aa, hepatitis C virus
IPKARRPEG

SEQ ID NO: 454 - protein, 9 aa, hepatitis C virus
HPITKYIMA

SEQ ID NO: 455 - protein, 9 aa, hepatitis C virus
HSKKKCDEL

SEQ ID NO: 456 - protein, 9 aa, hepatitis C virus
QRRGRTGRG

SEQ ID NO: 457 - protein, 9 aa, hepatitis C virus
NPKPQRKTK

SEQ ID NO: 458 - protein, 9 aa, hepatitis C virus
GKRVYYLTR

SEQ ID NO: 459 - protein, 9 aa, hepatitis C virus
SVRLRAYLN

SEQ ID NO: 460 - protein, 9 aa, hepatitis C virus
DLMGYIPLV

SEQ ID NO: 461 - protein, 9 aa, hepatitis C virus
NAVAYYRGL

SEQ ID NO: 462 - protein, 9 aa, hepatitis C virus
EGRTWAQPG

SEQ ID NO: 463 - protein, 9 aa, hepatitis C virus
ITKYIMACM

SEQ ID NO: 464 - protein, 9 aa, hepatitis C virus
GRRGIYRFV

SEQ ID NO: 465 - protein, 9 aa, hepatitis C virus
TLWARMILM

SEQ ID NO: 466 - protein, 9 aa, hepatitis C virus
DTRCFDSTV

SEQ ID NO: 467 - protein, 9 aa, hepatitis C virus
QMWKCLIRL

SEQ ID NO: 468 - protein, 9 aa, hepatitis C virus
LIRLKPTLH

SEQ ID NO: 469 - protein, 9 aa, hepatitis C virus
VAYYRGLDV

SEQ ID NO: 470 - protein, 9 aa, hepatitis C virus
LTHPITKYI

SEQ ID NO: 471 - protein, 9 aa, hepatitis C virus
WARMILMTH

SEQ ID NO: 472 - protein, 9 aa, hepatitis C virus
FPYLVAYQA

SEQ ID NO: 473 - protein, 9 aa, hepatitis C virus
AIRSLTERL

SEQ ID NO: 474 - protein, 9 aa, hepatitis C virus
EARQAIRSL

SEQ ID NO: 475 - protein, 9 aa, hepatitis C virus
YRRCRASGV

SEQ ID NO: 476 - protein, 9 aa, hepatitis C virus
QRKTKRNTN

SEQ ID NO: 477 - protein, 9 aa, hepatitis C virus
FCHSKKKCD

SEQ ID NO: 478 - protein, 9 aa, hepatitis C virus
AWKSKKCPM

SEQ ID NO: 479 - protein, 9 aa, hepatitis C virus
QPIPKARRP

SEQ ID NO: 480 - protein, 9 aa, hepatitis C virus
DHLEFWESV

SEQ ID NO: 481 - protein, 9 aa, hepatitis C virus
SGKRVYYLT

SEQ ID NO: 482 - protein, 9 aa, hepatitis C virus
YGKAIPIEA

SEQ ID NO: 483 - protein, 9 aa, hepatitis C virus
SGKSTKVPA

SEQ ID NO: 484 - protein, 9 aa, hepatitis C virus
TPLLYRLGA

SEQ ID NO: 485 - protein, 9 aa, hepatitis C virus
NIIMYAPTL

SEQ ID NO: 486 - protein, 9 aa, hepatitis C virus
YGKAIPIEV

SEQ ID NO: 487 - protein, 9 aa, hepatitis C virus
GVYLLPRRG

SEQ ID NO: 488 - protein, 9 aa, hepatitis C virus
EAMTRYSAP

SEQ ID NO: 489 - protein, 9 aa, hepatitis C virus
EGLGWAGWL

SEQ ID NO: 490 - protein, 9 aa, hepatitis C virus
TGRGRRGIY

SEQ ID NO: 491 - protein, 9 aa, hepatitis C virus
VIKGGRHLI

SEQ ID NO: 492 - protein, 9 aa, hepatitis C virus
GGRHLIFCH

SEQ ID NO: 493 - protein, 9 aa, hepatitis C virus
DAHFLSQTK

SEQ ID NO: 494 - protein, 9 aa, hepatitis C virus
TCYLKASAA

SEQ ID NO: 495 - protein, 9 aa, hepatitis C virus
SLRVFTEAM

SEQ ID NO: 496 - protein, 9 aa, hepatitis C virus
DAVSRSQRR

SEQ ID NO: 497 - protein, 9 aa, hepatitis C virus
DQMWKCLIR

SEQ ID NO: 498 - protein, 9 aa, hepatitis C virus
GVRVCEKMA

SEQ ID NO: 499 - protein, 9 aa, hepatitis C virus
STKVPAAYA

SEQ ID NO: 500 - protein, 9 aa, hepatitis C virus
LARAAWETA

SEQ ID NO: 501 - protein, 9 aa, hepatitis C virus
LLPRRGPRL

SEQ ID NO: 502 - protein, 9 aa, hepatitis C virus
STYGKFLAD

SEQ ID NO: 503 - protein, 9 aa, hepatitis C virus
GGRKPARLI

SEQ ID NO: 504 - protein, 9 aa, hepatitis C virus
AIKGGRHLI

SEQ ID NO: 505 - protein, 9 aa, hepatitis C virus
GRRQPIPKA

SEQ ID NO: 506 - protein, 9 aa, hepatitis C virus
CLIRLKPTL

SEQ ID NO: 507 - protein, 9 aa, hepatitis C virus
GRKPARLIV

SEQ ID NO: 508 - protein, 9 aa, hepatitis C virus
IMYAPTLWA

SEQ ID NO: 509 - protein, 9 aa, hepatitis C virus
RSRNLGKVI

SEQ ID NO: 510 - protein, 9 aa, hepatitis C virus
EIPFYGKAI

SEQ ID NO: 511 - protein, 9 aa, hepatitis C virus
NIRTGVRTI

SEQ ID NO: 512 - protein, 9 aa, hepatitis C virus
YLLPRRGPR

SEQ ID NO: 513 - protein, 9 aa, hepatitis C virus
AARALAHGV

SEQ ID NO: 514 - protein, 9 aa, hepatitis C virus
LPRRGPRLG

SEQ ID NO: 515 - protein, 9 aa, hepatitis C virus
LPGCSFSIF

SEQ ID NO: 516 - protein, 9 aa, hepatitis C virus
FSLDPTFTI

SEQ ID NO: 517 - protein, 9 aa, hepatitis C virus
AVMGSSYGF

SEQ ID NO: 518 - protein, 9 aa, hepatitis C virus
FPYLVAYQA

SEQ ID NO: 519 - protein, 9 aa, hepatitis C virus
FPGGGQIVG

SEQ ID NO: 520 - protein, 9 aa, hepatitis C virus
MGSSYGFQY

SEQ ID NO: 521 - protein, 9 aa, hepatitis C virus
TPAETSVRL

SEQ ID NO: 522 - protein, 9 aa, hepatitis C virus
RVLEDGVNY

SEQ ID NO: 523 - protein, 9 aa, hepatitis C virus
HPNIEEIGL

SEQ ID NO: 524 - protein, 9 aa, hepatitis C virus
DNFPYLVAY

SEQ ID NO: 525 - protein, 9 aa, hepatitis C virus
DASGKRVYY

SEQ ID NO: 526 - protein, 9 aa, hepatitis C virus
TCVTQTVDF

SEQ ID NO: 527 - protein, 9 aa, hepatitis C virus
HVVGVFRAA

SEQ ID NO: 528 - protein, 9 aa, hepatitis C virus
DAGCAWYEL

SEQ ID NO: 529 - protein, 9 aa, hepatitis C virus
NSWLGNIIM

SEQ ID NO: 530 - protein, 9 aa, hepatitis C virus
DALMTGYTG

SEQ ID NO: 531 - protein, 9 aa, hepatitis C virus
LSNNGEIPF

SEQ ID NO: 532 - protein, 9 aa, hepatitis C virus
YSPGQRVEF

SEQ ID NO: 533 - protein, 9 aa, hepatitis C virus
WARMILMTH

SEQ ID NO: 534 - protein, 9 aa, hepatitis C virus
VPHPNIEEI

SEQ ID NO: 535 - protein, 9 aa, hepatitis C virus
QPGYPWPLY

SEQ ID NO: 536 - protein, 9 aa, hepatitis C virus
SAACRAAKL

SEQ ID NO: 537 - protein, 9 aa, hepatitis C virus
APITYSTYG

SEQ ID NO: 538 - protein, 9 aa, hepatitis C virus
WPLYGNEGL

SEQ ID NO: 539 - protein, 9 aa, hepatitis C virus
GPTPLLYRL

SEQ ID NO: 540 - protein, 9 aa, hepatitis C virus
FSIFLLALL

SEQ ID NO: 541 - protein, 9 aa, hepatitis C virus
CPSGHVVGV

SEQ ID NO: 542 - protein, 9 aa, hepatitis C virus
CPMGFSYDT

SEQ ID NO: 543 - protein, 9 aa, hepatitis C virus
DPPQPEYDL

SEQ ID NO: 544 - protein, 9 aa, hepatitis C virus
VPQTFQVAH

SEQ ID NO: 545 - protein, 9 aa, hepatitis C virus
RALAHGVRV

SEQ ID NO: 546 - protein, 9 aa, hepatitis C virus
VVVATDALM

SEQ ID NO: 547 - protein, 9 aa, hepatitis C virus
IPLVGAPLG

SEQ ID NO: 548 - protein, 9 aa, hepatitis C virus
TPLLYRLGA

SEQ ID NO: 549 - protein, 9 aa, hepatitis C virus
VPAAYAAQG

SEQ ID NO: 550 - protein, 9 aa, hepatitis C virus
LGLNAVAYY

SEQ ID NO: 551 - protein, 9 aa, hepatitis C virus
QAGDNFPYL

SEQ ID NO: 552 - protein, 9 aa, hepatitis C virus
RPQDVKFPG

SEQ ID NO: 553 - protein, 9 aa, hepatitis C virus
RVCEKMALY

SEQ ID NO: 554 - protein, 9 aa, hepatitis C virus
LVVICESAG

SEQ ID NO: 555 - protein, 9 aa, hepatitis C virus
APLGGAARA

SEQ ID NO: 556 - protein, 9 aa, hepatitis C virus
CSFSIFLLA

SEQ ID NO: 557 - protein, 9 aa, hepatitis C virus
AATLGFGAY

SEQ ID NO: 558 - protein, 9 aa, hepatitis C virus
CGFADLMGY

SEQ ID NO: 559 - protein, 9 aa, hepatitis C virus
HDASGKRVY

SEQ ID NO: 560 - protein, 9 aa, hepatitis C virus
ATLGFGAYM

SEQ ID NO: 561 - protein, 9 aa, hepatitis C virus
IPIEVIKGG

SEQ ID NO: 562 - protein, 9 aa, hepatitis C virus
EDAASLRVF

SEQ ID NO: 563 - protein, 9 aa, hepatitis C virus
AQPGYPWPL

SEQ ID NO: 564 - protein, 9 aa, hepatitis C virus
LEFWESVFT

SEQ ID NO: 565 - protein, 9 aa, hepatitis C virus
NEGLGWAGW

SEQ ID NO: 566 - protein, 9 aa, hepatitis C virus
WLGNIIMYA

SEQ ID NO: 567 - protein, 9 aa, hepatitis C virus
RMILMTHFF

SEQ ID NO: 568 - protein, 9 aa, hepatitis C virus
TLWARMILM

SEQ ID NO: 569 - protein, 9 aa, hepatitis C virus
DELAAKLSA

SEQ ID NO: 570 - protein, 9 aa, hepatitis C virus
GQIVGGVYL

SEQ ID NO: 571 - protein, 9 aa, hepatitis C virus
WESVFTGLT

SEQ ID NO: 572 - protein, 9 aa, hepatitis C virus
NEVTLTHPI

SEQ ID NO: 573 - protein, 9 aa, hepatitis C virus
YLVAYQATV

SEQ ID NO: 574 - protein, 9 aa, hepatitis C virus
IEAIKGGRH

SEQ ID NO: 575 - protein, 9 aa, hepatitis C virus
IEVIKGGRH

SEQ ID NO: 576 - protein, 9 aa, hepatitis C virus
VEFLVNAWK

SEQ ID NO: 577 - protein, 9 aa, hepatitis C virus
WETARHTPV

SEQ ID NO: 578 - protein, 9 aa, hepatitis C virus
QMWKCLIRL

SEQ ID NO: 579 - protein, 9 aa, hepatitis C virus
GLGWAGWLL

SEQ ID NO: 580 - protein, 9 aa, hepatitis C virus
TERLYIGGP

SEQ ID NO: 581 - protein, 9 aa, hepatitis C virus
CEKMALYDV

SEQ ID NO: 582 - protein, 9 aa, hepatitis C virus
AETAGARLV

SEQ ID NO: 583 - protein, 9 aa, hepatitis C virus
IEEIGLSNN

SEQ ID NO: 584 - protein, 9 aa, hepatitis C virus
RLYIGGPLT

SEQ ID NO: 585 - protein, 9 aa, hepatitis C virus
ALGLNAVAY

SEQ ID NO: 586 - protein, 9 aa, hepatitis C virus
TEAMTRYSA

SEQ ID NO: 587 - protein, 9 aa, hepatitis C virus
PEYDLELIT

SEQ ID NO: 588 - protein, 9 aa, hepatitis C virus
LMTGYTGDF

SEQ ID NO: 589 - protein, 9 aa, hepatitis C virus
CECYDAGCA

SEQ ID NO: 590 - protein, 9 aa, hepatitis C virus
METTMRSPV

SEQ ID NO: 591 - protein, 9 aa, hepatitis C virus
IMYAPTLWA

SEQ ID NO: 592 - protein, 9 aa, hepatitis C virus
ATLGFGAYM

SEQ ID NO: 593 - protein, 9 aa, hepatitis C virus
SWDQMWKCL

SEQ ID NO: 594 - protein, 9 aa, hepatitis C virus
SFSIFLLAL

SEQ ID NO: 595 - protein, 9 aa, hepatitis C virus
FWESVFTGL

SEQ ID NO: 596 - protein, 9 aa, hepatitis C virus
TYSTYGKFL

SEQ ID NO: 597 - protein, 9 aa, hepatitis C virus
EYDLELITS

SEQ ID NO: 598 - protein, 9 aa, hepatitis C virus
AYAAQGYKV

SEQ ID NO: 599 - protein, 9 aa, hepatitis C virus
VFPDLGVRV

SEQ ID NO: 600 - protein, 9 aa, hepatitis C virus
GFADLMGYI

SEQ ID NO: 601 - protein, 9 aa, hepatitis C virus
HLEFWESVF

SEQ ID NO: 602 - protein, 9 aa, hepatitis C virus
CYDAGCAWY

SEQ ID NO: 603 - protein, 9 aa, hepatitis C virus
LYGNEGLGW

SEQ ID NO: 604 - protein, 9 aa, hepatitis C virus
RMILMTHFF

SEQ ID NO: 605 - protein, 9 aa, hepatitis C virus
AYMSKAHGV

SEQ ID NO: 606 - protein, 9 aa, hepatitis C virus
IMACMSADL

SEQ ID NO: 607 - protein, 9 aa, hepatitis C virus
WYELTPAET

SEQ ID NO: 608 - protein, 9 aa, hepatitis C virus
KFPGGGQIV

SEQ ID NO: 609 - protein, 9 aa, hepatitis C virus
GFSYDTRCF

SEQ ID NO: 610 - protein, 9 aa, hepatitis C virus
YYRGLDVSV

SEQ ID NO: 611 - protein, 9 aa, hepatitis C virus
TFTIETTTV

SEQ ID NO: 612 - protein, 9 aa, hepatitis C virus
VFTEAMTRY

SEQ ID NO: 613 - protein, 9 aa, hepatitis C virus
HPNIEEIGL

SEQ ID NO: 614 - protein, 9 aa, hepatitis C virus
VIDTLTCGF

SEQ ID NO: 615 - protein, 9 aa, hepatitis C virus
SYDTRCFDS

SEQ ID NO: 616 - protein, 9 aa, hepatitis C virus
QIVGGVYLL

SEQ ID NO: 617 - protein, 9 aa, hepatitis C virus
GLGWAGWLL

SEQ ID NO: 618 - protein, 9 aa, hepatitis C virus
TLTCGFADL

SEQ ID NO: 619 - protein, 9 aa, hepatitis C virus
GYIPLVGAP

SEQ ID NO: 620 - protein, 9 aa, hepatitis C virus
NLPGCSFSI

SEQ ID NO: 621 - protein, 9 aa, hepatitis C virus
TYSTYGKFL

SEQ ID NO: 622 - protein, 9 aa, hepatitis C virus
YAAQGYKVL

SEQ ID NO: 623 - protein, 9 aa, hepatitis C virus
YRRCRASGV

SEQ ID NO: 624 - protein, 9 aa, hepatitis C virus
IRSLTERLY

SEQ ID NO: 625 - protein, 9 aa, hepatitis C virus
GRRGIYRFV

SEQ ID NO: 626 - protein, 9 aa, hepatitis C virus
GRKPARLIV

SEQ ID NO: 627 - protein, 9 aa, hepatitis C virus
RMILMTHFF

SEQ ID NO: 628 - protein, 9 aa, hepatitis C virus
YYRGLDVSV

SEQ ID NO: 629 - protein, 9 aa, hepatitis C virus
QMWKCLIRL

SEQ ID NO: 630 - protein, 9 aa, hepatitis C virus
SFSIFLLAL

SEQ ID NO: 631 - protein, 9 aa, hepatitis C virus
SWDQMWKCL

SEQ ID NO: 632 - protein, 9 aa, hepatitis C virus
VRVCEKMAL

SEQ ID NO: 633 - protein, 9 aa, hepatitis C virus
IKGGRHLIF

SEQ ID NO: 634 - protein, 9 aa, hepatitis C virus
EARQAIRSL

SEQ ID NO: 635 - protein, 9 aa, hepatitis C virus
NAVAYYRGL

SEQ ID NO: 636 - protein, 9 aa, hepatitis C virus
EAIKGGRHL

SEQ ID NO: 637 - protein, 9 aa, hepatitis C virus
VAYYRGLDV

SEQ ID NO: 638 - protein, 9 aa, hepatitis C virus
ERLYIGGPL

SEQ ID NO: 639 - protein, 9 aa, hepatitis C virus
EVIKGGRHL

SEQ ID NO: 640 - protein, 9 aa, hepatitis C virus
AYMSKAHGV

SEQ ID NO: 641 - protein, 9 aa, hepatitis C virus
ARMILMTHF

SEQ ID NO: 642 - protein, 9 aa, hepatitis C virus
IMACMSADL

SEQ ID NO: 643 - protein, 9 aa, hepatitis C virus
ARLIVFPDL

SEQ ID NO: 644 - protein, 9 aa, hepatitis C virus
AYAAQGYKV

SEQ ID NO: 645 - protein, 9 aa, hepatitis C virus
ETSVRLRAY

SEQ ID NO: 646 - protein, 9 aa, hepatitis C virus
RKPARLIVF

SEQ ID NO: 647 - protein, 9 aa, hepatitis C virus
AAQGYKVLV

SEQ ID NO: 648 - protein, 9 aa, hepatitis C virus
FQYSPGQRV

SEQ ID NO: 649 - protein, 9 aa, hepatitis C virus
QIVGGVYLL

SEQ ID NO: 650 - protein, 9 aa, hepatitis C virus
FSIFLLALL

SEQ ID NO: 651 - protein, 9 aa, hepatitis C virus
FWESVFTGL

SEQ ID NO: 652 - protein, 9 aa, hepatitis C virus
DHLEFWESV

SEQ ID NO: 653 - protein, 9 aa, hepatitis C virus
FLLALLSCL

SEQ ID NO: 654 - protein, 9 aa, hepatitis C virus
PRRGPRLGV

SEQ ID NO: 655 - protein, 9 aa, hepatitis C virus
KKCPMGFSY

SEQ ID NO: 656 - protein, 9 aa, hepatitis C virus
MGSSYGFQY

SEQ ID NO: 657 - protein, 9 aa, hepatitis C virus
YAPTLWARM

SEQ ID NO: 658 - protein, 9 aa, hepatitis C virus
WPLYGNEGL

SEQ ID NO: 659 - protein, 9 aa, hepatitis C virus
DAGCAWYEL

SEQ ID NO: 660 - protein, 9 aa, hepatitis C virus
ASGKRVYYL

SEQ ID NO: 661 - protein, 9 aa, hepatitis C virus
TAGARLVVL

SEQ ID NO: 662 - protein, 9 aa, hepatitis C virus
DASGKRVYY

SEQ ID NO: 663 - protein, 9 aa, hepatitis C virus
YGKAIPIEV

SEQ ID NO: 664 - protein, 9 aa, hepatitis C virus
IMYAPTLWA

SEQ ID NO: 665 - protein, 9 aa, hepatitis C virus
SWLGNIIMY

SEQ ID NO: 666 - protein, 9 aa, hepatitis C virus
AQPGYPWPL

SEQ ID NO: 667 - protein, 9 aa, hepatitis C virus
WARMILMTH

SEQ ID NO: 668 - protein, 9 aa, hepatitis C virus
TLWARMILM

SEQ ID NO: 669 - protein, 9 aa, hepatitis C virus
LHGPTPLLY

SEQ ID NO: 670 - protein, 9 aa, hepatitis C virus
LTHPITKYI

SEQ ID NO: 671 - protein, 9 aa, hepatitis C virus
THPITKYIM

SEQ ID NO: 672 - protein, 9 aa, hepatitis C virus
LMTGYTGDF

SEQ ID NO: 673 - protein, 9 aa, hepatitis C virus
DPRRRSRNL

SEQ ID NO: 674 - protein, 9 aa, hepatitis C virus
GCSFSIFLL

SEQ ID NO: 675 - protein, 9 aa, hepatitis C virus
ALAHGVRVL

SEQ ID NO: 676 - protein, 9 aa, hepatitis C virus
HLEFWESVF

SEQ ID NO: 677 - protein, 9 aa, hepatitis C virus
AAKLSALGL

SEQ ID NO: 678 - protein, 9 aa, hepatitis C virus
YLVAYQATV

SEQ ID NO: 679 - protein, 9 aa, hepatitis C virus
GFSYDTRCF

SEQ ID NO: 680 - protein, 9 aa, hepatitis C virus
FPYLVAYQA

SEQ ID NO: 681 - protein, 9 aa, hepatitis C virus
RAYLNTPGL

SEQ ID NO: 682 - protein, 9 aa, hepatitis C virus
YRGLDVSVI

SEQ ID NO: 683 - protein, 9 aa, hepatitis C virus
KFPGGGQIV

SEQ ID NO: 684 - protein, 9 aa, hepatitis C virus
DNFPYLVAY

SEQ ID NO: 685 - protein, 9 aa, hepatitis C virus
NRRPQDVKF

SEQ ID NO: 686 - protein, 9 aa, hepatitis C virus
SAACRAAKL

SEQ ID NO: 687 - protein, 9 aa, hepatitis C virus
GPTPLLYRL

SEQ ID NO: 688 - protein, 9 aa, hepatitis C virus
YIPLVGAPL

SEQ ID NO: 689 - protein, 9 aa, hepatitis C virus
GQIVGGVYL

SEQ ID NO: 690 - protein, 9 aa, hepatitis C virus
VVGVFRAAV

SEQ ID NO: 691 - protein, 9 aa, hepatitis C virus
VFTEAMTRY

SEQ ID NO: 692 - protein, 9 aa, hepatitis C virus
DLMGYIPLV

SEQ ID NO: 693 - protein, 9 aa, hepatitis C virus
RAAVCTRGV

SEQ ID NO: 694 - protein, 9 aa, hepatitis C virus
SPGQRVEFL

SEQ ID NO: 695 - protein, 9 aa, hepatitis C virus
AVMGSSYGF

SEQ ID NO: 696 - protein, 9 aa, hepatitis C virus
ELAAKLSAL

SEQ ID NO: 697 - protein, 9 aa, hepatitis C virus
RALAHGVRV

SEQ ID NO: 698 - protein, 9 aa, hepatitis C virus
AQGYKVLVL

SEQ ID NO: 699 - protein, 9 aa, hepatitis C virus
RRSRNLGKV

SEQ ID NO: 700 - protein, 9 aa, hepatitis C virus
RHTPVNSWL

SEQ ID NO: 701 - protein, 9 aa, hepatitis C virus
VVSTLPQAV

SEQ ID NO: 702 - protein, 9 aa, hepatitis C virus
NIIMYAPTL

SEQ ID NO: 703 - protein, 9 aa, hepatitis C virus
MYAPTLWAR

SEQ ID NO: 704 - protein, 9 aa, hepatitis C virus
YSPGQRVEF

SEQ ID NO: 705 - protein, 9 aa, hepatitis C virus
QRVEFLVNA

SEQ ID NO: 706 - protein, 9 aa, hepatitis C virus
ALYDVVSTL

SEQ ID NO: 707 - protein, 9 aa, hepatitis C virus
GRTWAQPGY

SEQ ID NO: 708 - protein, 9 aa, hepatitis C virus
VFPDLGVRV

SEQ ID NO: 709 - protein, 9 aa, hepatitis C virus
LIVFPDLGV

SEQ ID NO: 710 - protein, 9 aa, hepatitis C virus
RRCRASGVL

SEQ ID NO: 711 - protein, 9 aa, hepatitis C virus
HFLSQTKQA

SEQ ID NO: 712 - protein, 9 aa, hepatitis C virus
VTQTVDFSL

SEQ ID NO: 713 - protein, 9 aa, hepatitis C virus
PTLWARMIL

SEQ ID NO: 714 - protein, 9 aa, hepatitis C virus
HDASGKRVY

SEQ ID NO: 715 - protein, 9 aa, hepatitis C virus
EGLGWAGWL

SEQ ID NO: 716 - protein, 9 aa, hepatitis C virus
TYSTYGKFL

SEQ ID NO: 717 - protein, 9 aa, hepatitis C virus
AYAAQGYKV

SEQ ID NO: 718 - protein, 9 aa, hepatitis C virus
YYRGLDVSV

SEQ ID NO: 719 - protein, 9 aa, hepatitis C virus
ARLIVFPDL

SEQ ID NO: 720 - protein, 9 aa, hepatitis C virus
SFSIFLLAL

SEQ ID NO: 721 - protein, 9 aa, hepatitis C virus
IRSLTERLY

SEQ ID NO: 722 - protein, 9 aa, hepatitis C virus
LYGNEGLGW

SEQ ID NO: 723 - protein, 9 aa, hepatitis C virus
GFADLMGYI

SEQ ID NO: 724 - protein, 9 aa, hepatitis C virus
GRTWAQPGY

SEQ ID NO: 725 - protein, 9 aa, hepatitis C virus
GFSYDTRCF

SEQ ID NO: 726 - protein, 9 aa, hepatitis C virus
SWLGNIIMY

SEQ ID NO: 727 - protein, 9 aa, hepatitis C virus
KYIMACMSA

SEQ ID NO: 728 - protein, 9 aa, hepatitis C virus
ARMILMTHF

SEQ ID NO: 729 - protein, 9 aa, hepatitis C virus
VRVCEKMAL

SEQ ID NO: 730 - protein, 9 aa, hepatitis C virus
YAPTLWARM

SEQ ID NO: 731 - protein, 9 aa, hepatitis C virus
TWAQPGYPW

SEQ ID NO: 732 - protein, 9 aa, hepatitis C virus
YYLTRDPTT

SEQ ID NO: 733 - protein, 9 aa, hepatitis C virus
MYAPTLWAR

SEQ ID NO: 734 - protein, 9 aa, hepatitis C virus
RHTPVNSWL

SEQ ID NO: 735 - protein, 9 aa, hepatitis C virus
YAAQGYKVL

SEQ ID NO: 736 - protein, 9 aa, hepatitis C virus
WPLYGNEGL

SEQ ID NO: 737 - protein, 9 aa, hepatitis C virus
HFLSQTKQA

SEQ ID NO: 738 - protein, 9 aa, hepatitis C virus
AYMSKAHGV

SEQ ID NO: 739 - protein, 9 aa, hepatitis C virus
FWESVFTGL

SEQ ID NO: 740 - protein, 9 aa, hepatitis C virus
RKPARLIVF

SEQ ID NO: 741 - protein, 9 aa, hepatitis C virus
RRCRASGVL

SEQ ID NO: 742 - protein, 9 aa, hepatitis C virus
RMILMTHFF

SEQ ID NO: 743 - protein, 9 aa, hepatitis C virus
SYGFQYSPG

SEQ ID NO: 744 - protein, 9 aa, hepatitis C virus
LHGPTPLLY

SEQ ID NO: 745 - protein, 9 aa, hepatitis C virus
ERLYIGGPL

SEQ ID NO: 746 - protein, 9 aa, hepatitis C virus
SWDQMWKCL

SEQ ID NO: 747 - protein, 9 aa, hepatitis C virus
FPYLVAYQA

SEQ ID NO: 748 - protein, 9 aa, hepatitis C virus
NRRPQDVKF

SEQ ID NO: 749 - protein, 9 aa, hepatitis C virus
VFPDLGVRV

SEQ ID NO: 750 - protein, 9 aa, hepatitis C virus
RRPQDVKFP

SEQ ID NO: 751 - protein, 9 aa, hepatitis C virus
YIPLVGAPL

SEQ ID NO: 752 - protein, 9 aa, hepatitis C virus
GYKVLVLNP

SEQ ID NO: 753 - protein, 9 aa, hepatitis C virus
THPITKYIM

SEQ ID NO: 754 - protein, 9 aa, hepatitis C virus
RYSAPPGDP

SEQ ID NO: 755 - protein, 9 aa, hepatitis C virus
GRRGIYRFV

SEQ ID NO: 756 - protein, 9 aa, hepatitis C virus
CYDAGCAWY

SEQ ID NO: 757 - protein, 9 aa, hepatitis C virus
GRGRRGIYR

SEQ ID NO: 758 - protein, 9 aa, hepatitis C virus
YRFVTPGER

SEQ ID NO: 759 - protein, 9 aa, hepatitis C virus
GYRRCRASG

SEQ ID NO: 760 - protein, 9 aa, hepatitis C virus
ARRPEGRTW

SEQ ID NO: 761 - protein, 9 aa, hepatitis C virus
KKCPMGFSY

SEQ ID NO: 762 - protein, 9 aa, hepatitis C virus
RRPEGRTWA

SEQ ID NO: 763 - protein, 9 aa, hepatitis C virus
VYLLPRRGP

SEQ ID NO: 764 - protein, 9 aa, hepatitis C virus
IMYAPTLWA

SEQ ID NO: 765 - protein, 9 aa, hepatitis C virus
RRSRNLGKV

SEQ ID NO: 766 - protein, 9 aa, hepatitis C virus
YRGLDVSVI

SEQ ID NO: 767 - protein, 9 aa, hepatitis C virus
ARLVVLATA

SEQ ID NO: 768 - protein, 9 aa, hepatitis C virus
ASGKRVYYL

SEQ ID NO: 769 - protein, 3010 aa, hepatitis C virus
  MISC_FEATURE (393)..(393): X is Ala or Gln
MSTNPKPQRKTKRNTNRRPQDVKFPGGGQIVGGVYLLPRRGPRLGVRATRKTSERSQPRGRRQPIPKARRPEGRTWAQPGYPWPLYGNEGLGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTIPASAYEVRNVSGVYHVTNDCSNSSIVYEAADMIMHTPGCVPCVRENNSSRCWVALTPTLAARNASVPTTTIRRHVDLLVGAAAFCSAMYVGDLCGSVFLVSQLFTFSPRRHETVQDCNCSIYPGHISGHRMAWDMMMNWSPTTALVVSQLLRIPQAVVDMVAGAHWGVLAGLAYYSMVGNWAKVLVVMLLFAGVDGTTHVTGGAAXRTTSGFTSLFSPGASQKIQLVNTNGSWHINRTALNCNDSLQTGFLAALFYTHRFNSSGCPERMASCRPIDKFAQGWGPITYAEPNSSDQRPYCWHYAPRPCGIVPASQVCGPVYCFTPSPVVVGTTDRFGVPTYSWGENETDVLLLNNTRPPQGNWFGCTWMNSTGFTKTCGGPPCNIGGVGNNTLTCPTDCFRKHPEATYTKCGSGPWLTPRCLVDYPYRLWHYPCTVNFTIFKVRMYVGGVEHRLNAACNWTRGERCDLEDRDRSELSPLLLSTTEWQILPCSFTTLPALSTGLIHLHQNIVDVQYLYGIGSAVVSFAIKWEYVLLLFLLLADARVCACLWMMLLIAQAEAALENLVVLNAASVAGAHGILSFLVFFCAAWYIKGRLVPGAAYAFYGVWPLLLLLLALPPRAYALDREMAASCGGAVFVGLALLTLSPYYKVFLARLIWWLQYFITRAEAHLQVWVPPLNVRGGRDAIILLTCAVHPELIFDITKLLLAILGPLMVLQAGITRVPYFVRAQGLIRACMLVRKVAGGHYVQMAFMKLAALTGTYVYDHLTPLRDWAHAGLRDLAVAVEPVVFSDMETKIITWGADTAACGDIILGLPVSARRGREILLGPADSLEGQGWRLLAPITAYSQQTRGLLGCIITSLTGRDKNQVEGEVQVVSTATQSFLATCVNGVCWTVYHGAGSKTLAGPKGPITQMYTNVDQDLVGWQAPPGARSMTPCTCGSSDLYLVTRHADVIPVRRRGDSRGSLLSPRPVSYLKGSSGGPLLCPSGHVVGVFRAAVCTRGVAKAVDFIPVESMETTMRSPVFTDNSSPPAVPQTFQVAHLHAPTGSGKSTKVPAAYAAQGYKVLVLNPSVAATLGFGAYMSKAHGVDPNIRTGVRTITTGAPITYSTYGKFLADGGCSGGAYDIIICDECHSTDSTTILGIGTVLDQAETAGARLVVLATATPPGSVTVPHPNIEEIGLSNNGEIPFYGKAIPIEAIKGGRHLIFCHSKKKCDELAAKLSALGLNAVAYYRGLDVSVIPTSGDVVVVATDALMTGYTGDFDSVIDCNTCVTQTVDFSLDPTFTIETTTVPQDAVSRSQRRGRTGRGRRGIYRFVTPGERPSGMFDSSVLCECYDAGCAWYELTPAETSVRLRAYLNTPGLPVCQDHLEFWESVFTGLTHIDAHFLSQTKQAGDNFPYLVAYQATVCARAQAPPPSWDQMWKCLIRLKPTLHGPTPLLYRLGAVQNEVTLTHPITKYIMACMSADLEVVTSTWVLVGGVLAALAAYCLTTGSVVIVGRIILSGRPAVIPDREVLYQEFDEMEECASHLPYIEQGMQLAEQFKQKALGLLQTATKQAEAAAPVVESKWRALETFWAKHMWNFISGIQYLAGLSTLPGNPAIASLMAFTASITSPLTTQHTLLFNILGGWVAAQLAPPSAASAFVGAGIAGAAVGSIGLGKVLVDILAGYGAGVAGALVAFKVMSGEMPSTEDLVNLLPAILSPGALVVGVVCAAILRRHVGPGEGAVQWMNRLIAFASRGNHVSPTHYVPESDAAARVTQILSSLTITQLLKRLHQWINEDCSTPCSGSWLRDVWDWICTVLTDFKTWLQSKLLPRLPGVPFFSCQRGYKGVWRGDGIMQTTCPCGAQITGHVKNGSMRIVGPKTCSNTWHGTFPINAYTTGPCTPSPAPNYSRALWRVAAEEYVEVTRVGDFHYVTGMTTDNVKCPCQVPAPEFFTEVDGVRLHRYAPACKPLLREEVTFQVGLNQYLVGSQLPCEPEPDVAVLTSMLTDPSHITAETAKRRLARGSPPSLASSSASQLSAPSLKATCTTHHDSPDADLIEANLLWRQEMGGNITRVESENKVVILDSFDPLRAEEDEREVSVAAEILRKSRKFPPALPIWARPDYNPPLLESWKDPDYVPPVVHGCPLPPTKAPPIPPPRRKRTVVLTESTVSSALAELATKTFGSSGSSAVDSGTATAPPDQASDDGDKGSDVESYSSMPPLEGEPGDPDLSDGSWSTVSEEASEDVVCCSMSYTWTGALITPCAAEESKLPINALSNSLLRHHNMVYATTSRSASLRQKKVTFDRLQVLDDHYRDVLKEMKAKASTVKAKLLSVEEACKLTPPHSAKSKFGYGAKDVRNLSSKAVNHIRSVWKDLLEDTETPIDTTIMAKNEVFCVQPEKGGRKPARLIVFPDLGVRVCEKMALYDVVSTLPQAVMGSSYGFQYSPGQRVEFLVNAWKSKKCPMGFSYDTRCFDSTVTENDIRVEESIYQCCDLAPEARQAIRSLTERLYIGGPLTNSKGQNCGYRRCRASGVLTTSCGNTLTCYLKASAACRAAKLQDCTMLVNGDDLVVICESAGTQEDAASLRVFTEAMTRYSAPPGDPPQPEYDLELITSCSSNVSVAHDASGKRVYYLTRDPTTPLARAAWETARHTPVNSWLGNIIMYAPTLWARMILMTHFFSILLAQEQLEKALDCQIYGACYSIEPLDLPQIIXRLHGLSAFSLHSYSPGEINRVASCLRKLGVPPLRVWRHRARSVRAKLLSQGGRAATCGKYLFNWAVRTKLKLTPIPAASQLDLSGWFVAGYSGGDIYHSLSRARPRWFMXCLLLLSVGVGIYLLPNR

SEQ ID NO: 770 - protein, 3010 aa, hepatitis C virus
MSTNPKPQRKTKRNTNRRPQDVKFPGGGQIVGGVYLLPRRGPRLGVRATRKTSERSQPRGRRQPIPKARRPEGRAWAQPGYPWPLYGNEGMGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTIPASAYEVRNVSGVYHVTNDCSNSSIVYEAADMIMHTPGCVPCVRENNSSRCWVALTPTLAARNSSVPTTTIRRHVDLLVGAAAFCSAMYVGDLCGSVFLVSQLFTFSPRRHETVQDCNCSIYPGHVSGHRMAWDMMMNWSPTTALVVSQLLRIPQAVVDMVAGAHWGVLAGLAYYSMVGNWAKVLIVMLLFAGVDGTTHVTGGAAARTTSGFTSLFSPGPSQKIQLVNTNGSWHINRTALNCNDSLQTGFLAALFYTHRFNASGCPERMASCRPIDKFAQGWGPITYAEPNSSDQRPYCWHYAPRPCGIVPASQVCGPVYCFTPSPVVVGTTDRFGVPTYSWGENETDVLLLNNTRPPQGNWFGCTWMNSTGFTKTCGGPPCNIGGVGNNTLTCPTDCFRKHPEATYTKCGSGPWLTPRCLVDYPYRLWHYPCTVNFTIFKVRMYVGGVEHRLNAACNWTRGERCDLEDRDRSELSPLLLSTTEWQILPCSFTTLPALSTGLIHLHQNIVDVQYLYGIGSAVVSFAIKWEYVLLLFLLLADARVCACLWMMLLIAQAEAALENLVVLNAASVAGAHGILSFLVFFCAAWYIKGRLVPGAAYALYGVWPLLLLLLALPPRAYAMDREMAASCGGAVFVGLALLTLSPYYKVFLARLIWWLQYFITRAEAHLQVWVPPLNVRGGRDAIILLTCAVHPELIFDITKLLLAILGPLMVLQAGITRVPYFVRAQGLIRACMLVRKVAGGHYVQMAFMKLAALTGTYVYDHLTPLRDWAHAGLRDLAVAVEPVVFSDMETKIITWGADTAACGDIILGLPVSARRGREILLGPADSLEGQGWRLLAPITAYSQQTRGLLGCIITSLTGRDKNQVEGEVQVVSTATQSFLATCVNGVCWTVYHGAGSKTLAGPKGPITQMYTNVDQDLVGWQAPPGARSLTPCTCGSSDLYLVTRHADVIPVRRRGDSRGSLLSPRPVSYLKGSSGGPLLCPSGHAVGIFRAAVCTRGVAKAVDFVPVESMETTMRSPVFTDNSSPPAVPQTFQVAHLHAPTGSGKSTKVPAAYAAQGYKVLVLNPSVAATLGFGAYMSKAHGVDPNIRTGVRTITTGAPITYSTYGKFLADGGCSGGAYDIIICDECHSTDSTTILGIGTVLDQAETAGARLVVLATATPPGSVTVPHPNIEEVALSNTGEIPFYGKAIPIEVIKGGRHLIFCHSKKKCDELAAKLSALGLNAVAYYRGLDVSVIPTSGDVVVVATDALMTGYTGDFDSVIDCNTCVTQTVDFSLDPTFTIETTTVPQDAVSRSQRRGRTGRGRRGIYRFVTPGERPSGMFDSSVLCECYDAGCAWYELTPAETSVRLRAYLNTPGLPVCQDHLEFWESVFTGLTHIDAHFLSQTKQAGDNFPYLVAYQATVCARAQAPPPSWDQMWKCLIRLKPTLHGPTPLLYRLGAVQNEVTLTHPITKYIMACMSADLEVVTSTWVLVGGVLAALAAYCLTTGSVVIVGRIILSGRPAVIPDREVLYREFDEMEECASHLPYIEQGMQLAEQFKQKALGLLQTATKQAEAAAPVVESKWRALETFWAKHMWNFISGIQYLAGLSTLPGNPAIASLMAFTASITSPLTTQHTLLFNILGGWVAAQLAPPSAASAFVGAGIAGAAVGSIGLGKVLVDILAGYGAGVAGALVAFKVMSGEMPSTEDLVNLLPAILSPGALVVGVVCAAILRRHVGPGEGAVQWMNRLIAFASRGNHVSPTHYVPESDAAARVTQILSSLTITQLLKRLHQWINEDCSTPCSGSWLRDVWDWICTVLTDFKTWLQSKLLPRLPGVPFFSCQRGYKGVWRGDGIMQTTCPCGAQITGHVKNGSMRIVGPKTCSNTWHGTFPINAYTTGPCTPSPAPNYSRALWRVAAEEYVEVTRVGDFHYVTGMTTDNVKCPCQVPAPEFFTEVDGVRLHRYAPACKPLLREEVTFQVGLNQYLVGSQLPCEPEPDVAVLTSMLTDPSHITAETAKRRLARGSPPSLASSSASQLSAPSLKATCTTHHDSPDADLIEANLLWRQEMGGNITRVESENKVVILDSFDPLRAEEDEREVSVAAEILRKSRKFPPALPIWARPDYNPPLLESWKDPDYVPPVVHGCPLPPTKAPPIPPPRRKRTVVLTESTVSSALAELATKTFGSSGSSAVDSGTATAPPDQASDDGDKGSDVESYSSMPPLEGEPGDPDLSDGSWSTVSEEASEDVVCCSMSYTWTGALITPCAAEESKLPINALSNSLLRHHNMVYATTSRSASLRQKKVTFDRLQVLDDHYRDVLKEMKAKASTVKAKLLSVEEACKLTPPHSAKSKFGYGAKDVRNLSSKAVNHIRSVWKDLLEDTETPIDTTIMAKNEVFCVQPEKGGRKPARLIVFPDLGVRVCEKMALYDVVSTLPQAVMGSSYGFQYSPGQRVEFLVNAWKSKKCPMGFSYDTRCFDSTVTENDIRVEESIYQCCDLAPEARQAIRSLTERLYIGGPLTNSKGQNCGYRRCRASGVLTTSCGNTLTCYLKASAACRAAKLQDCTMLVNGDDLVVICESAGTQEDAASLRVFTEAMTRYSAPPGDPPQPEYDLELITSCSSNVSVAHDASGKRVYYLTRDPTTPLARAAWETARHTPVNSWLGNIIMYAPTLWARMILMTHFFSILLAQEQLEKALDCQIYGACYSIEPLDLPQIIERLHGLSAFSLHSYSPGEINRVASCLRKLGVPPLRVWRHRARSVRAKLLSQGGRAATCGKYLFNWAVRTKLKLTPIPAASQLDLSGWFVAGYSGGDIYHSLSRARPRWFMLCLLLLSVGVGIYLLPNR

SEQ ID NO: 771 - protein, 3011 aa, hepatitis C virus
MSTNPKPQRKTKRNTNRRPQDVKFPGGGQIVGGVYLLPRRGPRLGVRATRKTSERSQPRGRRQPIPKARRPEGRTWAQPGYPWPLYGNEGCGWAGWLLSPRGSRPSWGPTDPRRRSRNLGKVIDTLTCGFADLMGYIPLVGAPLGGAARALAHGVRVLEDGVNYATGNLPGCSFSIFLLALLSCLTVPASAYQVRNSSGLYHVTNDCPNSSIVYEAADAILHTPGCVPCVREGNASRCWVAVTPTVATRDGKLPTTQLRRHIDLLVGSATLCSALYVGDLCGSVFLVGQLFTFSPRRHWTTQDCNCSIYPGHITGHRMAWDMMMNWSPTAALVVAQLLRIPQAILDMIAGAHWGVLAGIAYFSMVGNWAKVLVVLLLFAGVDAETHVTGGSAARTTAGLVSLLTPGAKQNIQLINTNGSWHINSTALNCNESLNTGWLAGLFYTHKFNSSGCPERLASCRRLTDFDQGWGPISYANGSGPDQRPYCWHYPPKPCGIVPAKSVCGPVYCFTPSPVVVGTTDRSGAPTYSWGANDTDVFVLNNTRPPLGNWFGCTWMNSTGFTKVCGAPPCVIGGVGNNTLHCPTDCFRKHPEATYSRCGSGPWITPRCLVDYPYRLWHYPCTINYTIFKVRMYVGGVEHRLEAACNWTRGERCDLEDRDRSELSPLLLSTTQWQVLPCSFTTLPALSTGLIHLHQNIVDVQYLYGVGSSIASWAIKWEYVVLLFLLLADARVCSCLWMMLLISQAEAALENLVILNAASLAGTHGLVSFLVFFCFAWYLKGRWVPGAVYALYGMWPLLLLLLALPQRAYALDTEVAASCGGVVLVGLMALTLSPYYKRYISWCLWWLQYFLTRVEAQLHVWVPPLNVRGGRDAVILLMCVVHPTLVFDITKLLLAVFGPLWILQASLLKVPYFVRVQGLLRICALARKMAGGHYVQMAIIKLGALTGTYVYNHLTPLRDWAHNGLRDLAVAVEPVVFSQMETKLITWGADTAACGDIINGLPVSARRGREILLGPADGMVSKGWRLLAPITAYAQQTRGLLGCIITSLTGRDKNQVEGEVQIVSTAAQTFLATCINGVCWTVYHGAGTRTIASPKGPVIQMYTNVDQDLVGWPAPQGARSLTPCTCGSSDLYLVTRHADVIPVRRRGDSRGSLLSPRPISYLKGSSGGPLLCPAGHAVGIFRAAVCTRGVAKAVDFIPVENLETTMRSPVFTDNSSPPAVPQSFQVAHLHAPTGSGKSTKVPAAYAAQGYKVLVLNPSVAATLGFGAYMSKAHGIDPNIRTGVRTITTGSPITYSTYGKFLADGGCSGGAYDIIICDECHSTDATSILGIGTVLDQAETAGARLVVLATATPPGSVTVPHPNIEEVALSTTGEIPFYGKAIPLEVIKGGRHLIFCHSKKKCDELAAKLVALGINAVAYYRGLDVSVIPTSGDVVVVATDALMTGFTGDFDSVIDCNTCVTQTVDFSLDPTFTIETTTLPQDAVSRTQRRGRTGRGKPGIYRFVAPGERPSGMFDSSVLCECYDAGCAWYELTPAETTVRLRAYMNTPGLPVCQDHLEFWEGVFTGLTHIDAHFLSQTKQSGENFPYLVAYQATVCARAQAPPPSWDQMWKCLIRLKPTLHGPTPLLYRLGAVQNEVTLTHPVTKYIMTCMSADLEVVTSTWVLVGGVLAALAAYCLSTGCVVIVGRIVLSGKPAIIPDREVLYREFDEMEECSQHLPYIEQGMMLAEQFKQKALGLLQTASRQAEVIAPAVQTNWQKLEAFWAKHMWNFISGIQYLAGLSTLPGNPAIASLMAFTAAVTSPLTTSQTLLFNILGGWVAAQLAAPGAATAFVGAGLAGAAIGSVGLGKVLVDILAGYGAGVAGALVAFKIMSGEVPSTEDLVNLLPAILSPGALVVGVVCAAILRRHVGPGEGAVQWMNRLIAFASRGNHVSPTHYVPESDAAARVTAILSSLTVTQLLRRLHQWISSECTTPCSGSWLRDIWDWICEVLSDFKTWLKAKLMPQLPGIPFVSCQRGYRGVWRGDGIMHTRCHCGAEITGHVKNGTMRIVGPKTCRNMWSGTFPINAYTTGPCTPLPAPNYTFALWRVSAEEYVEIRRVGDFHYVTGMTTDNLKCPCQVPSPEFFTELDGVRLHRFAPPCKPLLREEVSFRVGLHEYPVGSQLPCEPEPDVAVLTSMLTDPSHITAEAAGRRLARGSPPSMASSSASQLSAPSLKATCTANHDSPDAELIEANLLWRQEMGGNITRVESENKVVILDSFDPLVAEEDEREISVPAEILRKSRRFAQALPVWARPDYNPPLLETWKKPDYEPPVVHGCPLPPPRSPPVPPPRKKRTVVLTESTLSTALAELATKSFGSSSTSGITGDNTTTSSEPAPSGCPPDSDAESYSSMPPLEGEPGDPDLSDGSWSTVSSEASTEDVVCCSMSYSWTGALVTPCAAEEQKLPINALSNSLLRHHNLVYSTTSRSACQRQKKVTFDRLQVLDSHYQDVLKEVKAAASKVKANLLSVEEACSLTPPHSAKSKFGYGAKDVRCHARKAVNHINSVWKDLLEDSVTPIDTTIMAKNEVFCVQPEKGGRKPARLIVFPDLGVRVCEKMALYDVVSKLPLAVMGSSYGFQYSPGQRVEFLVQAWKSKKTPMGFSYDTRCFDSTVTESDIRTEEAIYQCCDLDPQARVAIKSLTERLYVGGPLTNSRGENCGYRRCRASGVLTTSCGNTLTCYIKARAACRAAGLQDCTMLVCGDDLVVICESAGVQEDAASLRAFTEAMTRYSAPPGDPPQPEYDLELITSCSSNVSVAHDGAGKRVYYLTRDPTTPLARAAWETARHTPVNSWLGNIIMFAPTLWARMILMTHFFSVLIARDQLEQALDCEIYGACYSIEPLDLPPIIQRLHGLSAFSLHSYSPGEINRVAACLRKLGVPPLRAWRHRARSVRARLLSRGGRAAICGKYLFNWAVRTKLKLTPIAAAGRLDLSGWFTAGYSGGDIYHSVSHARPRWFWFCLLLLAAGVGIYLLPNR

SEQ ID NO: 772 - protein, 3020 aa, hepatitis C virus
MSTLPKPQRKTKRNTIRRPQDVKFPGGGQIVGGVYVLPRRGPRLGVRATRKTSERSQPRGRRQPIPKARRSEGRSWAQPGYPWPLYGNEGCGWAGWLLSPRGSRPSWGPNDPRRRSRNLGKVIDTLTCGFADLMGYIPLVGAPVGGVARALAHGVRALEDGINFATGNLPGCSFSIFLLALFSCLIHPAASLEWRNTSGLYVLTNDCSNSSIVYEADDVILHTPGCVPCVQDGNTSTCWTPVTPTVAVRYVGATTASIRSHVDLLVGAATMCSALYVGDMCGAVFLVGQAFTFRPRRHQTVQTCNCSLYPGHLSGHRMAWDMMMNWSPAVGMVVAHVLRLPQTLFDIIAGAHWGILAGLAYYSMQGNWAKVAIIMVMFSGVDATTYTTGGSAARTTSGLTSLFSVGPQQKLQLVNTNGSWHINSTALNCNESINTGFIAGLFYYHKFNSTGCPQRLSSCKPITFFAQGWGPLTDANITGPSDDKPYCWHYAPRPCDIVPASNVCGPVYCFTPSPVVVGTTDAKGVPTYTWGENETDVFLLESLRPPSGRWFGCTWMNSTGFTKTCGAPPCNIYGGGGNPNESDLFCPTDCFRKHPEATYSRCGAGPWLTPRCMVDYPYRLWHYPCTVNFTLFKVRMFVGGFEHRFTAACNWTRGERCDIEDRDRSEQHPLLHSTTELAILPCSFTPMPALSTGLIHLHQNIVDVQYLYGVGSGMVGWALKWEFVILIFLLLADARVCVALWLMLMISQAEAALENLVTLNAVAAAGTHGIGWYLVAFCAAWYVRGKLVPLVTYSLTGLWSLALLVLLLPQRAYAWSGEDSATLGAGVLVLFGFFTLSPWYKHWIGRLIWWNQYTICRCESALQVWVPPLLARGSRDGVILLTSLLYPSLIFDITKLLIAVLGPLYLIQATITRTPYFVRAHVLVRLCMLVRSVMGGKYFQMIILSIGRWFNTYLYDHLAPMQHWAAAGLKDLAVATEPVIFSPMEIKVITWGADTAACGDILCGLPVSARLGREVLLGPADDYREMGWRLLAPITAYAQQTRGLLGTIVTSLTGRDKNVVTGEVQVLSTATQTFLGTTVGGVMWTVYHGAGSRTLAGAKHPALQMYTNVDQDLVGWPAPPGAKSLEPCACGSADLYLVTRDADVIPARRRGDSTASLLSPRPLACLKGSSGGPVMCPSGHVAGIFRAAVCTRGVAKALQFIPVETLSTQARSPSFSDNSTPPAVPQSYQVGYLHAPTGSGKSTKVPAAYVAQGYNVLVLNPSVAATLGFGSFMSRAYGIDPNIRTGNRTVTTGAKLTYSTYGKFLADGGCSGGAYDVIICDECHAQDATSILGIGTVLDQAETAGVRLTVLATATPPGSITVPHSNIEEVALGSEGEIPFYGKAIPIALLKGGRHLIFCHSKKKCDEIASKLRGMGLNAVAYYRGLDVSVIPTTGDVVVCATDALMTGFTGDFDSVIDCNVAVEQYVDFSLDPTFSIETRTAPQDAVSRSQRRGRTGRGRLGTYRYVAPGERPSGMFDSVVLCECYDAGCSWYDLQPAETTVRLRAYLSTPGLPVCQDHLDFWESVFTGLTHIDAHFLSQTKQQGLNFSYLTAYQATVCARAQAPPPSWDEMWKCLVRLKPTLHGPTPLLYRLGPVQNEVCLTHPITKYVMACMSADLEVTTSTWVLLGGVLAALAAYCLSVGCVVIVGHIELGGKPALVPDKEVLYQQYDEMEECSQAAPYIEQAQQIAHQFKEKVLGLLQRATQQQAVIEPIVATNWQKLEAFWHKHMWNFVSGIQYLAGLSTLPGNPAVASLMAFTASVTSPLTTNQTMFFNILGGWVATHLAGPQSSSAFVVSGLAGAAIGGIGLGRVLLDILAGYGAGVSGALVAFKIMGGELPTTEDMVNLLPAILSPGALVVGVICAAILRRHVGPGEGAVQWMNRLIAFASRGNHVSPTHYVPESDAAARVTALLSSLTVTSLLRRLHQWINEDYPSPCSDDWLRIIWDWVCSVLTDFKTWLSAKIMPALPGLPFISCQKGYKGVWRGDGVMSTRCPCGATITGHVKNGSMRLAGPRTCANMWHGTFPINEYTTGPSTPCPSPNYTRALWRVAANSYVEVRRVGDFHYITGATEDELKCPCQVPAAEFFTEVDGVRLHRYAPPCKPLLRDEITFMVGLNSYLIGSQLPCEPEPDVSVLTSMLRDPSHITAETAARRLARGSPPSEASSSASQLSAPSLKATCQTHRPHPDAELVDANLLWRQEMGSNITRVESETKVVILDSFEPLRAETDDAELSVAAECFKKPPKYPPALPIWARPDYNPPLLDRWKAPDYVPPTVHGCALPPRGAPPVPPPRRKRTIQLDGSNVSAALAALAEKSFPSSKPQEENSSSSGVDTQSSTTSKVPPSPGGESDSESCSSMPPLEGEPGDPDLSCDSWSTVSDSEEQSVVCCSMSYSWTGALITPCSAEEEKLPISPLSNSLLRHHNLVYSTSSRSASQRQKKVTFDRLQVLDDHYKTALKEVKERASRVKARMLTIEEACALVPPHSARSKFGYSAKDVRSLSSRAINQIRSVWEDLLEDTTTPIPTTIMAKNEVFCVDPAKGGRKPARLIVYPDLGVRVCEKRALYDVIQKLSIETMGSAYGFQYSPQQRVERLLKMWTSKKTPLGFSYDTRCFDSTVTEQDIRVEEEIYQCCNLEPEARKVISSLTERLYCGGPMFNSKGAQCGYRRCRASGVLPTSFGNTITCYIKATAAAKAAGLRNPDFLVCGDDLVVVAESDGVDEDRAALRAFTEAMTRYSAPPGDAPQPTYDLELITSCSSNVSVARDDKGKRYYYLTRDATTPLARAAWETARHTPVNSWLGNIIMYAPTIWVRMVMMTHFFSILQSQEILDRPLDFEMYGATYSVTPLDLPAIIERLHGLSAFTLHSYSPVELNRVAGTLRKLGCPPLRAWRHRARAVRAKLIAQGGKAKICGLYLFNWAVRTKTKLTPLPAAGQLDLSSWFTVGVGGNDIYHSVSRARTRYLLLCLLLLTVGVGIFLLPAR

SEQ ID NO: 773 - protein, 9 aa, hepatitis C virus
AAARVTQIL

SEQ ID NO: 774 - protein, 10 aa, hepatitis C virus
AACGDIILGL

SEQ ID NO: 775 - protein, 10 aa, hepatitis C virus
AAKLQDCTML

SEQ ID NO: 776 - protein, 9 aa, hepatitis C virus
AALENLVVL

SEQ ID NO: 777 - protein, 10 aa, hepatitis C virus
AAQGYKVLVL

SEQ ID NO: 778 - protein, 9 aa, hepatitis C virus
AARNSSVPT

SEQ ID NO: 779 - protein, 10 aa, hepatitis C virus
AARNSSVPTT

SEQ ID NO: 780 - protein, 9 aa, hepatitis C virus
AARTTSGFT

SEQ ID NO: 781 - protein, 10 aa, hepatitis C virus
AATLGFGAYM

SEQ ID NO: 782 - protein, 9 aa, hepatitis C virus
AAWYIKGRL

SEQ ID NO: 783 - protein, 10 aa, hepatitis C virus
AAYAAQGYKV

SEQ ID NO: 784 - protein, 9 aa, hepatitis C virus
ACGDIILGL

SEQ ID NO: 785 - protein, 10 aa, hepatitis C virus
ACYSIEPLDL

SEQ ID NO: 786 - protein, 9 aa, hepatitis C virus
ADLEVVTST

SEQ ID NO: 787 - protein, 9 aa, hepatitis C virus
AEAAARRLA

SEQ ID NO: 788 - protein, 9 aa, hepatitis C virus
AEAALEKLV

SEQ ID NO: 789 - protein, 10 aa, hepatitis C virus
AEAALEKLVI

SEQ ID NO: 790 - protein, 9 aa, hepatitis C virus
AEAALENLV

SEQ ID NO: 791 - protein, 10 aa, hepatitis C virus
AEAALENLVI

SEQ ID NO: 792 - protein, 10 aa, hepatitis C virus
AEILRKSRRF

SEQ ID NO: 793 - protein, 9 aa, hepatitis C virus
AELATKTFG

SEQ ID NO: 794 - protein, 9 aa, hepatitis C virus
AEQFKQKAL

SEQ ID NO: 795 - protein, 10 aa, hepatitis C virus
AEQFKQKALG

SEQ ID NO: 796 - protein, 10 aa, hepatitis C virus
AETAGARLVV

SEQ ID NO: 797 - protein, 9 aa, hepatitis C virus
AETAGVRLT

SEQ ID NO: 798 - protein, 10 aa, hepatitis C virus
AETAGVRLTV

SEQ ID NO: 799 - protein, 10 aa, hepatitis C virus
AETSVRLRAY

SEQ ID NO: 800 - protein, 9 aa, hepatitis C virus
AETTVRLRA

SEQ ID NO: 801 - protein, 9 aa, hepatitis C virus
AFKIMSGEK

SEQ ID NO: 802 - protein, 10 aa, hepatitis C virus
AFTASITSPL

SEQ ID NO: 803 - protein, 10 aa, hepatitis C virus
AFWAKHMWNF

SEQ ID NO: 804 - protein, 10 aa, hepatitis C virus
AGAAVGSIGL

SEQ ID NO: 805 - protein, 10 aa, hepatitis C virus
AGAHGILSFL

SEQ ID NO: 806 - protein, 10 aa, hepatitis C virus
AGPKGPITQM

SEQ ID NO: 807 - protein, 10 aa, hepatitis C virus
AGTQEDAASL

SEQ ID NO: 808 - protein, 10 aa, hepatitis C virus
AGVAGALVAF

SEQ ID NO: 809 - protein, 9 aa, hepatitis C virus
AGYSGGDIY

SEQ ID NO: 810 - protein, 10 aa, hepatitis C virus
AHDASGKRVY

SEQ ID NO: 811 - protein, 10 aa, hepatitis C virus
AHLQVWVPPL

SEQ ID NO: 812 - protein, 9 aa, hepatitis C virus
AHWGVLAGL

SEQ ID NO: 813 - protein, 9 aa, hepatitis C virus
AIKGGRHLI

SEQ ID NO: 814 - protein, 9 aa, hepatitis C virus
AIKWEYVLL

SEQ ID NO: 815 - protein, 10 aa, hepatitis C virus
AIKWEYVLLL

SEQ ID NO: 816 - protein, 9 aa, hepatitis C virus
AILGPLMVL

SEQ ID NO: 817 - protein, 10 aa, hepatitis C virus
AIRSLTERLY

SEQ ID NO: 818 - protein, 9 aa, hepatitis C virus
AITYSTYGK

SEQ ID NO: 819 - protein, 10 aa, hepatitis C virus
AKLQDCTMLV

SEQ ID NO: 820 - protein, 9 aa, hepatitis C virus
ALAAYCLSV

SEQ ID NO: 821 - protein, 9 aa, hepatitis C virus
ALAAYCLTT

SEQ ID NO: 822 - protein, 10 aa, hepatitis C virus
ALGLNAVAYY

SEQ ID NO: 823 - protein, 9 aa, hepatitis C virus
ALLGPAYLL

SEQ ID NO: 824 - protein, 9 aa, hepatitis C virus
ALQVWVPPL

SEQ ID NO: 825 - protein, 9 aa, hepatitis C virus
ALSTGLIHL

SEQ ID NO: 826 - protein, 9 aa, hepatitis C virus
ALSTGLLHL

SEQ ID NO: 827 - protein, 9 aa, hepatitis C virus
ALVVSQLLR

SEQ ID NO: 828 - protein, 9 aa, hepatitis C virus
ALYDITQKL

SEQ ID NO: 829 - protein, 9 aa, hepatitis C virus
ALYDVIQKL

SEQ ID NO: 830 - protein, 9 aa, hepatitis C virus
ALYGVWPLL

SEQ ID NO: 831 - protein, 10 aa, hepatitis C virus
ALYGVWPLLL

SEQ ID NO: 832 - protein, 9 aa, hepatitis C virus
ANLLWRQEM

SEQ ID NO: 833 - protein, 9 aa, hepatitis C virus
APACKPLLR

SEQ ID NO: 834 - protein, 10 aa, hepatitis C virus
APACKPLLRE

SEQ ID NO: 835 - protein, 10 aa, hepatitis C virus
APITAYSQQT

SEQ ID NO: 836 - protein, 10 aa, hepatitis C virus
APLGGAARAL

SEQ ID NO: 837 - protein, 10 aa, hepatitis C virus
APLGGVARAL

SEQ ID NO: 838 - protein, 9 aa, hepatitis C virus
APNYSRALW

SEQ ID NO: 839 - protein, 9 aa, hepatitis C virus
APPGARSLT

SEQ ID NO: 840 - protein, 10 aa, hepatitis C virus
APPGARSLTP

SEQ ID NO: 841 - protein, 9 aa, hepatitis C virus
APPGDPPQP

SEQ ID NO: 842 - protein, 10 aa, hepatitis C virus
APPGDPPQPE

SEQ ID NO: 843 - protein, 9 aa, hepatitis C virus
APPIPPPRR

SEQ ID NO: 844 - protein, 9 aa, hepatitis C virus
APPSAASAF

SEQ ID NO: 845 - protein, 10 aa, hepatitis C virus
APPSAASAFV

SEQ ID NO: 846 - protein, 9 aa, hepatitis C virus
APRPCGIVP

SEQ ID NO: 847 - protein, 10 aa, hepatitis C virus
APRPCGIVPA

SEQ ID NO: 848 - protein, 9 aa, hepatitis C virus
APSLKATCT

SEQ ID NO: 849 - protein, 10 aa, hepatitis C virus
APSLKATCTT

SEQ ID NO: 850 - protein, 9 aa, hepatitis C virus
APTIWVRMV

SEQ ID NO: 851 - protein, 10 aa, hepatitis C virus
APTIWVRMVL

SEQ ID NO: 852 - protein, 8 aa, hepatitis C virus
APTLWARM

SEQ ID NO: 853 - protein, 10 aa, hepatitis C virus
APTLWARMIL

SEQ ID NO: 854 - protein, 11 aa, hepatitis C virus
APTLWARMILM

SEQ ID NO: 855 - protein, 10 aa, hepatitis C virus
APVGGVARAL

SEQ ID NO: 856 - protein, 10 aa, hepatitis C virus
APVVESKWRA

SEQ ID NO: 857 - protein, 10 aa, hepatitis C virus
APYIEQAQAI

SEQ ID NO: 858 - protein, 10 aa, hepatitis C virus
AQAEAALENL

SEQ ID NO: 859 - protein, 9 aa, hepatitis C virus
AQGLIRACM

SEQ ID NO: 860 - protein, 10 aa, hepatitis C virus
AQGLIRACML

SEQ ID NO: 861 - protein, 10 aa, hepatitis C virus
AQPGYPWPLY

SEQ ID NO: 862 - protein, 10 aa, hepatitis C virus
ARALAHGVRV

SEQ ID NO: 863 - protein, 10 aa, hepatitis C virus
ARHTPVNSWL

SEQ ID NO: 864 - protein, 10 aa, hepatitis C virus
ARMILMTHFF

SEQ ID NO: 865 - protein, 9 aa, hepatitis C virus
ARRGREILL

SEQ ID NO: 866 - protein, 10 aa, hepatitis C virus
ARRPEGRAWA

SEQ ID NO: 867 - protein, 9 aa, hepatitis C virus
ARSVRAKLL

SEQ ID NO: 868 - protein, 10 aa, hepatitis C virus
ARVTQILSSL

SEQ ID NO: 869 - protein, 10 aa, hepatitis C virus
ASAACRAAKL

SEQ ID NO: 870 - protein, 10 aa, hepatitis C virus
ASGKRVYYLT

SEQ ID NO: 871 - protein, 10 aa, hepatitis C virus
ASLRVFTEAM

SEQ ID NO: 872 - protein, 9 aa, hepatitis C virus
ASQLSAPSL

SEQ ID NO: 873 - protein, 10 aa, hepatitis C virus
ASQLSAPSLK

SEQ ID NO: 874 - protein, 9 aa, hepatitis C virus
ASSVCGPVY

SEQ ID NO: 875 - protein, 9 aa, hepatitis C virus
ASTVKAKLL

SEQ ID NO: 876 - protein, 10 aa, hepatitis C virus
ASVAGAHGIL

SEQ ID NO: 877 - protein, 10 aa, hepatitis C virus
ATDALMTGYT

SEQ ID NO: 878 - protein, 10 aa, hepatitis C virus
ATLGFGAYMS

SEQ ID NO: 879 - protein, 9 aa, hepatitis C virus
ATTSRSASL

SEQ ID NO: 880 - protein, 10 aa, hepatitis C virus
AVAYYRGLDV

SEQ ID NO: 881 - protein, 9 aa, hepatitis C virus
AVCSRGVAK

SEQ ID NO: 882 - protein, 10 aa, hepatitis C virus
AVDFIPVESM

SEQ ID NO: 883 - protein, 10 aa, hepatitis C virus
AVDFVPVESM

SEQ ID NO: 884 - protein, 9 aa, hepatitis C virus
AVDLYLVTR

SEQ ID NO: 885 - protein, 10 aa, hepatitis C virus
AVEPVVFSDM

SEQ ID NO: 886 - protein, 9 aa, hepatitis C virus
AVFVGLALL

SEQ ID NO: 887 - protein, 9 aa, hepatitis C virus
AVGIFRAAV

SEQ ID NO: 888 - protein, 9 aa, hepatitis C virus
AVGSIGLGK

SEQ ID NO: 889 - protein, 9 aa, hepatitis C virus
AVGSVGLGK

SEQ ID NO: 890 - protein, 10 aa, hepatitis C virus
AVHPELIFDI

SEQ ID NO: 891 - protein, 9 aa, hepatitis C virus
AVIPDREVL

SEQ ID NO: 892 - protein, 9 aa, hepatitis C virus
AVLGPLYLI

SEQ ID NO: 893 - protein, 9 aa, hepatitis C virus
AVQWMNRLI

SEQ ID NO: 894 - protein, 9 aa, hepatitis C virus
AVRASLISR

SEQ ID NO: 895 - protein, 9 aa, hepatitis C virus
AVRTKLKLT

SEQ ID NO: 896 - protein, 9 aa, hepatitis C virus
AWAKVVVIL

SEQ ID NO: 897 - protein, 10 aa, hepatitis C virus
AWETARHTPV

SEQ ID NO: 898 - protein, 9 aa, hepatitis C virus
AWKSKKCPM

SEQ ID NO: 899 - protein, 10 aa, hepatitis C virus
AWKSKKCPMG

SEQ ID NO: 900 - protein, 10 aa, hepatitis C virus
AYAAQGYKVL

SEQ ID NO: 901 - protein, 10 aa, hepatitis C virus
AYALYGVWPL

SEQ ID NO: 902 - protein, 9 aa, hepatitis C virus
AYFSMQGAW

SEQ ID NO: 903 - protein, 10 aa, hepatitis C virus
AYLNTPGLPV

SEQ ID NO: 904 - protein, 9 aa, hepatitis C virus
AYMSKAHGI

SEQ ID NO: 905 - protein, 9 aa, hepatitis C virus
AYSQQTRGL

SEQ ID NO: 906 - protein, 10 aa, hepatitis C virus
AYSQQTRGLL

SEQ ID NO: 907 - protein, 10 aa, hepatitis C virus
AYYRGLDVSV

SEQ ID NO: 908 - protein, 10 aa, hepatitis C virus
CAAWYIKGRL

SEQ ID NO: 909 - protein, 9 aa, hepatitis C virus
CACLWMMLL

SEQ ID NO: 910 - protein, 10 aa, hepatitis C virus
CECYDAGCAW

SEQ ID NO: 911 - protein, 9 aa, hepatitis C virus
CEKMALYDI

SEQ ID NO: 912 - protein, 9 aa, hepatitis C virus
CEPEPDVAV

SEQ ID NO: 913 - protein, 10 aa, hepatitis C virus
CEPEPDVAVL

SEQ ID NO: 914 - protein, 9 aa, hepatitis C virus
CGAPPCNIY

SEQ ID NO: 915 - protein, 9 aa, hepatitis C virus
CGGAVFVGL

SEQ ID NO: 916 - protein, 10 aa, hepatitis C virus
CGSVFLVSQL

SEQ ID NO: 917 - protein, 10 aa, hepatitis C virus
CGYRRCRASG

SEQ ID NO: 918 - protein, 10 aa, hepatitis C virus
CHSKKKCDEL

SEQ ID NO: 919 - protein, 9 aa, hepatitis C virus
CINGVCWTV

SEQ ID NO: 920 - protein, 9 aa, hepatitis C virus
CLIDYPYRL

SEQ ID NO: 921 - protein, 10 aa, hepatitis C virus
CLRKLGVPPL

SEQ ID NO: 922 - protein, 9 aa, hepatitis C virus
CLVDYPYRL

SEQ ID NO: 923 - protein, 9 aa, hepatitis C virus
CLVHYPYRL

SEQ ID NO: 924 - protein, 9 aa, hepatitis C virus
CMVDYPYRL

SEQ ID NO: 925 - protein, 10 aa, hepatitis C virus
CPAGHAVGIF

SEQ ID NO: 926 - protein, 9 aa, hepatitis C virus
CPCGAQITG

SEQ ID NO: 927 - protein, 10 aa, hepatitis C virus
CPCQVPAPEF

SEQ ID NO: 928 - protein, 9 aa, hepatitis C virus
CPLPPTKAP

SEQ ID NO: 929 - protein, 9 aa, hepatitis C virus
CPRGHAVGI

SEQ ID NO: 930 - protein, 10 aa, hepatitis C virus
CPRGHAVGIF

SEQ ID NO: 931 - protein, 9 aa, hepatitis C virus
CPSGHAVGI

SEQ ID NO: 932 - protein, 10 aa, hepatitis C virus
CPSGHAVGIF

SEQ ID NO: 933 - protein, 9 aa, hepatitis C virus
CPSGHVVGI

SEQ ID NO: 934 - protein, 9 aa, hepatitis C virus
CPTDCFRKH

SEQ ID NO: 935 - protein, 10 aa, hepatitis C virus
CSFSIFLLAL

SEQ ID NO: 936 - protein, 9 aa, hepatitis C virus
CSFTTLPAL

SEQ ID NO: 937 - protein, 10 aa, hepatitis C virus
CSTPCSGSWL

SEQ ID NO: 938 - protein, 9 aa, hepatitis C virus
CTCGAVDLY

SEQ ID NO: 939 - protein, 9 aa, hepatitis C virus
CTCGSADLY

SEQ ID NO: 940 - protein, 9 aa, hepatitis C virus
CTCGSSDLY

SEQ ID NO: 941 - protein, 10 aa, hepatitis C virus
CTCGSSDLYL

SEQ ID NO: 942 - protein, 10 aa, hepatitis C virus
CTMLVNGDDL

SEQ ID NO: 943 - protein, 9 aa, hepatitis C virus
CTPSPAPNY

SEQ ID NO: 944 - protein, 9 aa, hepatitis C virus
CTVNFSIFK

SEQ ID NO: 945 - protein, 9 aa, hepatitis C virus
CTVNFTIFK

SEQ ID NO: 946 - protein, 9 aa, hepatitis C virus
CTVNFTLFK

SEQ ID NO: 947 - protein, 9 aa, hepatitis C virus
CTWMNSTGY

SEQ ID NO: 948 - protein, 10 aa, hepatitis C virus
CVRENNSSRC

SEQ ID NO: 949 - protein, 10 aa, hepatitis C virus
CVTQTVDFSL

SEQ ID NO: 950 - protein, 9 aa, hepatitis C virus
CWVALTPTL

SEQ ID NO: 951 - protein, 9 aa, hepatitis C virus
CYSIEPLDL

SEQ ID NO: 952 - protein, 10 aa, hepatitis C virus
DAAARVTQIL

SEQ ID NO: 953 - protein, 10 aa, hepatitis C virus
DAGCAWYELT

SEQ ID NO: 954 - protein, 10 aa, hepatitis C virus
DARVCACLWM

SEQ ID NO: 955 - protein, 10 aa, hepatitis C virus
DASGKRVYYL

SEQ ID NO: 956 - protein, 9 aa, hepatitis C virus
DELAAALRG

SEQ ID NO: 957 - protein, 10 aa, hepatitis C virus
DELAAKLSAL

SEQ ID NO: 958 - protein, 10 aa, hepatitis C virus
DFSLDPTFTI

SEQ ID NO: 959 - protein, 9 aa, hepatitis C virus
DGGCSGGAY

SEQ ID NO: 960 - protein, 10 aa, hepatitis C virus
DHLEFWESVF

SEQ ID NO: 961 - protein, 10 aa, hepatitis C virus
DITKLLLAIL

SEQ ID NO: 962 - protein, 8 aa, hepatitis C virus
DLCGSVFL

SEQ ID NO: 963 - protein, 9 aa, hepatitis C virus
DLCGSVFLV

SEQ ID NO: 964 - protein, 10 aa, hepatitis C virus
DLEDRDRSEL

SEQ ID NO: 965 - protein, 10 aa, hepatitis C virus
DLMGYIPLVG

SEQ ID NO: 966 - protein, 9 aa, hepatitis C virus
DLMGYIPVV

SEQ ID NO: 967 - protein, 9 aa, hepatitis C virus
DLPQIIERL

SEQ ID NO: 968 - protein, 9 aa, hepatitis C virus
DLVNLLPAI

SEQ ID NO: 969 - protein, 10 aa, hepatitis C virus
DLVNLLPAIL

SEQ ID NO: 970 - protein, 9 aa, hepatitis C virus
DMRPYCWHY

SEQ ID NO: 971 - protein, 9 aa, hepatitis C virus
DPDYVPPVV

SEQ ID NO: 972 - protein, 9 aa, hepatitis C virus
DPNIRTGVR

SEQ ID NO: 973 - protein, 10 aa, hepatitis C virus
DPNIRTGVRT

SEQ ID NO: 974 - protein, 8 aa, hepatitis C virus
DPRRRSRN

SEQ ID NO: 975 - protein, 10 aa, hepatitis C virus
DPRRRSRNLG

SEQ ID NO: 976 - protein, 9 aa, hepatitis C virus
DPSHITAET

SEQ ID NO: 977 - protein, 10 aa, hepatitis C virus
DPSHITAETA

SEQ ID NO: 978 - protein, 9 aa, hepatitis C virus
DPTFTIETT

SEQ ID NO: 979 - protein, 10 aa, hepatitis C virus
DPTFTIETTT

SEQ ID NO: 980 - protein, 9 aa, hepatitis C virus
DPTTPLARA

SEQ ID NO: 981 - protein, 10 aa, hepatitis C virus
DPTTPLARAA

SEQ ID NO: 982 - protein, 10 aa, hepatitis C virus
DQMWKCLIRL

SEQ ID NO: 983 - protein, 9 aa, hepatitis C virus
DQMWKCLTR

SEQ ID NO: 984 - protein, 9 aa, hepatitis C virus
DQRPYCWHY

SEQ ID NO: 985 - protein, 9 aa, hepatitis C virus
DREVLYREF

SEQ ID NO: 986 - protein, 9 aa, hepatitis C virus
DRSELSPLL

SEQ ID NO: 987 - protein, 9 aa, hepatitis C virus
DSVVLCECY

SEQ ID NO: 988 - protein, 10 aa, hepatitis C virus
DTLTCGFADL

SEQ ID NO: 989 - protein, 9 aa, hepatitis C virus
DVAVLTSML

SEQ ID NO: 990 - protein, 10 aa, hepatitis C virus
DVKFPGGGQI

SEQ ID NO: 991 - protein, 9 aa, hepatitis C virus
DVMWKCLTR

SEQ ID NO: 992 - protein, 9 aa, hepatitis C virus
DVRNLSSKA

SEQ ID NO: 993 - protein, 10 aa, hepatitis C virus
DVRNLSSKAV

SEQ ID NO: 994 - protein, 10 aa, hepatitis C virus
DVVVVATDAL

SEQ ID NO: 995 - protein, 10 aa, hepatitis C virus
DVWDWICTVL

SEQ ID NO: 996 - protein, 10 aa, hepatitis C virus
DYNPPLLETW

SEQ ID NO: 997 - protein, 9 aa, hepatitis C virus
DYPYRLWHY

SEQ ID NO: 998 - protein, 10 aa, hepatitis C virus
EAALENLVVL

SEQ ID NO: 999 - protein, 10 aa, hepatitis C virus
EAMTRYSAPP

SEQ ID NO: 1000 - protein, 10 aa, hepatitis C virus
EANLLWRQEM

SEQ ID NO: 1001 - protein, 10 aa, hepatitis C virus
EARQAIRSLT

SEQ ID NO: 1002 - protein, 10 aa, hepatitis C virus
ECYDAGCAWY

SEQ ID NO: 1003 - protein, 10 aa, hepatitis C virus
EEARTAIHSL

SEQ ID NO: 1004 - protein, 9 aa, hepatitis C virus
EEKLPINPL

SEQ ID NO: 1005 - protein, 9 aa, hepatitis C virus
EEKLPISPL

SEQ ID NO: 1006 - protein, 10 aa, hepatitis C virus
EEQSVVCCSM

SEQ ID NO: 1007 - protein, 10 aa, hepatitis C virus
EESIYQACSL

SEQ ID NO: 1008 - protein, 10 aa, hepatitis C virus
EESIYQCCDL

SEQ ID NO: 1009 - protein, 10 aa, hepatitis C virus
EESKLPINAL

SEQ ID NO: 1010 - protein, 10 aa, hepatitis C virus
EFWESVFTGL

SEQ ID NO: 1011 - protein, 9 aa, hepatitis C virus
EGMGWAGWL

SEQ ID NO: 1012 - protein, 10 aa, hepatitis C virus
EGMGWAGWLL

SEQ ID NO: 1013 - protein, 10 aa, hepatitis C virus
EILLGPADSL

SEQ ID NO: 1014 - protein, 9 aa, hepatitis C virus
EINRVASCL

SEQ ID NO: 1015 - protein, 10 aa, hepatitis C virus
EKGGRKPARL

SEQ ID NO: 1016 - protein, 9 aa, hepatitis C virus
ELAAKLSAL

SEQ ID NO: 1017 - protein, 10 aa, hepatitis C virus
ELDGVRLHRY

SEQ ID NO: 1018 - protein, 10 aa, hepatitis C virus
ELIFDITKLL

SEQ ID NO: 1019 - protein, 9 aa, hepatitis C virus
EMYGATYSV

SEQ ID NO: 1020 - protein, 9 aa, hepatitis C virus
EMYGAVYSV

SEQ ID NO: 1021 - protein, 10 aa, hepatitis C virus
ENKVVILDSF

SEQ ID NO: 1022 - protein, 9 aa, hepatitis C virus
EPDVAVLTS

SEQ ID NO: 1023 - protein, 10 aa, hepatitis C virus
EPDVAVLTSM

SEQ ID NO: 1024 - protein, 9 aa, hepatitis C virus
EPEPDVAVL

SEQ ID NO: 1025 - protein, 10 aa, hepatitis C virus
EPEPDVAVLT

SEQ ID NO: 1026 - protein, 9 aa, hepatitis C virus
EPGDPDLSD

SEQ ID NO: 1027 - protein, 9 aa, hepatitis C virus
EPLDLPQII

SEQ ID NO: 1028 - protein, 10 aa, hepatitis C virus
EPVVFSDMET

SEQ ID NO: 1029 - protein, 10 aa, hepatitis C virus
EQFKQKALGL

SEQ ID NO: 1030 - protein, 9 aa, hepatitis C virus
ESENKVVIL

SEQ ID NO: 1031 - protein, 9 aa, hepatitis C virus
ESKLPINAL

SEQ ID NO: 1032 - protein, 10 aa, hepatitis C virus
ETAGARLVVL

SEQ ID NO: 1033 - protein, 10 aa, hepatitis C virus
ETSVRLRAYL

SEQ ID NO: 1034 - protein, 9 aa, hepatitis C virus
ETTMRSPVF

SEQ ID NO: 1035 - protein, 9 aa, hepatitis C virus
ETTVRLRAY

SEQ ID NO: 1036 - protein, 10 aa, hepatitis C virus
EVDGVRLHRY

SEQ ID NO: 1037 - protein, 10 aa, hepatitis C virus
EVDGVRLHRY

SEQ ID NO: 1038 - protein, 10 aa, hepatitis C virus
EVFCVQPEKG

SEQ ID NO: 1039 - protein, 10 aa, hepatitis C virus
EVLYREFDEM

SEQ ID NO: 1040 - protein, 9 aa, hepatitis C virus
EVSVAAEIL

SEQ ID NO: 1041 - protein, 10 aa, hepatitis C virus
EVTLTHPITK

SEQ ID NO: 1042 - protein, 9 aa, hepatitis C virus
EVVTSTWVL

SEQ ID NO: 1043 - protein, 9 aa, hepatitis C virus
EWAILPCSY

SEQ ID NO: 1044 - protein, 9 aa, hepatitis C virus
EWVILLFLL

SEQ ID NO: 1045 - protein, 9 aa, hepatitis C virus
EWVVLLFLL

SEQ ID NO: 1046 - protein, 10 aa, hepatitis C virus
EYDLELITSC

SEQ ID NO: 1047 - protein, 9 aa, hepatitis C virus
EYVVLLFLL

SEQ ID NO: 1048 - protein, 10 aa, hepatitis C virus
FADLMGYIPL

SEQ ID NO: 1049 - protein, 9 aa, hepatitis C virus
FAIKWEYVL

SEQ ID NO: 1050 - protein, 10 aa, hepatitis C virus
FAIKWEYVLL

SEQ ID NO: 1051 - protein, 10 aa, hepatitis C virus
FCHSKKKCDE

SEQ ID NO: 1052 - protein, 10 aa, hepatitis C virus
FCSAMYVGDL

SEQ ID NO: 1053 - protein, 9 aa, hepatitis C virus
FDITKLLLA

SEQ ID NO: 1054 - protein, 10 aa, hepatitis C virus
FEMYGATYSV

SEQ ID NO: 1055 - protein, 10 aa, hepatitis C virus
FEMYGAVYSV

SEQ ID NO: 1056 - protein, 9 aa, hepatitis C virus
FFCAAWYIK

SEQ ID NO: 1057 - protein, 9 aa, hepatitis C virus
FFTLSPWYK

SEQ ID NO: 1058 - protein, 10 aa, hepatitis C virus
FIAGLFYYHK

SEQ ID NO: 1059 - protein, 9 aa, hepatitis C virus
FIPVESMET

SEQ ID NO: 1060 - protein, 9 aa, hepatitis C virus
FISGIQYLA

SEQ ID NO: 1061 - protein, 9 aa, hepatitis C virus
FITRAEAHL

SEQ ID NO: 1062 - protein, 9 aa, hepatitis C virus
FKQKALGLL

SEQ ID NO: 1063 - protein, 9 aa, hepatitis C virus
FLARLIWWL

SEQ ID NO: 1064 - protein, 10 aa, hepatitis C virus
FLASLFYTHK

SEQ ID NO: 1065 - protein, 9 aa, hepatitis C virus
FLATCINGV

SEQ ID NO: 1066 - protein, 9 aa, hepatitis C virus
FLGTSISGV

SEQ ID NO: 1067 - protein, 9 aa, hepatitis C virus
FLGTTVGGV

SEQ ID NO: 1068 - protein, 9 aa, hepatitis C virus
FLLALFSCL

SEQ ID NO: 1069 - protein, 9 aa, hepatitis C virus
FLLALLSCI

SEQ ID NO: 1070 - protein, 10 aa, hepatitis C virus
FLLALLSCLT

SEQ ID NO: 1071 - protein, 9 aa, hepatitis C virus
FLLLADARI

SEQ ID NO: 1072 - protein, 9 aa, hepatitis C virus
FLLLADARV

SEQ ID NO: 1073 - protein, 9 aa, hepatitis C virus
FLVCGDDLV

SEQ ID NO: 1074 - protein, 10 aa, hepatitis C virus
FLVCGDDLVV

SEQ ID NO: 1075 - protein, 9 aa, hepatitis C virus
FMGGDVTRI

SEQ ID NO: 1076 - protein, 9 aa, hepatitis C virus
FNWAVRTKL

SEQ ID NO: 1077 - protein, 10 aa, hepatitis C virus
FPGGGQIVGG

SEQ ID NO: 1078 - protein, 9 aa, hepatitis C virus
FPPALPIWA

SEQ ID NO: 1079 - protein, 10 aa, hepatitis C virus
FPYLVAYQAT

SEQ ID NO: 1080 - protein, 10 aa, hepatitis C virus
FQVAHLHAPT

SEQ ID NO: 1081 - protein, 10 aa, hepatitis C virus
FRAAVCTRGV

SEQ ID NO: 1082 - protein, 9 aa, hepatitis C virus
FRKHPEATY

SEQ ID NO: 1083 - protein, 10 aa, hepatitis C virus
FSILLAQEQL

SEQ ID NO: 1084 - protein, 9 aa, hepatitis C virus
FSMQGAWAK

SEQ ID NO: 1085 - protein, 9 aa, hepatitis C virus
FTASITSPL

SEQ ID NO: 1086 - protein, 10 aa, hepatitis C virus
FTDNSSPPAV

SEQ ID NO: 1087 - protein, 10 aa, hepatitis C virus
FTEAMTRYSA

SEQ ID NO: 1088 - protein, 9 aa, hepatitis C virus
FTIFKIRMY

SEQ ID NO: 1089 - protein, 9 aa, hepatitis C virus
FTIFKVRMY

SEQ ID NO: 1090 - protein, 9 aa, hepatitis C virus
FVGLALLTL

SEQ ID NO: 1091 - protein, 9 aa, hepatitis C virus
FVRAHALLR

SEQ ID NO: 1092 - protein, 10 aa, hepatitis C virus
FVRAQGLIRA

SEQ ID NO: 1093 - protein, 9 aa, hepatitis C virus
FVSGIQYLA

SEQ ID NO: 1094 - protein, 9 aa, hepatitis C virus
FVVSGLAGA

SEQ ID NO: 1095 - protein, 9 aa, hepatitis C virus
FWAKHMWNF

SEQ ID NO: 1096 - protein, 10 aa, hepatitis C virus
FWAKHMWNFI

SEQ ID NO: 1097 - protein, 9 aa, hepatitis C virus
FWANDMWNF

SEQ ID NO: 1098 - protein, 9 aa, hepatitis C virus
FWARHMWNF

SEQ ID NO: 1099 - protein, 9 aa, hepatitis C virus
FWEAVFTGL

SEQ ID NO: 1100 - protein, 10 aa, hepatitis C virus
FYGKAIPIEV

SEQ ID NO: 1101 - protein, 9 aa, hepatitis C virus
FYPGVVFDI

SEQ ID NO: 1102 - protein, 9 aa, hepatitis C virus
GAAVGSIGL

SEQ ID NO: 1103 - protein, 9 aa, hepatitis C virus
GACYSIEPL

SEQ ID NO: 1104 - protein, 9 aa, hepatitis C virus
GAHGILSFL

SEQ ID NO: 1105 - protein, 10 aa, hepatitis C virus
GAHWGVLAGL

SEQ ID NO: 1106 - protein, 10 aa, hepatitis C virus
GARLVVLATA

SEQ ID NO: 1107 - protein, 9 aa, hepatitis C virus
GAVFVGLAL

SEQ ID NO: 1108 - protein, 10 aa, hepatitis C virus
GAVFVGLALL

SEQ ID NO: 1109 - protein, 9 aa, hepatitis C virus
GAVQWMNRL

SEQ ID NO: 1110 - protein, 10 aa, hepatitis C virus
GAYMSKAHGV

SEQ ID NO: 1111 - protein, 8 aa, hepatitis C virus
GCSFSIFL

SEQ ID NO: 1112 - protein, 10 aa, hepatitis C virus
GDNFPYLVAY

SEQ ID NO: 1113 - protein, 9 aa, hepatitis C virus
GEDVVCCSM

SEQ ID NO: 1114 - protein, 10 aa, hepatitis C virus
GEGAVQWMNR

SEQ ID NO: 1115 - protein, 10 aa, hepatitis C virus
GEINRVASCL

SEQ ID NO: 1116 - protein, 9 aa, hepatitis C virus
GEIPFYGKA

SEQ ID NO: 1117 - protein, 10 aa, hepatitis C virus
GEIPFYGKAI

SEQ ID NO: 1118 - protein, 9 aa, hepatitis C virus
GEIPFYGRA

SEQ ID NO: 1119 - protein, 10 aa, hepatitis C virus
GEIPFYGRAI

SEQ ID NO: 1120 - protein, 9 aa, hepatitis C virus
GEIQVLSTV

SEQ ID NO: 1121 - protein, 10 aa, hepatitis C virus
GEIQVLSTVT

SEQ ID NO: 1122 - protein, 9 aa, hepatitis C virus
GENFAYLTA

SEQ ID NO: 1123 - protein, 10 aa, hepatitis C virus
GENFAYLTAY

SEQ ID NO: 1124 - protein, 9 aa, hepatitis C virus
GENFPYLVA

SEQ ID NO: 1125 - protein, 10 aa, hepatitis C virus
GENFPYLVAY

SEQ ID NO: 1126 - protein, 9 aa, hepatitis C virus
GENLPYLVA

SEQ ID NO: 1127 - protein, 10 aa, hepatitis C virus
GENLPYLVAY

SEQ ID NO: 1128 - protein, 9 aa, hepatitis C virus
GEPGDPDLS

SEQ ID NO: 1129 - protein, 9 aa, hepatitis C virus
GEVQVLSTA

SEQ ID NO: 1130 - protein, 10 aa, hepatitis C virus
GEVQVLSTAT

SEQ ID NO: 1131 - protein, 9 aa, hepatitis C virus
GEVQVVSTA

SEQ ID NO: 1132 - protein, 10 aa, hepatitis C virus
GFADLMGYIP

SEQ ID NO: 1133 - protein, 10 aa, hepatitis C virus
GFFTLSPWYK

SEQ ID NO: 1134 - protein, 9 aa, hepatitis C virus
GFIAGLFYY

SEQ ID NO: 1135 - protein, 10 aa, hepatitis C virus
GGAVFVGLAL

SEQ ID NO: 1136 - protein, 9 aa, hepatitis C virus
GGGQIVGGV

SEQ ID NO: 1137 - protein, 9 aa, hepatitis C virus
GGQIVGGVY

SEQ ID NO: 1138 - protein, 9 aa, hepatitis C virus
GGRDAIILL

SEQ ID NO: 1139 - protein, 10 aa, hepatitis C virus
GGRKPARLIV

SEQ ID NO: 1140 - protein, 10 aa, hepatitis C virus
GIFRAAVCSR

SEQ ID NO: 1141 - protein, 10 aa, hepatitis C virus
GIFRAAVCTR

SEQ ID NO: 1142 - protein, 9 aa, hepatitis C virus
GILAGLAYY

SEQ ID NO: 1143 - protein, 9 aa, hepatitis C virus
GINAVAYYR

SEQ ID NO: 1144 - protein, 9 aa, hepatitis C virus
GIPFISCQK

SEQ ID NO: 1145 - protein, 10 aa, hepatitis C virus
GISGALVAFK

SEQ ID NO: 1146 - protein, 10 aa, hepatitis C virus
GKSTKVPAAY

SEQ ID NO: 1147 - protein, 9 aa, hepatitis C virus
GLDAFSLHT

SEQ ID NO: 1148 - protein, 10 aa, hepatitis C virus
GLDAFSLHTY

SEQ ID NO: 1149 - protein, 10 aa, hepatitis C virus
GLGKVLVDIL

SEQ ID NO: 1150 - protein, 9 aa, hepatitis C virus
GLGWAGWLL

SEQ ID NO: 1151 - protein, 10 aa, hepatitis C virus
GLLGCIITSL

SEQ ID NO: 1152 - protein, 9 aa, hepatitis C virus
GLPFISCQK

SEQ ID NO: 1153 - protein, 10 aa, hepatitis C virus
GLSAFSLHSY

SEQ ID NO: 1154 - protein, 10 aa, hepatitis C virus
GLSAFSLHSY

SEQ ID NO: 1155 - protein, 10 aa, hepatitis C virus
GLSAFTLHSY

SEQ ID NO: 1156 - protein, 9 aa, hepatitis C virus
GLSPAITKY

SEQ ID NO: 1157 - protein, 9 aa, hepatitis C virus
GLYLFNWAV

SEQ ID NO: 1158 - protein, 10 aa, hepatitis C virus
GLYLFNWAVR

SEQ ID NO: 1159 - protein, 9 aa, hepatitis C virus
GMGLNAVAY

SEQ ID NO: 1160 - protein, 10 aa, hepatitis C virus
GNIIMYAPTL

SEQ ID NO: 1161 - protein, 9 aa, hepatitis C virus
GPCTPSPAP

SEQ ID NO: 1162 - protein, 9 aa, hepatitis C virus
GPGEGAVQW

SEQ ID NO: 1163 - protein, 10 aa, hepatitis C virus
GPGEGAVQWM

SEQ ID NO: 1164 - protein, 10 aa, hepatitis C virus
GPITQMYTNV

SEQ ID NO: 1165 - protein, 9 aa, hepatitis C virus
GPKGPITQM

SEQ ID NO: 1166 - protein, 9 aa, hepatitis C virus
GPKGPVTQM

SEQ ID NO: 1167 - protein, 10 aa, hepatitis C virus
GPLLCPSGHA

SEQ ID NO: 1168 - protein, 10 aa, hepatitis C virus
GPLMVLQAGI

SEQ ID NO: 1169 - protein, 9 aa, hepatitis C virus
GPPCNIGGV

SEQ ID NO: 1170 - protein, 10 aa, hepatitis C virus
GPRLGVRATR

SEQ ID NO: 1171 - protein, 9 aa, hepatitis C virus
GPSQKIQLI

SEQ ID NO: 1172 - protein, 9 aa, hepatitis C virus
GPTDPRRRS

SEQ ID NO: 1173 - protein, 9 aa, hepatitis C virus
GPWLTPRCL

SEQ ID NO: 1174 - protein, 10 aa, hepatitis C virus
GPWLTPRCLV

SEQ ID NO: 1175 - protein, 9 aa, hepatitis C virus
GPWLTPRCM

SEQ ID NO: 1176 - protein, 9 aa, hepatitis C virus
GQAEAALEK

SEQ ID NO: 1177 - protein, 9 aa, hepatitis C virus
GQAFTFRPR

SEQ ID NO: 1178 - protein, 10 aa, hepatitis C virus
GQIVGGVYLL

SEQ ID NO: 1179 - protein, 9 aa, hepatitis C virus
GRAAICGKY

SEQ ID NO: 1180 - protein, 10 aa, hepatitis C virus
GRAWAQPGYP

SEQ ID NO: 1181 - protein, 10 aa, hepatitis C virus
GRGRRGIYRF

SEQ ID NO: 1182 - protein, 10 aa, hepatitis C virus
GRKPARLIVF

SEQ ID NO: 1183 - protein, 10 aa, hepatitis C virus
GRRGIYRFVT

SEQ ID NO: 1184 - protein, 10 aa, hepatitis C virus
GRTGRGRRGI

SEQ ID NO: 1185 - protein, 9 aa, hepatitis C virus
GSEGEIPFY

SEQ ID NO: 1186 - protein, 9 aa, hepatitis C virus
GSIGLGKVL

SEQ ID NO: 1187 - protein, 10 aa, hepatitis C virus
GSIGLGKVLV

SEQ ID NO: 1188 - protein, 9 aa, hepatitis C virus
GSMRITGPK

SEQ ID NO: 1189 - protein, 9 aa, hepatitis C virus
GSVFLVSQL

SEQ ID NO: 1190 - protein, 10 aa, hepatitis C virus
GSWHINRTAL

SEQ ID NO: 1191 - protein, 9 aa, hepatitis C virus
GVAGALVAF

SEQ ID NO: 1192 - protein, 10 aa, hepatitis C virus
GVAGALVAFK

SEQ ID NO: 1193 - protein, 10 aa, hepatitis C virus
GVAGALVAFK

SEQ ID NO: 1194 - protein, 9 aa, hepatitis C virus
GVICAAILR

SEQ ID NO: 1195 - protein, 10 aa, hepatitis C virus
GVICAAILRR

SEQ ID NO: 1196 - protein, 9 aa, hepatitis C virus
GVLAALAAY

SEQ ID NO: 1197 - protein, 9 aa, hepatitis C virus
GVLAGLAYY

SEQ ID NO: 1198 - protein, 10 aa, hepatitis C virus
GVMWTVYHGA

SEQ ID NO: 1199 - protein, 9 aa, hepatitis C virus
GVNAVAYYR

SEQ ID NO: 1200 - protein, 10 aa, hepatitis C virus
GVRATRKTSE

SEQ ID NO: 1201 - protein, 10 aa, hepatitis C virus
GVRLHRYAPA

SEQ ID NO: 1202 - protein, 9 aa, hepatitis C virus
GVRTITTGA

SEQ ID NO: 1203 - protein, 10 aa, hepatitis C virus
GVRVCEKMAL

SEQ ID NO: 1204 - protein, 10 aa, hepatitis C virus
GVSGALVAFK

SEQ ID NO: 1205 - protein, 9 aa, hepatitis C virus
GVVCAAILR

SEQ ID NO: 1206 - protein, 10 aa, hepatitis C virus
GVVCAAILRR

SEQ ID NO: 1207 - protein, 9 aa, hepatitis C virus
GVWPLLLLL

SEQ ID NO: 1208 - protein, 10 aa, hepatitis C virus
GVWPLLLLLL

SEQ ID NO: 1209 - protein, 9 aa, hepatitis C virus
GVWRGDGIM

SEQ ID NO: 1210 - protein, 10 aa, hepatitis C virus
GYGAGVAGAL

SEQ ID NO: 1211 - protein, 10 aa, hepatitis C virus
GYIPLVGAPL

SEQ ID NO: 1212 - protein, 10 aa, hepatitis C virus
GYRRCRASGV

SEQ ID NO: 1213 - protein, 10 aa, hepatitis C virus
GYTGDFDSVI

SEQ ID NO: 1214 - protein, 9 aa, hepatitis C virus
GYTSKGWKL

SEQ ID NO: 1215 - protein, 10 aa, hepatitis C virus
HAVGIFRAAV

SEQ ID NO: 1216 - protein, 10 aa, hepatitis C virus
HDASGKRVYY

SEQ ID NO: 1217 - protein, 9 aa, hepatitis C virus
HELTRVAAA

SEQ ID NO: 1218 - protein, 10 aa, hepatitis C virus
HELTRVAAAL

SEQ ID NO: 1219 - protein, 10 aa, hepatitis C virus
HGPTPLLYRL

SEQ ID NO: 1220 - protein, 9 aa, hepatitis C virus
HHDSPDADL

SEQ ID NO: 1221 - protein, 9 aa, hepatitis C virus
HIDAHFLSQ

SEQ ID NO: 1222 - protein, 10 aa, hepatitis C virus
HIDAHFLSQT

SEQ ID NO: 1223 - protein, 9 aa, hepatitis C virus
HIRSVWKDL

SEQ ID NO: 1224 - protein, 10 aa, hepatitis C virus
HIRSVWKDLL

SEQ ID NO: 1225 - protein, 10 aa, hepatitis C virus
HLEFWESVFT

SEQ ID NO: 1226 - protein, 10 aa, hepatitis C virus
HLHAPTGSGK

SEQ ID NO: 1227 - protein, 9 aa, hepatitis C virus
HLHQNIVDV

SEQ ID NO: 1228 - protein, 10 aa, hepatitis C virus
HLIFCHSKKK

SEQ ID NO: 1229 - protein, 9 aa, hepatitis C virus
HLIFCHSRK

SEQ ID NO: 1230 - protein, 9 aa, hepatitis C virus
HLLLCLLLL

SEQ ID NO: 1231 - protein, 9 aa, hepatitis C virus
HLPGCVPCV

SEQ ID NO: 1232 - protein, 9 aa, hepatitis C virus
HLQVWVPPL

SEQ ID NO: 1233 - protein, 9 aa, hepatitis C virus
HMWNFISGI

SEQ ID NO: 1234 - protein, 9 aa, hepatitis C virus
HMWNFVSGI

SEQ ID NO: 1235 - protein, 9 aa, hepatitis C virus
HPELIFDIT

SEQ ID NO: 1236 - protein, 10 aa, hepatitis C virus
HPITKYIMAC

SEQ ID NO: 1237 - protein, 9 aa, hepatitis C virus
HPNIEEVAL

SEQ ID NO: 1238 - protein, 9 aa, hepatitis C virus
HPVTKYIMA

SEQ ID NO: 1239 - protein, 9 aa, hepatitis C virus
HQNIVDVQY

SEQ ID NO: 1240 - protein, 10 aa, hepatitis C virus
HQNIVDVQYL

SEQ ID NO: 1241 - protein, 9 aa, hepatitis C virus
HSAKSKFGY

SEQ ID NO: 1242 - protein, 9 aa, hepatitis C virus
HSARSKFGY

SEQ ID NO: 1243 - protein, 10 aa, hepatitis C virus
HSKKKCDELA

SEQ ID NO: 1244 - protein, 9 aa, hepatitis C virus
HSTDSTTIL

SEQ ID NO: 1245 - protein, 9 aa, hepatitis C virus
HWIGRLIWW

SEQ ID NO: 1246 - protein, 9 aa, hepatitis C virus
HYAPRPCGI

SEQ ID NO: 1247 - protein, 10 aa, hepatitis C virus
HYPCTVNFTI

SEQ ID NO: 1248 - protein, 10 aa, hepatitis C virus
HYPCTVNFTL

SEQ ID NO: 1249 - protein, 10 aa, hepatitis C virus
HYPCTVNYTI

SEQ ID NO: 1250 - protein, 9 aa, hepatitis C virus
HYPPKPCGI

SEQ ID NO: 1251 - protein, 9 aa, hepatitis C virus
HYPPRPCGI

SEQ ID NO: 1252 - protein, 9 aa, hepatitis C virus
HYPYRLWHY

SEQ ID NO: 1253 - protein, 9 aa, hepatitis C virus
HYRDVLKEM

SEQ ID NO: 1254 - protein, 10 aa, hepatitis C virus
HYVPESDAAA

SEQ ID NO: 1255 - protein, 10 aa, hepatitis C virus
IAFASRGNHV

SEQ ID NO: 1256 - protein, 10 aa, hepatitis C virus
IDAHFLSQTK

SEQ ID NO: 1257 - protein, 9 aa, hepatitis C virus
IELGGKPAL

SEQ ID NO: 1258 - protein, 9 aa, hepatitis C virus
IEPLDLPQI

SEQ ID NO: 1259 - protein, 9 aa, hepatitis C virus
IERLHGLDA

SEQ ID NO: 1260 - protein, 10 aa, hepatitis C virus
IERLHGLDAF

SEQ ID NO: 1261 - protein, 9 aa, hepatitis C virus
IERLHGLEA

SEQ ID NO: 1262 - protein, 10 aa, hepatitis C virus
IERLHGLEAF

SEQ ID NO: 1263 - protein, 9 aa, hepatitis C virus
IERLHGLSA

SEQ ID NO: 1264 - protein, 10 aa, hepatitis C virus
IERLHGLSAF

SEQ ID NO: 1265 - protein, 10 aa, hepatitis C virus
IEVIKGGRHL

SEQ ID NO: 1266 - protein, 10 aa, hepatitis C virus
IFLLALLSCL

SEQ ID NO: 1267 - protein, 10 aa, hepatitis C virus
IGLGKVLVDI

SEQ ID NO: 1268 - protein, 10 aa, hepatitis C virus
IIMYAPTLWA

SEQ ID NO: 1269 - protein, 9 aa, hepatitis C virus
ILAGYGAGV

SEQ ID NO: 1270 - protein, 10 aa, hepatitis C virus
ILGGWVAAQL

SEQ ID NO: 1271 - protein, 9 aa, hepatitis C virus
ILLGPADSL

SEQ ID NO: 1272 - protein, 10 aa, hepatitis C virus
ILLNIMGGWL

SEQ ID NO: 1273 - protein, 9 aa, hepatitis C virus
ILMTHFFSI

SEQ ID NO: 1274 - protein, 10 aa, hepatitis C virus
ILMTHFFSIL

SEQ ID NO: 1275 - protein, 9 aa, hepatitis C virus
ILSPGALVV

SEQ ID NO: 1276 - protein, 10 aa, hepatitis C virus
ILSSLTITQL

SEQ ID NO: 1277 - protein, 9 aa, hepatitis C virus
ILTLSPHYK

SEQ ID NO: 1278 - protein, 10 aa, hepatitis C virus
IMAKNEVFCV

SEQ ID NO: 1279 - protein, 10 aa, hepatitis C virus
IMYAPTLWAR

SEQ ID NO: 1280 - protein, 9 aa, hepatitis C virus
IPAASQLDL

SEQ ID NO: 1281 - protein, 10 aa, hepatitis C virus
IPASAYEVRN

SEQ ID NO: 1282 - protein, 9 aa, hepatitis C virus
IPDREVLYR

SEQ ID NO: 1283 - protein, 8 aa, hepatitis C virus
IPFYGKAI

SEQ ID NO: 1284 - protein, 10 aa, hepatitis C virus
IPFYGKAIPI

SEQ ID NO: 1285 - protein, 10 aa, hepatitis C virus
IPFYGKAIPL

SEQ ID NO: 1286 - protein, 8 aa, hepatitis C virus
IPKARRPE

SEQ ID NO: 1287 - protein, 10 aa, hepatitis C virus
IPKARRPEGR

SEQ ID NO: 1288 - protein, 8 aa, hepatitis C virus
IPLVGAPL

SEQ ID NO: 1289 - protein, 8 aa, hepatitis C virus
IPLVGAPL

SEQ ID NO: 1290 - protein, 10 aa, hepatitis C virus
IPLVGAPLGG

SEQ ID NO: 1291 - protein, 9 aa, hepatitis C virus
IPPPRRKRT

SEQ ID NO: 1292 - protein, 10 aa, hepatitis C virus
IPPPRRKRTV

SEQ ID NO: 1293 - protein, 9 aa, hepatitis C virus
IPQAVVDMV

SEQ ID NO: 1294 - protein, 10 aa, hepatitis C virus
IPQAVVDMVA

SEQ ID NO: 1295 - protein, 10 aa, hepatitis C virus
IPTSGDVVVV

SEQ ID NO: 1296 - protein, 10 aa, hepatitis C virus
IPVESMETTM

SEQ ID NO: 1297 - protein, 9 aa, hepatitis C virus
IPVRRRGDS

SEQ ID NO: 1298 - protein, 10 aa, hepatitis C virus
IQRLHGLSAF

SEQ ID NO: 1299 - protein, 9 aa, hepatitis C virus
IQYAPTIWV

SEQ ID NO: 1300 - protein, 10 aa, hepatitis C virus
IQYAPTIWVR

SEQ ID NO: 1301 - protein, 10 aa, hepatitis C virus
IRSLTERLYI

SEQ ID NO: 1302 - protein, 9 aa, hepatitis C virus
ISGALVAFK

SEQ ID NO: 1303 - protein, 10 aa, hepatitis C virus
ISGIQYLAGL

SEQ ID NO: 1304 - protein, 9 aa, hepatitis C virus
ISGVLWTVY

SEQ ID NO: 1305 - protein, 9 aa, hepatitis C virus
ITAEAAARR

SEQ ID NO: 1306 - protein, 10 aa, hepatitis C virus
ITAETAKRRL

SEQ ID NO: 1307 - protein, 9 aa, hepatitis C virus
ITAYAQQTR

SEQ ID NO: 1308 - protein, 9 aa, hepatitis C virus
ITKLLLAIL

SEQ ID NO: 1309 - protein, 9 aa, hepatitis C virus
ITTGSPITY

SEQ ID NO: 1310 - protein, 10 aa, hepatitis C virus
ITYSTYGKFL

SEQ ID NO: 1311 - protein, 9 aa, hepatitis C virus
IVDVQYLYG

SEQ ID NO: 1312 - protein, 10 aa, hepatitis C virus
IVFPDLGVRV

SEQ ID NO: 1313 - protein, 10 aa, hepatitis C virus
IVYEAADMIM

SEQ ID NO: 1314 - protein, 10 aa, hepatitis C virus
IYRFVTPGER

SEQ ID NO: 1315 - protein, 9 aa, hepatitis C virus
KARRPEGRA

SEQ ID NO: 1316 - protein, 9 aa, hepatitis C virus
KASTVKAKL

SEQ ID NO: 1317 - protein, 10 aa, hepatitis C virus
KASTVKAKLL

SEQ ID NO: 1318 - protein, 9 aa, hepatitis C virus
KCDELAAKL

SEQ ID NO: 1319 - protein, 10 aa, hepatitis C virus
KCLIRLKPTL

SEQ ID NO: 1320 - protein, 10 aa, hepatitis C virus
KEVRSLSRRA

SEQ ID NO: 1321 - protein, 10 aa, hepatitis C virus
KFPGGGQIVG

SEQ ID NO: 1322 - protein, 9 aa, hepatitis C virus
KFPPALPIW

SEQ ID NO: 1323 - protein, 9 aa, hepatitis C virus
KGGRKPARL

SEQ ID NO: 1324 - protein, 10 aa, hepatitis C virus
KGGRKPARLI

SEQ ID NO: 1325 - protein, 9 aa, hepatitis C virus
KGSSGGPLL

SEQ ID NO: 1326 - protein, 9 aa, hepatitis C virus
KGVWRGDGI

SEQ ID NO: 1327 - protein, 10 aa, hepatitis C virus
KKCDELAAKL

SEQ ID NO: 1328 - protein, 9 aa, hepatitis C virus
KKDPMGFSY

SEQ ID NO: 1329 - protein, 9 aa, hepatitis C virus
KLGALTGTY

SEQ ID NO: 1330 - protein, 9 aa, hepatitis C virus
KLLAPITAY

SEQ ID NO: 1331 - protein, 10 aa, hepatitis C virus
KLLLAILGPL

SEQ ID NO: 1332 - protein, 10 aa, hepatitis C virus
KLLLAVLGPL

SEQ ID NO: 1333 - protein, 9 aa, hepatitis C virus
KLQDCTMLV

SEQ ID NO: 1334 - protein, 9 aa, hepatitis C virus
KLTYSTYGK

SEQ ID NO: 1335 - protein, 10 aa, hepatitis C virus
KPAPNFKTAI

SEQ ID NO: 1336 - protein, 8 aa, hepatitis C virus
KPARLIVF

SEQ ID NO: 1337 - protein, 9 aa, hepatitis C virus
KPARLIVFP

SEQ ID NO: 1338 - protein, 9 aa, hepatitis C virus
KPLLREEVT

SEQ ID NO: 1339 - protein, 10 aa, hepatitis C virus
KPLLREEVTF

SEQ ID NO: 1340 - protein, 9 aa, hepatitis C virus
KPQRKTKRN

SEQ ID NO: 1341 - protein, 10 aa, hepatitis C virus
KPQRKTKRNT

SEQ ID NO: 1342 - protein, 9 aa, hepatitis C virus
KPTLHGPTP

SEQ ID NO: 1343 - protein, 10 aa, hepatitis C virus
KPTLHGPTPL

SEQ ID NO: 1344 - protein, 10 aa, hepatitis C virus
KPTLQGPTPL

SEQ ID NO: 1345 - protein, 10 aa, hepatitis C virus
KPTLVGPTPL

SEQ ID NO: 1346 - protein, 10 aa, hepatitis C virus
KQAGDNFPYL

SEQ ID NO: 1347 - protein, 9 aa, hepatitis C virus
KQSGENFPY

SEQ ID NO: 1348 - protein, 9 aa, hepatitis C virus
KSRKFPPAL

SEQ ID NO: 1349 - protein, 10 aa, hepatitis C virus
KVAGGHYVQM

SEQ ID NO: 1350 - protein, 10 aa, hepatitis C virus
KVLVDILAGY

SEQ ID NO: 1351 - protein, 9 aa, hepatitis C virus
KVRMYVGGV

SEQ ID NO: 1352 - protein, 10 aa, hepatitis C virus
KVTFDRLQVL

SEQ ID NO: 1353 - protein, 9 aa, hepatitis C virus
KWEWVVLLF

SEQ ID NO: 1354 - protein, 9 aa, hepatitis C virus
KWEYVVLLF

SEQ ID NO: 1355 - protein, 9 aa, hepatitis C virus
KYPPALPIW

SEQ ID NO: 1356 - protein, 10 aa, hepatitis C virus
LAAKLSALGL

SEQ ID NO: 1357 - protein, 9 aa, hepatitis C virus
LAALAAYCL

SEQ ID NO: 1358 - protein, 10 aa, hepatitis C virus
LADARVCACL

SEQ ID NO: 1359 - protein, 11 aa, hepatitis C virus
LADGGCSGGAY

SEQ ID NO: 1360 - protein, 9 aa, hepatitis C virus
LAGGVLAAV

SEQ ID NO: 1361 - protein, 10 aa, hepatitis C virus
LAILGPLMVL

SEQ ID NO: 1362 - protein, 10 aa, hepatitis C virus
LAQEQLEKAL

SEQ ID NO: 1363 - protein, 9 aa, hepatitis C virus
LARGSPPSL

SEQ ID NO: 1364 - protein, 10 aa, hepatitis C virus
LARGSPPSLA

SEQ ID NO: 1365 - protein, 9 aa, hepatitis C virus
LASSSASQL

SEQ ID NO: 1366 - protein, 9 aa, hepatitis C virus
LCECYDAGC

SEQ ID NO: 1367 - protein, 9 aa, hepatitis C virus
LDAFSLHTY

SEQ ID NO: 1368 - protein, 9 aa, hepatitis C virus
LEDRDRSEL

SEQ ID NO: 1369 - protein, 9 aa, hepatitis C virus
LEFWEGVFT

SEQ ID NO: 1370 - protein, 10 aa, hepatitis C virus
LEFWEGVFTG

SEQ ID NO: 1371 - protein, 10 aa, hepatitis C virus
LEFWESVFTG

SEQ ID NO: 1372 - protein, 9 aa, hepatitis C virus
LELITSCSS

SEQ ID NO: 1373 - protein, 9 aa, hepatitis C virus
LENLVILNA

SEQ ID NO: 1374 - protein, 10 aa, hepatitis C virus
LEQFWAKHMW

SEQ ID NO: 1375 - protein, 9 aa, hepatitis C virus
LETTMRSPV

SEQ ID NO: 1376 - protein, 10 aa, hepatitis C virus
LEVFWAKHMW

SEQ ID NO: 1377 - protein, 9 aa, hepatitis C virus
LEVVTSTWV

SEQ ID NO: 1378 - protein, 10 aa, hepatitis C virus
LEVVTSTWVL

SEQ ID NO: 1379 - protein, 9 aa, hepatitis C virus
LFNWAVKTK

SEQ ID NO: 1380 - protein, 9 aa, hepatitis C virus
LFNWAVRTK

SEQ ID NO: 1381 - protein, 10 aa, hepatitis C virus
LFNWAVRTKL

SEQ ID NO: 1382 - protein, 9 aa, hepatitis C virus
LGKVLVDIL

SEQ ID NO: 1383 - protein, 10 aa, hepatitis C virus
LGKVLVDILA

SEQ ID NO: 1384 - protein, 9 aa, hepatitis C virus
LIAVLGPLY

SEQ ID NO: 1385 - protein, 9 aa, hepatitis C virus
LIFCHSRKK

SEQ ID NO: 1386 - protein, 9 aa, hepatitis C virus
LIFDITKLL

SEQ ID NO: 1387 - protein, 10 aa, hepatitis C virus
LIRLKPTLHG

SEQ ID NO: 1388 - protein, 10 aa, hepatitis C virus
LKGSSGGPLL

SEQ ID NO: 1389 - protein, 9 aa, hepatitis C virus
LLALLGPAY

SEQ ID NO: 1390 - protein, 10 aa, hepatitis C virus
LLALLSCLTV

SEQ ID NO: 1391 - protein, 9 aa, hepatitis C virus
LLAPITAYA

SEQ ID NO: 1392 - protein, 9 aa, hepatitis C virus
LLAQEQLEK

SEQ ID NO: 1393 - protein, 9 aa, hepatitis C virus
LLCLLLLTV

SEQ ID NO: 1394 - protein, 10 aa, hepatitis C virus
LLCPTDCFRK

SEQ ID NO: 1395 - protein, 9 aa, hepatitis C virus
LLFLLLADA

SEQ ID NO: 1396 - protein, 10 aa, hepatitis C virus
LLFNILGGWV

SEQ ID NO: 1397 - protein, 9 aa, hepatitis C virus
LLGCIITSL

SEQ ID NO: 1398 - protein, 9 aa, hepatitis C virus
LLIAVLGPL

SEQ ID NO: 1399 - protein, 10 aa, hepatitis C virus
LLLADARVCV

SEQ ID NO: 1400 - protein, 9 aa, hepatitis C virus
LLLAIFGPL

SEQ ID NO: 1401 - protein, 9 aa, hepatitis C virus
LLLAVLGPL

SEQ ID NO: 1402 - protein, 9 aa, hepatitis C virus
LLLCLLLLT

SEQ ID NO: 1403 - protein, 10 aa, hepatitis C virus
LLLCLLLLTV

SEQ ID NO: 1404 - protein, 9 aa, hepatitis C virus
LLLLGLLLL

SEQ ID NO: 1405 - protein, 10 aa, hepatitis C virus
LLLLTVGVGI

SEQ ID NO: 1406 - protein, 9 aa, hepatitis C virus
LLPRRGPRL

SEQ ID NO: 1407 - protein, 9 aa, hepatitis C virus
LLRIPYFVR

SEQ ID NO: 1408 - protein, 9 aa, hepatitis C virus
LLSPRPISY

SEQ ID NO: 1409 - protein, 10 aa, hepatitis C virus
LLSPRPVSYL

SEQ ID NO: 1410 - protein, 10 aa, hepatitis C virus
LLSVEEACKL

SEQ ID NO: 1411 - protein, 9 aa, hepatitis C virus
LLSVGVGIY

SEQ ID NO: 1412 - protein, 10 aa, hepatitis C virus
LLSVGVGIYL

SEQ ID NO: 1413 - protein, 10 aa, hepatitis C virus
LLTCAVHPEL

SEQ ID NO: 1414 - protein, 9 aa, hepatitis C virus
LMTHFFSIL

SEQ ID NO: 1415 - protein, 10 aa, hepatitis C virus
LMTHFFSILL

SEQ ID NO: 1416 - protein, 10 aa, hepatitis C virus
LNAVAYYRGL

SEQ ID NO: 1417 - protein, 9 aa, hepatitis C virus
LPAILSPGA

SEQ ID NO: 1418 - protein, 10 aa, hepatitis C virus
LPAILSPGAL

SEQ ID NO: 1419 - protein, 9 aa, hepatitis C virus
LPALSTGLI

SEQ ID NO: 1420 - protein, 9 aa, hepatitis C virus
LPALSTGLL

SEQ ID NO: 1421 - protein, 9 aa, hepatitis C virus
LPCEPEPDV

SEQ ID NO: 1422 - protein, 10 aa, hepatitis C virus
LPCEPEPDVA

SEQ ID NO: 1423 - protein, 10 aa, hepatitis C virus
LPCSFSDLPA

SEQ ID NO: 1424 - protein, 10 aa, hepatitis C virus
LPCSFTTLPA

SEQ ID NO: 1425 - protein, 8 aa, hepatitis C virus
LPGCSFSI

SEQ ID NO: 1426 - protein, 10 aa, hepatitis C virus
LPGCSFSIFL

SEQ ID NO: 1427 - protein, 9 aa, hepatitis C virus
LPGNPAIAS

SEQ ID NO: 1428 - protein, 10 aa, hepatitis C virus
LPGNPAIASL

SEQ ID NO: 1429 - protein, 9 aa, hepatitis C virus
LPGVPFFSC

SEQ ID NO: 1430 - protein, 10 aa, hepatitis C virus
LPINALSNSL

SEQ ID NO: 1431 - protein, 9 aa, hepatitis C virus
LPIWARPDY

SEQ ID NO: 1432 - protein, 9 aa, hepatitis C virus
LPKSRFPPA

SEQ ID NO: 1433 - protein, 10 aa, hepatitis C virus
LPKSRFPPAL

SEQ ID NO: 1434 - protein, 9 aa, hepatitis C virus
LPPRAYAMD

SEQ ID NO: 1435 - protein, 9 aa, hepatitis C virus
LPPTKAPPI

SEQ ID NO: 1436 - protein, 9 aa, hepatitis C virus
LPQAVMGSS

SEQ ID NO: 1437 - protein, 10 aa, hepatitis C virus
LPQAVMGSSY

SEQ ID NO: 1438 - protein, 9 aa, hepatitis C virus
LPQIIERLH

SEQ ID NO: 1439 - protein, 9 aa, hepatitis C virus
LPRLPGVPF

SEQ ID NO: 1440 - protein, 10 aa, hepatitis C virus
LPRLPGVPFF

SEQ ID NO: 1441 - protein, 8 aa, hepatitis C virus
LPRRGPRL

SEQ ID NO: 1442 - protein, 8 aa, hepatitis C virus
LPRRGPRL

SEQ ID NO: 1443 - protein, 9 aa, hepatitis C virus
LPTSFGNTI

SEQ ID NO: 1444 - protein, 10 aa, hepatitis C virus
LPVCQDHLEF

SEQ ID NO: 1445 - protein, 8 aa, hepatitis C virus
LPYIEQGM

SEQ ID NO: 1446 - protein, 9 aa, hepatitis C virus
LPYIEQGMQ

SEQ ID NO: 1447 - protein, 10 aa, hepatitis C virus
LPYIEQGMQL

SEQ ID NO: 1448 - protein, 10 aa, hepatitis C virus
LQGPTPLLYR

SEQ ID NO: 1449 - protein, 9 aa, hepatitis C virus
LQSKLLPRL

SEQ ID NO: 1450 - protein, 9 aa, hepatitis C virus
LQTGFLAAL

SEQ ID NO: 1451 - protein, 9 aa, hepatitis C virus
LQTGFLASL

SEQ ID NO: 1452 - protein, 9 aa, hepatitis C virus
LQVLDDHYK

SEQ ID NO: 1453 - protein, 9 aa, hepatitis C virus
LQVWVPPLL

SEQ ID NO: 1454 - protein, 10 aa, hepatitis C virus
LRAYLNTPGL

SEQ ID NO: 1455 - protein, 9 aa, hepatitis C virus
LRKLGVPPL

SEQ ID NO: 1456 - protein, 9 aa, hepatitis C virus
LSAFSLHSY

SEQ ID NO: 1457 - protein, 9 aa, hepatitis C virus
LSAFTLHSY

SEQ ID NO: 1458 - protein, 10 aa, hepatitis C virus
LSCLTIPASA

SEQ ID NO: 1459 - protein, 10 aa, hepatitis C virus
LSNTGEIPFY

SEQ ID NO: 1460 - protein, 9 aa, hepatitis C virus
LSPRPVSYL

SEQ ID NO: 1461 - protein, 9 aa, hepatitis C virus
LSPYYKVFL

SEQ ID NO: 1462 - protein, 10 aa, hepatitis C virus
LSRARPRWFM

SEQ ID NO: 1463 - protein, 9 aa, hepatitis C virus
LSVGVGIYL

SEQ ID NO: 1464 - protein, 9 aa, hepatitis C virus
LTCAVHPEL

SEQ ID NO: 1465 - protein, 11 aa, hepatitis C virus
LTCGFADLMGY

SEQ ID NO: 1466 - protein, 10 aa, hepatitis C virus
LTCYLKASAA

SEQ ID NO: 1467 - protein, 9 aa, hepatitis C virus
LTDPSHITA

SEQ ID NO: 1468 - protein, 10 aa, hepatitis C virus
LTDPSHITAE

SEQ ID NO: 1469 - protein, 10 aa, hepatitis C virus
LTHPITKYIM

SEQ ID NO: 1470 - protein, 10 aa, hepatitis C virus
LTITQLLKRL

SEQ ID NO: 1471 - protein, 10 aa, hepatitis C virus
LTPAETSVRL

SEQ ID NO: 1472 - protein, 10 aa, hepatitis C virus
LTPIPAASQL

SEQ ID NO: 1473 - protein, 9 aa, hepatitis C virus
LTPRCLIDY

SEQ ID NO: 1474 - protein, 9 aa, hepatitis C virus
LTPRCLVDY

SEQ ID NO: 1475 - protein, 9 aa, hepatitis C virus
LTRDPTTPL

SEQ ID NO: 1476 - protein, 9 aa, hepatitis C virus
LTRVPYFVR

SEQ ID NO: 1477 - protein, 9 aa, hepatitis C virus
LTVGVGIFL

SEQ ID NO: 1478 - protein, 10 aa, hepatitis C virus
LVDILAGYGA

SEQ ID NO: 1479 - protein, 9 aa, hepatitis C virus
LVGGVLAAL

SEQ ID NO: 1480 - protein, 10 aa, hepatitis C virus
LVNGDDLVVI

SEQ ID NO: 1481 - protein, 9 aa, hepatitis C virus
LVNLLPAIL

SEQ ID NO: 1482 - protein, 9 aa, hepatitis C virus
LVPGAAYAL

SEQ ID NO: 1483 - protein, 11 aa, hepatitis C virus
LWARMILMTHF

SEQ ID NO: 1484 - protein, 10 aa, hepatitis C virus
LWHYPCTVNF

SEQ ID NO: 1485 - protein, 10 aa, hepatitis C virus
LWRQEMGGNI

SEQ ID NO: 1486 - protein, 10 aa, hepatitis C virus
LYDVIQKLSI

SEQ ID NO: 1487 - protein, 9 aa, hepatitis C virus
LYGNEGLGW

SEQ ID NO: 1488 - protein, 10 aa, hepatitis C virus
LYGNEGMGWA

SEQ ID NO: 1489 - protein, 9 aa, hepatitis C virus
LYPSLIFDI

SEQ ID NO: 1490 - protein, 10 aa, hepatitis C virus
MACMSADLEV

SEQ ID NO: 1491 - protein, 9 aa, hepatitis C virus
MAFMKLAAL

SEQ ID NO: 1492 - protein, 10 aa, hepatitis C virus
MALYDVVSTL

SEQ ID NO: 1493 - protein, 10 aa, hepatitis C virus
METTMRSPVF

SEQ ID NO: 1494 - protein, 10 aa, hepatitis C virus
MGFSYDTRCF

SEQ ID NO: 1495 - protein, 9 aa, hepatitis C virus
MGSAYGFQY

SEQ ID NO: 1496 - protein, 9 aa, hepatitis C virus
MGSSYGFQY

SEQ ID NO: 1497 - protein, 9 aa, hepatitis C virus
MLLISQAEA

SEQ ID NO: 1498 - protein, 9 aa, hepatitis C virus
MMMNWSPTA

SEQ ID NO: 1499 - protein, 10 aa, hepatitis C virus
MMMNWSPTAA

SEQ ID NO: 1500 - protein, 9 aa, hepatitis C virus
MMMNWSPTT

SEQ ID NO: 1501 - protein, 10 aa, hepatitis C virus
MMMNWSPTTA

SEQ ID NO: 1502 - protein, 10 aa, hepatitis C virus
MMNWSPTTAL

SEQ ID NO: 1503 - protein, 9 aa, hepatitis C virus
MNWSPTTAL

SEQ ID NO: 1504 - protein, 9 aa, hepatitis C virus
MPSTEDLVN

SEQ ID NO: 1505 - protein, 10 aa, hepatitis C virus
MPSTEDLVNL

SEQ ID NO: 1506 - protein, 10 aa, hepatitis C virus
MQLAEQFKQK

SEQ ID NO: 1507 - protein, 10 aa, hepatitis C virus
MSATLCSALY

SEQ ID NO: 1508 - protein, 9 aa, hepatitis C virus
MTHFFSILL

SEQ ID NO: 1509 - protein, 10 aa, hepatitis C virus
MVAGAHWGVL

SEQ ID NO: 1510 - protein, 9 aa, hepatitis C virus
MVGNWAKVL

SEQ ID NO: 1511 - protein, 10 aa, hepatitis C virus
MYAPTLWARM

SEQ ID NO: 1512 - protein, 9 aa, hepatitis C virus
MYTNVDQDL

SEQ ID NO: 1513 - protein, 10 aa, hepatitis C virus
MYVGGVEHRL

SEQ ID NO: 1514 - protein, 10 aa, hepatitis C virus
NAWKSKKCPM

SEQ ID NO: 1515 - protein, 10 aa, hepatitis C virus
NDIRVEESIY

SEQ ID NO: 1516 - protein, 10 aa, hepatitis C virus
NEGLGWAGWL

SEQ ID NO: 1517 - protein, 10 aa, hepatitis C virus
NEGMGWAGWL

SEQ ID NO: 1518 - protein, 9 aa, hepatitis C virus
NEITLTHPV

SEQ ID NO: 1519 - protein, 10 aa, hepatitis C virus
NEVTLTHPIT

SEQ ID NO: 1520 - protein, 9 aa, hepatitis C virus
NEVVLTHPI

SEQ ID NO: 1521 - protein, 9 aa, hepatitis C virus
NFISGIQYL

SEQ ID NO: 1522 - protein, 10 aa, hepatitis C virus
NFPYLVAYQA

SEQ ID NO: 1523 - protein, 9 aa, hepatitis C virus
NIVDVQYLY

SEQ ID NO: 1524 - protein, 10 aa, hepatitis C virus
NLFMGGDVTR

SEQ ID NO: 1525 - protein, 9 aa, hepatitis C virus
NMWHGTFPI

SEQ ID NO: 1526 - protein, 10 aa, hepatitis C virus
NNSSRCWVAL

SEQ ID NO: 1527 - protein, 9 aa, hepatitis C virus
NPAIASLMA

SEQ ID NO: 1528 - protein, 10 aa, hepatitis C virus
NPAIASLMAF

SEQ ID NO: 1529 - protein, 10 aa, hepatitis C virus
NPAVASLMAF

SEQ ID NO: 1530 - protein, 10 aa, hepatitis C virus
NPAVASMMAF

SEQ ID NO: 1531 - protein, 10 aa, hepatitis C virus
NPKPQRKTKR

SEQ ID NO: 1532 - protein, 10 aa, hepatitis C virus
NPSVAATLGF

SEQ ID NO: 1533 - protein, 9 aa, hepatitis C virus
NSSRCWVAL

SEQ ID NO: 1534 - protein, 10 aa, hepatitis C virus
NSWLGNIIMY

SEQ ID NO: 1535 - protein, 10 aa, hepatitis C virus
NTGEIPFYGK

SEQ ID NO: 1536 - protein, 9 aa, hepatitis C virus
NTWHGTFPI

SEQ ID NO: 1537 - protein, 9 aa, hepatitis C virus
NTWQGTFPI

SEQ ID NO: 1538 - protein, 9 aa, hepatitis C virus
NVRGGRDAI

SEQ ID NO: 1539 - protein, 10 aa, hepatitis C virus
NVRGGRDAII

SEQ ID NO: 1540 - protein, 10 aa, hepatitis C virus
NWAVKTKLKL

SEQ ID NO: 1541 - protein, 10 aa, hepatitis C virus
NWAVRTKLKL

SEQ ID NO: 1542 - protein, 9 aa, hepatitis C virus
NWQKLEAFW

SEQ ID NO: 1543 - protein, 9 aa, hepatitis C virus
NYTIFKIRM

SEQ ID NO: 1544 - protein, 9 aa, hepatitis C virus
PAAYVAQGY

SEQ ID NO: 1545 - protein, 10 aa, hepatitis C virus
PARLIVFPDL

SEQ ID NO: 1546 - protein, 10 aa, hepatitis C virus
PEARQAIRSL

SEQ ID NO: 1547 - protein, 10 aa, hepatitis C virus
PEFFSWVDGV

SEQ ID NO: 1548 - protein, 10 aa, hepatitis C virus
PEGRSWAQPG

SEQ ID NO: 1549 - protein, 10 aa, hepatitis C virus
PEGRTWAQPG

SEQ ID NO: 1550 - protein, 9 aa, hepatitis C virus
PEKGGRKPA

SEQ ID NO: 1551 - protein, 10 aa, hepatitis C virus
PESDAAARVT

SEQ ID NO: 1552 - protein, 10 aa, hepatitis C virus
PEYDLELITS

SEQ ID NO: 1553 - protein, 9 aa, hepatitis C virus
PFYGKAIPI

SEQ ID NO: 1554 - protein, 9 aa, hepatitis C virus
PFYGKAIPL

SEQ ID NO: 1555 - protein, 10 aa, hepatitis C virus
PHPNIEEVAL

SEQ ID NO: 1556 - protein, 9 aa, hepatitis C virus
PITYSTYGK

SEQ ID NO: 1557 - protein, 9 aa, hepatitis C virus
PLLLLLLAL

SEQ ID NO: 1558 - protein, 9 aa, hepatitis C virus
PLLYRLGAV

SEQ ID NO: 1559 - protein, 10 aa, hepatitis C virus
PLRDWAHAGL

SEQ ID NO: 1560 - protein, 9 aa, hepatitis C virus
PLSNSLLRY

SEQ ID NO: 1561 - protein, 9 aa, hepatitis C virus
PMEIKVITW

SEQ ID NO: 1562 - protein, 9 aa, hepatitis C virus
PPALPIWAR

SEQ ID NO: 1563 - protein, 9 aa, hepatitis C virus
PPAVPQTFQ

SEQ ID NO: 1564 - protein, 10 aa, hepatitis C virus
PPAVPQTFQV

SEQ ID NO: 1565 - protein, 9 aa, hepatitis C virus
PPGDPPQPE

SEQ ID NO: 1566 - protein, 10 aa, hepatitis C virus
PPGDPPQPEY

SEQ ID NO: 1567 - protein, 9 aa, hepatitis C virus
PPGSVTVPH

SEQ ID NO: 1568 - protein, 9 aa, hepatitis C virus
PPHSAKSKF

SEQ ID NO: 1569 - protein, 9 aa, hepatitis C virus
PPHSARSKF

SEQ ID NO: 1570 - protein, 9 aa, hepatitis C virus
PPIPPPRRK

SEQ ID NO: 1571 - protein, 10 aa, hepatitis C virus
PPLRVWRHRA

SEQ ID NO: 1572 - protein, 9 aa, hepatitis C virus
PPPRRKRTV

SEQ ID NO: 1573 - protein, 10 aa, hepatitis C virus
PPPRRKRTVV

SEQ ID NO: 1574 - protein, 10 aa, hepatitis C virus
PPQGNWFGCT

SEQ ID NO: 1575 - protein, 10 aa, hepatitis C virus
PPQPEYDLEL

SEQ ID NO: 1576 - protein, 9 aa, hepatitis C virus
PPRAYAMDR

SEQ ID NO: 1577 - protein, 9 aa, hepatitis C virus
PPRKKRTVV

SEQ ID NO: 1578 - protein, 9 aa, hepatitis C virus
PPRRKRTVV

SEQ ID NO: 1579 - protein, 10 aa, hepatitis C virus
PPRRKRTVVL

SEQ ID NO: 1580 - protein, 9 aa, hepatitis C virus
PPSAASAFV

SEQ ID NO: 1581 - protein, 9 aa, hepatitis C virus
PPSLASSSA

SEQ ID NO: 1582 - protein, 9 aa, hepatitis C virus
PPVVHGCPL

SEQ ID NO: 1583 - protein, 9 aa, hepatitis C virus
PRRKRTVVL

SEQ ID NO: 1584 - protein, 10 aa, hepatitis C virus
PSGHAVGIFR

SEQ ID NO: 1585 - protein, 10 aa, hepatitis C virus
PSWDQMWKCL

SEQ ID NO: 1586 - protein, 10 aa, hepatitis C virus
PTDPRRRSRN

SEQ ID NO: 1587 - protein, 11 aa, hepatitis C virus
PTLHGPTPLLY

SEQ ID NO: 1588 - protein, 10 aa, hepatitis C virus
PVESMETTMR

SEQ ID NO: 1589 - protein, 10 aa, hepatitis C virus
PVSARRGREI

SEQ ID NO: 1590 - protein, 10 aa, hepatitis C virus
PWPLYGNEGM

SEQ ID NO: 1591 - protein, 10 aa, hepatitis C virus
PYFVRAHALL

SEQ ID NO: 1592 - protein, 9 aa, hepatitis C virus
PYFVRAHVL

SEQ ID NO: 1593 - protein, 9 aa, hepatitis C virus
PYIEQAQAI

SEQ ID NO: 1594 - protein, 10 aa, hepatitis C virus
PYLVAYQATV

SEQ ID NO: 1595 - protein, 10 aa, hepatitis C virus
QAETAGARLV

SEQ ID NO: 1596 - protein, 10 aa, hepatitis C virus
QAGDNFPYLV

SEQ ID NO: 1597 - protein, 10 aa, hepatitis C virus
QAIRSLTERL

SEQ ID NO: 1598 - protein, 9 aa, hepatitis C virus
QAPPGARSL

SEQ ID NO: 1599 - protein, 10 aa, hepatitis C virus
QAVMGSSYGF

SEQ ID NO: 1600 - protein, 10 aa, hepatitis C virus
QDHLEFWESV

SEQ ID NO: 1601 - protein, 10 aa, hepatitis C virus
QEDAASLRVF

SEQ ID NO: 1602 - protein, 9 aa, hepatitis C virus
QFKQKALGL

SEQ ID NO: 1603 - protein, 10 aa, hepatitis C virus
QFKQKALGLL

SEQ ID NO: 1604 - protein, 10 aa, hepatitis C virus
QFWAKHMWNF

SEQ ID NO: 1605 - protein, 10 aa, hepatitis C virus
QIHRFAPTPK

SEQ ID NO: 1606 - protein, 10 aa, hepatitis C virus
QILPCSFTTL

SEQ ID NO: 1607 - protein, 9 aa, hepatitis C virus
QKKVTFDRL

SEQ ID NO: 1608 - protein, 9 aa, hepatitis C virus
QLAEQFKQK

SEQ ID NO: 1609 - protein, 9 aa, hepatitis C virus
QLFTFSPRR

SEQ ID NO: 1610 - protein, 10 aa, hepatitis C virus
QMWKCLIRLK

SEQ ID NO: 1611 - protein, 10 aa, hepatitis C virus
QMYTNVDQDL

SEQ ID NO: 1612 - protein, 10 aa, hepatitis C virus
QPEKGGRKPA

SEQ ID NO: 1613 - protein, 9 aa, hepatitis C virus
QPEYDLELI

SEQ ID NO: 1614 - protein, 10 aa, hepatitis C virus
QPEYDLELIT

SEQ ID NO: 1615 - protein, 8 aa, hepatitis C virus
QPRGRRQP

SEQ ID NO: 1616 - protein, 10 aa, hepatitis C virus
QPRGRRQPIP

SEQ ID NO: 1617 - protein, 9 aa, hepatitis C virus
QPRRRRQPI

SEQ ID NO: 1618 - protein, 10 aa, hepatitis C virus
QRKTKRNTNR

SEQ ID NO: 1619 - protein, 10 aa, hepatitis C virus
QRRGRTGRGR

SEQ ID NO: 1620 - protein, 10 aa, hepatitis C virus
QTGFLASLFY

SEQ ID NO: 1621 - protein, 9 aa, hepatitis C virus
QTRGLLGCI

SEQ ID NO: 1622 - protein, 10 aa, hepatitis C virus
QTRGLLGCII

SEQ ID NO: 1623 - protein, 9 aa, hepatitis C virus
QWMNRLIAF

SEQ ID NO: 1624 - protein, 10 aa, hepatitis C virus
QWMNRLIAFA

SEQ ID NO: 1625 - protein, 9 aa, hepatitis C virus
QYLAGLSTL

SEQ ID NO: 1626 - protein, 10 aa, hepatitis C virus
QYSPGQRVEF

SEQ ID NO: 1627 - protein, 9 aa, hepatitis C virus
RAATCGKYL

SEQ ID NO: 1628 - protein, 9 aa, hepatitis C virus
RALAHGVRV

SEQ ID NO: 1629 - protein, 10 aa, hepatitis C virus
RALAHGVRVL

SEQ ID NO: 1630 - protein, 10 aa, hepatitis C virus
RAQGLIRACM

SEQ ID NO: 1631 - protein, 9 aa, hepatitis C virus
RARPRWFML

SEQ ID NO: 1632 - protein, 9 aa, hepatitis C virus
RARSVRAKL

SEQ ID NO: 1633 - protein, 10 aa, hepatitis C virus
RARSVRAKLL

SEQ ID NO: 1634 - protein, 10 aa, hepatitis C virus
RAWAQPGYPW

SEQ ID NO: 1635 - protein, 10 aa, hepatitis C virus
RCLVDYPYRL

SEQ ID NO: 1636 - protein, 9 aa, hepatitis C virus
RDRSELSPL

SEQ ID NO: 1637 - protein, 10 aa, hepatitis C virus
RDRSELSPLL

SEQ ID NO: 1638 - protein, 10 aa, hepatitis C virus
REILLGPADG

SEQ ID NO: 1639 - protein, 9 aa, hepatitis C virus
REISVPAEI

SEQ ID NO: 1640 - protein, 10 aa, hepatitis C virus
REISVPAEIL

SEQ ID NO: 1641 - protein, 9 aa, hepatitis C virus
REMAASCGG

SEQ ID NO: 1642 - protein, 9 aa, hepatitis C virus
REPSIPSEY

SEQ ID NO: 1643 - protein, 10 aa, hepatitis C virus
REPSIPSEYL

SEQ ID NO: 1644 - protein, 9 aa, hepatitis C virus
REVSVAAEI

SEQ ID NO: 1645 - protein, 10 aa, hepatitis C virus
REVSVAAEIL

SEQ ID NO: 1646 - protein, 9 aa, hepatitis C virus
REVSVPAEI

SEQ ID NO: 1647 - protein, 10 aa, hepatitis C virus
REVSVPAEIL

SEQ ID NO: 1648 - protein, 9 aa, hepatitis C virus
RGDSRGSLL

SEQ ID NO: 1649 - protein, 10 aa, hepatitis C virus
RGGRDAIILL

SEQ ID NO: 1650 - protein, 10 aa, hepatitis C virus
RGRRGIYRFV

SEQ ID NO: 1651 - protein, 10 aa, hepatitis C virus
RGRRQPIPKA

SEQ ID NO: 1652 - protein, 10 aa, hepatitis C virus
RGRTGRGRRG

SEQ ID NO: 1653 - protein, 10 aa, hepatitis C virus
RGVAKAVDFI

SEQ ID NO: 1654 - protein, 9 aa, hepatitis C virus
RIAEMLKSK

SEQ ID NO: 1655 - protein, 10 aa, hepatitis C virus
RKSRKFPPAL

SEQ ID NO: 1656 - protein, 9 aa, hepatitis C virus
RLGAVQNEV

SEQ ID NO: 1657 - protein, 10 aa, hepatitis C virus
RLHGLDAFSL

SEQ ID NO: 1658 - protein, 10 aa, hepatitis C virus
RLHGLEAFSL

SEQ ID NO: 1659 - protein, 10 aa, hepatitis C virus
RLHGLSAFSL

SEQ ID NO: 1660 - protein, 9 aa, hepatitis C virus
RLHGLSAFT

SEQ ID NO: 1661 - protein, 10 aa, hepatitis C virus
RLIVFPDLGV

SEQ ID NO: 1662 - protein, 9 aa, hepatitis C virus
RLLAPITAY

SEQ ID NO: 1663 - protein, 10 aa, hepatitis C virus
RLLDLSSWFT

SEQ ID NO: 1664 - protein, 10 aa, hepatitis C virus
RLLLLGLLLL

SEQ ID NO: 1665 - protein, 10 aa, hepatitis C virus
RLQVLDDHYK

SEQ ID NO: 1666 - protein, 10 aa, hepatitis C virus
RLVPGAAYAL

SEQ ID NO: 1667 - protein, 9 aa, hepatitis C virus
RLWHYPCTI

SEQ ID NO: 1668 - protein, 9 aa, hepatitis C virus
RLWHYPCTL

SEQ ID NO: 1669 - protein, 9 aa, hepatitis C virus
RLWHYPCTV

SEQ ID NO: 1670 - protein, 10 aa, hepatitis C virus
RLYIGGPLTN

SEQ ID NO: 1671 - protein, 9 aa, hepatitis C virus
RMVLMTHFF

SEQ ID NO: 1672 - protein, 10 aa, hepatitis C virus
RMYVGGVEHR

SEQ ID NO: 1673 - protein, 10 aa, hepatitis C virus
RNLGKVIDTL

SEQ ID NO: 1674 - protein, 10 aa, hepatitis C virus
RPAVIPDREV

SEQ ID NO: 1675 - protein, 9 aa, hepatitis C virus
RPCGIVPAL

SEQ ID NO: 1676 - protein, 9 aa, hepatitis C virus
RPCGIVPAS

SEQ ID NO: 1677 - protein, 9 aa, hepatitis C virus
RPDYNPPLL

SEQ ID NO: 1678 - protein, 10 aa, hepatitis C virus
RPEGRAWAQP

SEQ ID NO: 1679 - protein, 9 aa, hepatitis C virus
RPIDKFAQG

SEQ ID NO: 1680 - protein, 9 aa, hepatitis C virus
RPPQGNWFG

SEQ ID NO: 1681 - protein, 10 aa, hepatitis C virus
RPQDVKFPGG

SEQ ID NO: 1682 - protein, 9 aa, hepatitis C virus
RPRLLLLGL

SEQ ID NO: 1683 - protein, 10 aa, hepatitis C virus
RPRLLLLGLL

SEQ ID NO: 1684 - protein, 9 aa, hepatitis C virus
RPRWFMLCL

SEQ ID NO: 1685 - protein, 10 aa, hepatitis C virus
RPRWFMLCLL

SEQ ID NO: 1686 - protein, 9 aa, hepatitis C virus
RPSGMFDSS

SEQ ID NO: 1687 - protein, 10 aa, hepatitis C virus
RPSGMFDSSV

SEQ ID NO: 1688 - protein, 9 aa, hepatitis C virus
RPSGMFDSV

SEQ ID NO: 1689 - protein, 10 aa, hepatitis C virus
RPSGMFDSVV

SEQ ID NO: 1690 - protein, 10 aa, hepatitis C virus
RPSWGPTDPR

SEQ ID NO: 1691 - protein, 9 aa, hepatitis C virus
RPYCWHYAP

SEQ ID NO: 1692 - protein, 10 aa, hepatitis C virus
RQEMGGNITR

SEQ ID NO: 1693 - protein, 10 aa, hepatitis C virus
RQEMGSNITR

SEQ ID NO: 1694 - protein, 9 aa, hepatitis C virus
RQKKVTFDR

SEQ ID NO: 1695 - protein, 10 aa, hepatitis C virus
RQKKVTFDRL

SEQ ID NO: 1696 - protein, 10 aa, hepatitis C virus
RRPQDVKFPG

SEQ ID NO: 1697 - protein, 10 aa, hepatitis C virus
RRRGDSRGSL

SEQ ID NO: 1698 - protein, 10 aa, hepatitis C virus
RRRSRNLGKV

SEQ ID NO: 1699 - protein, 10 aa, hepatitis C virus
RRSRNLGKVI

SEQ ID NO: 1700 - protein, 9 aa, hepatitis C virus
RSELSPLLL

SEQ ID NO: 1701 - protein, 10 aa, hepatitis C virus
RSELSPLLLS

SEQ ID NO: 1702 - protein, 10 aa, hepatitis C virus
RSLTERLYIG

SEQ ID NO: 1703 - protein, 10 aa, hepatitis C virus
RTALNCNDSL

SEQ ID NO: 1704 - protein, 10 aa, hepatitis C virus
RTGRGRRGIY

SEQ ID NO: 1705 - protein, 9 aa, hepatitis C virus
RTKLKLTPI

SEQ ID NO: 1706 - protein, 9 aa, hepatitis C virus
RTTSGFTSL

SEQ ID NO: 1707 - protein, 9 aa, hepatitis C virus
RVASCLRKL

SEQ ID NO: 1708 - protein, 9 aa, hepatitis C virus
RVCACLWMM

SEQ ID NO: 1709 - protein, 10 aa, hepatitis C virus
RVCACLWMML

SEQ ID NO: 1710 - protein, 9 aa, hepatitis C virus
RVEFLVNAW

SEQ ID NO: 1711 - protein, 10 aa, hepatitis C virus
RVEFLVNAWK

SEQ ID NO: 1712 - protein, 10 aa, hepatitis C virus
RVFTEAMTRY

SEQ ID NO: 1713 - protein, 9 aa, hepatitis C virus
RVLEDGINY

SEQ ID NO: 1714 - protein, 9 aa, hepatitis C virus
RVTQILSSL

SEQ ID NO: 1715 - protein, 9 aa, hepatitis C virus
RYAPPCKPL

SEQ ID NO: 1716 - protein, 10 aa, hepatitis C virus
RYAPPCKPLL

SEQ ID NO: 1717 - protein, 10 aa, hepatitis C virus
SALGLNAVAY

SEQ ID NO: 1718 - protein, 9 aa, hepatitis C virus
SARRGREIL

SEQ ID NO: 1719 - protein, 10 aa, hepatitis C virus
SARRGREILL

SEQ ID NO: 1720 - protein, 10 aa, hepatitis C virus
SASQLSAPSL

SEQ ID NO: 1721 - protein, 10 aa, hepatitis C virus
SCGGAVFVGL

SEQ ID NO: 1722 - protein, 9 aa, hepatitis C virus
SCGNTLTCY

SEQ ID NO: 1723 - protein, 10 aa, hepatitis C virus
SEASSSASQL

SEQ ID NO: 1724 - protein, 9 aa, hepatitis C virus
SEDVVCCSM

SEQ ID NO: 1725 - protein, 9 aa, hepatitis C virus
SELSPLLLS

SEQ ID NO: 1726 - protein, 10 aa, hepatitis C virus
SELSPLLLST

SEQ ID NO: 1727 - protein, 10 aa, hepatitis C virus
SEYLLPKSRF

SEQ ID NO: 1728 - protein, 10 aa, hepatitis C virus
SFLVFFCAAW

SEQ ID NO: 1729 - protein, 10 aa, hepatitis C virus
SFSIFLLALF

SEQ ID NO: 1730 - protein, 10 aa, hepatitis C virus
SFSIFLLALL

SEQ ID NO: 1731 - protein, 9 aa, hepatitis C virus
SGGDIYHSL

SEQ ID NO: 1732 - protein, 9 aa, hepatitis C virus
SGIQYLAGL

SEQ ID NO: 1733 - protein, 10 aa, hepatitis C virus
SGKRVYYLTR

SEQ ID NO: 1734 - protein, 10 aa, hepatitis C virus
SGKSTKVPAA

SEQ ID NO: 1735 - protein, 10 aa, hepatitis C virus
SGPWLTPRCL

SEQ ID NO: 1736 - protein, 10 aa, hepatitis C virus
SIPSEYLLPK

SEQ ID NO: 1737 - protein, 9 aa, hepatitis C virus
SISGVLWTV

SEQ ID NO: 1738 - protein, 9 aa, hepatitis C virus
SITYSTYGK

SEQ ID NO: 1739 - protein, 10 aa, hepatitis C virus
SKKCPMGFSY

SEQ ID NO: 1740 - protein, 10 aa, hepatitis C virus
SLASSSASQL

SEQ ID NO: 1741 - protein, 10 aa, hepatitis C virus
SLDPTFTIET

SEQ ID NO: 1742 - protein, 10 aa, hepatitis C virus
SLGLNAVAYY

SEQ ID NO: 1743 - protein, 10 aa, hepatitis C virus
SLLRHHNMVY

SEQ ID NO: 1744 - protein, 9 aa, hepatitis C virus
SLLRIPYFV

SEQ ID NO: 1745 - protein, 10 aa, hepatitis C virus
SLLRIPYFVR

SEQ ID NO: 1746 - protein, 9 aa, hepatitis C virus
SLMAFTASV

SEQ ID NO: 1747 - protein, 9 aa, hepatitis C virus
SLTITSLLR

SEQ ID NO: 1748 - protein, 9 aa, hepatitis C virus
SLTVTQLLR

SEQ ID NO: 1749 - protein, 9 aa, hepatitis C virus
SLTVTSLLR

SEQ ID NO: 1750 - protein, 10 aa, hepatitis C virus
SMETTMRSPV

SEQ ID NO: 1751 - protein, 9 aa, hepatitis C virus
SMMAFSAAL

SEQ ID NO: 1752 - protein, 9 aa, hepatitis C virus
SMQGAWAKV

SEQ ID NO: 1753 - protein, 9 aa, hepatitis C virus
SMVGNWAKV

SEQ ID NO: 1754 - protein, 10 aa, hepatitis C virus
SMVGNWAKVL

SEQ ID NO: 1755 - protein, 9 aa, hepatitis C virus
SPAPNYSRA

SEQ ID NO: 1756 - protein, 10 aa, hepatitis C virus
SPAPNYSRAL

SEQ ID NO: 1757 - protein, 9 aa, hepatitis C virus
SPAQRVEFL

SEQ ID NO: 1758 - protein, 9 aa, hepatitis C virus
SPDADLIEA

SEQ ID NO: 1759 - protein, 9 aa, hepatitis C virus
SPGALVVGV

SEQ ID NO: 1760 - protein, 10 aa, hepatitis C virus
SPGALVVGVV

SEQ ID NO: 1761 - protein, 9 aa, hepatitis C virus
SPGEINRVA

SEQ ID NO: 1762 - protein, 10 aa, hepatitis C virus
SPGEINRVAS

SEQ ID NO: 1763 - protein, 9 aa, hepatitis C virus
SPGPSQKIQ

SEQ ID NO: 1764 - protein, 10 aa, hepatitis C virus
SPGPSQKIQL

SEQ ID NO: 1765 - protein, 10 aa, hepatitis C virus
SPGQRVEFLV

SEQ ID NO: 1766 - protein, 9 aa, hepatitis C virus
SPLTTNQTM

SEQ ID NO: 1767 - protein, 9 aa, hepatitis C virus
SPLTTQHTL

SEQ ID NO: 1768 - protein, 10 aa, hepatitis C virus
SPLTTQHTLL

SEQ ID NO: 1769 - protein, 9 aa, hepatitis C virus
SPMEKKVIV

SEQ ID NO: 1770 - protein, 9 aa, hepatitis C virus
SPPAVPQTF

SEQ ID NO: 1771 - protein, 10 aa, hepatitis C virus
SPPSLASSSA

SEQ ID NO: 1772 - protein, 9 aa, hepatitis C virus
SPRGSRPNW

SEQ ID NO: 1773 - protein, 10 aa, hepatitis C virus
SPRGSRPSWG

SEQ ID NO: 1774 - protein, 9 aa, hepatitis C virus
SPRGSRPTW

SEQ ID NO: 1775 - protein, 9 aa, hepatitis C virus
SPRPVSYLK

SEQ ID NO: 1776 - protein, 10 aa, hepatitis C virus
SPRPVSYLKG

SEQ ID NO: 1777 - protein, 9 aa, hepatitis C virus
SPRRHETVQ

SEQ ID NO: 1778 - protein, 10 aa, hepatitis C virus
SPRRHETVQD

SEQ ID NO: 1779 - protein, 9 aa, hepatitis C virus
SPTHYVPES

SEQ ID NO: 1780 - protein, 9 aa, hepatitis C virus
SPYYKVFLA

SEQ ID NO: 1781 - protein, 9 aa, hepatitis C virus
SQLSAPSLK

SEQ ID NO: 1782 - protein, 9 aa, hepatitis C virus
SQLSAPSLR

SEQ ID NO: 1783 - protein, 10 aa, hepatitis C virus
SQRRGRTGRG

SEQ ID NO: 1784 - protein, 10 aa, hepatitis C virus
SSALAELATK

SEQ ID NO: 1785 - protein, 9 aa, hepatitis C virus
SSLTITQLL

SEQ ID NO: 1786 - protein, 10 aa, hepatitis C virus
STALAELAAK

SEQ ID NO: 1787 - protein, 9 aa, hepatitis C virus
STEDLVNLL

SEQ ID NO: 1788 - protein, 10 aa, hepatitis C virus
STEDLVNLLP

SEQ ID NO: 1789 - protein, 9 aa, hepatitis C virus
STIPKPQRK

SEQ ID NO: 1790 - protein, 10 aa, hepatitis C virus
STKVPAAYAA

SEQ ID NO: 1791 - protein, 9 aa, hepatitis C virus
STTGEIPFY

SEQ ID NO: 1792 - protein, 10 aa, hepatitis C virus
STVSSALAEL

SEQ ID NO: 1793 - protein, 9 aa, hepatitis C virus
STWVLVGGV

SEQ ID NO: 1794 - protein, 10 aa, hepatitis C virus
STWVLVGGVL

SEQ ID NO: 1795 - protein, 10 aa, hepatitis C virus
STYGKFLADG

SEQ ID NO: 1796 - protein, 9 aa, hepatitis C virus
SVAGAHGIL

SEQ ID NO: 1797 - protein, 10 aa, hepatitis C virus
SVAHDASGKR

SEQ ID NO: 1798 - protein, 10 aa, hepatitis C virus
SVALDPRGRR

SEQ ID NO: 1799 - protein, 9 aa, hepatitis C virus
SVGVGIYLL

SEQ ID NO: 1800 - protein, 9 aa, hepatitis C virus
SVPAEILRK

SEQ ID NO: 1801 - protein, 9 aa, hepatitis C virus
SVRAKLLSR

SEQ ID NO: 1802 - protein, 10 aa, hepatitis C virus
SVRLRAYLNT

SEQ ID NO: 1803 - protein, 10 aa, hepatitis C virus
SVVIVGRIIL

SEQ ID NO: 1804 - protein, 10 aa, hepatitis C virus
SWDQMWKCLI

SEQ ID NO: 1805 - protein, 10 aa, hepatitis C virus
SWDVMWKCLI

SEQ ID NO: 1806 - protein, 10 aa, hepatitis C virus
SWLGNIIMYA

SEQ ID NO: 1807 - protein, 9 aa, hepatitis C virus
SWLRDVWDW

SEQ ID NO: 1808 - protein, 10 aa, hepatitis C virus
SYDTRCFDST

SEQ ID NO: 1809 - protein, 9 aa, hepatitis C virus
SYSWTGALI

SEQ ID NO: 1810 - protein, 9 aa, hepatitis C virus
SYTWTGALI

SEQ ID NO: 1811 - protein, 9 aa, hepatitis C virus
TAACGDIIL

SEQ ID NO: 1812 - protein, 9 aa, hepatitis C virus
TALNCNDSL

SEQ ID NO: 1813 - protein, 9 aa, hepatitis C virus
TALVVSQLL

SEQ ID NO: 1814 - protein, 10 aa, hepatitis C virus
TAYSQQTRGL

SEQ ID NO: 1815 - protein, 10 aa, hepatitis C virus
TCGFADLMGY

SEQ ID NO: 1816 - protein, 9 aa, hepatitis C virus
TCGSSDLYL

SEQ ID NO: 1817 - protein, 10 aa, hepatitis C virus
TDPRRRSRNL

SEQ ID NO: 1818 - protein, 10 aa, hepatitis C virus
TEDLVNLLPA

SEQ ID NO: 1819 - protein, 9 aa, hepatitis C virus
TEPVIFSPM

SEQ ID NO: 1820 - protein, 10 aa, hepatitis C virus
TERLYIGGPL

SEQ ID NO: 1821 - protein, 9 aa, hepatitis C virus
TEVLASMLT

SEQ ID NO: 1822 - protein, 10 aa, hepatitis C virus
TEWAILPCSY

SEQ ID NO: 1823 - protein, 9 aa, hepatitis C virus
TFTIETTTL

SEQ ID NO: 1824 - protein, 10 aa, hepatitis C virus
TFWAKHMWNF

SEQ ID NO: 1825 - protein, 10 aa, hepatitis C virus
TGRGRRGIYR

SEQ ID NO: 1826 - protein, 9 aa, hepatitis C virus
TILGIGTVL

SEQ ID NO: 1827 - protein, 9 aa, hepatitis C virus
TIMAKNEVF

SEQ ID NO: 1828 - protein, 9 aa, hepatitis C virus
TIRRHVDLL

SEQ ID NO: 1829 - protein, 10 aa, hepatitis C virus
TITTGAPITY

SEQ ID NO: 1830 - protein, 9 aa, hepatitis C virus
TLFKVRMYV

SEQ ID NO: 1831 - protein, 10 aa, hepatitis C virus
TLGFGAYMSK

SEQ ID NO: 1832 - protein, 10 aa, hepatitis C virus
TLGFGTYMSK

SEQ ID NO: 1833 - protein, 10 aa, hepatitis C virus
TLHGPTPLLY

SEQ ID NO: 1834 - protein, 10 aa, hepatitis C virus
TLLCPTDCFR

SEQ ID NO: 1835 - protein, 9 aa, hepatitis C virus
TLPALSTGL

SEQ ID NO: 1836 - protein, 10 aa, hepatitis C virus
TLSPYYKVFL

SEQ ID NO: 1837 - protein, 10 aa, hepatitis C virus
TLWARMILMT

SEQ ID NO: 1838 - protein, 10 aa, hepatitis C virus
TNRRPQDVKF

SEQ ID NO: 1839 - protein, 9 aa, hepatitis C virus
TPCAAEESK

SEQ ID NO: 1840 - protein, 10 aa, hepatitis C virus
TPCAAEESKL

SEQ ID NO: 1841 - protein, 9 aa, hepatitis C virus
TPCSGSWLR

SEQ ID NO: 1842 - protein, 9 aa, hepatitis C virus
TPCTCGSSD

SEQ ID NO: 1843 - protein, 10 aa, hepatitis C virus
TPCTCGSSDL

SEQ ID NO: 1844 - protein, 9 aa, hepatitis C virus
TPGCVPCVR

SEQ ID NO: 1845 - protein, 10 aa, hepatitis C virus
TPGERPSGMF

SEQ ID NO: 1846 - protein, 9 aa, hepatitis C virus
TPGLPVCQD

SEQ ID NO: 1847 - protein, 9 aa, hepatitis C virus
TPIDTTIMA

SEQ ID NO: 1848 - protein, 9 aa, hepatitis C virus
TPIPAASQL

SEQ ID NO: 1849 - protein, 9 aa, hepatitis C virus
TPLARAAWE

SEQ ID NO: 1850 - protein, 10 aa, hepatitis C virus
TPLARAAWET

SEQ ID NO: 1851 - protein, 10 aa, hepatitis C virus
TPLLYRLGAV

SEQ ID NO: 1852 - protein, 9 aa, hepatitis C virus
TPLRDWAHA

SEQ ID NO: 1853 - protein, 9 aa, hepatitis C virus
TPPGSVTVP

SEQ ID NO: 1854 - protein, 10 aa, hepatitis C virus
TPPGSVTVPH

SEQ ID NO: 1855 - protein, 9 aa, hepatitis C virus
TPPHSAKSK

SEQ ID NO: 1856 - protein, 10 aa, hepatitis C virus
TPPHSAKSKF

SEQ ID NO: 1857 - protein, 10 aa, hepatitis C virus
TPRCLVDYPY

SEQ ID NO: 1858 - protein, 10 aa, hepatitis C virus
TPRCMVDYPY

SEQ ID NO: 1859 - protein, 9 aa, hepatitis C virus
TPSPAPNYS

SEQ ID NO: 1860 - protein, 9 aa, hepatitis C virus
TPSPVVVGT

SEQ ID NO: 1861 - protein, 10 aa, hepatitis C virus
TPSPVVVGTT

SEQ ID NO: 1862 - protein, 10 aa, hepatitis C virus
TPVNSWLGNI

SEQ ID NO: 1863 - protein, 10 aa, hepatitis C virus
TPYFVRAHVL

SEQ ID NO: 1864 - protein, 9 aa, hepatitis C virus
TQDCNCSIY

SEQ ID NO: 1865 - protein, 10 aa, hepatitis C virus
TRDPTTPLAR

SEQ ID NO: 1866 - protein, 10 aa, hepatitis C virus
TRGVAKAVDF

SEQ ID NO: 1867 - protein, 10 aa, hepatitis C virus
TSCGNTLTCY

SEQ ID NO: 1868 - protein, 10 aa, hepatitis C virus
TSFGNTITCY

SEQ ID NO: 1869 - protein, 10 aa, hepatitis C virus
TSPLTTQHTL

SEQ ID NO: 1870 - protein, 9 aa, hepatitis C virus
TSVRLRAYL

SEQ ID NO: 1871 - protein, 10 aa, hepatitis C virus
TTALVVSQLL

SEQ ID NO: 1872 - protein, 10 aa, hepatitis C virus
TTDRSGAPTY

SEQ ID NO: 1873 - protein, 10 aa, hepatitis C virus
TTGEIPFYGK

SEQ ID NO: 1874 - protein, 9 aa, hepatitis C virus
TTIRRHVDL

SEQ ID NO: 1875 - protein, 10 aa, hepatitis C virus
TTIRRHVDLL

SEQ ID NO: 1876 - protein, 10 aa, hepatitis C virus
TTLPALSTGL

SEQ ID NO: 1877 - protein, 10 aa, hepatitis C virus
TTQDCNCSIY

SEQ ID NO: 1878 - protein, 9 aa, hepatitis C virus
TTSRSASLR

SEQ ID NO: 1879 - protein, 9 aa, hepatitis C virus
TTSRSASQR

SEQ ID NO: 1880 - protein, 10 aa, hepatitis C virus
TVLTDFKTWL

SEQ ID NO: 1881 - protein, 9 aa, hepatitis C virus
TVSSALAEL

SEQ ID NO: 1882 - protein, 9 aa, hepatitis C virus
TVYHGAGNK

SEQ ID NO: 1883 - protein, 9 aa, hepatitis C virus
TWAQPGYPW

SEQ ID NO: 1884 - protein, 10 aa, hepatitis C virus
TYIYNHLTPL

SEQ ID NO: 1885 - protein, 8 aa, hepatitis C virus
TYSTYGKF

SEQ ID NO: 1886 - protein, 10 aa, hepatitis C virus
TYSTYGKFLA

SEQ ID NO: 1887 - protein, 10 aa, hepatitis C virus
VAATLGFGAY

SEQ ID NO: 1888 - protein, 10 aa, hepatitis C virus
VAFKVMSGEM

SEQ ID NO: 1889 - protein, 9 aa, hepatitis C virus
VAGAHWGVL

SEQ ID NO: 1890 - protein, 9 aa, hepatitis C virus
VAGALVAFK

SEQ ID NO: 1891 - protein, 10 aa, hepatitis C virus
VAGALVAFKV

SEQ ID NO: 1892 - protein, 10 aa, hepatitis C virus
VAHDASGKRV

SEQ ID NO: 1893 - protein, 10 aa, hepatitis C virus
VAKAVDFIPV

SEQ ID NO: 1894 - protein, 10 aa, hepatitis C virus
VATDALMTGY

SEQ ID NO: 1895 - protein, 10 aa, hepatitis C virus
VCAAILRRHV

SEQ ID NO: 1896 - protein, 10 aa, hepatitis C virus
VCACLWMMLL

SEQ ID NO: 1897 - protein, 10 aa, hepatitis C virus
VCEKMALYDV

SEQ ID NO: 1898 - protein, 10 aa, hepatitis C virus
VEDVVNLLPA

SEQ ID NO: 1899 - protein, 10 aa, hepatitis C virus
VEESIYQCCD

SEQ ID NO: 1900 - protein, 10 aa, hepatitis C virus
VEFLVNAWKS

SEQ ID NO: 1901 - protein, 9 aa, hepatitis C virus
VESENKVVI

SEQ ID NO: 1902 - protein, 10 aa, hepatitis C virus
VESENKVVIL

SEQ ID NO: 1903 - protein, 9 aa, hepatitis C virus
VESENKVVV

SEQ ID NO: 1904 - protein, 10 aa, hepatitis C virus
VESENKVVVL

SEQ ID NO: 1905 - protein, 10 aa, hepatitis C virus
VESETKVVIL

SEQ ID NO: 1906 - protein, 9 aa, hepatitis C virus
VFDITKWLL

SEQ ID NO: 1907 - protein, 9 aa, hepatitis C virus
VFFCAAWYI

SEQ ID NO: 1908 - protein, 10 aa, hepatitis C virus
VFFCAAWYIK

SEQ ID NO: 1909 - protein, 10 aa, hepatitis C virus
VFWAKHMWNF

SEQ ID NO: 1910 - protein, 10 aa, hepatitis C virus
VGDLCGSVFL

SEQ ID NO: 1911 - protein, 10 aa, hepatitis C virus
VGSIGLGKVL

SEQ ID NO: 1912 - protein, 9 aa, hepatitis C virus
VGVVCAAIL

SEQ ID NO: 1913 - protein, 9 aa, hepatitis C virus
VICAAILRR

SEQ ID NO: 1914 - protein, 10 aa, hepatitis C virus
VIDTLTCGFA

SEQ ID NO: 1915 - protein, 9 aa, hepatitis C virus
VIFSPMEIK

SEQ ID NO: 1916 - protein, 9 aa, hepatitis C virus
VIKGGRHLI

SEQ ID NO: 1917 - protein, 10 aa, hepatitis C virus
VIKGGRHLIF

SEQ ID NO: 1918 - protein, 9 aa, hepatitis C virus
VILDSFDPL

SEQ ID NO: 1919 - protein, 9 aa, hepatitis C virus
VILLFLLLA

SEQ ID NO: 1920 - protein, 10 aa, hepatitis C virus
VKFPGGGQIV

SEQ ID NO: 1921 - protein, 10 aa, hepatitis C virus
VLAALAAYCL

SEQ ID NO: 1922 - protein, 9 aa, hepatitis C virus
VLAGGVLAA

SEQ ID NO: 1923 - protein, 10 aa, hepatitis C virus
VLAGGVLAAV

SEQ ID NO: 1924 - protein, 9 aa, hepatitis C virus
VLAGYGAGI

SEQ ID NO: 1925 - protein, 9 aa, hepatitis C virus
VLAGYGAGV

SEQ ID NO: 1926 - protein, 9 aa, hepatitis C virus
VLALPQQAY

SEQ ID NO: 1927 - protein, 10 aa, hepatitis C virus
VLEDGVNYAT

SEQ ID NO: 1928 - protein, 9 aa, hepatitis C virus
VLMTHFFSI

SEQ ID NO: 1929 - protein, 10 aa, hepatitis C virus
VLMTHFFSIL

SEQ ID NO: 1930 - protein, 10 aa, hepatitis C virus
VLTTSCGNTL

SEQ ID NO: 1931 - protein, 9 aa, hepatitis C virus
VLVDILAGY

SEQ ID NO: 1932 - protein, 10 aa, hepatitis C virus
VLVDILAGYG

SEQ ID NO: 1933 - protein, 9 aa, hepatitis C virus
VLVGGVLAA

SEQ ID NO: 1934 - protein, 10 aa, hepatitis C virus
VLVGGVLAAL

SEQ ID NO: 1935 - protein, 9 aa, hepatitis C virus
VLWTVYHGA

SEQ ID NO: 1936 - protein, 9 aa, hepatitis C virus
VMACMSADL

SEQ ID NO: 1937 - protein, 10 aa, hepatitis C virus
VMFGLAYFSM

SEQ ID NO: 1938 - protein, 8 aa, hepatitis C virus
VMGSSYGF

SEQ ID NO: 1939 - protein, 10 aa, hepatitis C virus
VMGSSYGFQY

SEQ ID NO: 1940 - protein, 9 aa, hepatitis C virus
VMWKCLIRL

SEQ ID NO: 1941 - protein, 9 aa, hepatitis C virus
VMWKCLTRL

SEQ ID NO: 1942 - protein, 9 aa, hepatitis C virus
VMWTVYHGA

SEQ ID NO: 1943 - protein, 10 aa, hepatitis C virus
VPAAYAAQGY

SEQ ID NO: 1944 - protein, 10 aa, hepatitis C virus
VPAPEFFTEV

SEQ ID NO: 1945 - protein, 10 aa, hepatitis C virus
VPARSVCGPV

SEQ ID NO: 1946 - protein, 10 aa, hepatitis C virus
VPASQVCGPV

SEQ ID NO: 1947 - protein, 10 aa, hepatitis C virus
VPASSVCGPV

SEQ ID NO: 1948 - protein, 10 aa, hepatitis C virus
VPESDAAARV

SEQ ID NO: 1949 - protein, 9 aa, hepatitis C virus
VPGAAYALY

SEQ ID NO: 1950 - protein, 9 aa, hepatitis C virus
VPHPNIEEV

SEQ ID NO: 1951 - protein, 10 aa, hepatitis C virus
VPHPNIEEVA

SEQ ID NO: 1952 - protein, 9 aa, hepatitis C virus
VPPVVHGCP

SEQ ID NO: 1953 - protein, 10 aa, hepatitis C virus
VPPVVHGCPL

SEQ ID NO: 1954 - protein, 10 aa, hepatitis C virus
VPQTFQVAHL

SEQ ID NO: 1955 - protein, 9 aa, hepatitis C virus
VPTTTIRRH

SEQ ID NO: 1956 - protein, 10 aa, hepatitis C virus
VPTTTIRRHV

SEQ ID NO: 1957 - protein, 9 aa, hepatitis C virus
VPVESMETT

SEQ ID NO: 1958 - protein, 10 aa, hepatitis C virus
VPVESMETTM

SEQ ID NO: 1959 - protein, 10 aa, hepatitis C virus
VPYFVRAHAL

SEQ ID NO: 1960 - protein, 10 aa, hepatitis C virus
VPYFVRAQGL

SEQ ID NO: 1961 - protein, 9 aa, hepatitis C virus
VQDCNCSIY

SEQ ID NO: 1962 - protein, 9 aa, hepatitis C virus
VQECNCSIY

SEQ ID NO: 1963 - protein, 10 aa, hepatitis C virus
VQWMNRLIAF

SEQ ID NO: 1964 - protein, 10 aa, hepatitis C virus
VRATRKTSER

SEQ ID NO: 1965 - protein, 9 aa, hepatitis C virus
VRGGRDAII

SEQ ID NO: 1966 - protein, 10 aa, hepatitis C virus
VRGGRDAIIL

SEQ ID NO: 1967 - protein, 10 aa, hepatitis C virus
VRVCEKMALY

SEQ ID NO: 1968 - protein, 10 aa, hepatitis C virus
VRVLEDGVNY

SEQ ID NO: 1969 - protein, 9 aa, hepatitis C virus
VSARRGREI

SEQ ID NO: 1970 - protein, 10 aa, hepatitis C virus
VSARRGREIL

SEQ ID NO: 1971 - protein, 9 aa, hepatitis C virus
VSGALVAFK

SEQ ID NO: 1972 - protein, 10 aa, hepatitis C virus
VSRSQRRGRT

SEQ ID NO: 1973 - protein, 9 aa, hepatitis C virus
VSTATQSFL

SEQ ID NO: 1974 - protein, 10 aa, hepatitis C virus
VSVPAEILRK

SEQ ID NO: 1975 - protein, 9 aa, hepatitis C virus
VTFDRLQVL

SEQ ID NO: 1976 - protein, 10 aa, hepatitis C virus
VTLTHPITKY

SEQ ID NO: 1977 - protein, 10 aa, hepatitis C virus
VTQILSSLTI

SEQ ID NO: 1978 - protein, 9 aa, hepatitis C virus
VVCAAILRR

SEQ ID NO: 1979 - protein, 9 aa, hepatitis C virus
VVESKWRAL

SEQ ID NO: 1980 - protein, 10 aa, hepatitis C virus
VVGVVCAAIL

SEQ ID NO: 1981 - protein, 10 aa, hepatitis C virus
VVILDSFDPL

SEQ ID NO: 1982 - protein, 9 aa, hepatitis C virus
VVIVGRIIL

SEQ ID NO: 1983 - protein, 9 aa, hepatitis C virus
VVLLFLLLA

SEQ ID NO: 1984 - protein, 9 aa, hepatitis C virus
VVLTHPITK

SEQ ID NO: 1985 - protein, 10 aa, hepatitis C virus
VVSTATQSFL

SEQ ID NO: 1986 - protein, 10 aa, hepatitis C virus
VVSTLPQAVM

SEQ ID NO: 1987 - protein, 10 aa, hepatitis C virus
VVVATDALMT

SEQ ID NO: 1988 - protein, 9 aa, hepatitis C virus
VVVVATDAL

SEQ ID NO: 1989 - protein, 10 aa, hepatitis C virus
VVVVATDALM

SEQ ID NO: 1990 - protein, 10 aa, hepatitis C virus
VYLLPRRGPR

SEQ ID NO: 1991 - protein, 10 aa, hepatitis C virus
VYYLTRDPTT

SEQ ID NO: 1992 - protein, 9 aa, hepatitis C virus
WAHAGLRDL

SEQ ID NO: 1993 - protein, 9 aa, hepatitis C virus
WAKHMWNFI

SEQ ID NO: 1994 - protein, 9 aa, hepatitis C virus
WAKVLIVML

SEQ ID NO: 1995 - protein, 10 aa, hepatitis C virus
WAKVLIVMLL

SEQ ID NO: 1996 - protein, 10 aa, hepatitis C virus
WAQPGYPWPL

SEQ ID NO: 1997 - protein, 10 aa, hepatitis C virus
WARMILMTHF

SEQ ID NO: 1998 - protein, 10 aa, hepatitis C virus
WARPDYNPPL

SEQ ID NO: 1999 - protein, 9 aa, hepatitis C virus
WAVRTKLKL

SEQ ID NO: 2000 - protein, 9 aa, hepatitis C virus
WEAVFTGLT

SEQ ID NO: 2001 - protein, 9 aa, hepatitis C virus
WEGVFTGLT

SEQ ID NO: 2002 - protein, 10 aa, hepatitis C virus
WESVFTGLTH

SEQ ID NO: 2003 - protein, 9 aa, hepatitis C virus
WETARHTPI

SEQ ID NO: 2004 - protein, 10 aa, hepatitis C virus
WETARHTPVN

SEQ ID NO: 2005 - protein, 9 aa, hepatitis C virus
WETVRHSPV

SEQ ID NO: 2006 - protein, 9 aa, hepatitis C virus
WETVRHTPV

SEQ ID NO: 2007 - protein, 9 aa, hepatitis C virus
WEWVILLFL

SEQ ID NO: 2008 - protein, 10 aa, hepatitis C virus
WEWVILLFLL

SEQ ID NO: 2009 - protein, 9 aa, hepatitis C virus
WEWVVLLFL

SEQ ID NO: 2010 - protein, 10 aa, hepatitis C virus
WEWVVLLFLL

SEQ ID NO: 2011 - protein, 9 aa, hepatitis C virus
WEYVVLLFL

SEQ ID NO: 2012 - protein, 10 aa, hepatitis C virus
WEYVVLLFLL

SEQ ID NO: 2013 - protein, 10 aa, hepatitis C virus
WKSKKCPMGF

SEQ ID NO: 2014 - protein, 10 aa, hepatitis C virus
WLQSKLLPRL

SEQ ID NO: 2015 - protein, 9 aa, hepatitis C virus
WMNRLIAFA

SEQ ID NO: 2016 - protein, 9 aa, hepatitis C virus
WMNSTGFTK

SEQ ID NO: 2017 - protein, 9 aa, hepatitis C virus
WPLLLLLLA

SEQ ID NO: 2018 - protein, 10 aa, hepatitis C virus
WPLLLLLLAL

SEQ ID NO: 2019 - protein, 9 aa, hepatitis C virus
WPLYGNEGM

SEQ ID NO: 2020 - protein, 10 aa, hepatitis C virus
WPLYGNEGMG

SEQ ID NO: 2021 - protein, 10 aa, hepatitis C virus
WQAPPGARSL

SEQ ID NO: 2022 - protein, 9 aa, hepatitis C virus
WTRGERCDL

SEQ ID NO: 2023 - protein, 10 aa, hepatitis C virus
WYLVAFCAAW

SEQ ID NO: 2024 - protein, 10 aa, hepatitis C virus
YAAQGYKVLV

SEQ ID NO: 2025 - protein, 9 aa, hepatitis C virus
YALYGVWPL

SEQ ID NO: 2026 - protein, 10 aa, hepatitis C virus
YALYGVWPLL

SEQ ID NO: 2027 - protein, 9 aa, hepatitis C virus
YAPACKPLL

SEQ ID NO: 2028 - protein, 10 aa, hepatitis C virus
YAPTLWARMI

SEQ ID NO: 2029 - protein, 10 aa, hepatitis C virus
YATTSRSASL

SEQ ID NO: 2030 - protein, 10 aa, hepatitis C virus
YDAGCAWYEL

SEQ ID NO: 2031 - protein, 9 aa, hepatitis C virus
YELTPAETT

SEQ ID NO: 2032 - protein, 9 aa, hepatitis C virus
YFVRAHALL

SEQ ID NO: 2033 - protein, 10 aa, hepatitis C virus
YGACYSIEPL

SEQ ID NO: 2034 - protein, 9 aa, hepatitis C virus
YGKAIPIEV

SEQ ID NO: 2035 - protein, 10 aa, hepatitis C virus
YGKAIPIEVI

SEQ ID NO: 2036 - protein, 9 aa, hepatitis C virus
YGVWPLLLL

SEQ ID NO: 2037 - protein, 10 aa, hepatitis C virus
YIMACMSADL

SEQ ID NO: 2038 - protein, 9 aa, hepatitis C virus
YIPLVGAPL

SEQ ID NO: 2039 - protein, 9 aa, hepatitis C virus
YIYDHLTPM

SEQ ID NO: 2040 - protein, 9 aa, hepatitis C virus
YIYNHLTPL

SEQ ID NO: 2041 - protein, 10 aa, hepatitis C virus
YKVLVLNPSV

SEQ ID NO: 2042 - protein, 10 aa, hepatitis C virus
YLFNWAVKTK

SEQ ID NO: 2043 - protein, 9 aa, hepatitis C virus
YLFNWAVRT

SEQ ID NO: 2044 - protein, 10 aa, hepatitis C virus
YLFNWAVRTK

SEQ ID NO: 2045 - protein, 10 aa, hepatitis C virus
YLKASAACRA

SEQ ID NO: 2046 - protein, 10 aa, hepatitis C virus
YLKGSSGGPL

SEQ ID NO: 2047 - protein, 10 aa, hepatitis C virus
YLLPRRGPRL

SEQ ID NO: 2048 - protein, 9 aa, hepatitis C virus
YLNTPGLPV

SEQ ID NO: 2049 - protein, 9 aa, hepatitis C virus
YLSTPGLPV

SEQ ID NO: 2050 - protein, 9 aa, hepatitis C virus
YLTAYQATV

SEQ ID NO: 2051 - protein, 10 aa, hepatitis C virus
YLTRDPTTPL

SEQ ID NO: 2052 - protein, 9 aa, hepatitis C virus
YLVAYQATV

SEQ ID NO: 2053 - protein, 9 aa, hepatitis C virus
YLVTRHADV

SEQ ID NO: 2054 - protein, 10 aa, hepatitis C virus
YLVTRHADVI

SEQ ID NO: 2055 - protein, 9 aa, hepatitis C virus
YLVTRNADV

SEQ ID NO: 2056 - protein, 9 aa, hepatitis C virus
YLYGVGSAV

SEQ ID NO: 2057 - protein, 10 aa, hepatitis C virus
YLYGVGSAVV

SEQ ID NO: 2058 - protein, 9 aa, hepatitis C virus
YPCTVNFSI

SEQ ID NO: 2059 - protein, 9 aa, hepatitis C virus
YPCTVNFTI

SEQ ID NO: 2060 - protein, 10 aa, hepatitis C virus
YPCTVNFTIF

SEQ ID NO: 2061 - protein, 9 aa, hepatitis C virus
YPCTVNFTL

SEQ ID NO: 2062 - protein, 9 aa, hepatitis C virus
YPGHVSGHR

SEQ ID NO: 2063 - protein, 10 aa, hepatitis C virus
YPGHVSGHRM

SEQ ID NO: 2064 - protein, 10 aa, hepatitis C virus
YPWPLYGNEG

SEQ ID NO: 2065 - protein, 10 aa, hepatitis C virus
YRLGAVQNEV

SEQ ID NO: 2066 - protein, 10 aa, hepatitis C virus
YRRCRASGVL

SEQ ID NO: 2067 - protein, 10 aa, hepatitis C virus
YSGGDIYHSL

SEQ ID NO: 2068 - protein, 10 aa, hepatitis C virus
YSPGQRVEFL

SEQ ID NO: 2069 - protein, 9 aa, hepatitis C virus
YSQQTRGLL

SEQ ID NO: 2070 - protein, 9 aa, hepatitis C virus
YTIFKIRMY

SEQ ID NO: 2071 - protein, 10 aa, hepatitis C virus
YTKCGSGPWL

SEQ ID NO: 2072 - protein, 9 aa, hepatitis C virus
YVGGVEHRL

SEQ ID NO: 2073 - protein, 9 aa, hepatitis C virus
YVLLLFLLL

SEQ ID NO: 2074 - protein, 9 aa, hepatitis C virus
YVQMAFMKL

SEQ ID NO: 2075 - protein, 9 aa, hepatitis C virus
YVVLLFLLL

SEQ ID NO: 2076 - protein, 10 aa, hepatitis C virus
YVVLLFLLLA

SEQ ID NO: 2077 - protein, 9 aa, hepatitis C virus
YVYDHLTPL

SEQ ID NO: 2078 - protein, 10 aa, hepatitis C virus
YVYDHLTPLR

SEQ ID NO: 2079 - protein, 9 aa, hepatitis C virus
YVYNHLTPL

SEQ ID NO: 2080 - protein, 10 aa, hepatitis C virus
YVYNHLTPLR

SEQ ID NO: 2081 - protein, 10 aa, hepatitis C virus
YYLTRDPTTP

SEQ ID NO: 2082 - protein, 9 aa, hepatitis C virus
YYRGLDVSI

SEQ ID NO: 2083 - protein, 10 aa, hepatitis C virus
YYRGLDVSVI

SEQ ID NO: 2084 - protein, 9 aa, hepatitis C virus
GEMPSTEDL

SEQ ID NO: 2085 - protein, 9 aa, hepatitis C virus
ADLMGYIPL

SEQ ID NO: 2086 - protein, 9 aa, hepatitis C virus
GALVAFKVM

SEQ ID NO: 2087 - protein, 9 aa, hepatitis C virus
TRVPYFVRA

SEQ ID NO: 2088 - protein, 9 aa, hepatitis C virus
SDVESYSSM

SEQ ID NO: 2089 - protein, 9 aa, hepatitis C virus
ESYSSMPPL

SEQ ID NO: 2090 - protein, 9 aa, hepatitis C virus
VLYREFDEM

SEQ ID NO: 2091 - protein, 9 aa, hepatitis C virus
QIIERLHGL

SEQ ID NO: 2092 - protein, 9 aa, hepatitis C virus
LSVEEACKL

SEQ ID NO: 2093 - protein, 9 aa, hepatitis C virus
WHYPCTVNF

SEQ ID NO: 2094 - protein, 9 aa, hepatitis C virus
CQVPAPEFF

SEQ ID NO: 2095 - protein, 9 aa, hepatitis C virus
HGILSFLVF

SEQ ID NO: 2096 - protein, 9 aa, hepatitis C virus
STLPGNPAI

SEQ ID NO: 2097 - protein, 9 aa, hepatitis C virus
AKVLIVMLL

SEQ ID NO: 2098 - protein, 9 aa, hepatitis C virus
QNIVDVQYL

SEQ ID NO: 2099 - protein, 9 aa, hepatitis C virus
NQYLVGSQL

SEQ ID NO: 2100 - protein, 9 aa, hepatitis C virus
FQVGLNQYL

SEQ ID NO: 2101 - protein, 9 aa, hepatitis C virus
TMLVNGDDL

SEQ ID NO: 2102 - protein, 15 aa, hepatitis C virus
AAQLAPPSAASAFVG

SEQ ID NO: 2103 - protein, 15 aa, hepatitis C virus
ACKLTPPHSAKSKFG

SEQ ID NO: 2104 - protein, 15 aa, hepatitis C virus
ADLIEANLLWRQEMG

SEQ ID NO: 2105 - protein, 15 aa, hepatitis C virus
ADLMGYIPLVGAPLG

SEQ ID NO: 2106 - protein, 15 aa, hepatitis C virus
APTLWARMILMTHFF

SEQ ID NO: 2107 - protein, 15 aa, hepatitis C virus
AQGYKVLVLNPSVAA

SEQ ID NO: 2108 - protein, 15 aa, hepatitis C virus
ARAAWETARHTPVNS

SEQ ID NO: 2109 - protein, 15 aa, hepatitis C virus
ARLVVLATATPPGSV

SEQ ID NO: 2110 - protein, 15 aa, hepatitis C virus
ASCLRKLGVPPLRVW

SEQ ID NO: 2111 - protein, 15 aa, hepatitis C virus
ASQLSAPSLKATCTT

SEQ ID NO: 2112 - protein, 15 aa, hepatitis C virus
AVGIFRAAVCTRGVA

SEQ ID NO: 2113 - protein, 15 aa, hepatitis C virus
AVQWMNRLIAFASRG

SEQ ID NO: 2114 - protein, 15 aa, hepatitis C virus
AWDMMMNWSPTTALV

SEQ ID NO: 2115 - protein, 15 aa, hepatitis C virus
AYCLTTGSVVIVGRI

SEQ ID NO: 2116 - protein, 15 aa, hepatitis C virus
CQIYGACYSIEPLDL

SEQ ID NO: 2117 - protein, 15 aa, hepatitis C virus
DADLIEANLLWRQEM

SEQ ID NO: 2118 - protein, 15 aa, hepatitis C virus
DAHFLSQTKQAGDNF

SEQ ID NO: 2119 - protein, 15 aa, hepatitis C virus
DIIICDECHSTDSTT

SEQ ID NO: 2120 - protein, 15 aa, hepatitis C virus
DLAVAVEPVVFSDME

SEQ ID NO: 2121 - protein, 14 aa, hepatitis C virus
DLVNLLPAILSPGA

SEQ ID NO: 2122 - protein, 15 aa, hepatitis C virus
DPTFTIETTTVPQDA

SEQ ID NO: 2123 - protein, 15 aa, hepatitis C virus
DSSVLCECYDAGCAW

SEQ ID NO: 2124 - protein, 15 aa, hepatitis C virus
DVVVVATDALMTGFT

SEQ ID NO: 2125 - protein, 15 aa, hepatitis C virus
DVVVVATDALMTGYT

SEQ ID NO: 2126 - protein, 14 aa, hepatitis C virus
EDLVNLLPAILSPG

SEQ ID NO: 2127 - protein, 15 aa, hepatitis C virus
EPDVAVLTSMLTDPS

SEQ ID NO: 2128 - protein, 15 aa, hepatitis C virus
FNILGGWVAAQLAPP

SEQ ID NO: 2129 - protein, 15 aa, hepatitis C virus
FPYLVAYQATVCARA

SEQ ID NO: 2130 - protein, 15 aa, hepatitis C virus
FSIFLLALLSCLTIP

SEQ ID NO: 2131 - protein, 15 aa, hepatitis C virus
FSIFLLALLSCLTVP

SEQ ID NO: 2132 - protein, 15 aa, hepatitis C virus
FTTLPALSTGLIHLH

SEQ ID NO: 2133 - protein, 15 aa, hepatitis C virus
GACYSIEPLDLPQII

SEQ ID NO: 2134 - protein, 15 aa, hepatitis C virus
GAGVAGALVAFKIMS

SEQ ID NO: 2135 - protein, 15 aa, hepatitis C virus
GAGVAGALVAFKVMS

SEQ ID NO: 2136 - protein, 15 aa, hepatitis C virus
GALVVGVVCAAILRR

SEQ ID NO: 2137 - protein, 15 aa, hepatitis C virus
GARLVVLATATPPGS

SEQ ID NO: 2138 - protein, 15 aa, hepatitis C virus
GCGWAGWLLSPRGSR

SEQ ID NO: 2139 - protein, 15 aa, hepatitis C virus
GCSFSIFLLALLSCL

SEQ ID NO: 2140 - protein, 15 aa, hepatitis C virus
GDNFPYLVAYQATVC

SEQ ID NO: 2141 - protein, 15 aa, hepatitis C virus
GGVLAALAAYCLTTG

SEQ ID NO: 2142 - protein, 15 aa, hepatitis C virus
GHRMAWDMMMNWSPT

SEQ ID NO: 2143 - protein, 15 aa, hepatitis C virus
GIQYLAGLSTLPGNP

SEQ ID NO: 2144 - protein, 15 aa, hepatitis C virus
GKYLFNWAVRTKLKL

SEQ ID NO: 2145 - protein, 15 aa, hepatitis C virus
GLGWAGWLLSPRGSR

SEQ ID NO: 2146 - protein, 15 aa, hepatitis C virus
GLPVCQDHLEFWESV

SEQ ID NO: 2147 - protein, 15 aa, hepatitis C virus
GMGWAGWLLSPRGSR

SEQ ID NO: 2148 - protein, 15 aa, hepatitis C virus
GMQLAEQFKQKALGL

SEQ ID NO: 2149 - protein, 15 aa, hepatitis C virus
GPRLGVRATRKTSER

SEQ ID NO: 2150 - protein, 15 aa, hepatitis C virus
GQGWRLLAPITAYSQ

SEQ ID NO: 2151 - protein, 15 aa, hepatitis C virus
GQIVGGVYLLPRRGP

SEQ ID NO: 2152 - protein, 15 aa, hepatitis C virus
GSQLPCEPEPDVAVL

SEQ ID NO: 2153 - protein, 15 aa, hepatitis C virus
GSSYGFQYSPGQRVE

SEQ ID NO: 2154 - protein, 15 aa, hepatitis C virus
GTVLDQAETAGARLV

SEQ ID NO: 2155 - protein, 15 aa, hepatitis C virus
GVNYATGNLPGCSFS

SEQ ID NO: 2156 - protein, 15 aa, hepatitis C virus
GVRVLEDGVNYATGN

SEQ ID NO: 2157 - protein, 15 aa, hepatitis C virus
GYKVLVLNPSVAATL

SEQ ID NO: 2158 - protein, 15 aa, hepatitis C virus
HLIFCHSKKKCDELA

SEQ ID NO: 2159 - protein, 15 aa, hepatitis C virus
HQWINEDCSTPCSGS

SEQ ID NO: 2160 - protein, 15 aa, hepatitis C virus
IERLHGLSAFSLHSY

SEQ ID NO: 2161 - protein, 15 aa, hepatitis C virus
IQRLHGLSAFSLHSY

SEQ ID NO: 2162 - protein, 15 aa, hepatitis C virus
IQYLAGLSTLPGNPA

SEQ ID NO: 2163 - protein, 15 aa, hepatitis C virus
ITRVESENKVVILDS

SEQ ID NO: 2164 - protein, 15 aa, hepatitis C virus
KPTLHGPTPLLYRLG

SEQ ID NO: 2165 - protein, 15 aa, hepatitis C virus
KVLVLNPSVAATLGF

SEQ ID NO: 2166 - protein, 15 aa, hepatitis C virus
LAGYGAGVAGALVAF

SEQ ID NO: 2167 - protein, 15 aa, hepatitis C virus
LFLLLADARVCACLW

SEQ ID NO: 2168 - protein, 15 aa, hepatitis C virus
LFNILGGWVAAQLAP

SEQ ID NO: 2169 - protein, 15 aa, hepatitis C virus
LGKVLVDILAGYGAG

SEQ ID NO: 2170 - protein, 15 aa, hepatitis C virus
LHSYSPGEINRVASC

SEQ ID NO: 2171 - protein, 15 aa, hepatitis C virus
LIRLKPTLHGPTPLL

SEQ ID NO: 2172 - protein, 15 aa, hepatitis C virus
LPAILSPGALVVGVV

SEQ ID NO: 2173 - protein, 15 aa, hepatitis C virus
LPALSTGLIHLHQNI

SEQ ID NO: 2174 - protein, 15 aa, hepatitis C virus
LSTLPGNPAIASLMA

SEQ ID NO: 2175 - protein, 15 aa, hepatitis C virus
LTHIDAHFLSQTKQA

SEQ ID NO: 2176 - protein, 15 aa, hepatitis C virus
LTHIDAHFLSQTKQS

SEQ ID NO: 2177 - protein, 15 aa, hepatitis C virus
LTSMLTDPSHITAET

SEQ ID NO: 2178 - protein, 15 aa, hepatitis C virus
LVAYQATVCARAQAP

SEQ ID NO: 2179 - protein, 15 aa, hepatitis C virus
LVNLLPAILSPGALV

SEQ ID NO: 2180 - protein, 15 aa, hepatitis C virus
LVVLATATPPGSVTV

SEQ ID NO: 2181 - protein, 15 aa, hepatitis C virus
MACMSADLEVVTSTW

SEQ ID NO: 2182 - protein, 15 aa, hepatitis C virus
MNRLIAFASRGNHVS

SEQ ID NO: 2183 - protein, 13 aa, hepatitis C virus
MPPLEGEPGDPDL

SEQ ID NO: 2184 - protein, 12 aa, hepatitis C virus
MSTNPKPQRKTK

SEQ ID NO: 2185 - protein, 15 aa, hepatitis C virus
MTGFTGDFDSVIDCN

SEQ ID NO: 2186 - protein, 15 aa, hepatitis C virus
NPAIASLMAFTASIT

SEQ ID NO: 2187 - protein, 15 aa, hepatitis C virus
NSWLGNIIMYAPTLW

SEQ ID NO: 2188 - protein, 15 aa, hepatitis C virus
NVSVAHDASGKRVYY

SEQ ID NO: 2189 - protein, 15 aa, hepatitis C virus
PCSFTTLPALSTGLI

SEQ ID NO: 2190 - protein, 15 aa, hepatitis C virus
PGALVVGVVCAAILR

SEQ ID NO: 2191 - protein, 15 aa, hepatitis C virus
PMGFSYDTRCFDSTV

SEQ ID NO: 2192 - protein, 15 aa, hepatitis C virus
PQTFQVAHLHAPTGS

SEQ ID NO: 2193 - protein, 15 aa, hepatitis C virus
PTHYVPESDAAARVT

SEQ ID NO: 2194 - protein, 15 aa, hepatitis C virus
PTLWARMILMTHFFS

SEQ ID NO: 2195 - protein, 15 aa, hepatitis C virus
PYLVAYQATVCARAQ

SEQ ID NO: 2196 - protein, 15 aa, hepatitis C virus
QDAVSRSQRRGRTGR

SEQ ID NO: 2197 - protein, 15 aa, hepatitis C virus
QKKVTFDRLQVLDDH

SEQ ID NO: 2198 - protein, 15 aa, hepatitis C virus
QPEYDLELITSCSSN

SEQ ID NO: 2199 - protein, 15 aa, hepatitis C virus
RAAVCTRGVAKAVDF

SEQ ID NO: 2200 - protein, 15 aa, hepatitis C virus
RGLLGCIITSLTGRD

SEQ ID NO: 2201 - protein, 15 aa, hepatitis C virus
RLGVRATRKTSERSQ

SEQ ID NO: 2202 - protein, 15 aa, hepatitis C virus
RLIVFPDLGVRVCEK

SEQ ID NO: 2203 - protein, 15 aa, hepatitis C virus
RLVVLATATPPGSVT

SEQ ID NO: 2204 - protein, 15 aa, hepatitis C virus
RPEYDLELITSCSSN

SEQ ID NO: 2205 - protein, 15 aa, hepatitis C virus
RQEMGGNITRVESEN

SEQ ID NO: 2206 - protein, 15 aa, hepatitis C virus
RSELSPLLLSTTEWQ

SEQ ID NO: 2207 - protein, 15 aa, hepatitis C virus
RSPVFTDNSSPPAVP

SEQ ID NO: 2208 - protein, 15 aa, hepatitis C virus
SAMYVGDLCGSVFLV

SEQ ID NO: 2209 - protein, 15 aa, hepatitis C virus
SDLYLVTRHADVIPV

SEQ ID NO: 2210 - protein, 15 aa, hepatitis C virus
SFSIFLLALLSCLTI

SEQ ID NO: 2211 - protein, 15 aa, hepatitis C virus
SFSIFLLALLSCLTV

SEQ ID NO: 2212 - protein, 15 aa, hepatitis C virus
SIFLLALLSCLTIPA

SEQ ID NO: 2213 - protein, 15 aa, hepatitis C virus
SKGWRLLAPITAYAQ

SEQ ID NO: 2214 - protein, 15 aa, hepatitis C virus
SLRVFTEAMTRYSAP

SEQ ID NO: 2215 - protein, 15 aa, hepatitis C virus
SRCWVALTPTLAARN

SEQ ID NO: 2216 - protein, 15 aa, hepatitis C virus
STKVPAAYAAQGYKV

SEQ ID NO: 2217 - protein, 15 aa, hepatitis C virus
STTILGIGTVLDQAE

SEQ ID NO: 2218 - protein, 15 aa, hepatitis C virus
STWVLVGGVLAALAA

SEQ ID NO: 2219 - protein, 15 aa, hepatitis C virus
SYTWTGALITPCAAE

SEQ ID NO: 2220 - protein, 15 aa, hepatitis C virus
TFQVAHLHAPTGSGK

SEQ ID NO: 2221 - protein, 15 aa, hepatitis C virus
TMLVCGDDLVVICES

SEQ ID NO: 2222 - protein, 15 aa, hepatitis C virus
TPCAAEESKLPINAL

SEQ ID NO: 2223 - protein, 15 aa, hepatitis C virus
TPCAAEESKLPINPL

SEQ ID NO: 2224 - protein, 15 aa, hepatitis C virus
TPLLYRLGAVQNEVT

SEQ ID NO: 2225 - protein, 15 aa, hepatitis C virus
TRGLLGCIITSLTGR

SEQ ID NO: 2226 - protein, 15 aa, hepatitis C virus
TTIMAKNEVFCVQPE

SEQ ID NO: 2227 - protein, 15 aa, hepatitis C virus
TTTVPQDAVSRSQRR

SEQ ID NO: 2228 - protein, 15 aa, hepatitis C virus
TVDFSLDPTFTIETT

SEQ ID NO: 2229 - protein, 15 aa, hepatitis C virus
TWVLVGGVLAALAAY

SEQ ID NO: 2230 - protein, 15 aa, hepatitis C virus
VDILAGYGAGVAGAL

SEQ ID NO: 2231 - protein, 15 aa, hepatitis C virus
VESMETTMRSPVFTD

SEQ ID NO: 2232 - protein, 15 aa, hepatitis C virus
VFCVQPEKGGRKPAR

SEQ ID NO: 2233 - protein, 15 aa, hepatitis C virus
VGGVLAALAAYCLTT

SEQ ID NO: 2234 - protein, 15 aa, hepatitis C virus
VGIFRAAVCTRGVAK

SEQ ID NO: 2235 - protein, 15 aa, hepatitis C virus
VLVLNPSVAATLGFG

SEQ ID NO: 2236 - protein, 15 aa, hepatitis C virus
VNLLPAILSPGALVV

SEQ ID NO: 2237 - protein, 15 aa, hepatitis C virus
VNSWLGNIIMYAPTL

SEQ ID NO: 2238 - protein, 15 aa, hepatitis C virus
VQWMNRLIAFASRGN

SEQ ID NO: 2239 - protein, 15 aa, hepatitis C virus
VVGVVCAAILRRHVG

SEQ ID NO: 2240 - protein, 15 aa, hepatitis C virus
VVVVATDALMTGFTG

SEQ ID NO: 2241 - protein, 15 aa, hepatitis C virus
VVVVATDALMTGYTG

SEQ ID NO: 2242 - protein, 15 aa, hepatitis C virus
VWPLLLLLLALPPRA

SEQ ID NO: 2243 - protein, 15 aa, hepatitis C virus
VYYLTRDPTTPLARA

SEQ ID NO: 2244 - protein, 15 aa, hepatitis C virus
WDQMWKCLIRLKPTL

SEQ ID NO: 2245 - protein, 15 aa, hepatitis C virus
WESVFTGLTHIDAHF

SEQ ID NO: 2246 - protein, 15 aa, hepatitis C virus
WKCLIRLKPTLHGPT

SEQ ID NO: 2247 - protein, 15 aa, hepatitis C virus
WVLVGGVLAALAAYC

SEQ ID NO: 2248 - protein, 15 aa, hepatitis C virus
YDIIICDECHSTDST

SEQ ID NO: 2249 - protein, 15 aa, hepatitis C virus
YGKFLADGGCSGGAY

SEQ ID NO: 2250 - protein, 15 aa, hepatitis C virus
YGVWPLLLLLLALPP

SEQ ID NO: 2251 - protein, 15 aa, hepatitis C virus
YIPLVGAPLGGAARA

SEQ ID NO: 2252 - protein, 15 aa, Hepatitis C virus
YKVLVLNPSVAATLG

SEQ ID NO: 2253 - protein, 15 aa, Hepatitis C virus
YYSMVGNWAKVLIVM

SEQ ID NO: 2254 - protein, 26 aa, hepatitis C virus
QIVGGVYLLPRRGPRLGVRATRKSER

SEQ ID NO: 2255 - protein, 20 aa, hepatitis C virus
KTSERSQPRGRRQPIPKARR

SEQ ID NO: 2256 - protein, 14 aa, hepatitis C virus
LYGNEGLGWAGWLL

SEQ ID NO: 2257 - protein, 30 aa, hepatitis C virus
VIDTLTCGFADLMGYIPLVGAPLGGAARAL

SEQ ID NO: 2258 - protein, 19 aa, hepatitis C virus
NLPGCSFSIFLLALLSCLT

SEQ ID NO: 2259 - protein, 33 aa, hepatitis C virus
AAYAAQGYKVLVLNPSVAATLGFGAYMSKAHGV

SEQ ID NO: 2260 - protein, 12 aa, hepatitis C virus
GEIPFYGKAIPI

SEQ ID NO: 2261 - protein, 14 aa, hepatitis C virus
HLIFCHSKKKCDEL

SEQ ID NO: 2262 - protein, 16 aa, hepatitis C virus
GLNAVAYYRGLDVSVI

SEQ ID NO: 2263 - protein, 19 aa, hepatitis C virus
TPGERPSGMFDSSVLCECY

SEQ ID NO: 2264 - protein, 19 aa, hepatitis C virus
LRAYLNTPGLPVCQDHLEF

SEQ ID NO: 2265 - protein, 18 aa, hepatitis C virus
EFWESVFTGLTHIDAHFL

SEQ ID NO: 2266 - protein, 18 aa, hepatitis C virus
EFWESVFTGLTHIDAHFL

SEQ ID NO: 2267 - protein, 33 aa, hepatitis C virus
APPPSWDQMWKCLIRLKPTLHGPTPLLYRLGAV

SEQ ID NO: 2268 - protein, 13 aa, hepatitis C virus
VTLTHPITKYIMA

SEQ ID NO: 2269 - protein, 34 aa, hepatitis C virus
FWAKHMWNFISGIQYLAGLSTLPGNPAIASLMAF

SEQ ID NO: 2270 - protein, 22 aa, hepatitis C virus
KVLVDILAGYGAGVAGALVAFK

SEQ ID NO: 2271 - protein, 17 aa, hepatitis C virus
VNLLPAILSPGALVVGV

SEQ ID NO: 2272 - protein, 30 aa, hepatitis C virus
GRKPARLIVFPDLGVRVCEKMALYDVVSTL

SEQ ID NO: 2273 - protein, 34 aa, hepatitis C virus
VMGSSYGFQYSPGQRVEFLVNAWKSKKCPMGFSY

SEQ ID NO: 2274 - protein, 20 aa, hepatitis C virus
EARQAIRSLTERLYIGGPLT

SEQ ID NO: 2275 - protein, 10 aa, hepatitis C virus
YRRCRASGVL

SEQ ID NO: 2276 - protein, 28 aa, hepatitis C virus
PVNSWLGNIIMYAPTLWARMILMTHFFS

SEQ ID NO: 2277 - protein, 27 aa, hepatitis C virus
GQIVGGVYLLPRRGPRLGVRATRKSER

SEQ ID NO: 2278 - protein, 27 aa, hepatitis C virus
FWAKHMWNFISGIQYLAGLSTLPGNPA